(19) United States
(12) Patent Application Publication
(10) Pub. No.: US 2026/0294909 A1
(43) Pub. Date: Oct. 1, 2026

(54) SALTS AND POLYMORPHIC FORMS OF MENIN INHIBITORS AND PHARMACEUTICAL COMPOSITIONS THEREOF

(71) Applicant: Syndax Pharmaceuticals, Inc., New York, NY (US)

(72) Inventors: Ariane MAROLEWSKI, Collegeville, PA (US); Venkata A. KATTUBOINA, Westford, MA (US); Suniket FULZELE, Plymouth, MN (US)

(21) Appl. No.: 19/692,563

(22) Filed: May 29, 2026

Related U.S. Application Data
(63) Continuation of application No. 18/935,892, filed on Nov. 4, 2024.
(60) Provisional application No. 63/547,350, filed on Nov. 4, 2023.
(60) Provisional application No. 63/674,598, filed on Jul. 23, 2024.

(51) Int. Cl. A61K 31/506; A61K 9/00; A61K 9/20; C07D 471/10

(52) U.S. Cl. CPC A61K 31/506; A61K 9/0053; A61K 9/2009; A61K 9/2013; A61K 9/2027; A61K 9/2054; A61K 9/2095; C07D 471/10

(57) ABSTRACT

The present disclosure relates to a novel salt and salt forms of Compound A.

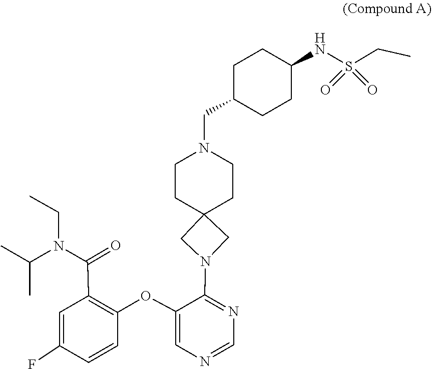 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

The disclosure is also directed to pharmaceutical compositions containing at Compound A citrate salt or salt forms and to the therapeutic use of such salts, salt forms, and compositions thereof.

### CROSS-REFERENCE TO RELATED APPLICATIONS

This application is a continuation of U.S. application Ser. No. 18/935,892, filed on Nov. 4, 2024, which claims priority to, and the benefit of, U.S. Provisional Application No. 63/547,350, filed on Nov. 4, 2023 and U.S. Provisional Application No. 63/674,598, filed on Jul. 23, 2024, the entire contents of each of which are incorporated herein by reference.

### BACKGROUND

Inhibitors of the menin-MLL interaction have shown to be active in the treatment of various cancers, specifically various leukemias, among others. Additionally, formulations effective in administering such inhibitors are needed. One compound, trans N-ethyl-2-((4-(7-((4-(ethylsulfonamido) cyclohexyl)methyl)-2,7-diazaspiro[3.5]nonan-2-yl)pyrimidin-5-yl)oxy)-5-fluoro-N-isopropylbenzamide (“Compound A”) has been shown to be an effective in the treatment in diseases and disorders where the menin-MLL interaction plays a role. Compound A is described in PCT Patent Appl. Publication No. WO 2017/214367 which is incorporated by reference in its entirety.

Currently known salts and polymorphs of Compound A suffer from (i) a lack of stability both in solution and solid form, and (ii) a propensity to form undesirable solvates with organic solvents. Compound A salts, Compound A polymorphs, and formulations comprising improved stability without forming organic solvates remains a significant unmet need which is addressed by the present application.

Additionally, pharmaceutical compositions comprising Compound A have suffered from low solubility in tablet form or very bad taste in liquid formulation. These challenges remain an unmet clinical need, each addressed by the formulations described herein.

### SUMMARY

In some aspects, the present disclosure is directed to a Compound A monocitrate monohydrate Form A salt, wherein Compound A is of the following formula:

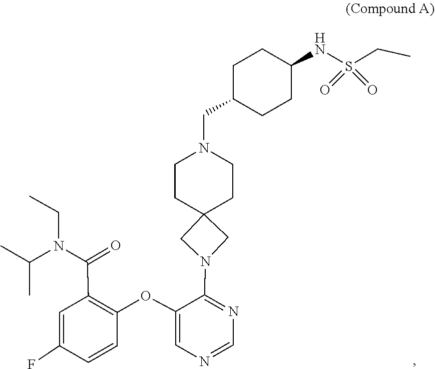 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

characterized by PXRD peaks at 11.8°2θ, and 16.9°2θ (±0.2°2θ; ±0.1°2θ; or ±0.0°2θ; Cu Kα1 radiation).

In some aspects, the present disclosure is directed to a pharmaceutical composition including: from about 15% to about 30% wt/wt of Compound A as the Compound A monocitrate monohydrate Form A salt (calculated as free base equivalents)

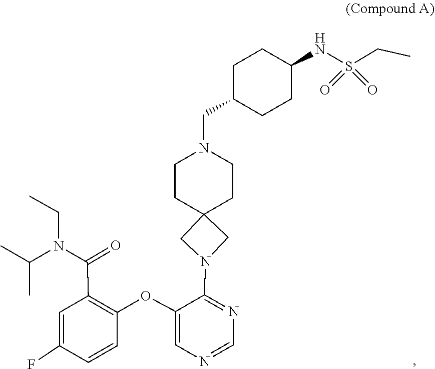 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

from about 30% to about 60% wt/wt of microcrystalline cellulose, from about 5% to about 15% wt/wt crospovidone, from about 1% to about 10% wt/wt sodium bicarbonate, from about 0.1% to about 2% wt/wt hydrophobic colloidal silica, and from about 0.1% to about 7% wt/wt magnesium stearate.

In some aspects, the present disclosure is directed to a pharmaceutical composition including an intragranular phase and an extragranular phase, wherein: (a) the intragranular phase includes: from about 15% to about 30% wt/wt of Compound A as the Compound A monocitrate monohydrate Form A salt;

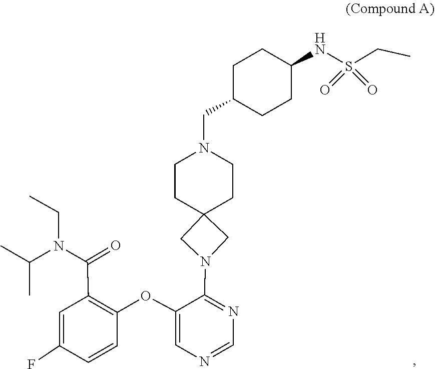 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

and
(b) the extragranular phase includes: from about 0.5% to about 5% wt/wt magnesium stearate.

In some aspects, the present disclosure is directed to a tablet comprising Compound A monocitrate monohydrate Form A.

In some aspects, the present disclosure is directed to a liquid formulation including Compound A, and a pharmaceutically acceptable carrier.

In some aspects, the present disclosure is directed to a method of treating a disease where menin-MLL interaction plays a role including administering the Compound A monocitrate monohydrate Form A salt, tablet, or liquid formulation to a subject in need thereof.

In some aspects, the present disclosure is directed to a method for preparing tablets including Compound A monocitrate monohydrate Form A salt, the method including: (a) blending Compound A monocitrate monohydrate Form A salt and a pharmaceutically acceptable excipient; (b) adding a carbonate; (c) blending; and (d) compressing the blend into tablets.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A and FIG. 1B each depicts an XRPD of Compound A monocitrate monohydrate Form A salt. FIG. 1C depicts a simulated XRPD of Compound A monocitrate monohydrate Form A salt.

FIG. 2 depicts an FT-IR (Fourier transform infrared) of Compound A monocitrate monohydrate Form A salt.

FIG. 3 depicts a DSC of Compound A monocitrate monohydrate Form A salt.

FIG. 4 depicts a DVS of Compound A monocitrate monohydrate Form A salt at 25° C. and RH up to 90%. The figure shows Compound A monocitrate monohydrate Form A salt absorbed about 1.9% of water from 0% to 90% relative humidity, which is consistent with the approximate water content of the monohydrate. Compound A monocitrate monohydrate Form A salt is not hygroscopic.

FIG. 5 depicts a PSD of Compound A monocitrate monohydrate Form A salt measured by laser diffraction.

FIG. 6A depicts an XRPD of Compound A monocitrate monohydrate Form B salt. FIG. 6B depicts a simulated XRPD of Compound A monocitrate monohydrate Form B salt.

FIG. 7 depicts an XRPD of Compound A monocitrate monohydrate Form C salt.

FIG. 8 depicts a Polarized Light Microscopy (PLM) image of Compound A monocitrate monohydrate Form A salt.

FIG. 9 depicts a PLM of plate-like crystals of Compound A monocitrate monomethanolate.

FIG. 10 depicts an Oak Ridge Thermal Ellipsoid Plot (ORTEP) drawing of molecular structure of Compound A monocitrate monomethanolate, with non-hydrogen atoms in ellipsoids of 35% probability and hydrogen atoms in small circles. Hydrogen bonds are present between Compound A and citrate (N—H . . . O) and between Compound A and methanol.

FIG. 11 depicts an ORTEP drawing of molecular structure of Compound A monocitrate monohydrate Form A salt, with non-hydrogen atoms in ellipsoids of 35% probability and hydrogen atoms in small circles. Hydrogen bonds between Compound A and citrate (N—H . . . O) and between Compound A and water are shown.

FIG. 12 depicts crystal packing of Compound A monocitrate monomethanolate (upper, lower left) and Compound A monocitrate monohydrate Form A salt (upper, lower right), viewed along the crystallographic b-axis (top) and c-axis (bottom). Methanol (pink) and water (red) shown in ball-stick presentations.

FIG. 13 depicts simulated XRPD patterns of Compound A salts: a) monocitrate monomethanolate (223 K), b) monocitrate monohydrate (273 K), c) air-dried bulk sample from which single crystal was isolated, and d) oven-dried bulk sample prepared from acetone-water (90:10 v/v).

FIG. 14 depicts XRPD patterns of materials isolated from polymorph screening of Compound A citrate monohydrate salt: a) free base reference, b) Form A reference, and c) Form C obtained from MeCN.

FIG. 15 depicts an overlay of DSC/TGA thermograms of Compound A citrate monohydrate salt (Form C) from MeCN: a) Form C reference; b) Form C dry solid.

FIG. 16 depicts XRPD patterns of materials isolated from polymorph screening of Compound A citrate monohydrate salt in: a) Form B reference, b) MeOH at 50° C., c) Form A reference, and d) MeOH.

FIG. 17 depicts XRPD patterns of materials isolated from polymorph screening of Compound A citrate monohydrate salt in: a) Form A reference, b) EtOAc, c) MEK, d) THF, e) DCM, and f) toluene.

FIG. 18 depicts XRPD patterns of materials isolated from polymorph screening of Compound A citrate monohydrate salt in: a) Form A ref, b) IPAc, c) MTBE, d) H₂O, e) 2-Me-THF, f) Acetone/H₂O (7:3), g) Propanol/H₂O (7:3), and h) 1,4-dioxane/H₂O (7:3).

FIG. 19 depicts XRPD patterns of materials isolated from polymorph screening of Compound A monocitrate monohydrate salt by slow evaporation in: a) Form A reference, b) Propanol/H₂O (7:3), c) 1,4-dioxane/H₂O (7:3), and d) THF/H₂O (7:3).

FIG. 20 depicts scale-up preparation of Compound A monocitrate Form C XRPD overlay: a) Form C reference; b) Wet cake isolated from acetonitrile; c) Dry solid isolated from acetonitrile.

FIG. 21 depicts sorption isotherm of Compound A monocitrate Form C reference.

FIG. 22 depicts XRPD overlay of Compound A monocitrate Form C: a) Form C solid before DVS; b) Form C solid after DVS; c) Form A reference.

FIG. 23 depicts DSC/TGA overlay of Compound A monocitrate Form C: a) Form C solid before DVS; b) Form C solid after DVS; c) Form-A reference.

FIG. 24 depicts PLM images of scale-up preparation of Compound A monocitrate salt Form C: a) & b) scale-up product of Form C; c) Form C solid before DVS; d) Form C solid after Dynamic Vapor Sorption (DVS). It should be noted that the samples in c) and d) are reference Form C and they are not scale-up samples.

FIG. 25 depicts the dissolution profiles for Compound A AIC and BIC Drug Product in 0.1N HCl Media (n=6 capsules each, standard deviation error bars).

FIG. 26 depicts the dissolution profiles for Compound A sesquifumarate tablets with varying levels of sodium bicarbonate (1.0%, 2.5%, 5.0%, and 7.5%) in 0.1 N HCl media (n=3 tablets each).

FIG. 27 depicts the dissolution profiles for Compound A citrate tablets manufactured with and without 5.0% sodium bicarbonate in 0.1 N HCl Media (n=6 tablets each). The figure also shows the dissolution profile of Compound A citrate tablets manufactured with 5.0% sodium bicarbonate at pH 6.8 phosphate media. The tablets without sodium bicarbonate dissolve significantly more slowly than ones with bicarbonate.

FIG. 28 depicts the dissolution profiles of 160 mg Compound A citrate tablets uncoated and coated at 3%, 5%, and 7% coat level in 0.1N HCl (n=6 tablets each).

FIG. 29 depicts the dissolution profiles of Compound A tablets and capsules. Standard deviation error bars shown for each pull point, Dissolution was performed according to commercial method conditions except 113 mg capsule bath rotation speed 75 rpm. n=12 tablets and n=6 capsules of each lot tested.

FIG. 30 depicts Compound A tablet manufacturing flow diagram.

FIG. 31 depicts dissolution profiles of Compound A monocitrate monohydrate engineering batch tablets and GMP capsules.

FIG. 32 depicts Impurity A formation as a function of temperature, pH, and time in solution.

FIG. 33 depicts the comparison of dissolution profiles for tablet formulations with and without bicarbonate.

FIG. 34 depicts a phase map of Compound A monocitrate monohydrate.

### DETAILED DESCRIPTION

The present disclosure provides salts and polymorphic salt forms of Compound A that are useful in the preparation of a medicament and/or as pharmaceutical agents. In some embodiments, one or more of the salts and/or salt forms described herein can be formulated into a pharmaceutical composition. In some embodiments, the pharmaceutical composition further comprises an alkali bicarbonate. In some embodiments, the pharmaceutical composition further comprises sodium or potassium bicarbonate. In some embodiments, the pharmaceutical composition further comprises sodium bicarbonate. In some embodiments, the pharmaceutical composition further comprises potassium bicarbonate. In some embodiments, the present disclosure is directed to a tablet comprising Compound A citrate salt and/or Compound A citrate salt polymorphic form(s). In some embodiments, the present disclosure is directed to a liquid formulation comprising Compound A.

### Definitions

The terms “powder X-ray diffraction pattern”, “PXRD pattern”, “PXRD signal” “X-ray powder diffraction pattern”, “XRPD pattern” and “XRPD signal” are used interchangeably and refer to the experimentally observed diffractogram or parameters derived therefrom. Powder X-ray diffraction patterns are typically characterized by signal/peak position (abscissa) and signal/peak intensities (ordinate). The term “peak intensities” or “signal intensities” refers to relative signal intensities within a given X-ray diffraction pattern. Factors which can affect the relative peak/signal intensities are sample thickness and preferred orientation (i.e., the crystalline particles are not distributed randomly). The term “peak positions” or “signal positions” as used herein refers to X-ray reflection positions as measured and observed in powder X-ray diffraction experiments. Peak/signal positions are directly related to the dimensions of the unit cell. The signals, identified by their respective positions, are extracted from the diffraction patterns for the various polymorphic forms of salts of Compound A.

The term “2 theta value”, “2θ” or “2θ” refers to the signal position in degrees based on the experimental setup of the X-ray diffraction experiment and is a common abscissa unit in diffraction patterns. In general, the experimental setup requires that if a reflection is diffracted when the incoming beam forms an angle theta (θ) with a certain lattice plane, the reflected beam is recorded at an angle 2 theta (2θ). It should be understood that reference herein to specific 2θ values for a specific polymorphic form is intended to mean the 2θ values (in degrees) as measured using the X-ray diffraction experimental conditions as described herein.

“Preferred orientation effects” refer to variable signal intensities or relative intensity differences between different PXRD measurements of the same samples that can be due to the orientation of the particles. Without wishing to be bound by theory, in PXRD it can be desirable to have a sample in which particles are oriented randomly (e.g., a powder). However, it can be difficult or in some cases impossible to achieve truly random particle orientations in practice. As particle size increases, the randomness of particle orientation can decrease, leading to increased challenges with achieving a preferred orientation. Without wishing to be bound by theory, a smaller particle size can reduce technical challenges associated with preferred orientation and allow for more accurate representation of peaks/signals. However, one of skill in the art will understand how to reduce or mitigate preferred orientation effects and will recognize preferred orientation effects that can exist even between two different measurements of the same sample. For instance, in some embodiments, differences in resolution or relative signal intensities can be attributed to preferred orientation effects.

As used herein, the term “substantially pure” with reference to a particular salt (or to a mixture of two or more salts) of a compound indicates the salt (or a mixture) includes less than 10%, less than 5%, less than 3%, less than 1%, less than 0.5%, less than 0.2%, or less than 0.1% by weight of impurities, including other salt forms of the compound. Such purity may be determined, for example, by powder X-ray diffraction.

As used herein, “substantially pure,” when used in reference to a polymorph form of a compound A salt, means a sample of a polymorph form of the compound having a purity greater than about 90%, including greater than about 90%, about 91%, about 92%, about 93%, about 94%, about 95%, about 96%, about 97%, about 98%, and about 99%, and also including equal to about 100% of the compound, based on the weight of the compound. The remaining material comprises other form(s) of the compound, and/or reaction impurities and/or processing impurities arising from its preparation. For example, a polymorph form of the Compound A salt may be deemed substantially pure in that it has a purity greater than about 90% of a polymorph form of the Compound A salt, as measured by means that are at this time known and generally accepted in the art, where the remaining less than about 10% of material comprises other form(s) of the compound of the Compound A salt and/or reaction impurities and/or processing impurities. The presence of reaction impurities and/or processing impurities may be determined by analytical techniques known in the art, such as, for example, chromatography, nuclear magnetic resonance spectroscopy, mass spectrometry, powder X-ray diffraction or spectroscopy.

As used herein, the term “polymorph” or “salt form” refers to different crystalline forms of the same compound and other solid state molecular forms including pseudo-polymorphs, such as hydrates (e.g., bound water present in the crystalline structure) and solvates (e.g., bound solvents other than water) of the same compound. Different crystalline polymorphs have different crystal structures due to a different packing of the molecules in the lattice. This results in a different crystal symmetry and/or unit cell parameters which directly influences its physical properties such as the X-ray diffraction characteristics of crystals or powders. A different polymorph, for example, will in general diffract at a different set of angles and will give different values for the intensities. Therefore, X-ray powder diffraction can be used to identify different polymorphs, or a solid form that comprises more than one polymorph, in a reproducible and reliable way (S. Byrn et al, Pharmaceutical Solids: A Strategic Approach to Regulatory Considerations, Pharmaceutical research, Vol. 12, No. 7, p. 945-954, 1995; J. K. Haleblian and W. McCrone, Pharmaceutical Applications of Polymorphism, Journal of Pharmaceutical Sciences, Vol. 58, No. 8, p. 911-929, 1969).

Crystalline polymorphic forms are of interest to the pharmaceutical industry and especially to those involved in the development of suitable dosage forms. If the polymorphic form is not held constant during clinical or stability studies, the exact dosage form used or studied may not be comparable from one lot to another. It is also desirable to have processes for producing a compound with the selected polymorphic form in high purity when the compound is used in clinical studies or commercial products since impurities present may produce undesired toxicological effects. Certain polymorphic forms may exhibit enhanced thermodynamic stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies.

The term “amorphous” refers to any solid substance which (i) lacks order in three dimensions, or (ii) exhibits order in less than three dimensions, order only over short distances (e.g., less than 10 A), or both. Thus, amorphous substances include partially crystalline materials and crystalline mesophases with, e.g., one- or two-dimensional translational order (liquid crystals), orientational disorder (orientationally disordered crystals), or conformational disorder (conformationally disordered crystals). Amorphous solids may be characterized by known techniques, including powder X-ray diffraction (PXRD) crystallography, solid state nuclear magnet resonance (ssNMR) spectroscopy, differential scanning calorimetry (DSC), or some combination of these techniques. Amorphous solids give diffuse PXRD patterns, typically comprised of one or two broad signals (i.e., peaks/signals having base widths of about 5°2Θ or greater).

The term “crystalline” refers to any solid substance exhibiting three-dimensional order, which in contrast to an amorphous solid substance, gives a distinctive PXRD pattern with sharply defined signals.

In some embodiments, the term “Compound A or a pharmaceutically acceptable salt thereof” is a crystalline, an amorphous, or a polymorphic form.

The term “ambient temperature” refers to a temperature condition typically encountered in a laboratory setting. This includes the approximate temperature range of about 20° C. to about 30° C.

The term “detectable amount” refers to an amount or amount per unit volume that can be detected using conventional techniques, such as X-ray powder diffraction, differential scanning calorimetry, HPLC, Fourier Transform Infrared Spectroscopy (FT-IR), Raman spectroscopy, and the like.

The term “solvate” describes a molecular complex comprising the drug substance and a stoichiometric or non-stoichiometric amount of one or more solvent molecules (e.g., ethanol). When the solvent is tightly bound to the drug the resulting complex will have a well-defined stoichiometry that is independent of humidity. When, however, the solvent is weakly bound, as in channel solvates and hygroscopic compounds, the solvent content will be dependent on humidity and drying conditions. In such cases, the complex may be non-stoichiometric.

The term “hydrate” describes a solvate comprising the drug substance and a stoichiometric or non-stoichiometric amount of water.

The term “relative humidity” refers to the ratio of the amount of water vapor in air at a given temperature to the maximum amount of water vapor that can be held at that temperature and pressure, expressed as a percentage.

The term “relative intensity” refers to an intensity value derived from a sample X-ray diffraction pattern. The complete ordinate range scale for a diffraction pattern is assigned a value of 100. A peak/signal having intensity falling between about 50% to about 100% on this scale intensity is termed very strong (vs); a peak having intensity falling between about 50% to about 25% is termed strong (s). Additional weaker peaks/signals are present in typical diffraction patterns and are also characteristic of a given polymorph, wherein the additional peaks/signals are termed medium (m), weak (w) and very weak (vw).

The term “slurry” refers to a solid substance suspended in a liquid medium, typically water or an organic solvent.

The term “under vacuum” refers to typical pressures obtainable by a laboratory oil or oil-free diaphragm vacuum pump.

The term “pharmaceutical composition” refers to a composition comprising Compound A, a salt thereof, or a polymorphic form thereof described herein, and other chemical components, such as physiologically/pharmaceutically acceptable carriers, diluents, vehicles and/or excipients. The purpose of a pharmaceutical composition is to facilitate administration of a compound to an organism, such as a human or other mammals. For avoidance of doubt, as used herein, the term “pharmaceutical composition” (e.g., liquid pharmaceutical composition) is equivalent to and substitutable with the term “formulation” (e.g., liquid formulation).

The term “pharmaceutically acceptable” “carrier”, “diluent”, “vehicle”, or “excipient” refers to a material (or materials) that may be included with a particular pharmaceutical agent to form a pharmaceutical composition, and may be solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropyl methylcellulose, methylmethacrylate and the like.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

The term “treating”, as used herein, unless otherwise indicated, means reversing, alleviating, or inhibiting the progress of the disorder or condition to which such term applies, or one or more symptoms of such disorder or condition. The term “treatment”, as used herein, unless otherwise indicated, refers to the act of “treating” as defined immediately above. For example, the terms “treat”, “treating” and “treatment” can refer to a method of alleviating or abrogating a particular disorder and/or one or more of its attendant symptoms.

As used herein, “subject” means a human or animal (in the case of an animal, the subject can be a mammal). In some embodiments, the subject is a human. In some embodiments, the subject is a male. In some embodiments, the subject is a female. In some embodiments, the subject is an adult or a pediatric patient.

As used herein, the term “wt/wt” or “weight by weight” refers to a ratio between the weight of the designated component versus the weight of the entire composition.

As used herein, the term “calculated as free base equivalents” would be appreciated by a person of ordinary skill in the art to reference an amount of a salt (e.g., a monocitrate monohydrate salt) that would need to be present in a dosage form when provided with a specific amount of the free base.

Unless explicitly indicated otherwise, the terms “approximately” and “about” are synonymous. In some embodiments, “approximately” and “about” refer to recited amount, value, or duration ±10%, ±8%, ±6%, ±5%, ±4%, ±2%, ±1%, or ±0.5%. In another embodiment, “approximately” and “about” refer to listed amount, value, or duration ±10%, ±8%, ±6%, ±5%, ±4%, or ±2%. In yet another embodiment, “approximately” and “about” refer to listed amount, value, or duration ±5%. In yet another embodiment, “approximately” and “about” refer to listed amount, value, or duration ±2% or ±1%.

When the terms “approximately” and “about” are used when reciting XRPD peaks/signals, these terms refer to the recited X-ray powder diffraction peak ±0.3°2θ, ±0.2°2θ, or ±0.1°2θ. In some embodiments, the terms “approximately” and “about” refer to the listed X-ray powder diffraction peak/signal ±0.2°2θ. In another embodiment, the terms “approximately” and “about” refer to the listed X-ray powder diffraction peak/signal ±0.1°2θ.

When the terms “approximately” and “about” are used when reciting temperature or temperature range, these terms refer to the recited temperature or temperature range ±5° C., ±2° C., or ±1° C. In another embodiment, the terms “approximately” and “about” refer to the recited temperature or temperature range ±2° C.

Where a range of values is provided, it is understood that each intervening value, to the tenth of the unit of the lower limit unless the context clearly dictates otherwise, between the upper and lower limit of that range and any other stated or intervening value in that stated range is encompassed within the present disclosure. The upper and lower limits of these smaller ranges may independently be included in the smaller ranges subject to any specifically excluded limit in the stated range. Where the stated range includes one or both of the limits, ranges excluding either or both of those included limits are also included in the disclosure. When a range is expressed as being between two values, both endpoints are understood to be included in the range unless context dictates otherwise.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent compound formed, for example, from non-toxic inorganic organic acids.

It is to be understood that all references to pharmaceutically acceptable salts include solvent addition forms (solvates) or crystal forms (polymorphs) as defined herein, of the same salt.

A wide variety of pharmaceutically acceptable salts is formed from the menin inhibitor and includes: acid addition salts formed by reacting the menin inhibitor with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like; acid addition salts formed by reacting the menin inhibitor with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to the menin inhibitor refers to a salt of the menin inhibitor, which does not cause significant irritation to a mammal to which it is administered and does not substantially abrogate the biological activity and properties of the compound.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In some embodiments, solvates are formed using, but limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol.

In some embodiments, solvates of Compound A, or pharmaceutically acceptable salts thereof, are conveniently prepared or formed during the processes described herein. In some embodiments, solvates of Compound A are anhydrous. In some embodiments, Compound A, or pharmaceutically acceptable salts thereof, exist in unsolvated form. In some embodiments, Compound A, or pharmaceutically acceptable salts thereof, exist in unsolvated form and are anhydrous.

In some embodiments, Compound A, or a pharmaceutically acceptable salt thereof, is prepared in various forms, including but not limited to, amorphous phase, crystalline forms, milled forms and nano-particulate forms. In some embodiments, Compound A, or a pharmaceutically acceptable salt thereof, is amorphous. In some embodiments, Compound A, or a pharmaceutically acceptable salt thereof, is amorphous and anhydrous. In some embodiments, Compound A, or a pharmaceutically acceptable salt thereof, is crystalline. In some embodiments, Compound A, or a pharmaceutically acceptable salt thereof, is crystalline and anhydrous.

In one aspect, the present disclosure is directed to a Compound A monocitrate monohydrate Form A salt, wherein Compound A is of the following formula:

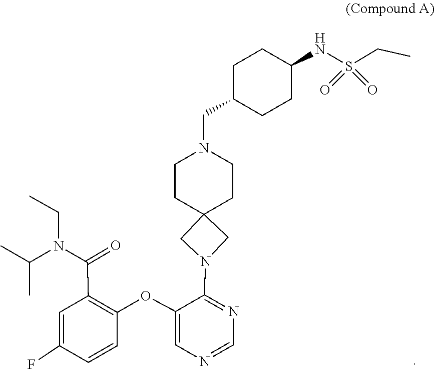 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

In some embodiments, the present disclosure is directed to a pharmaceutical composition comprising:
from about 10% to about 30% wt/wt of a Compound A

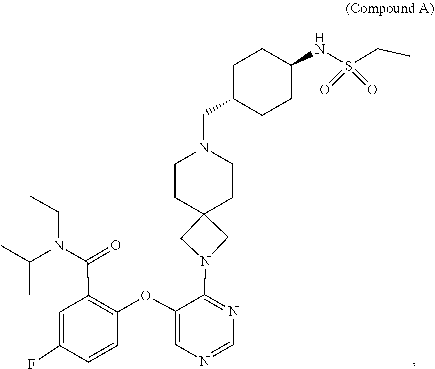 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

and
from about 0.1% to about 10% wt/wt sodium bicarbonate.

In some embodiments, the present disclosure is directed to a pharmaceutical composition comprising:
from about 15% to about 30% wt/wt of a Compound A monocitrate monohydrate Form A salt

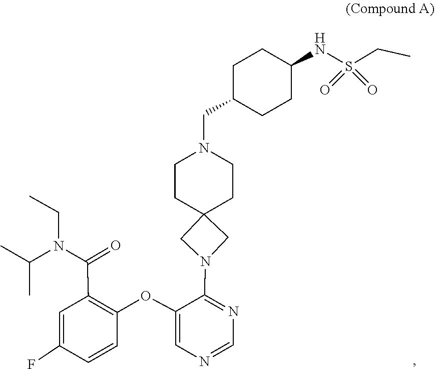 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

and
from about 0.1% to about 10% wt/wt sodium bicarbonate.

In some embodiments, the present disclosure is directed to a tablet comprising:
from about 15% to about 30% wt/wt of a Compound A monocitrate monohydrate Form A salt

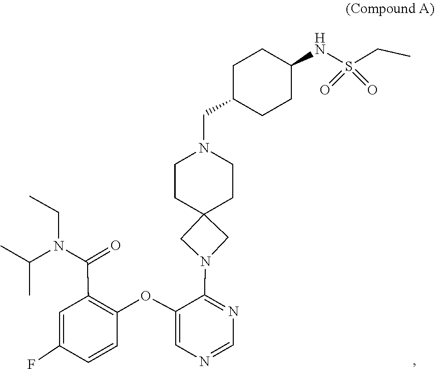 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

and
from about 0.1% to about 10% wt/wt sodium bicarbonate.

In some embodiments, the pharmaceutical composition further comprises from about 30% to about 60% wt/wt of a binder.

In some embodiments, the pharmaceutical composition further comprises from about 30% to about 60% wt/wt of microcrystalline cellulose.

In some embodiments, the pharmaceutical composition further comprises from about 45% to about 60% wt/wt of microcrystalline cellulose.

In some embodiments, the pharmaceutical composition further comprises from about 1% to about 10% wt/wt of a bicarbonate.

In some embodiments, the pharmaceutical composition further comprises from about 1% to about 10% wt/wt sodium bicarbonate.

In some embodiments, the pharmaceutical composition further comprises from about 0.1% to about 2% wt/wt hydrophobic colloidal silica.

In some embodiments, the pharmaceutical composition further comprises from about 5% to about 15% wt/wt crospovidone.

In some embodiments, the pharmaceutical composition further comprises from about 5% to about 20% wt/wt hypromellose.

In some embodiments, the pharmaceutical composition further comprises from about 0.1% to about 7% wt/wt magnesium stearate.

In some embodiments, the present disclosure is directed to a pharmaceutical composition comprising:
from about 15% to about 30% wt/wt of a Compound A monocitrate monohydrate Form A salt

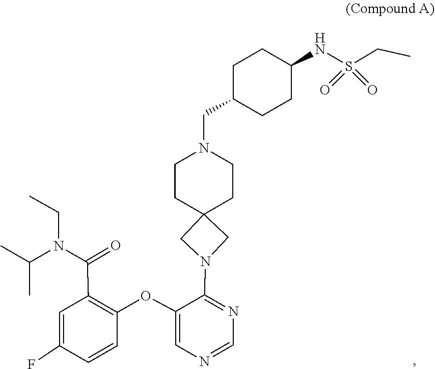 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

from about 30% to about 60% wt/wt of microcrystalline cellulose,
from about 5% to about 15% wt/wt crospovidone,
from about 1% to about 10% wt/wt sodium bicarbonate,
from about 0.1% to about 10% wt/wt hydrophobic colloidal silica, and
from about 0.1% to about 15% wt/wt magnesium stearate.

In some embodiments, the pharmaceutical composition further comprises from about 5% to about 20% wt/wt hypromellose.

In some embodiments, the present disclosure is directed to a pharmaceutical composition comprising an intragranular phase and an extragranular phase, wherein:
(a) the intragranular phase comprises:
   from about 15% to about 30% wt/wt of a Compound A monocitrate monohydrate Form A salt

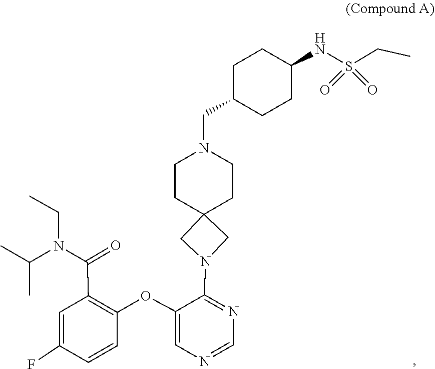 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

and
(b) the extragranular phase comprises:
   from about 0.5% to about 5% wt/wt of a glidant.

In some embodiments, the intragranular phase of the pharmaceutical composition further comprises about 1% to about 10% wt/wt of sodium bicarbonate.

In some embodiments, the intragranular phase of the pharmaceutical composition further comprises:
from about 30% to about 60% wt/wt of microcrystalline cellulose;
from about 5% to about 15% wt/wt crospovidone;
from about 5% to about 20% wt/wt hypromellose;
from about 0.1% to about 2% wt/wt hydrophobic colloidal silica;
from about 0.1% to about 2% wt/wt magnesium stearate.

In some embodiments, the intragranular phase of the pharmaceutical composition further comprises:
from about 45% to about 60% wt/wt of microcrystalline cellulose;
from about 5% to about 15% wt/wt crospovidone;
from about 5% to about 20% wt/wt hypromellose;
from about 0.1% to about 2% wt/wt hydrophobic colloidal silica;
from about 0.1% to about 2% wt/wt magnesium stearate.

In some embodiments, the present disclosure is directed to a pharmaceutical composition comprising:
from about 15% to about 30% wt/wt of a Compound A monocitrate monohydrate Form A salt calculated as free base equivalents

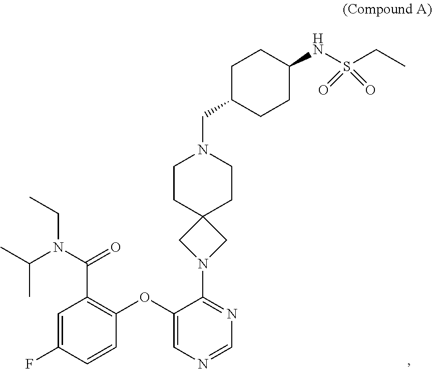 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

and
from about 1% to about 10% wt/wt sodium bicarbonate.

In some embodiments, the pharmaceutical composition comprises from about 30% to about 60% wt/wt of microcrystalline cellulose.

In some embodiments, the pharmaceutical composition comprises from about 45% to about 60% wt/wt of microcrystalline cellulose.

In some embodiments, the pharmaceutical composition comprises from about 1% to about 10% wt/wt sodium bicarbonate.

In some embodiments, the pharmaceutical composition comprises from about 0.1% to about 2% wt/wt hydrophobic colloidal silica.

In some embodiments, the pharmaceutical composition comprises from about 5% to about 15% wt/wt crospovidone.

In some embodiments, the pharmaceutical composition comprises from about 5% to about 20% wt/wt hypromellose.

In some embodiments, the pharmaceutical composition comprises from about 0.1% to about 7% wt/wt magnesium stearate.

In some embodiments, the present disclosure is directed to a pharmaceutical composition comprising:
from about 15% to about 30% wt/wt of a Compound A monocitrate monohydrate Form A salt calculated as free base equivalents

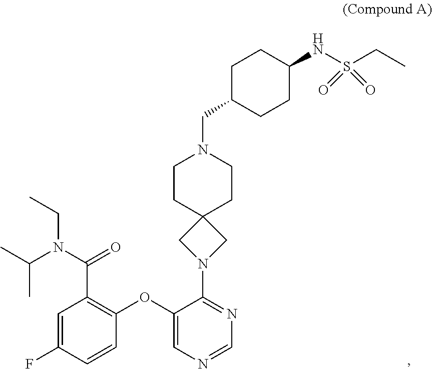 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

from about 30% to about 60% wt/wt of microcrystalline cellulose,
from about 5% to about 15% wt/wt crospovidone,
from about 1% to about 10% wt/wt sodium bicarbonate,
from about 0.1% to about 2% wt/wt hydrophobic colloidal silica, and
from about 0.1% to about 7% wt/wt magnesium stearate.

In some embodiments, the present disclosure is directed to a pharmaceutical composition comprising:
from about 15% to about 30% wt/wt of a Compound A monocitrate monohydrate Form A salt calculated as free base equivalents

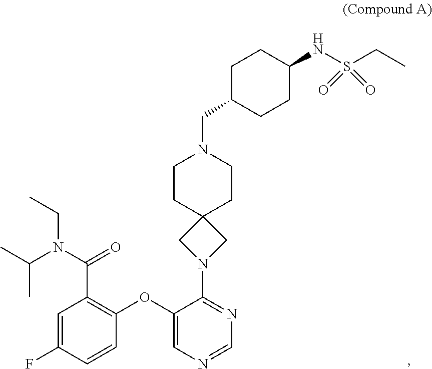 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

from about 45% to about 60% wt/wt of microcrystalline cellulose,
from about 5% to about 15% wt/wt crospovidone,
from about 1% to about 10% wt/wt sodium bicarbonate,
from about 0.1% to about 2% wt/wt hydrophobic colloidal silica, and
from about 0.1% to about 7% wt/wt magnesium stearate.

In some embodiments, the pharmaceutical composition further comprises from about 5% to about 20% wt/wt hypromellose.

In some embodiments, the pharmaceutical composition is a tablet.

In some embodiments, the pharmaceutical composition is a film-coated tablet.

In some embodiments, the present disclosure is directed to a pharmaceutical composition comprising an intragranular phase and an extragranular phase, wherein:
(a) the intragranular phase comprises:
   from about 15% to about 30% wt/wt of a Compound A monocitrate monohydrate Form A salt calculated as free base equivalents

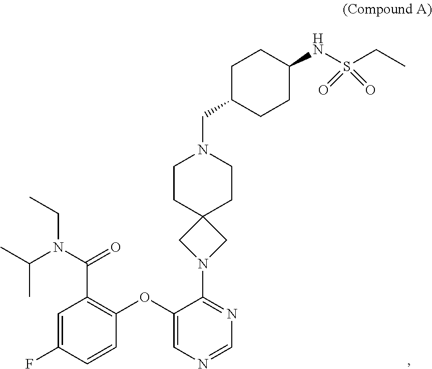 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

and
(b) the extragranular phase comprises:
   from about 0.5% to about 5% wt/wt magnesium stearate.

In some embodiments, the intragranular phase of the pharmaceutical composition further comprises about 1% to about 10% wt/wt of sodium bicarbonate.

In some embodiments, the present disclosure is directed to a method for preparing tablets comprising Compound A monocitrate monohydrate Form A salt using steps comprising:
(a) blending Compound A monocitrate monohydrate Form A salt with a glidant (e.g., hydrophobic colloidal silica) to form a pre-blend,
(b) blending the pre-blend with remaining excipients to form an intragranular blend.
(c) roller compacting and milling the intragranular blend to generate milled granules,
(d) blending the milled granules with a lubricant (e.g., magnesium stearate) to obtain a final blend,
(e) compressing the final blend into tablets,
(f) film coating the tablets to form coated tablets, and
(g) packaging the coated tablets (e.g., into bottles or blisters).

In some embodiments, the pharmaceutical composition is a liquid formulation.

In some embodiments, the Compound A is present in the liquid formulation from about 1% to about 10% wt/wt.

In some embodiments, the disclosure of the application relates to a method of treating a cancer comprising administering Compound A, a salt thereof, or a polymorphic form thereof (e.g., Compound A monocitrate monohydrate Form A salt), or a pharmaceutical composition (e.g. liquid formulation or solid formulation such as a tablet) thereof of any one of the preceding embodiments to a subject in need thereof.

### Compound A Crystalline Forms

In some embodiments, Compound A can form a salt with citric acid. In some embodiments, the Compound A citrate salt described herein exist in a crystalline form. All PXRD signals described herein are in °2θ (±0.2°2θ; ±0.1°2θ; or ±0.0°2θ; Cu Kα1 radiation). Additionally, PXRD spectra are obtained using Cu Kα1 X-rays at a wavelength of 1.5406 Å.

### Compound A Citrate Salt and Polymorphic Forms Thereof

In some embodiments, the present disclosure provides a Compound A citrate salt.

In some embodiments, the Compound A citrate salt (e.g. Compound A monocitrate monohydrate Form A salt) is amorphous.

In some embodiments, the Compound A citrate salt (e.g. Compound A monocitrate monohydrate Form A salt) is crystalline.

In some embodiments, the Compound A citrate salt is a crystalline polymorphic form.

In some embodiments, the Compound A citrate salt is a crystalline polymorphic Form A.

In some embodiments, the Compound A citrate salt is a crystalline polymorphic Form B.

In some embodiments, the Compound A citrate salt is a crystalline polymorphic Form C.

In some embodiments, the Compound A citrate salt is a monocitrate salt.

In some embodiments, the Compound A citrate salt is a monocitrate hydrate salt.

In some embodiments, the Compound A citrate salt is a monocitrate monohydrate salt (also referred to as Compound A citrate hydrate salt).

In some embodiments, the Compound A citrate salt has improved stability compared to Compound A fumarate salt.

In some embodiments, the Compound A citrate salt is a hydrate.

In some embodiments, the Compound A citrate salt is anhydrous.

In some embodiments, the Compound A citrate salt is monocitrate salt.

In some embodiments, the Compound A citrate salt is monocitrate monohydrate salt.

In some embodiments, the Compound A citrate salt is a 1:2 Compound A:citrate salt.

In some embodiments, the Compound A citrate salt is a 2:1 Compound A:citrate salt.

In some embodiments, the Compound A citrate salt is a 1:1 Compound A:citrate salt.

In some embodiments, the Compound A monocitrate monohydrate Form A salt is a 1:1 Compound A:citrate salt.

In some embodiments, the Compound A monocitrate salt is a hydrate.

In some embodiments, the Compound A monocitrate salt is a monohydrate.

In some embodiments, the Compound A citrate salt is characterized by a melting point from about 165° C. to about 175° C., or from about 177° C. to about 187° C. as measured by DSC.

In some embodiments, the Compound A citrate salt is characterized by a melting point between approximately 165° C. and approximately 175° C., and between approximately 177° C. and approximately 187° C. as measured by DSC.

In some embodiments, the Compound A citrate salt is characterized by DSC having a melting point at about 169.9° C. or about 182.6° C.

In some embodiments, the Compound A citrate salt is characterized by DSC having melting peaks at about 169.9° C. and about 182.6° C.

In some embodiments, the Compound A citrate salt is characterized by DSC having melting point at about 169.9° C.

In some embodiments, the Compound A citrate salt is characterized by DSC having melting point at about 182.6° C. In some embodiments, Compound A citrate salt is non-hygroscopic.

In some embodiments, Compound A citrate salt displays non-hygroscopicity between 0 and 90% RH at between 25° C. and 45° C. (e.g., less than 0.2% w/w water uptake).

In some embodiments, particle size Dx(10) of Compound A citrate salt ranges from about 1-100 μm, about 1-50 μm, about 1-25 μm, about 1-24 μm, about 1-23 μm, about 1-22 μm, about 1-21 μm, about 1-20 μm, about 1-19 μm, about 1-18 μm, about 1-17 μm, about 1-16 μm, about 1-15 μm, about 1-14 μm, about 1-13 μm, about 1-12 μm, about 1-11 μm, about 1-10 μm, about 1-9 μm, about 1-8 μm, about 1-7 μm, about 1-6 μm, about 1-5 μm, about 1-4 μm, about 1-2 μm, about 2-25 μm, about 2-24 μm, about 2-23 μm, about 2-22 μm, about 2-21 μm, about 2-20 μm, about 2-19 μm, about 2-18 μm, about 2-17 μm, about 2-16 μm, about 2-15 μm, about 2-14 μm, about 2-13 μm, about 2-12 μm, about 2-11 μm, about 2-10 μm, about 2-9 μm, about 2-8 μm, about 2-7 μm, about 2-6 μm, about 2-5 μm, about 2-4 μm, or about 2-3 μm.

In some embodiments, particle size Dx(10) of Compound A citrate salt is about 1 μm, about 2 μm, about 3 μm, about 4 μm, or about 5 μm.

In some embodiments, particle size Dx(50) of Compound A citrate salt ranges from about 1-100 μm, about 1-50 μm, about 1-25 μm, about 1-24 μm, about 1-23 μm, about 1-22 μm, about 1-21 μm, about 1-20 μm, about 1-19 μm, about 1-18 μm, about 1-17 μm, about 1-16 μm, about 1-15 μm, about 1-14 μm, about 1-13 μm, about 1-12 μm, about 1-11 μm, about 1-10 μm, about 1-9 μm, about 1-8 μm, about 1-7 μm, about 1-6 μm, about 1-5 μm, about 1-4 μm, about 1-2 μm, about 3-20 μm, about 3-19 μm, about 3-18 μm, about 3-17 μm, about 3-16 μm, about 3-15 μm, about 3-14 μm, about 3-13 μm, about 3-12 μm, about 3-11 μm, about 3-10 μm, about 3-9 μm, about 3-8 μm, about 3-7 μm, about 3-6 μm, about 3-5 μm, about 3-4 μm, about 5-25 μm, about 5-24 μm, about 5-23 μm, about 5-22 μm, about 5-21 μm, about 5-20 μm, about 5-19 μm, about 5-18 μm, about 5-17 μm, about 5-16 μm, about 5-15 μm, about 5-14 μm, about 5-13 μm, about 5-12 μm, about 5-11 μm, about 5-10 μm, about 5-9 μm, about 5-8 μm, about 5-7 μm, or about 5-6 μm.

In some embodiments, particle size Dx(50) of Compound A citrate salt is about 2 μm, about 3 μm, about 4 μm, about 5 μm, about 6 μm, about 7 μm, about 8 μm, about 9 μm, about 10 μm, about 11 μm, or about 12 μm.

In some embodiments, particle size Dx(90) of Compound A citrate salt ranges from about 1-100 μm, about 1-50 μm, about 1-25 μm, about 1-24 μm, about 1-23 μm, about 1-22 μm, about 1-21 μm, about 1-20 μm, about 1-19 μm, about 1-18 μm, about 1-17 μm, about 1-16 μm, about 1-15 μm, about 1-14 μm, about 1-13 μm, about 1-12 μm, about 1-11 μm, about 1-10 μm, about 1-9 μm, about 1-8 μm, about 1-7 μm, about 1-6 μm, about 1-5 μm, about 1-4 μm, about 1-2 μm, about 5-25 μm, about 5-24 μm, about 5-23 μm, about 5-22 μm, about 5-21 μm, about 5-20 μm, about 5-19 μm, about 5-18 μm, about 5-17 μm, about 5-16 μm, about 5-15 μm, about 5-14 μm, about 5-13 μm, about 5-12 μm, about 5-11 μm, about 5-10 μm, about 5-9 μm, about 5-8 μm, about 5-7 μm, about 5-6 μm, about 10-25 μm, about 10-24 μm, about 10-23 μm, about 10-22 μm, about 10-21 μm, about 10-20 μm, about 10-19 μm, about 10-18 μm, about 10-17 μm, about 10-16 μm, about 10-15 μm, about 10-14 μm, about 10-13 μm, about 10-12 μm, about 10-11 μm, about 15-25 μm, about 15-24 μm, about 15-23 μm, about 15-22 μm, about 15-21 μm, about 15-20 μm, about 15-19 μm, about 15-18 μm, about 15-17 μm, about 15-16 μm, about 20-25 μm, about 20-24 μm, about 20-23 μm, about 20-22 μm, or about 20-21 μm.

In some embodiments, particle size Dx(90) of Compound A citrate salt is about 15 μm, about 16 μm, about 17 μm, about 18 μm, about 19 μm, about 20 μm, about 21 μm, about 22 μm, about 23 μm, about 24 μm, or about 25 μm.

In some embodiments, Compound A citrate salt has an average particle size ranging from about 1-100 μm, about 1-50 μm, about 1-25 μm, about 1-24 μm, about 1-23 μm, about 1-22 μm, about 1-21 μm, about 1-20 μm, about 1-19 μm, about 1-18 μm, about 1-17 μm, about 1-16 μm, about 1-15 μm, about 1-14 μm, about 1-13 μm, about 1-12 μm, about 1-11 μm, about 1-10 μm, about 1-9 μm, about 1-8 μm, about 1-7 μm, about 1-6 μm, about 1-5 μm, about 1-4 μm, about 1-2 μm, about 5-25 μm, about 5-24 μm, about 5-23 μm, about 5-22 μm, about 5-21 μm, about 5-20 μm, about 5-19 μm, about 5-18 μm, about 5-17 μm, about 5-16 μm, about 5-15 μm, about 5-14 μm, about 5-13 μm, about 5-12 μm, about 5-11 μm, about 5-10 μm, about 5-9 μm, about 5-8 μm, about 5-7 μm, about 5-6 μm, about 10-25 μm, about 10-24 μm, about 10-23 μm, about 10-22 μm, about 10-21 μm, about 10-20 μm, about 10-19 μm, about 10-18 μm, about 10-17 μm, about 10-16 μm, about 10-15 μm, about 10-14 μm, about 10-13 μm, about 10-12 μm, about 10-11 μm, about 15-25 μm, about 15-24 μm, about 15-23 μm, about 15-22 μm, about 15-21 μm, about 15-20 μm, about 15-19 μm, about 15-18 μm, about 15-17 μm, about 15-16 μm, about 20-25 μm, about 20-24 μm, about 20-23 μm, about 20-22 μm, or about 20-21 μm.

In some embodiments, the average particle size of Compound A citrate salt is about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, or about 25 μm.

In some embodiments, Compound A citrate salt has a median particle size ranging from about 1-100 μm, about 1-50 μm, about 1-25 μm, about 1-24 μm, about 1-23 μm, about 1-22 μm, about 1-21 μm, about 1-20 μm, about 1-19 μm, about 1-18 μm, about 1-17 μm, about 1-16 μm, about 1-15 μm, about 1-14 μm, about 1-13 μm, about 1-12 μm, about 1-11 μm, about 1-10 μm, about 1-9 μm, about 1-8 μm, about 1-7 μm, about 1-6 μm, about 1-5 μm, about 1-4 μm, about 1-2 μm, about 5-25 μm, about 5-24 μm, about 5-23 μm, about 5-22 μm, about 5-21 μm, about 5-20 μm, about 5-19 μm, about 5-18 μm, about 5-17 μm, about 5-16 μm, about 5-15 μm, about 5-14 μm, about 5-13 μm, about 5-12 μm, about 5-11 μm, about 5-10 μm, about 5-9 μm, about 5-8 μm, about 5-7 μm, about 5-6 μm, about 10-25 μm, about 10-24 μm, about 10-23 μm, about 10-22 μm, about 10-21 μm, about 10-20 μm, about 10-19 μm, about 10-18 μm, about 10-17 μm, about 10-16 μm, about 10-15 μm, about 10-14 μm, about 10-13 μm, about 10-12 μm, about 10-11 μm, about 15-25 μm, about 15-24 μm, about 15-23 μm, about 15-22 μm, about 15-21 μm, about 15-20 μm, about 15-19 μm, about 15-18 μm, about 15-17 μm, about 15-16 μm, about 20-25 μm, about 20-24 μm, about 20-23 μm, about 20-22 μm, or about 20-21 μm.

In some embodiments, the median particle size of Compound A citrate salt is about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, or about 25 μm.

In some embodiments, the Compound A monocitrate monohydrate Form A salt has a particle size Dₓ(90) of about 15 μm to about 25 μm.

In some embodiments, the Compound A monocitrate monohydrate Form A salt has a median particle size of about 8 μm to about 25 μm.

In some embodiments, the Compound A monocitrate monohydrate Form A salt has an average particle size of about 8 μm to about 25 μm.

In some embodiments, the Compound A monocitrate monohydrate Form A salt has a particle size Dₓ(50) of about 5 μm to about 10 μm.

In some embodiments, the Compound A monocitrate monohydrate Form A salt has a particle size Dₓ(10) of about 1 μm to about 5 μm.

### Compound A Monocitrate Monohydrate Form A Salt

In some embodiments, the Compound A citrate salt is a crystalline Compound A monocitrate monohydrate Form A salt characterized by the PXRD signals in Table 1A.

TABLE 1A
Compound A monocitrate monohydrate Form A salt
PXRD (±0.2 °2θ; ±0.1 °2θ;
or ±0.0 °2θ; Cu Kα1 radiation).

| Position [°2θ] | Rel. Int. [%] |
|---|---|
| 3.4 | 5.73 |
| 3.5 | 3.05 |
| 3.7 | 6.64 |
| 4.3 | 3.81 |
| 4.7 | 5.07 |
| 5.2 | 38.22 |
| 6.0 | 2.59 |
| 6.4 | 2.45 |
| 6.8 | 2.91 |
| 7.6 | 2.80 |
| 7.9 | 3.12 |
| 8.3 | 2.19 |
| 8.7 | 1.82 |
| 9.5 | 1.90 |
| 10.0 | 2.35 |
| 10.1 | 3.16 |
| 10.2 | 3.69 |
| 10.4 | 6.07 |
| 11.2 | 21.52 |
| 11.5 | 5.95 |
| 11.8 | 100.00 |
| 12.4 | 10.25 |
| 12.6 | 23.90 |
| 12.8 | 4.57 |
| 13.1 | 3.24 |
| 13.4 | 4.21 |
| 13.7 | 3.15 |
| 14.2 | 16.31 |
| 14.5 | 1.85 |
| 14.8 | 1.58 |
| 15.3 | 8.87 |
| 15.6 | 1.68 |
| 16.2 | 37.81 |
| 16.9 | 70.43 |
| 17.2 | 27.94 |
| 17.8 | 12.63 |
| 18.2 | 12.88 |
| 18.7 | 2.32 |
| 19.0 | 4.19 |
| 19.2 | 34.36 |
| 19.9 | 12.21 |
| 20.2 | 10.88 |
| 20.4 | 23.04 |
| 20.7 | 15.84 |
| 20.9 | 28.59 |
| 21.2 | 20.12 |
| 21.6 | 2.93 |
| 22.3 | 6.79 |
| 23.0 | 8.69 |
| 23.5 | 2.72 |
| 23.8 | 6.15 |
| 24.2 | 4.60 |
| 24.6 | 3.95 |
| 24.9 | 10.58 |
| 25.3 | 1.73 |
| 25.5 | 2.85 |
| 26.1 | 9.16 |
| 26.5 | 9.29 |
| 26.7 | 5.95 |
| 26.9 | 7.79 |
| 27.5 | 3.75 |
| 28.1 | 3.12 |
| 28.5 | 8.89 |
| 29.1 | 1.01 |
| 29.3 | 1.20 |
| 29.8 | 4.47 |
| 30.3 | 1.34 |
| 30.7 | 2.95 |
| 30.9 | 1.57 |
| 31.2 | 1.67 |
| 32.0 | 4.46 |
| 32.6 | 2.60 |
| 33.3 | 3.47 |
| 33.7 | 1.67 |
| 34.6 | 0.41 |
| 35.0 | 1.37 |
| 35.7 | 4.60 |
| 36.1 | 1.30 |
| 36.9 | 3.51 |
| 37.3 | 1.55 |
| 38.4 | 3.88 |
| 38.9 | 2.07 |
| 39.5 | 0.52 |
| 39.9 | 0.54 |
| 41.0 | 1.15 |
| 41.4 | 1.12 |
| 42.0 | 1.28 |
| 42.7 | 1.16 |
| 44.0 | 0.58 |

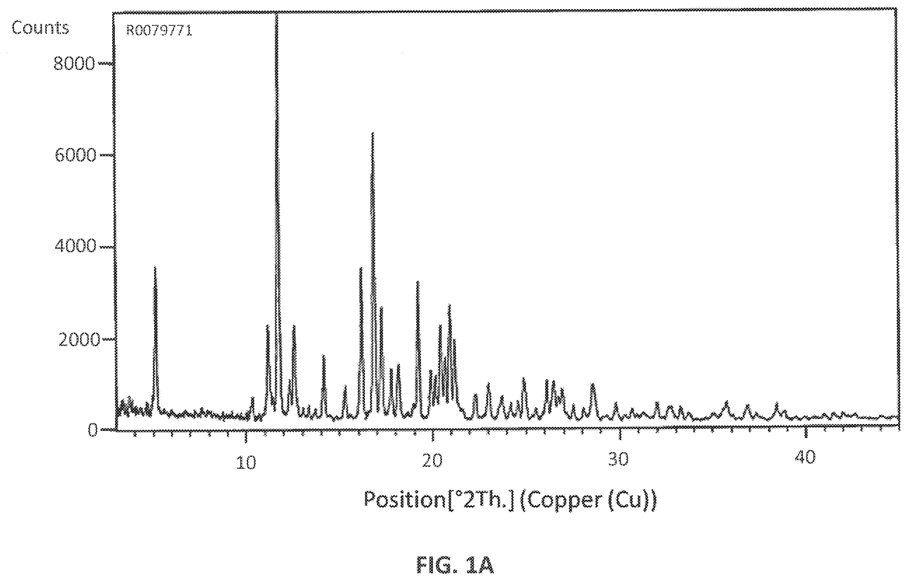

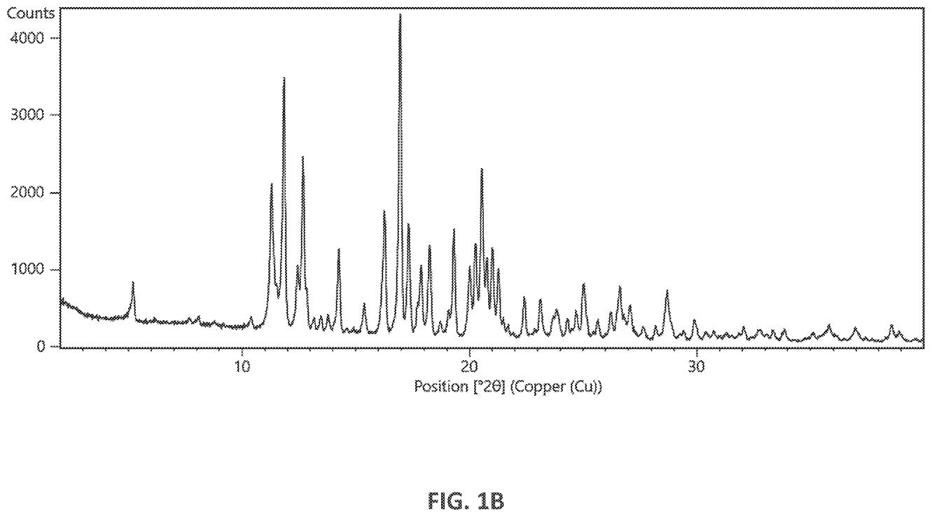

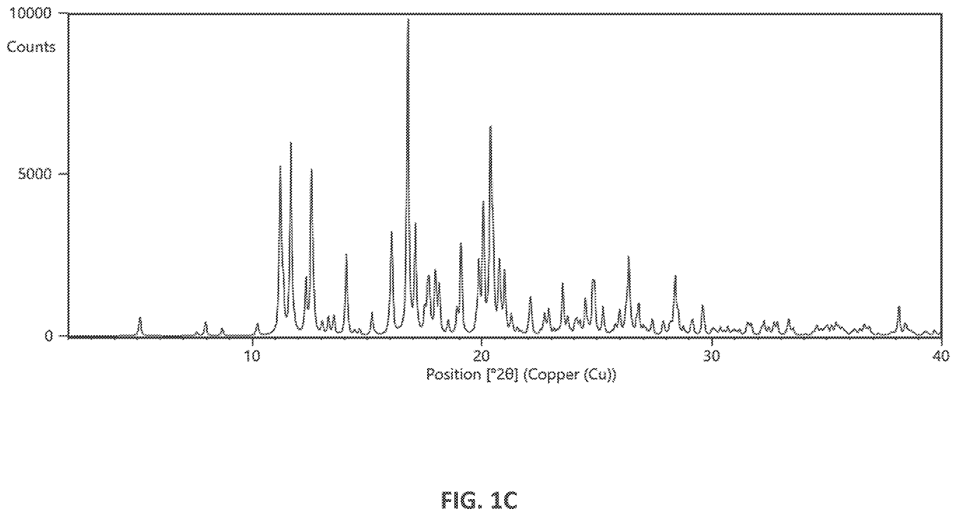

In some embodiments, the Compound A citrate salt is a crystalline Compound A monocitrate monohydrate Form A salt characterized by PXRD spectrum substantially similar to that shown in FIG. 1A.

In some embodiments, the Compound A citrate salt is a crystalline Compound A monocitrate monohydrate Form A salt characterized by PXRD spectrum that is about 95% similar to that shown in FIG. 1A.

In some embodiments, the Compound A citrate salt is a crystalline Compound A monocitrate monohydrate Form A salt characterized by PXRD spectrum that is about 90% similar to that shown in FIG. 1A.

In some embodiments, the Compound A citrate salt is a crystalline Compound A monocitrate monohydrate Form A salt characterized by PXRD spectrum that is about 85% similar to that shown in FIG. 1A.

In some embodiments, the Compound A citrate salt is a crystalline Compound A monocitrate monohydrate Form A salt characterized by a PXRD signal at 11.8°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by a PXRD signal at 11.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by a PXRD signal at 12.6°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by a PXRD signal at 16.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.8°2θ and 16.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two, or three PXRD signals selected from the group consisting of 5.2°2θ, 11.8°2θ, and 16.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.8°2θ, and 16.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.8°2θ, 16.2°2θ, and 16.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.8°2θ, 16.2°2θ, and 16.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.8°2θ, 16.2°2θ, 16.9°2θ, and 19.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.8°2θ, 16.2°2θ, 16.9°2θ, and 19.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.8°2θ, 16.2°2θ, 16.9°2θ, 19.2°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.8°2θ, 16.2°2θ, 16.9°2θ, 19.2°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.8°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.8°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.8°2θ, 12.6°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.8°2θ, 12.6°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.2°2θ, 11.8°2θ, 12.6°2θ, 16.2°2θ, 16.9°2θ, 17.2°2θ, 19.2°2θ, 20.4°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.2°2θ, 11.8°2θ, 12.6°2θ, 16.2°2θ, 16.9°2θ, 17.2°2θ, 19.2°2θ, 20.4°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.2°2θ, 11.8°2θ, 12.6°2θ, 14.2°2θ, 16.2°2θ, 16.9°2θ, 17.2°2θ, 19.2°2θ, 20.4°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.2°2θ, 11.8°2θ, 12.6°2θ, 14.2°2θ, 16.2°2θ, 16.9°2θ, 17.2°2θ, 19.2°2θ, 20.4°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.2°2θ, 11.8°2θ, 12.6°2θ, 14.2°2θ, 16.2°2θ, 16.9°2θ, 17.2°2θ, 19.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.2°2θ, 11.8°2θ, 12.6°2θ, 14.2°2θ, 16.2°2θ, 16.9°2θ, 17.2°2θ, 19.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.2°2θ, 11.8°2θ, 12.6°2θ, 14.2°2θ, 16.2°2θ, 16.9°2θ, 17.2°2θ, 18.2°2θ, 19.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.2°2θ, 11.8°2θ, 12.6°2θ, 14.2°2θ, 16.2°2θ, 16.9°2θ, 17.2°2θ, 18.2°2θ, 19.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 19.9°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 19.9°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 19.9°2θ, 20.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 19.9°2θ, 20.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 19.9°2θ, 20.2°2θ, 20.4°2θ, 20.7°2θ, 20.9°2θ, 21.2°2θ, and 24.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 19.9°2θ, 20.2°2θ, 20.4°2θ, 20.7°2θ, 20.9°2θ, 21.2°2θ, and 24.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 5.2°2θ, 11.2°2θ, 11.8°2θ, 12.4°2θ, 12.6°2θ, 14.2°2θ, 16.2°2θ, 16.9°2θ, 17.2°2θ, 17.8°2θ, 18.2°2θ, 19.2°2θ, 19.9°2θ, 20.2°2θ, 20.4°2θ, 20.7°2θ, 20.9°2θ, 21.2°2θ, and 24.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 5.2°2θ, 11.2°2θ, 11.8°2θ, 12.4°2θ, 12.6°2θ, 14.2°2θ, 16.2°2θ, 16.9°2θ, 17.2°2θ, 17.8°2θ, 18.2°2θ, 19.2°2θ, 19.9°2θ, 20.2°2θ, 20.4°2θ, 20.7°2θ, 20.9°2θ, 21.2°2θ, and 24.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.8°2θ, 16.2°2θ, and 16.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.8°2θ, 16.2°2θ, and 16.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.8°2θ, 16.2°2θ, 16.9°2θ, and 19.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.8°2θ, 16.2°2θ, 16.9°2θ, and 19.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.8°2θ, 16.2°2θ, 16.9°2θ, 19.2°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.8°2θ, 16.2°2θ, 16.9°2θ, 19.2°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.8°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.8°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.8°2θ, 12.6°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.8°2θ, 12.6°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.8°2θ, 12.6°2θ, 16.2°2θ, 16.9°2θ, 17.2°2θ, 19.2°2θ, 20.4°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.8°2θ, 12.6°2θ, 16.2°2θ, 16.9°2θ, 17.2°2θ, 19.2°2θ, 20.4°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.8°2θ, 12.6°2θ, 14.2°2θ, 16.2°2θ, 16.9°2θ, 17.2°2θ, 19.2°2θ, 20.4°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.8°2θ, 12.6°2θ, 14.2°2θ, 16.2°2θ, 16.9°2θ, 17.2°2θ, 19.2°2θ, 20.4°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.8°2θ, 12.6°2θ, 14.2°2θ, 16.2°2θ, 16.9°2θ, 17.2°2θ, 19.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.8°2θ, 12.6°2θ, 14.2°2θ, 16.2°2θ, 16.9°2θ, 17.2°2θ, 19.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.8°2θ, 12.6°2θ, 14.2°2θ, 16.2°2θ, 16.9°2θ, 17.2°2θ, 18.2°2θ, 19.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.8°2θ, 12.6°2θ, 14.2°2θ, 16.2°2θ, 16.9°2θ, 17.2°2θ, 18.2°2θ, 19.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 19.9°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 19.9°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 19.9°2θ, 20.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 19.9°2θ, 20.2°2θ, 20.4°2θ, 20.7°2θ, 20.9°2θ, and 21.2°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 19.9°2θ, 20.2°2θ, 20.4°2θ, 20.7°2θ, 20.9°2θ, 21.2°2θ, and 24.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.8°2θ, 12.6°2θ, 14.2°2θ, 16.2°2θ, 16.9°2θ, 17.2°2θ, 17.8°2θ, 18.2°2θ, 19.2°2θ, 19.9°2θ, 20.2°2θ, 20.4°2θ, 20.7°2θ, 20.9°2θ, 21.2°2θ, and 24.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.8°2θ, 12.4°2θ, 12.6°2θ, 14.2°2θ, 16.2°2θ, 16.9°2θ, 17.2°2θ, 17.8°2θ, 18.2°2θ, 19.2°2θ, 19.9°2θ, 20.2°2θ, 20.4°2θ, 20.7°2θ, 20.9°2θ, 21.2°2θ, and 24.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.8°2θ, 12.4°2θ, 12.6°2θ, 14.2°2θ, 16.2°2θ, 16.9°2θ, 17.2°2θ, 17.8°2θ, 18.2°2θ, 19.2°2θ, 19.9°2θ, 20.2°2θ, 20.4°2θ, 20.7°2θ, 20.9°2θ, 21.2°2θ, and 24.9°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-one, fifty-two, fifty-three, fifty-four, fifty-five, fifty-six, fifty-seven, fifty-eight, fifty-nine, sixty, sixty-one, sixty-two, sixty-three, sixty-four, sixty-five, sixty-six, sixty-seven, sixty-eight, sixty-nine, seventy, seventy-one, seventy-two, seventy-three, seventy-four, seventy-five, seventy-six, seventy-seven, seventy-eight, seventy-nine, eighty, eighty-one, eighty-two, eighty-three, eighty-four, eighty-five, eighty-six, eighty-seven, eighty-eight, or eighty-nine XRPD signals selected from those set forth in Table 1A.

In some embodiments, the Compound A citrate salt is a crystalline Compound A monocitrate monohydrate Form A salt characterized by one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more, thirty-nine or more, forty or more, forty-one or more, forty-two or more, forty-three or more, forty-four or more, forty-five or more, forty-six or more, forty-seven or more, forty-eight or more, forty-nine or more, fifty or more, fifty-one or more, fifty-two or more, fifty-three or more, fifty-four or more, fifty-five or more, fifty-six or more, fifty-seven or more, fifty-eight or more, fifty-nine or more, sixty or more, sixty-one or more, sixty-two or more, sixty-three or more, sixty-four or more, sixty-five or more, sixty-six or more, sixty-seven or more, sixty-eight or more, sixty-nine or more, seventy or more, seventy-one or more, seventy-two or more, seventy-three or more, seventy-four or more, seventy-five or more, seventy-six or more, seventy-seven or more, seventy-eight or more, seventy-nine or more, eighty or more, eighty-one or more, eighty-two or more, eighty-three or more, eighty-four or more, eighty-five or more, eighty-six or more, eighty-seven or more, eighty-eight, or eighty-nine XRPD signals selected from those set forth in Table 1A.

In some embodiments, the Compound A citrate salt is a crystalline Compound A monocitrate monohydrate Form A salt characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-one, fifty-two, fifty-three, fifty-four, fifty-five, fifty-six, fifty-seven, fifty-eight, fifty-nine, sixty, sixty-one, sixty-two, sixty-three, sixty-four, sixty-five, sixty-six, sixty-seven, sixty-eight, sixty-nine, seventy, seventy-one, seventy-two, seventy-three, seventy-four, seventy-five, seventy-six, seventy-seven, seventy-eight, seventy-nine, eighty, eighty-one, eighty-two, eighty-three, eighty-four, eighty-five, eighty-six, eighty-seven, eighty-eight, or eighty-nine XRPD signals selected from 3.4°2θ, 3.5°2θ, 3.7°2θ, 4.3°2θ, 4.7°2θ, 5.2°2θ, 6.0°2θ, 6.4°2θ, 6.8°2θ, 7.6°2θ, 7.9°2θ, 8.3°2θ, 8.7°2θ, 9.5°2θ, 10.0°2θ, 10.1°2θ, 10.2°2θ, 10.4°2θ, 11.2°2θ, 11.5°2θ, 11.8°2θ, 12.4°2θ, 12.6°2θ, 12.8°2θ, 13.1°2θ, 13.4°2θ, 13.7°2θ, 14.2°2θ, 14.5°2θ, 14.8°2θ, 15.3°2θ, 15.6°2θ, 16.2°2θ, 16.9°2θ, 17.2°2θ, 17.8°2θ, 18.2°2θ, 18.7°2θ, 19.0°2θ, 19.2°2θ, 19.9°2θ, 20.2°2θ, 20.4°2θ, 20.7°2θ, 20.9°2θ, 21.2°2θ, 21.6°2θ, 22.3°2θ, 23.0°2θ, 23.5°2θ, 23.8°2θ, 24.2°2θ, 24.6°2θ, 24.9°2θ, 25.3°2θ, 25.5°2θ, 26.1°2θ, 26.5°2θ, 26.7°2θ, 26.9°2θ, 27.5°2θ, 28.1°2θ, 28.5°2θ, 29.1°2θ, 29.3°2θ, 29.8°2θ, 30.3°2θ, 30.7°2θ, 30.9°2θ, 31.2°2θ, 32.0°2θ, 32.6°2θ, 33.3°2θ, 33.7°2θ, 34.6°2θ, 35.0°2θ, 35.7°2θ, 36.1°2θ, 36.9°2θ, 37.3°2θ, 38.4°2θ, 38.9°2θ, 39.5°2θ, 39.9°2θ, 41.0°2θ, 41.4°2θ, 42°2θ, 42.7°2θ, and 44.0°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more, thirty-nine or more, forty or more, forty-one or more, forty-two or more, forty-three or more, forty-four or more, forty-five or more, forty-six or more, forty-seven or more, forty-eight or more, forty-nine or more, fifty or more, fifty-one or more, fifty-two or more, fifty-three or more, fifty-four or more, fifty-five or more, fifty-six or more, fifty-seven or more, fifty-eight or more, fifty-nine or more, sixty or more, sixty-one or more, sixty-two or more, sixty-three or more, sixty-four or more, sixty-five or more, sixty-six or more, sixty-seven or more, sixty-eight or more, sixty-nine or more, seventy or more, seventy-one or more, seventy-two or more, seventy-three or more, seventy-four or more, seventy-five or more, seventy-six or more, seventy-seven or more, seventy-eight or more, seventy-nine or more, eighty or more, eighty-one or more, eighty-two or more, eighty-three or more, eighty-four or more, eighty-five or more, eighty-six or more, eighty-seven or more, eighty-eight, or eighty-nine XRPD signals selected from 3.4°2θ, 3.5°2θ, 3.7°2θ, 4.3°2θ, 4.7°2θ, 5.2°2θ, 6.0°2θ, 6.4°2θ, 6.8°2θ, 7.6°2θ, 7.9°2θ, 8.3°2θ, 8.7°2θ, 9.5°2θ, 10.0°2θ, 10.1°2θ, 10.2°2θ, 10.4°2θ, 11.2°2θ, 11.5°2θ, 11.8°2θ, 12.4°2θ, 12.6°2θ, 12.8°2θ, 13.1°2θ, 13.4°2θ, 13.7°2θ, 14.2°2θ, 14.5°2θ, 14.8°2θ, 15.3°2θ, 15.6°2θ, 16.2°2θ, 16.9°2θ, 17.2°2θ, 17.8°2θ, 18.2°2θ, 18.7°2θ, 19.0°2θ, 19.2°2θ, 19.9°2θ, 20.2°2θ, 20.4°2θ, 20.7°2θ, 20.9°2θ, 21.2°2θ, 21.6°2θ, 22.3°2θ, 23.0°2θ, 23.5°2θ, 23.8°2θ, 24.2°2θ, 24.6°2θ, 24.9°2θ, 25.3°2θ, 25.5°2θ, 26.1°2θ, 26.5°2θ, 26.7°2θ, 26.9°2θ, 27.5°2θ, 28.1°2θ, 28.5°2θ, 29.1°2θ, 29.3°2θ, 29.8°2θ, 30.3°2θ, 30.7°2θ, 30.9°2θ, 31.2°2θ, 32.0°2θ, 32.6°2θ, 33.3°2θ, 33.7°2θ, 34.6°2θ, 35.0°2θ, 35.7°2θ, 36.1°2θ, 36.9°2θ, 37.3°2θ, 38.4°2θ, 38.9°2θ, 39.5°2θ, 39.9°2θ, 41.0°2θ, 41.4°2θ, 42°2θ, 42.7°2θ, and 44.0°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by the PXRD signals in Table 1B.

TABLE 1B
Compound A monocitrate monohydrate Form A salt PXRD, simulated
(±0.2 °2θ; ±0.1 °2θ; or ±0.0 °2θ;
Cu Kα1 radiation).

| Pos. [°2θ] | Rel. Int. [%] |
|---|---|
| 5.2 | 12.11 |
| 8.1 | 2.64 |
| 10.4 | 3.09 |
| 11.3 | 45.58 |
| 11.8 | 78.63 |
| 12.4 | 20.00 |
| 12.7 | 53.78 |
| 12.9 | 11.94 |
| 13.2 | 3.22 |
| 13.5 | 4.05 |
| 13.8 | 4.43 |
| 14.3 | 25.49 |
| 15.4 | 8.65 |
| 16.3 | 37.75 |
| 17.0 | 100.00 |
| 17.3 | 33.98 |
| 17.9 | 19.59 |
| 18.3 | 27.32 |
| 18.7 | 3.49 |
| 19.1 | 6.90 |
| 19.3 | 32.53 |
| 20.0 | 20.93 |
| 20.3 | 28.13 |
| 20.5 | 51.65 |
| 20.8 | 23.96 |
| 21.0 | 26.96 |
| 21.3 | 20.68 |
| 21.5 | 5.09 |
| 21.7 | 2.73 |
| 22.4 | 11.72 |
| 23.1 | 10.73 |
| 23.7 | 6.04 |
| 23.8 | 8.18 |
| 24.3 | 5.06 |
| 24.7 | 7.77 |
| 25.0 | 16.01 |
| 25.6 | 5.09 |
| 26.2 | 7.69 |
| 26.6 | 15.84 |
| 27.1 | 9.86 |
| 27.6 | 3.18 |
| 28.2 | 3.79 |
| 28.7 | 14.05 |
| 29.9 | 5.59 |
| 30.7 | 2.36 |
| 32.0 | 3.74 |
| 32.7 | 2.90 |
| 33.3 | 3.06 |
| 33.9 | 3.15 |
| 35.8 | 4.70 |
| 36.9 | 3.89 |
| 38.6 | 4.97 |

In some embodiments, the Compound A citrate salt is a crystalline Compound A monocitrate monohydrate Form A salt characterized by PXRD spectrum substantially similar to that shown in FIG. 1B.

In some embodiments, the Compound A citrate salt is a crystalline Compound A monocitrate monohydrate Form A salt characterized by PXRD spectrum that is about 95% similar to that shown in FIG. 1B.

In some embodiments, the Compound A citrate salt is a crystalline Compound A monocitrate monohydrate Form A salt characterized by PXRD spectrum that is about 90% similar to that shown in FIG. 1B.

In some embodiments, the Compound A citrate salt is a crystalline Compound A monocitrate monohydrate Form A salt characterized by PXRD spectrum that is about 85% similar to that shown in FIG. 1B.

In some embodiments, the Compound A citrate salt is a crystalline Compound A monocitrate monohydrate Form A salt characterized by one, two, three, four, five, six, seven, eight, nine, ten, eleven, twelve, thirteen, fourteen, or fifteen PXRD signals selected from those set forth in Table 1B.

In some embodiments, the Compound A citrate salt is a crystalline Compound A monocitrate monohydrate Form A salt characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-one, or fifty-two XRPD signals selected from those set forth in Table 1B.

In some embodiments, the Compound A citrate salt is a crystalline Compound A monocitrate monohydrate Form A salt characterized by one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more, thirty-nine or more, forty or more, forty-one or more, forty-two or more, forty-three or more, forty-four or more, forty-five or more, forty-six or more, forty-seven or more, forty-eight or more, forty-nine or more, fifty or more, fifty-one, or fifty-two XRPD signals selected from those set forth in Table 1B.

In some embodiments, the Compound A citrate salt is a crystalline Compound A monocitrate monohydrate Form A salt characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-one, or fifty-two XRPD signals selected from 5.2°2θ, 8.1°2θ, 10.4°2θ, 11.3°2θ, 11.8°2θ, 12.4°2θ, 12.7°2θ, 12.9°2θ, 13.2°2θ, 13.5°2θ, 13.8°2θ, 14.3°2θ, 15.4°2θ, 16.3°2θ, 17.0°2θ, 17.3°2θ, 17.9°2θ, 18.3°2θ, 18.7°2θ, 19.1°2θ, 19.3°2θ, 20.0°2θ, 20.3°2θ, 20.5°2θ, 20.8°2θ, 21.0°2θ, 21.3°2θ, 21.5°2θ, 21.7°2θ, 22.4°2θ, 23.1°2θ, 23.7°2θ, 23.8°2θ, 24.3°2θ, 24.7°2θ, 25.0°2θ, 25.6°2θ, 26.2°2θ, 26.6°2θ, 27.1°2θ, 27.6°2θ, 28.2°2θ, 28.7°2θ, 29.9°2θ, 30.7°2θ, 32.0°2θ, 32.7°2θ, 33.3°2θ, 33.9°2θ, 35.8°2θ, 36.9°2θ, and 38.6°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more, thirty-nine or more, forty or more, forty-one, or forty-two XRPD signals selected from 5.2°2θ, 8.1°2θ, 10.4°2θ, 11.3°2θ, 11.8°2θ, 12.4°2θ, 12.7°2θ, 12.9°2θ, 13.2°2θ, 13.5°2θ, 13.8°2θ, 14.3°2θ, 15.4°2θ, 16.3°2θ, 17.0°2θ, 17.3°2θ, 17.9°2θ, 18.3°2θ, 18.7°2θ, 19.1°2θ, 19.3°2θ, 20.0°2θ, 20.3°2θ, 20.5°2θ, 20.8°2θ, 21.0°2θ, 21.3°2θ, 21.5°2θ, 21.7°2θ, 22.4°2θ, 23.1°2θ, 23.7°2θ, 23.8°2θ, 24.3°2θ, 24.7°2θ, 25.0°2θ, 25.6°2θ, 26.2°2θ, 26.6°2θ, 27.1°2θ, 27.6°2θ, 28.2°2θ, 28.7°2θ, 29.9°2θ, 30.7°2θ, 32.0°2θ, 32.7°2θ, 33.3°2θ, 33.9°2θ, 35.8°2θ, 36.9°2θ, and 38.6°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two, or three PXRD signals selected from the group consisting of 11.8°2θ, 12.7°2θ, and 17.0°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.8°2θ, 12.7°2θ, and 17.0°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.8°2θ, 12.7°2θ, 17.0°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.8°2θ, 12.7°2θ, 17.0°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.3°2θ, 11.8°2θ, 12.7°2θ, 17.0°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.3°2θ, 11.8°2θ, 12.7°2θ, 17.0°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.3°2θ, 11.8°2θ, 12.7°2θ, 16.3°2θ, 17.0°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.3°2θ, 11.8°2θ, 12.7°2θ, 16.3°2θ, 17.0°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.3°2θ, 11.8°2θ, 12.7°2θ, 16.3°2θ, 17.0°2θ, 17.3°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.3°2θ, 11.8°2θ, 12.7°2θ, 16.3°2θ, 17.0°2θ, 17.3°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.3°2θ, 11.8°2θ, 12.7°2θ, 16.3°2θ, 17.0°2θ, 17.3°2θ, 19.3°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.3°2θ, 11.8°2θ, 12.7°2θ, 16.3°2θ, 17.0°2θ, 17.3°2θ, 19.3°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.3°2θ, 11.8°2θ, 12.7°2θ, 16.3°2θ, 17.0°2θ, 17.3°2θ, 19.3°2θ, 20.3°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.3°2θ, 11.8°2θ, 12.7°2θ, 16.3°2θ, 17.0°2θ, 17.3°2θ, 19.3°2θ, 20.3°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.3°2θ, 11.8°2θ, 12.7°2θ, 16.3°2θ, 17.0°2θ, 17.3°2θ, 18.3°2θ, 19.3°2θ, 20.3°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.3°2θ, 11.8°2θ, 12.7°2θ, 16.3°2θ, 17.0°2θ, 17.3°2θ, 18.3°2θ, 19.3°2θ, 20.3°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.3°2θ, 11.8°2θ, 12.7°2θ, 16.3°2θ, 17.0°2θ, 17.3°2θ, 18.3°2θ, 19.3°2θ, 20.3°2θ, 20.5°2θ, and 21.0°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.3°2θ, 11.8°2θ, 12.7°2θ, 16.3°2θ, 17.0°2θ, 17.3°2θ, 18.3°2θ, 19.3°2θ, 20.3°2θ, 20.5°2θ, and 21.0°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.3°2θ, 11.8°2θ, 12.7°2θ, 14.3°2θ, 16.3°2θ, 17.0°2θ, 17.3°2θ, 18.3°2θ, 19.3°2θ, 20.3°2θ, 20.5°2θ, and 21.0°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.3°2θ, 11.8°2θ, 12.7°2θ, 14.3°2θ, 16.3°2θ, 17.0°2θ, 17.3°2θ, 18.3°2θ, 19.3°2θ, 20.3°2θ, 20.5°2θ, and 21.0°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD spectrum substantially similar to that shown in FIG. 1C.

In some embodiments, the Compound A citrate salt is a crystalline Compound A monocitrate monohydrate Form A salt characterized by PXRD spectrum that is about 95% similar to that shown in FIG. 1C.

In some embodiments, the Compound A citrate salt is a crystalline Compound A monocitrate monohydrate Form A salt characterized by PXRD spectrum that is about 90% similar to that shown in FIG. 1C.

In some embodiments, the Compound A citrate salt is a crystalline Compound A monocitrate monohydrate Form A salt characterized by PXRD spectrum that is about 85% similar to that shown in FIG. 1C.

In some embodiments, the Compound A citrate salt is a crystalline Compound A monocitrate monohydrate Form A salt characterized by the PXRD signals in Table 1C.

TABLE 1C
Compound A monocitrate monohydrate Form A salt PXRD, simulated
(±0.2 °2θ; ±0.1 °2θ; or ±0.0° 2θ;
Cu Kα1 radiation).

| Pos. [°2θ] | Rel. Int. [%] |
|---|---|
| 5.1 | 5.87 |
| 7.6 | 1.02 |
| 8.0 | 4.23 |
| 8.7 | 2.34 |
| 10.2 | 3.7 |
| 11.2 | 52.55 |
| 11.7 | 60.35 |
| 12.3 | 18.19 |
| 12.6 | 51.55 |
| 13.0 | 4.59 |
| 13.3 | 6.15 |
| 13.6 | 6.41 |
| 14.1 | 25.31 |
| 14.7 | 2.3 |
| 15.2 | 7.33 |
| 16.1 | 32.75 |
| 16.8 | 100 |
| 17.1 | 34.98 |
| 17.7 | 18.53 |
| 18.0 | 20.52 |
| 18.1 | 16.45 |
| 18.5 | 5.05 |
| 18.9 | 9 |
| 19.1 | 29.03 |
| 19.9 | 24.04 |
| 20.1 | 41.56 |
| 20.4 | 66.37 |
| 20.5 | 36.21 |
| 20.8 | 24.39 |
| 21.0 | 20.68 |
| 21.3 | 7.04 |
| 21.5 | 2.75 |
| 22.1 | 11.93 |
| 22.7 | 7.06 |
| 22.9 | 8.44 |
| 23.5 | 16.33 |
| 23.7 | 6.2 |
| 24.1 | 5.48 |
| 24.5 | 11.65 |
| 24.9 | 17.22 |
| 25.3 | 9.14 |
| 25.8 | 3.86 |
| 26.0 | 8.11 |
| 26.4 | 24.73 |
| 26.8 | 10.31 |
| 27.4 | 5.25 |
| 27.9 | 4.56 |
| 28.4 | 18.72 |
| 28.8 | 2.95 |
| 29.1 | 5.12 |
| 29.6 | 9.5 |
| 30.1 | 2.48 |
| 30.4 | 2.75 |
| 30.7 | 2.95 |
| 31.6 | 4.23 |
| 32.3 | 4.72 |
| 32.5 | 2.92 |
| 32.7 | 4.25 |
| 33.3 | 5.08 |
| 33.5 | 2.57 |
| 34.6 | 3.27 |
| 35.0 | 3.37 |
| 35.4 | 4.1 |
| 36.9 | 2.89 |
| 38.1 | 9.15 |
| 38.4 | 3.83 |
| 39.3 | 1.39 |
| 39.7 | 1.66 |

In some embodiments, the Compound A citrate salt is a crystalline Compound A monocitrate monohydrate Form A salt characterized by one, two, three, four, five, six, seven, eight, nine, ten, eleven, twelve, thirteen, fourteen, fifteen, sixteen, seventeen, eighteen, or nineteen PXRD signals selected from those set forth in Table 1C.

In some embodiments, the Compound A citrate salt is a crystalline Compound A monocitrate monohydrate Form A salt characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-one, fifty-two, fifty-three, fifty-four, fifty-five, fifty-six, fifty-seven, fifty-eight, fifty-nine, sixty, sixty-one, sixty-two, sixty-three, sixty-four, sixty-five, sixty-six, sixty-seven or sixty-eight XRPD signals selected from those set forth in Table 1C.

In some embodiments, the Compound A citrate salt is a crystalline Compound A monocitrate monohydrate Form A salt characterized by one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more, thirty-nine or more, forty or more, forty-one or more, forty-two or more, forty-three or more, forty-four or more, forty-five or more, forty-six or more, forty-seven or more, forty-eight or more, forty-nine or more, fifty or more, fifty-one or more, fifty-two or more, fifty-three or more, fifty-four or more, fifty-five or more, fifty-six or more, fifty-seven or more, fifty-eight or more, fifty-nine or more, sixty or more, sixty-one or more, sixty-two or more, sixty-three or more, sixty-four or more, or sixty-five or more, sixty-six or more, sixty-seven, or sixty-eight XRPD signals selected from those set forth in Table 1C.

In some embodiments, the Compound A citrate salt is a crystalline Compound A monocitrate monohydrate Form A salt characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-one, fifty-two, fifty-three, fifty-four, fifty-five, fifty-six, fifty-seven, fifty-eight, fifty-nine, sixty, sixty-one, sixty-two, sixty-three, sixty-four, sixty-five, sixty-six, sixty-seven or sixty-eight XRPD signals selected from 5.1°2θ, 7.6°2θ, 8.0°2θ, 8.7°2θ, 10.2°2θ, 11.2°2θ, 11.7°2θ, 12.3°2θ, 12.6°2θ, 13.0°2θ, 13.3°2θ, 13.6°2θ, 14.1°2θ, 14.7°2θ, 15.2°2θ, 16.1°2θ, 16.8°2θ, 17.1°2θ, 17.7°2θ, 18.0°2θ, 18.1°2θ, 18.5°2θ, 18.9°2θ, 19.1°2θ, 19.9°2θ, 20.1°2θ, 20.4°2θ, 20.5°2θ, 20.8°2θ, 21.0°2θ, 21.3°2θ, 21.5°2θ, 22.1°2θ, 22.7°2θ, 22.9°2θ, 23.5°2θ, 23.7°2θ, 24.1°2θ, 24.5°2θ, 24.9°2θ, 25.3°2θ, 25.8°2θ, 26.0°2θ, 26.4°2θ, 26.8°2θ, 27.4°2θ, 27.9°2θ, 28.4°2θ, 28.8°2θ, 29.1°2θ, 29.6°2θ, 30.1°2θ, 30.4°2θ, 30.7°2θ, 31.6°2θ, 32.3°2θ, 32.5°2θ, 32.7°2θ, 33.3°2θ, 33.5°2θ, 34.6°2θ, 35.0°2θ, 35.4°2θ, 36.9°2θ, 38.1°2θ, 38.4°2θ, 39.3°2θ, and 39.7°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more, thirty-nine or more, forty or more, forty-one or more, forty-two or more, forty-three or more, forty-four or more, forty-five or more, forty-six or more, forty-seven or more, forty-eight or more, forty-nine or more, fifty or more, fifty-one or more, fifty-two or more, fifty-three or more, fifty-four or more, fifty-five or more, fifty-six or more, fifty-seven or more, fifty-eight or more, fifty-nine or more, sixty or more, sixty-one or more, sixty-two or more, sixty-three or more, sixty-four or more, or sixty-five or more, sixty-six or more, sixty-seven, or sixty-eight XRPD signals selected from 5.1°2θ, 7.6°2θ, 8.0°2θ, 8.7°2θ, 10.2°2θ, 11.2°2θ, 11.7°2θ, 12.3°2θ, 12.6°2θ, 13.0°2θ, 13.3°2θ, 13.6°2θ, 14.1°2θ, 14.7°2θ, 15.2°2θ, 16.1°2θ, 16.8°2θ, 17.1°2θ, 17.7°2θ, 18.0°2θ, 18.1°2θ, 18.5°2θ, 18.9°2θ, 19.1°2θ, 19.9°2θ, 20.1°2θ, 20.4°2θ, 20.5°2θ, 20.8°2θ, 21.0°2θ, 21.3°2θ, 21.5°2θ, 22.1°2θ, 22.7°2θ, 22.9°2θ, 23.5°2θ, 23.7°2θ, 24.1°2θ, 24.5°2θ, 24.9°2θ, 25.3°2θ, 25.8°2θ, 26.0°2θ, 26.4°2θ, 26.8°2θ, 27.4°2θ, 27.9°2θ, 28.4°2θ, 28.8°2θ, 29.1°2θ, 29.6°2θ, 30.1°2θ, 30.4°2θ, 30.7°2θ, 31.6°2θ, 32.3°2θ, 32.5°2θ, 32.7°2θ, 33.3°2θ, 33.5°2θ, 34.6°2θ, 35.0°2θ, 35.4°2θ, 36.9°2θ, 38.1°2θ, 38.4°2θ, 39.3°2θ, and 39.7°2θ (±0.2°2θ; 0.1°2θ; or ±0.0°2θ; Cu Kα1 radiation).

In some embodiments, the Compound A citrate salt is a crystalline Compound A monocitrate monohydrate Form A salt characterized by two, or three PXRD signals selected from the group consisting of 11.7°2θ, 16.8°2θ, and 20.4°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.7°2θ, 16.8°2θ, and 20.4°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.7°2θ, 16.8°2θ, and 20.4°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.7°2θ, 16.8°2θ, and 20.4°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.7°2θ, 12.6°2θ, 16.8°2θ, and 20.4°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.7°2θ, 12.6°2θ, 16.8°2θ, and 20.4°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.7°2θ, 12.6°2θ, 16.8°2θ, 20.1°2θ, and 20.4°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.7°2θ, 12.6°2θ, 16.8°2θ, 20.1°2θ, and 20.4°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.7°2θ, 12.6°2θ, 16.8°2θ, 20.1°2θ, 20.4°2θ and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.7°2θ, 12.6°2θ, 16.8°2θ, 20.1°2θ, 20.4°2θ and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.7°2θ, 12.6°2θ, 16.8°2θ, 17.1°2θ, 20.1°2θ, 20.4°2θ and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.7°2θ, 12.6°2θ, 16.8°2θ, 17.1°2θ, 20.1°2θ, 20.4°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.7°2θ, 12.6°2θ, 16.1°2θ, 16.8°2θ, 17.1°2θ, 20.1°2θ, 20.4°2θ, and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.7°2θ, 12.6°2θ, 16.1°2θ, 16.8°2θ, 17.1°2θ, 20.1°2θ, 20.4°2θ and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.7°2θ, 12.6°2θ, 16.1°2θ, 16.8°2θ, 17.1°2θ, 19.1°2θ, 20.1°2θ, 20.4°2θ and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.7°2θ, 12.6°2θ, 16.1°2θ, 16.8°2θ, 17.1°2θ, 19.1°2θ, 20.1°2θ, 20.4°2θ and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.7°2θ, 12.6°2θ, 14.1°2θ, 16.1°2θ, 16.8°2θ, 17.1°2θ, 19.1°2θ, 20.1°2θ, 20.4°2θ and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.7°2θ, 12.6°2θ, 14.1°2θ, 16.1°2θ, 16.8°2θ, 17.1°2θ, 19.1°2θ, 20.1°2θ, 20.4°2θ and 20.5°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by two or more, or three or more PXRD signals selected from the group consisting of 11.2°2θ, 11.7°2θ, 12.6°2θ, 14.1°2θ, 16.1°2θ, 16.8°2θ, 17.1°2θ, 19.1°2θ, 20.1°2θ, 20.4°2θ, 20.5°2θ and 26.4°2θ (±0.2°2θ; ±0.1°2θ; or ±0.0°2θ; Cu Kα1 radiation).

In some embodiments, the Compound A citrate salt is a crystalline Compound A monocitrate monohydrate Form A salt characterized by PXRD signals at 11.2°2θ, 11.7°2θ, 12.6°2θ, 14.1°2θ, 16.1°2θ, 16.8°2θ, 17.1°2θ, 19.1°2θ, 20.1°2θ, 20.4°2θ, 20.5°2θ and 26.4°2θ (±0.2°2θ; ±0.1°2θ; or ±0.0°2θ; Cu Kα1 radiation).

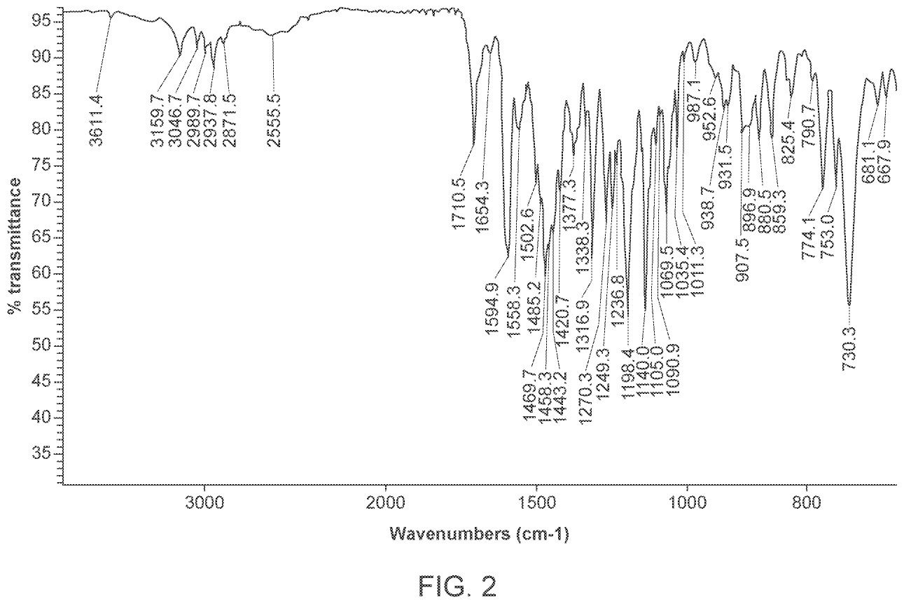

In some embodiments, the Compound A citrate salt is a crystalline Compound A monocitrate monohydrate Form A salt and is characterized by FT-IR substantially similar to that shown in FIG. 2.

In some embodiments, the Compound A citrate salt is a crystalline Compound A monocitrate monohydrate Form A salt and is characterized by FT-IR that is about 95% similar to that shown in FIG. 2.

In some embodiments, the Compound A citrate salt is a crystalline Compound A monocitrate monohydrate Form A salt and is characterized by FT-IR that is about 90% similar to that shown in FIG. 2.

In some embodiments, the Compound A citrate salt is a crystalline Compound A monocitrate monohydrate Form A salt and is characterized by FT-IR that is about 85% similar to that shown in FIG. 2.

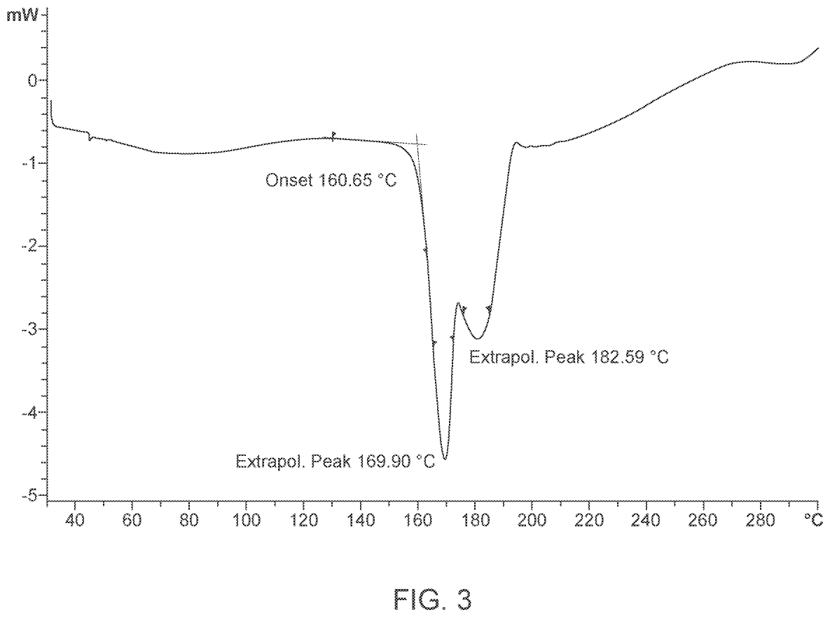

In some embodiments, the Compound A citrate salt is a crystalline Compound A monocitrate monohydrate Form A salt and is characterized by DSC substantially similar to that shown in FIG. 3.

In some embodiments, the Compound A citrate salt is a crystalline Compound A monocitrate monohydrate Form A salt and is characterized by DSC that is about 95% similar to that shown in FIG. 3.

In some embodiments, the Compound A citrate salt is a crystalline Compound A monocitrate monohydrate Form A salt and is characterized by DSC that is about 90% similar to that shown in FIG. 3.

In some embodiments, the Compound A citrate salt is a crystalline Compound A monocitrate monohydrate Form A salt and is characterized by DSC that is about 85% similar to that shown in FIG. 3.

In some embodiments, the Compound A citrate salt is a crystalline Compound A monocitrate monohydrate Form A salt and is characterized by a melting point from about 165° C. to about 175° C., or from about 177° C. to about 187° C. as measured by DSC.

In some embodiments, the Compound A citrate salt is a crystalline Compound A monocitrate monohydrate Form A salt and is characterized by a melting point between approximately 165° C. and approximately 175° C., and between approximately 177° C. and approximately 187° C. as measured by DSC.

In some embodiments, the Compound A citrate salt is a crystalline Compound A monocitrate monohydrate Form A salt and is characterized by DSC having a melting point at about 169.9° C. or about 182.6° C.

In some embodiments, the Compound A citrate salt is a crystalline Compound A monocitrate monohydrate Form A salt and is characterized by DSC having melting peaks at about 169.9° C. and about 182.6° C.

In some embodiments, the Compound A citrate salt is a crystalline Compound A monocitrate monohydrate Form A salt and is characterized by DSC having melting point at about 169.9° C.

In some embodiments, the Compound A citrate salt is a crystalline Compound A monocitrate monohydrate Form A salt and is characterized by DSC having melting point at about 182.6° C.

In some embodiments, the Compound A citrate salt is a crystalline Compound A monocitrate monohydrate Form A salt and is characterized by DSC having endothermic events occurring at about 50.3° C. and about 169.0° C.

In some embodiments, the Compound A citrate salt is a crystalline Compound A monocitrate monohydrate Form A salt and is characterized by TGA showing a weight loss of 1.9 wt % between RT-100° C.

In some embodiments, the Compound A citrate salt is a crystalline Compound A monocitrate monohydrate Form A salt characterized by one, two, three, four, five, six, seven, eight, nine, ten, eleven, twelve, thirteen, fourteen, fifteen, sixteen, seventeen, eighteen, or nineteen PXRD signals selected from those set forth in Table 1A.

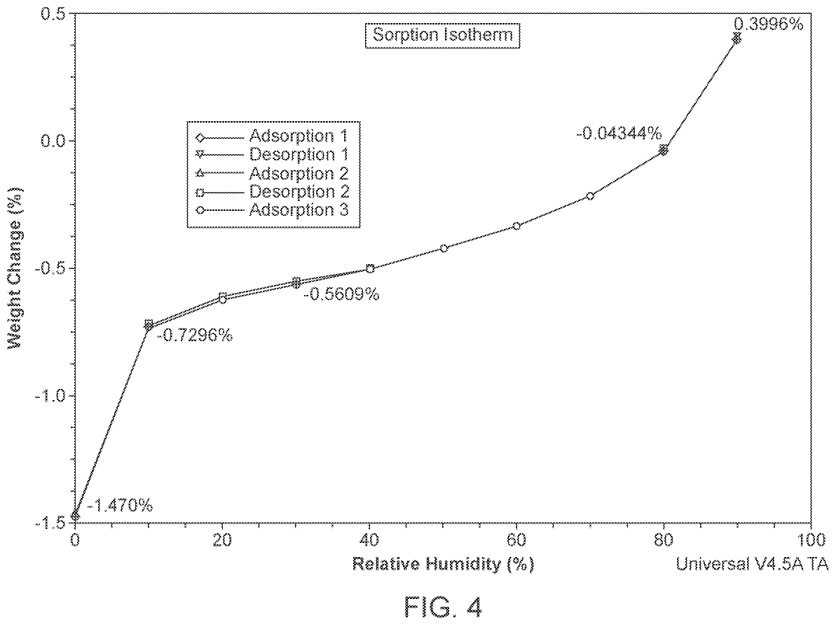

In some embodiments, the Compound A citrate salt is a crystalline Compound A monocitrate monohydrate Form A salt and displays DVS isotherm at 25° C. and RH up to 90% substantially similar to that set forth in FIG. 4.

In some embodiments, the Compound A citrate salt is a crystalline Compound A monocitrate monohydrate Form A salt and displays DVS isotherm at 25° C. and RH up to about 95% similar to that set forth in FIG. 4.

In some embodiments, the Compound A citrate salt is a crystalline Compound A monocitrate monohydrate Form A salt and displays DVS isotherm at 25° C. and RH up to about 90% similar to that set forth in FIG. 4.

In some embodiments, the Compound A citrate salt is a crystalline Compound A monocitrate monohydrate Form A salt and displays DVS isotherm at 25° C. and RH up to about 85% similar to that set forth in FIG. 4.

In some embodiments, Compound A citrate salt is a crystalline Compound A monocitrate monohydrate Form A salt and is non-hygroscopic. In some embodiments, Compound A citrate salt is a crystalline Compound A monocitrate monohydrate Form A salt and displays non-hygroscopicity between 0 and 90% RH at between 25° C. and 45° C. (e.g., less than 0.2% w/w water uptake).

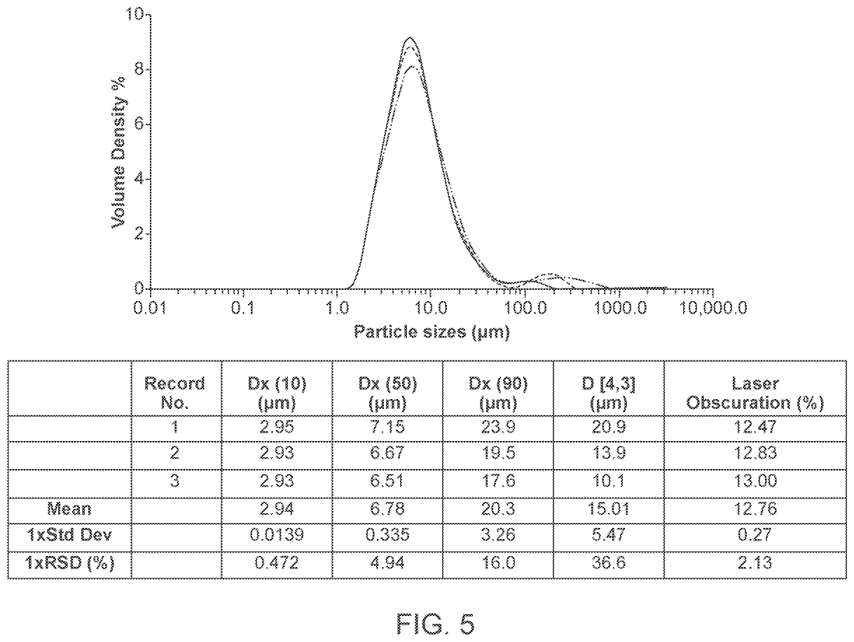

In some embodiments, Compound A citrate salt is a crystalline Compound A monocitrate monohydrate Form A salt and displays particle size distribution (PSD) substantially similar to that set forth in FIG. 5. The related particle size data of FIG. 5 sets forth in Table 2.

TABLE 2
Compound A Monocitrate Monohydrate Form
A Salt Particle Size Dispersity

| | | | | | | Laser |
|---|---|---|---|---|---|---|
| | Batch | Dx (10) | Dx (50) | Dx (90) | D [4, 3] | Obscuration |
| | No. | (μm) | (μm) | (μm) | (μm) | (%) |
| | 1 | 2.95 | 7.15 | 23.9 | 20.9 | 12.47 |
| | 2 | 2.93 | 6.67 | 19.5 | 13.9 | 12.83 |
| | 3 | 2.93 | 6.51 | 17.6 | 10.1 | 13.00 |
| Mean | | 2.94 | 6.78 | 20.3 | 15.01 | 12.76 |
| 1 × Std Dev | | 0.0139 | 0.335 | 3.26 | 5.47 | 0.27 |
| 1 × RSD | | 0.472 | 4.94 | 16.0 | 36.6 | 2.13 |
| (%) | | | | | | |

In some embodiments, Compound A citrate salt is a crystalline Compound A monocitrate monohydrate Form A salt and displays particle size distribution (PSD) that is about 95% similar to that set forth in FIG. 5.

In some embodiments, Compound A citrate salt is a crystalline Compound A monocitrate monohydrate Form A salt and displays particle size distribution (PSD) that is about 90% similar to that set forth in FIG. 5.

In some embodiments, Compound A citrate salt is a crystalline Compound A monocitrate monohydrate Form A salt and displays particle size distribution (PSD) that is about 85% similar to that set forth in FIG. 5.

In some embodiments, particle size Dx(10) of Compound A monocitrate monohydrate Form A salt ranges from about 1-100 μm, about 1-50 μm, about 1-25 μm, about 1-24 μm, about 1-23 μm, about 1-22 μm, about 1-21 μm, about 1-20 μm, about 1-19 μm, about 1-18 μm, about 1-17 μm, about 1-16 μm, about 1-15 μm, about 1-14 μm, about 1-13 μm, about 1-12 μm, about 1-11 μm, about 1-10 μm, about 1-9 μm, about 1-8 μm, about 1-7 μm, about 1-6 μm, about 1-5 μm, about 1-4 μm, about 1-2 μm, about 2-25 μm, about 2-24 μm, about 2-23 μm, about 2-22 μm, about 2-21 μm, about 2-20 μm, about 2-19 μm, about 2-18 μm, about 2-17 μm, about 2-16 μm, about 2-15 μm, about 2-14 μm, about 2-13 μm, about 2-12 μm, about 2-11 μm, about 2-10 μm, about 2-9 μm, about 2-8 μm, about 2-7 μm, about 2-6 μm, about 2-5 μm, about 2-4 μm, or about 2-3 μm.

In some embodiments, particle size Dx(10) of Compound A monocitrate monohydrate Form A salt is about 1 μm, about 2 μm, about 3 μm, about 4 μm, or about 5 μm.

In some embodiments, particle size Dx(50) of Compound A monocitrate monohydrate Form A salt ranges from about 1-100 μm, about 1-50 μm, about 1-25 μm, about 1-24 μm, about 1-23 μm, about 1-22 μm, about 1-21 μm, about 1-20 μm, about 1-19 μm, about 1-18 μm, about 1-17 μm, about 1-16 μm, about 1-15 μm, about 1-14 μm, about 1-13 μm, about 1-12 μm, about 1-11 μm, about 1-10 μm, about 1-9 μm, about 1-8 μm, about 1-7 μm, about 1-6 μm, about 1-5 μm, about 1-4 μm, about 1-2 μm, about 3-20 μm, about 3-19 μm, about 3-18 μm, about 3-17 μm, about 3-16 μm, about 3-15 μm, about 3-14 μm, about 3-13 μm, about 3-12 μm, about 3-11 μm, about 3-10 μm, about 3-9 μm, about 3-8 μm, about 3-7 μm, about 3-6 μm, about 3-5 μm, about 3-4 μm, about 5-25 μm, about 5-24 μm, about 5-23 μm, about 5-22 μm, about 5-21 μm, about 5-20 μm, about 5-19 μm, about 5-18 μm, about 5-17 μm, about 5-16 μm, about 5-15 μm, about 5-14 μm, about 5-13 μm, about 5-12 μm, about 5-11 μm, about 5-10 μm, about 5-9 μm, about 5-8 μm, about 5-7 μm, or about 5-6 μm.

In some embodiments, particle size Dx(50) of Compound A monocitrate monohydrate Form A salt is about 2 μm, about 3 μm, about 4 μm, about 5 μm, about 6 μm, about 7 μm, about 8 μm, about 9 μm, about 10 μm, about 11 μm, or about 12 μm.

In some embodiments, particle size Dx(90) of Compound A monocitrate monohydrate Form A salt ranges from about 1-100 μm, about 1-50 μm, about 1-25 μm, about 1-24 μm, about 1-23 μm, about 1-22 μm, about 1-21 μm, about 1-20 μm, about 1-19 μm, about 1-18 μm, about 1-17 μm, about 1-16 μm, about 1-15 μm, about 1-14 μm, about 1-13 μm, about 1-12 μm, about 1-11 μm, about 1-10 μm, about 1-9 μm, about 1-8 μm, about 1-7 μm, about 1-6 μm, about 1-5 μm, about 1-4 μm, about 1-2 μm, about 5-25 μm, about 5-24 μm, about 5-23 μm, about 5-22 μm, about 5-21 μm, about 5-20 μm, about 5-19 μm, about 5-18 μm, about 5-17 μm, about 5-16 μm, about 5-15 μm, about 5-14 μm, about 5-13 μm, about 5-12 μm, about 5-11 μm, about 5-10 μm, about 5-9 μm, about 5-8 μm, about 5-7 μm, about 5-6 μm, about 10-25 μm, about 10-24 μm, about 10-23 μm, about 10-22 μm, about 10-21 μm, about 10-20 μm, about 10-19 μm, about 10-18 μm, about 10-17 μm, about 10-16 μm, about 10-15 μm, about 10-14 μm, about 10-13 μm, about 10-12 μm, about 10-11 μm, about 15-25 μm, about 15-24 μm, about 15-23 μm, about 15-22 μm, about 15-21 μm, about 15-20 μm, about 15-19 μm, about 15-18 μm, about 15-17 μm, about 15-16 μm, about 20-25 μm, about 20-24 μm, about 20-23 μm, about 20-22 μm, or about 20-21 μm.

In some embodiments, particle size Dx(90) of Compound A monocitrate monohydrate Form A salt is about 15 μm, about 16 μm, about 17 μm, about 18 μm, about 19 μm, about 20 μm, about 21 μm, about 22 μm, about 23 μm, about 24 μm, or about 25 μm.

In some embodiments, Compound A monocitrate monohydrate Form A salt has an average particle size ranging from about 1-100 μm, about 1-50 μm, about 1-25 μm, about 1-24 μm, about 1-23 μm, about 1-22 μm, about 1-21 μm, about 1-20 μm, about 1-19 μm, about 1-18 μm, about 1-17 μm, about 1-16 μm, about 1-15 μm, about 1-14 μm, about 1-13 μm, about 1-12 μm, about 1-11 μm, about 1-10 μm, about 1-9 μm, about 1-8 μm, about 1-7 μm, about 1-6 μm, about 1-5 μm, about 1-4 μm, about 1-2 μm, about 5-25 μm, about 5-24 μm, about 5-23 μm, about 5-22 μm, about 5-21 μm, about 5-20 μm, about 5-19 μm, about 5-18 μm, about 5-17 μm, about 5-16 μm, about 5-15 μm, about 5-14 μm, about 5-13 μm, about 5-12 μm, about 5-11 μm, about 5-10 μm, about 5-9 μm, about 5-8 μm, about 5-7 μm, about 5-6 μm, about 10-25 μm, about 10-24 μm, about 10-23 μm, about 10-22 μm, about 10-21 μm, about 10-20 μm, about 10-19 μm, about 10-18 μm, about 10-17 μm, about 10-16 μm, about 10-15 μm, about 10-14 μm, about 10-13 μm, about 10-12 μm, about 10-11 μm, about 15-25 μm, about 15-24 μm, about 15-23 μm, about 15-22 μm, about 15-21 μm, about 15-20 μm, about 15-19 μm, about 15-18 μm, about 15-17 μm, about 15-16 μm, about 20-25 μm, about 20-24 μm, about 20-23 μm, about 20-22 μm, or about 20-21 μm.

In some embodiments, the average particle size of Compound A monocitrate monohydrate Form A salt is about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, or about 25 μm.

In some embodiments, Compound A monocitrate monohydrate Form A salt has a median particle size ranging from about 1-100 μm, about 1-50 μm, about 1-25 μm, about 1-24 μm, about 1-23 μm, about 1-22 μm, about 1-21 μm, about 1-20 μm, about 1-19 μm, about 1-18 μm, about 1-17 μm, about 1-16 μm, about 1-15 μm, about 1-14 μm, about 1-13 μm, about 1-12 μm, about 1-11 μm, about 1-10 μm, about 1-9 μm, about 1-8 μm, about 1-7 μm, about 1-6 μm, about 1-5 μm, about 1-4 μm, about 1-2 μm, about 5-25 μm, about 5-24 μm, about 5-23 μm, about 5-22 μm, about 5-21 μm, about 5-20 μm, about 5-19 μm, about 5-18 μm, about 5-17 μm, about 5-16 μm, about 5-15 μm, about 5-14 μm, about 5-13 μm, about 5-12 μm, about 5-11 μm, about 5-10 μm, about 5-9 μm, about 5-8 μm, about 5-7 μm, about 5-6 μm, about 10-25 μm, about 10-24 μm, about 10-23 μm, about 10-22 μm, about 10-21 μm, about 10-20 μm, about 10-19 μm, about 10-18 μm, about 10-17 μm, about 10-16 μm, about 10-15 μm, about 10-14 μm, about 10-13 μm, about 10-12 μm, about 10-11 μm, about 15-25 μm, about 15-24 μm, about 15-23 μm, about 15-22 μm, about 15-21 μm, about 15-20 μm, about 15-19 μm, about 15-18 μm, about 15-17 μm, about 15-16 μm, about 20-25 μm, about 20-24 μm, about 20-23 μm, about 20-22 μm, or about 20-21 μm.

In some embodiments, the median particle size of Compound A monocitrate monohydrate Form A salt is about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, or about 25 μm.

Compound A monocitrate monohydrate has at least two advantageously and unexpected characteristics. Both alone and in comparison, with Compound A salts known in the art (e.g. Compound A fumarate salts). First, the fumarate counterion reacts with the pyrimidine ring of Compound A resulting in degradation; whereas the citrate counterion does not react with the pyrimidine ring of Compound A. Solution stability studies at accelerated conditions confirmed the absence of this type of degradation. Second, in the manufacturing process, Compound A fumarate requires a separate slurrying step which results in a solvate with an organic solvent. Compound A monocitrate monohydrate salt is isolated directly from ethanol:water, no slurrying step is required. Accordingly, this manufacturing process does not require multiple steps and results in increased yields (no losses during the slurrying step). Additionally, the Compound A monocitrate forms a monohydrate instead of a solvate with one or more organic molecules.

### Compound A Citrate Salt (Form B)

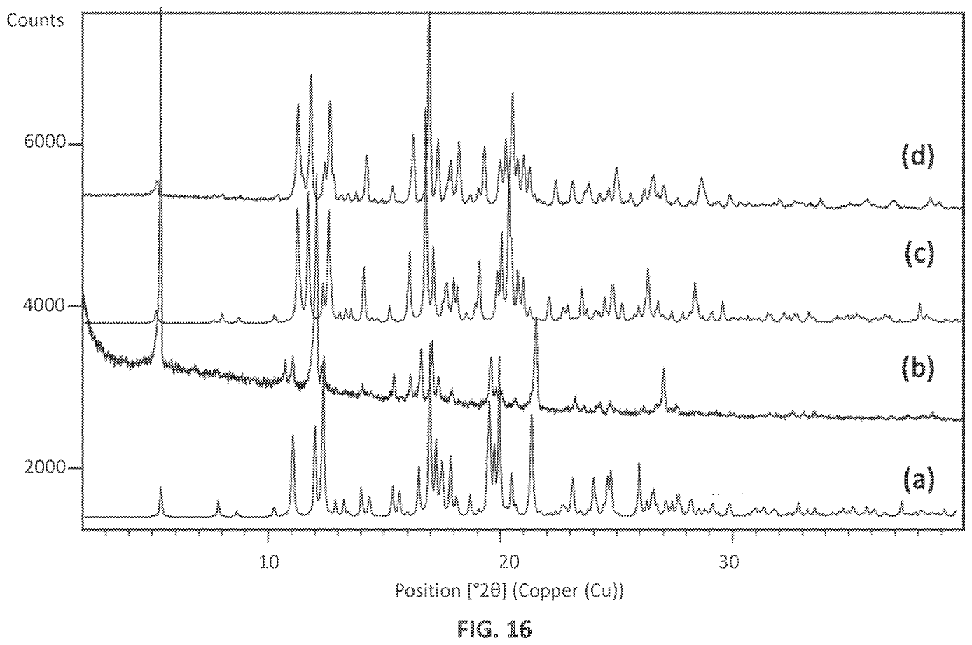

In some embodiments, the Compound A citrate salt is a crystalline polymorphic Form B characterized by XRPD pattern as set forth in FIG. 16 (trace a).

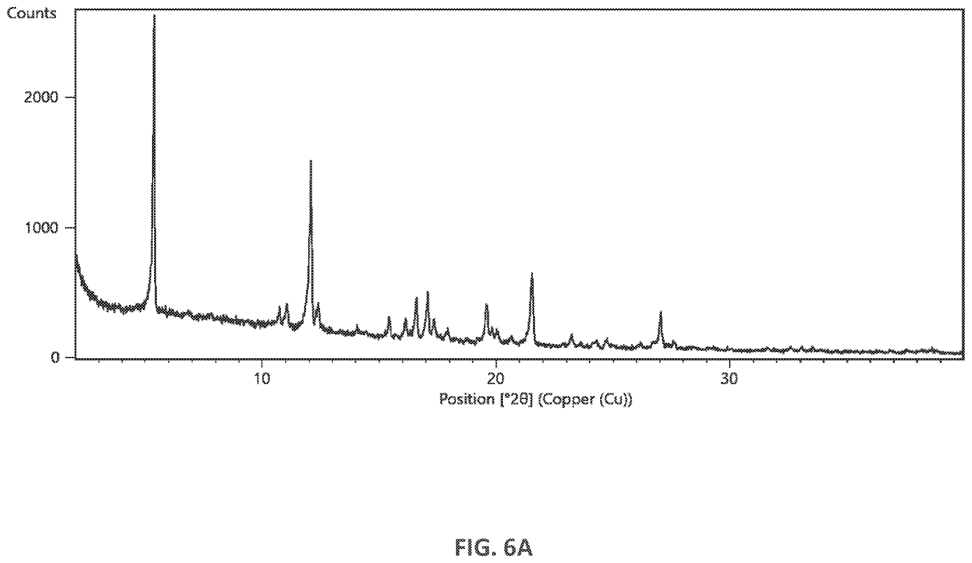

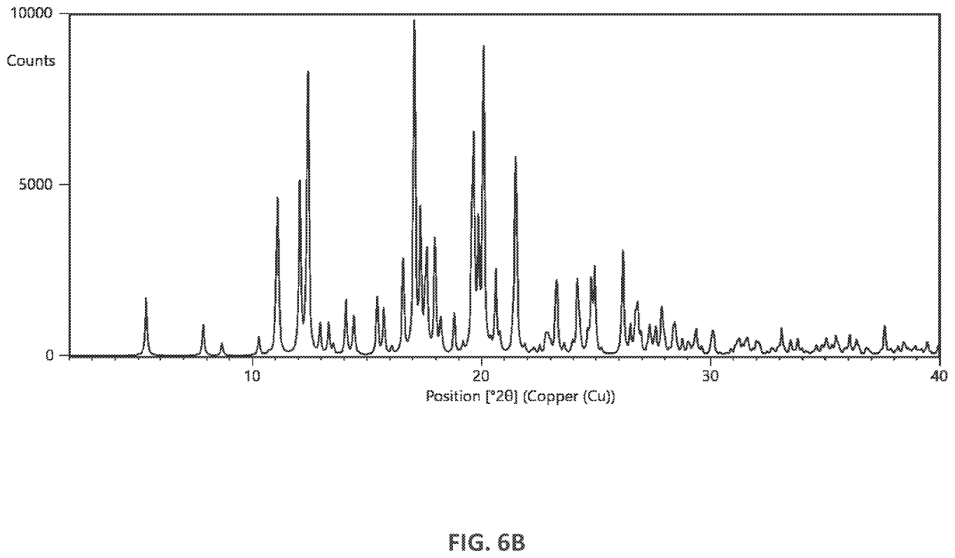

In some embodiments, the Compound A citrate salt is a crystalline polymorphic Form B characterized by XRPD pattern as set forth in FIG. 6A.

In some embodiments, the Compound A citrate salt is a crystalline polymorphic Form B characterized by the PXRD signals in Table 3A.

TABLE 3A
Compound A salt is a crystalline polymorphic Form
B PXRD (±0.2 °2θ; ±0.1 °2θ;
or ±0.0 °2θ; Cu Kα1 radiation).

| Pos. [°2θ] | Rel. Int. [%] |
|---|---|
| 5.4 | 100 |
| 10.7 | 6.84 |
| 11.1 | 7.86 |
| 12.1 | 56.76 |
| 12.4 | 9.17 |
| 15.4 | 5.76 |
| 16.1 | 6.28 |
| 16.6 | 13.06 |
| 17.1 | 15.85 |
| 17.3 | 6.73 |
| 17.9 | 3.29 |
| 19.6 | 12.73 |
| 19.8 | 4.67 |
| 20.0 | 3.68 |
| 21.5 | 22.77 |
| 23.2 | 3.22 |
| 24.3 | 1.99 |
| 24.7 | 2.61 |
| 27.0 | 12.51 |
| 27.6 | 1.95 |
| 32.6 | 1.47 |
| 33.1 | 1.44 |
| 33.5 | 1.24 |
| 38.6 | 1 |

In some embodiments, the Compound A citrate salt is a crystalline polymorphic Form B characterized by PXRD spectrum substantially similar to that shown in FIG. 6A.

In some embodiments, the Compound A citrate salt is a crystalline polymorphic Form B characterized by PXRD spectrum that is about 95% similar to that shown in FIG. 6A.

In some embodiments, the Compound A citrate salt is a crystalline polymorphic Form B characterized by PXRD spectrum that is about 90% similar to that shown in FIG. 6A.

In some embodiments, the Compound A citrate salt is a crystalline polymorphic Form B characterized by PXRD spectrum that is about 85% similar to that shown in FIG. 6A.

In some embodiments, the Compound A citrate salt is a crystalline polymorphic Form B characterized by one, two, three, four, five, six, seven, eight, nine, ten, eleven, twelve, thirteen, fourteen, fifteen, sixteen, seventeen, eighteen, or nineteen PXRD signals selected from those set forth in Table 3A.

In some embodiments, the crystalline Compound A salt is a crystalline polymorphic Form B characterized by a PXRD signal at 5.4°2θ (±0.2°2θ; ±0.1°2θ; or ±0.0°2θ; Cu Kα1 radiation).

In some embodiments, the Compound A salt is a crystalline polymorphic Form B characterized by PXRD signals at 5.4°2θ and 12.1°2θ (±0.2°2θ; ±0.1°2θ; or ±0.0°2θ; Cu Kα1 radiation).

In some embodiments, the Compound A salt is a crystalline polymorphic Form B characterized by two, or three PXRD signals selected from the group consisting of 5.4°2θ, 12.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by PXRD signals at 5.4°2θ, 12.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 5.4°2θ, 12.1°2θ, 17.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by PXRD signals at 5.4°2θ, 12.1°2θ, 17.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 5.4°2θ, 12.1°2θ, 16.6°2θ, 17.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by PXRD signals at 5.4°2θ, 12.1°2θ, 16.6°2θ, 17.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 5.4°2θ, 12.1°2θ, 16.6°2θ, 17.1°2θ, 19.6°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by PXRD signals at 5.4°2θ, 12.1°2θ, 16.6°2θ, 17.1°2θ, 19.6°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 5.4°2θ, 12.1°2θ, 16.6°2θ, 17.1°2θ, 19.6°2θ, 21.5°2θ, and 27.0°2θ (±0.2°2θ; ±0.1°2θ; or ±0.0°2θ; Cu Kα1 radiation).

In some embodiments, the Compound A salt is a crystalline polymorphic Form B characterized by PXRD signals at 5.4°2θ, 12.1°2θ, 16.6°2θ, 17.1°2θ, 19.6°2θ, 21.5°2θ, and 27.0°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 5.4°2θ, 12.1°2θ, 12.4°2θ, 16.6°2θ, 17.1°2θ, 19.6°2θ, 21.5°2θ, and 27.0°2θ (±0.2°2θ; ±0.1°2θ; or ±0.0°2θ; Cu Kα1 radiation).

In some embodiments, the Compound A salt is a crystalline polymorphic Form B characterized by PXRD signals at 5.4°2θ, 12.1°2θ, 12.4°2θ, 16.6°2θ, 17.1°2θ, 19.6°2θ, 21.5°2θ, and 27.0°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 5.4°2θ, 11.1°2θ, 12.1°2θ, 12.4°2θ, 16.6°2θ, 17.1°2θ, 19.6°2θ, 21.5°2θ, and 27.0°2θ (±0.2°2θ; ±0.1°2θ; or ±0.0°2θ; Cu Kα1 radiation).

In some embodiments, the Compound A salt is a crystalline polymorphic Form B characterized by PXRD signals at 5.4°2θ, 11.1°2θ, 12.1°2θ, 12.4°2θ, 16.6°2θ, 17.1°2θ, 19.6°2θ, 21.5°2θ, and 27.0°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 5.4°2θ, 10.7°2θ, 11.1°2θ, 12.1°2θ, 12.4°2θ, 16.6°2θ, 17.1°2θ, 19.6°2θ, 21.5°2θ, and 27.0°2θ (±0.2°2θ; ±0.1°2θ; or ±0.0°2θ; Cu Kα1 radiation).

In some embodiments, the Compound A salt is a crystalline polymorphic Form B characterized by PXRD signals at 5.4°2θ, 10.7°2θ, 11.1°2θ, 12.1°2θ, 12.4°2θ, 16.6°2θ, 17.1°2θ, 19.6°2θ, 21.5°2θ, and 27.0°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 5.4°2θ, 10.7°2θ, 11.1°2θ, 12.1°2θ, 12.4°2θ, 16.6°2θ, 17.1°2θ, 17.3°2θ, 19.6°2θ, 21.5°2θ, and 27.0°2θ (±0.2°2θ; ±0.1°2θ; or ±0.0°2θ; Cu Kα1 radiation).

In some embodiments, the Compound A salt is a crystalline polymorphic Form B characterized by PXRD signals at 5.4°2θ, 10.7°2θ, 11.1°2θ, 12.1°2θ, 12.4°2θ, 16.6°2θ, 17.1°2θ, 17.3°2θ, 19.6°2θ, 21.5°2θ, and 27.0°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 5.4°2θ, 10.7°2θ, 11.1°2θ, 12.1°2θ, 12.4°2θ, 16.1°2θ, 16.6°2θ, 17.1°2θ, 17.3°2θ, 19.6°2θ, 21.5°2θ, and 27.0°2θ (±0.2°2θ; ±0.1°2θ; or ±0.0°2θ; Cu Kα1 radiation).

In some embodiments, the Compound A salt is a crystalline polymorphic Form B characterized by PXRD signals at 5.4°2θ, 10.7°2θ, 11.1°2θ, 12.1°2θ, 12.4°2θ, 16.1°2θ, 16.6°2θ, 17.1°2θ, 17.3°2θ, 19.6°2θ, 21.5°2θ, and 27.0°2θ (±0.2°2θ; ±0.1°2θ; or ±0.0°2θ; Cu Kα1 radiation).

In some embodiments, the Compound A salt is a crystalline polymorphic Form B characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four XRPD signals selected from those set forth in Table 3A.

In some embodiments, the Compound A salt is a crystalline polymorphic Form B characterized by one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three, or twenty-four XRPD signals selected from those set forth in Table 3A.

In some embodiments, the Compound A salt is a crystalline polymorphic Form B characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four XRPD signals selected from 5.4°2θ, 10.7 20, 11.1°2θ, 12.1°2θ, 12.4°2θ, 15.4°2θ, 16.1°2θ, 16.6°2θ, 17.1°2θ, 17.3°2θ, 17.9°2θ, 19.6°2θ, 19.8°2θ, 20.0°2θ, 21.5°2θ, 23.2°2θ, 24.3°2θ, 24.7°2θ, 27.0°2θ, 27.6°2θ, 32.6°2θ, 33.1°2θ, 33.5°2θ, and 38.6°2θ (±0.2°2θ; ±0.1°2θ; or ±0.0°2θ; Cu Kα1 radiation).

In some embodiments, the Compound A salt is a crystalline polymorphic Form B characterized by one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three, or twenty-four XRPD signals selected from 5.4°2θ, 10.7 20, 11.1°2θ, 12.1°2θ, 12.4°2θ, 15.4°2θ, 16.1°2θ, 16.6°2θ, 17.1°2θ, 17.3°2θ, 17.9°2θ, 19.6°2θ, 19.8°2θ, 20.0°2θ, 21.5°2θ, 23.2°2θ, 24.3°2θ, 24.7°2θ, 27.0°2θ, 27.6°2θ, 32.6°2θ, 33.1°2θ, 33.5°2θ, and 38.6°2θ (±0.2°2θ; ±0.1°2θ; or ±0.0°2θ; Cu Kα1 radiation). (±0.2°2θ; ±0.1°2θ; or ±0.0°2θ; Cu Kα1 radiation).

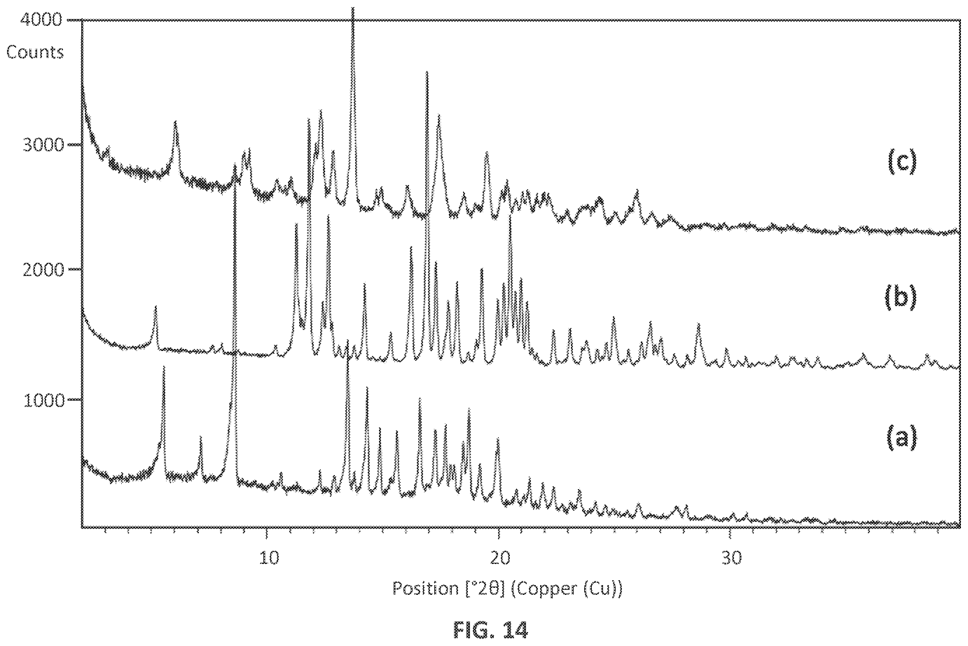

In some embodiments, the Compound A citrate salt is a crystalline polymorphic Form B characterized by PXRD spectrum substantially similar to that shown in FIG. 14 (trace a).

In some embodiments, the Compound A citrate salt is a crystalline polymorphic Form B characterized by PXRD spectrum that is about 95% similar to that shown in FIG. 14 (trace a).

In some embodiments, the Compound A citrate salt is a crystalline polymorphic Form B characterized by PXRD spectrum that is about 90% similar to that shown in FIG. 14 (trace a).

In some embodiments, the Compound A citrate salt is a crystalline polymorphic Form B characterized by PXRD spectrum that is about 85% similar to that shown in FIG. 14 (trace a).

In some embodiments, the Compound A citrate salt is a crystalline polymorphic Form B characterized by the PXRD signals in Table 3B.

TABLE 3B
Compound A salt is a crystalline polymorphic Form B PXRD, simulated
(±0.2 °2θ; ±0.1 °2θ; or ±0.0 °2θ;
Cu Kα1 radiation).

| Pos. [°2θ] | Rel. Int. [%] |
|---|---|
| 5.3 | 17.03 |
| 7.8 | 9.17 |
| 8.7 | 3.53 |
| 10.3 | 5.59 |
| 11.1 | 46.91 |
| 12.1 | 52.71 |
| 12.4 | 85.29 |
| 12.9 | 9.58 |
| 13.3 | 9.86 |
| 13.5 | 3.5 |
| 14.1 | 16.79 |
| 14.4 | 11.74 |
| 15.4 | 17.73 |
| 15.7 | 14.14 |
| 16.6 | 29.09 |
| 17.1 | 100 |
| 17.3 | 44.23 |
| 17.6 | 32.05 |
| 18.0 | 34.86 |
| 18.2 | 11.41 |
| 18.8 | 12.53 |
| 19.2 | 4.25 |
| 19.6 | 66.62 |
| 19.9 | 42.02 |
| 20.1 | 91.66 |
| 20.6 | 25.82 |
| 21.5 | 58.71 |
| 21.9 | 3.49 |
| 22.5 | 3.17 |
| 22.8 | 6.57 |
| 23.3 | 22.34 |
| 23.6 | 3.64 |
| 24.0 | 4.59 |
| 24.2 | 22.64 |
| 24.8 | 23.44 |
| 24.9 | 26.88 |
| 26.2 | 31.46 |
| 26.5 | 9.52 |
| 26.7 | 13.06 |
| 26.8 | 15.8 |
| 27.3 | 9.11 |
| 27.6 | 8.58 |
| 27.9 | 14.55 |
| 28.4 | 9.65 |
| 28.8 | 5.07 |
| 29.0 | 4.21 |
| 29.4 | 7.87 |
| 30.1 | 7.47 |
| 30.9 | 1.96 |
| 31.2 | 5 |
| 31.6 | 5.22 |
| 32.1 | 4.05 |
| 33.1 | 8.16 |
| 33.5 | 4.48 |
| 33.8 | 5.03 |
| 34.6 | 2.89 |
| 35.1 | 5.1 |
| 35.5 | 5.79 |
| 36.1 | 6.21 |
| 36.4 | 4.58 |
| 36.8 | 2.26 |
| 37.6 | 8.76 |
| 38.4 | 3.92 |
| 39.5 | 3.99 |

In some embodiments, the Compound A citrate salt is a crystalline polymorphic Form B characterized by PXRD spectrum substantially similar to that shown in FIG. 6B.

In some embodiments, the Compound A citrate salt is a crystalline polymorphic Form B characterized by PXRD spectrum that is about 95% similar to that shown in FIG. 6B.

In some embodiments, the Compound A citrate salt is a crystalline polymorphic Form B characterized by PXRD spectrum that is about 90% similar to that shown in FIG. 6B.

In some embodiments, the Compound A citrate salt is a crystalline polymorphic Form B characterized by PXRD spectrum that is about 85% similar to that shown in FIG. 6B.

In some embodiments, the Compound A citrate salt is a crystalline polymorphic Form B characterized by one, two, three, four, five, six, seven, eight, nine, ten, eleven, twelve, thirteen, fourteen, fifteen, sixteen, seventeen, eighteen, or nineteen PXRD signals selected from those set forth in Table 3B.

In some embodiments, the crystalline Compound A salt is a crystalline polymorphic Form B characterized by a PXRD signal at 17.1°2θ (±0.2°2θ; ±0.1°2θ; or ±0.0°2θ; Cu Kα1 radiation).

In some embodiments, the Compound A salt is a crystalline polymorphic Form B characterized by PXRD signals at 17.1°2θ, and 20.1°2θ (±0.2°2θ; ±0.1°2θ; or ±0.0°2θ; Cu Kα1 radiation).

In some embodiments, the Compound A salt is a crystalline polymorphic Form B characterized by two, or three PXRD signals selected from the group consisting of 12.4°2θ, 17.1°2θ, and 20.1°2θ (±0.2°2θ; ±0.1°2θ; or ±0.0°2θ; Cu Kα1 radiation).

In some embodiments, the Compound A salt is a crystalline polymorphic Form B characterized by PXRD signals 12.4°2θ, 17.1°2θ, and 20.1°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 12.4°2θ, 17.1°2θ, 19.6°2θ, and 20.1°2θ (±0.2°2θ; ±0.1°2θ; or ±0.0°2θ; Cu Kα1 radiation).

In some embodiments, the Compound A salt is a crystalline polymorphic Form B characterized by PXRD signals at 12.4°2θ, 17.1°2θ, 19.6°2θ, and 20.1°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 12.4°2θ, 17.1°2θ, 19.6°2θ, 20.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by PXRD signals at 12.4°2θ, 17.1°2θ, 19.6°2θ, 20.1°2θ, and 21.5 020°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 12.1°2θ, 12.4°2θ, 17.1°2θ, 19.6°2θ, 20.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by PXRD signals at 12.1°2θ, 12.4°2θ, 17.1°2θ, 19.6°2θ, 20.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 11.1°2θ, 12.1°2θ, 12.4°2θ, 17.1°2θ, 19.6°2θ, 20.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by PXRD signals at 11.1°2θ, 12.1°2θ, 12.4°2θ, 17.1°2θ, 19.6°2θ, 20.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 11.1°2θ, 12.1°2θ, 12.4°2θ, 17.1°2θ, 17.3°2θ, 19.6°2θ, 20.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by PXRD signals at 11.1°2θ, 12.1°2θ, 12.4°2θ, 17.1°2θ, 17.3°2θ, 19.6°2θ, 20.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 11.1°2θ, 12.1°2θ, 12.4°2θ, 17.1°2θ, 17.3°2θ, 19.6°2θ, 19.9°2θ, 20.1°2θ, and 21.5°2θ °2θ (±0.2°2θ; ±0.1°2θ; or ±0.0°2θ; Cu Kα1 radiation).

In some embodiments, the Compound A salt is a crystalline polymorphic Form B characterized by PXRD signals at 11.1°2θ, 12.1°2θ, 12.4°2θ, 17.1°2θ, 17.3°2θ, 19.6°2θ, 19.9°2θ, 20.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 11.1°2θ, 12.1°2θ, 12.4°2θ, 17.1°2θ, 17.3°2θ, 18.0°2θ, 19.6°2θ, 19.9°2θ, 20.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by PXRD signals at 11.1°2θ, 12.1°2θ, 12.4°2θ, 17.1°2θ, 17.3°2θ, 18.0°2θ, 19.6°2θ, 19.9°2θ, 20.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by two or more, or three or more PXRD signals selected from the group consisting of 11.1°2θ, 12.1°2θ, 12.4°2θ, 17.1°2θ, 17.3°2θ, 17.6°2θ, 18.0°2θ, 19.6°2θ, 19.9°2θ, 20.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by PXRD signals at 11.1°2θ, 12.1°2θ, 12.4°2θ, 17.1°2θ, 17.3°2θ, 17.6°2θ, 18.0°2θ, 19.6°2θ, 19.9°2θ, 20.1°2θ, and 21.5°2θ (±0.2°2θ; ±0.1°2θ; or ±0.0°2θ; Cu Kα1 radiation).

In some embodiments, the Compound A salt is a crystalline polymorphic Form B characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-one, fifty-two, fifty-three, fifty-four, fifty-five, fifty-six, fifty-seven, fifty-eight, fifty-nine, sixty, sixty-one, sixty-two, sixty-three, or sixty-four XRPD signals selected from those set forth in Table 3B.

In some embodiments, the Compound A salt is a crystalline polymorphic Form B characterized by one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more, thirty-nine or more, forty or more, forty-one or more, forty-two or more, forty-three or more, forty-four or more, forty-five or more, forty-six or more, forty-seven or more, forty-eight or more, forty-nine or more, fifty or more, fifty-one or more, fifty-two or more, fifty-three or more, fifty-four or more, fifty-five or more, fifty-six or more, fifty-seven or more, fifty-eight or more, fifty-nine or more, sixty or more, sixty-one or more, sixty-two or more, sixty-three, or sixty-four XRPD signals selected from those set forth in Table 3B.

In some embodiments, the Compound A salt is a crystalline polymorphic Form B characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-one, fifty-two, fifty-three, fifty-four, fifty-five, fifty-six, fifty-seven, fifty-eight, fifty-nine, sixty, sixty-one, sixty-two, sixty-three, or sixty-four XRPD signals selected from 5.3°2θ, 7.8°2θ, 8.7°2θ, 10.3°2θ, 11.1°2θ, 12.1°2θ, 12.4°2θ, 12.9°2θ, 13.3°2θ, 13.5°2θ, 14.1°2θ, 14.4°2θ, 15.4°2θ, 15.7°2θ, 16.6°2θ, 17.1°2θ, 17.3°2θ, 17.6°2θ, 18.0°2θ, 18.2°2θ, 18.8°2θ, 19.2°2θ, 19.6°2θ, 19.9°2θ, 20.1°2θ, 20.6°2θ, 21.5°2θ, 21.9°2θ, 22.5°2θ, 22.8°2θ, 23.3°2θ, 23.6°2θ, 24.0°2θ, 24.2°2θ, 24.8°2θ, 24.9°2θ, 26.2°2θ, 26.5°2θ, 26.7°2θ, 26.8°2θ, 27.3°2θ, 27.6°2θ, 27.9°2θ, 28.4°2θ, 28.8°2θ, 29.0°2θ, 29.4°2θ, 30.1°2θ, 30.9°2θ, 31.2°2θ, 31.6°2θ, 32.1°2θ, 33.1°2θ, 33.5°2θ, 33.8°2θ, 34.6°2θ, 35.1°2θ, 35.5°2θ, 36.1°2θ, 36.4°2θ, 36.8°2θ, 37.6°2θ, 38.4°2θ, and 39.5°2θ (±0.2°2θ; ±0.1°2θ; or ±0.0°2θ; Cu Kα1 radiation).

In some embodiments, the Compound A salt is a crystalline polymorphic Form B characterized by one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more, thirty-nine or more, forty or more, forty-one or more, forty-two or more, forty-three or more, forty-four or more, forty-five or more, forty-six or more, forty-seven or more, forty-eight or more, forty-nine or more, fifty or more, fifty-one or more, fifty-two or more, fifty-three or more, fifty-four or more, fifty-five or more, fifty-six or more, fifty-seven or more, fifty-eight or more, fifty-nine or more, sixty or more, sixty-one or more, sixty-two or more, sixty-three, or sixty-four XRPD signals selected from 5.3°2θ, 7.8°2θ, 8.7°2θ, 10.3°2θ, 11.1°2θ, 12.1°2θ, 12.4°2θ, 12.9°2θ, 13.3°2θ, 13.5°2θ, 14.1°2θ, 14.4°2θ, 15.4°2θ, 15.7°2θ, 16.6°2θ, 17.1°2θ, 17.3°2θ, 17.6°2θ, 18.0°2θ, 18.2°2θ, 18.8°2θ, 19.2°2θ, 19.6°2θ, 19.9°2θ, 20.1°2θ, 20.6°2θ, 21.5°2θ, 21.9°2θ, 22.5°2θ, 22.8°2θ, 23.3°2θ, 23.6°2θ, 24.0°2θ, 24.2°2θ, 24.8°2θ, 24.9°2θ, 26.2°2θ, 26.5°2θ, 26.7°2θ, 26.8°2θ, 27.3°2θ, 27.6°2θ, 27.9°2θ, 28.4°2θ, 28.8°2θ, 29.0°2θ, 29.4°2θ, 30.1°2θ, 30.9°2θ, 31.2°2θ, 31.6°2θ, 32.1°2θ, 33.1°2θ, 33.5°2θ, 33.8°2θ, 34.6°2θ, 35.1°2θ, 35.5°2θ, 36.1°2θ, 36.4°2θ, 36.8°2θ, 37.6°2θ, 38.4°2θ, and 39.5°2θ (±0.2°2θ; ±0.1°2θ; or ±0.0°2θ; Cu Kα1 radiation). (±0.2°2θ; ±0.1°2θ; or ±0.0°2θ; Cu Kα1 radiation).

In some embodiments, Form B is unstable form and converts to Form A upon drying. In some embodiments, the conversion to more stable Form A is so rapid that it changes during the analysis.

In some embodiments, Form B is isolated from either MeOH or EtOH and readily converts to Form A upon drying of the wet cake.

### Compound A Citrate Salt (Form C)

In some embodiments, Form C is observed when using acetonitrile solvent.

In some embodiments, Form C is unstable and converts to Form A in presence of water.

In some embodiments, Form C is isolated from acetonitrile solvent.

In some embodiments, the Compound A citrate salt is a crystalline polymorphic Form C characterized by the PXRD signals in Table 4.

In some embodiments, the Compound A citrate salt is a crystalline polymorphic Form C characterized by XRPD pattern as set forth in FIG. 14 (trace c).

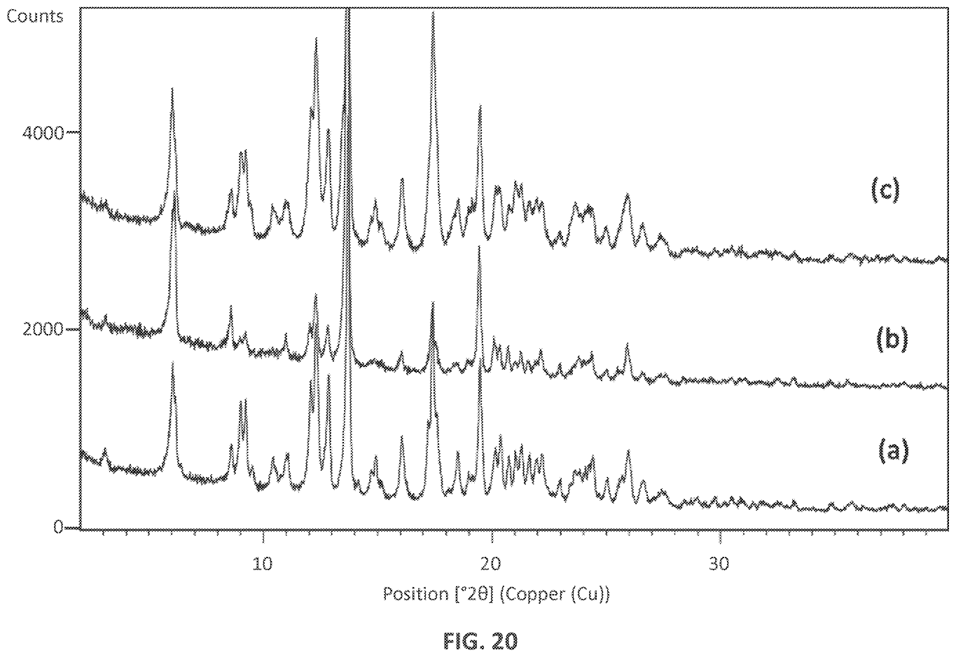

In some embodiments, the Compound A citrate salt is a crystalline polymorphic Form C characterized by XRPD pattern as set forth in FIG. 20.

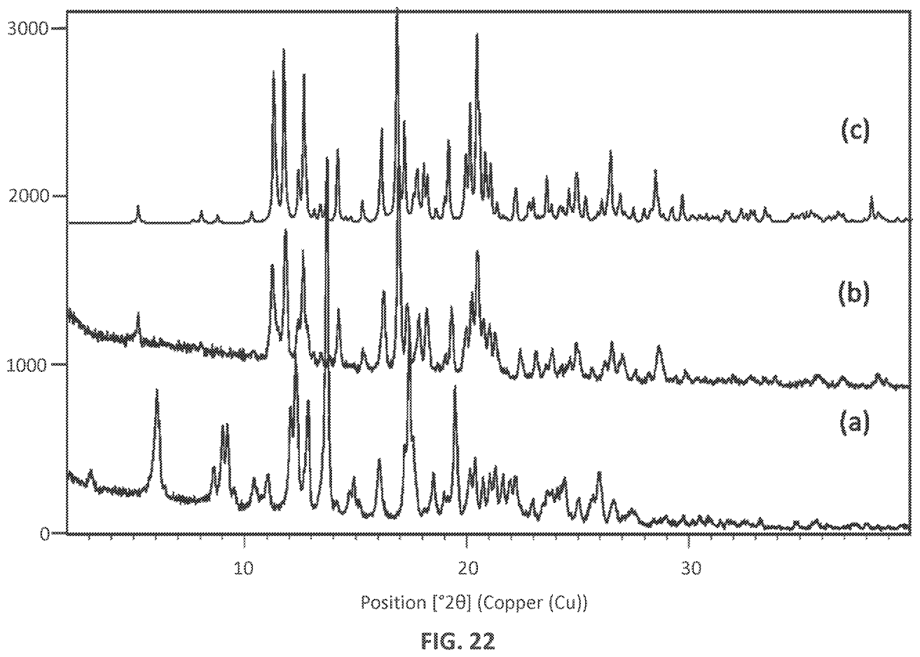

In some embodiments, the Compound A citrate salt is a crystalline polymorphic Form C characterized XRPD before DVS as shown in FIG. 22 (trace a).

In some embodiments, the Compound A citrate salt is a crystalline polymorphic Form C characterized XRPD after DVS as shown in FIG. 22 (trace b).

In some embodiments, the Compound A citrate salt is a crystalline polymorphic Form C characterized by XRPD pattern substantially similar to that shown in FIG. 14 (trace c).

In some embodiments, the Compound A citrate salt is a crystalline polymorphic Form C characterized by XRPD pattern substantially similar to that shown in FIG. 20.

In some embodiments, the Compound A citrate salt is a crystalline polymorphic Form C characterized XRPD before DVS substantially similar to that shown in FIG. 22 (trace a).

In some embodiments, the Compound A citrate salt is a crystalline polymorphic Form C characterized XRPD after DVS substantially similar to that shown in FIG. 22 (trace b).

In some embodiments, the Compound A citrate salt is a crystalline polymorphic Form C characterized by XRPD pattern that is about 95% similar to that shown in FIG. 14 (trace c).

In some embodiments, the Compound A citrate salt is a crystalline polymorphic Form C characterized by XRPD pattern that is about 95% similar to that shown in FIG. 20.

In some embodiments, the Compound A citrate salt is a crystalline polymorphic Form C characterized by XRPD pattern that is about 90% similar to that shown in FIG. 14 (trace c).

In some embodiments, the Compound A citrate salt is a crystalline polymorphic Form C characterized by XRPD pattern that is about 85% similar to that shown in FIG. 20.

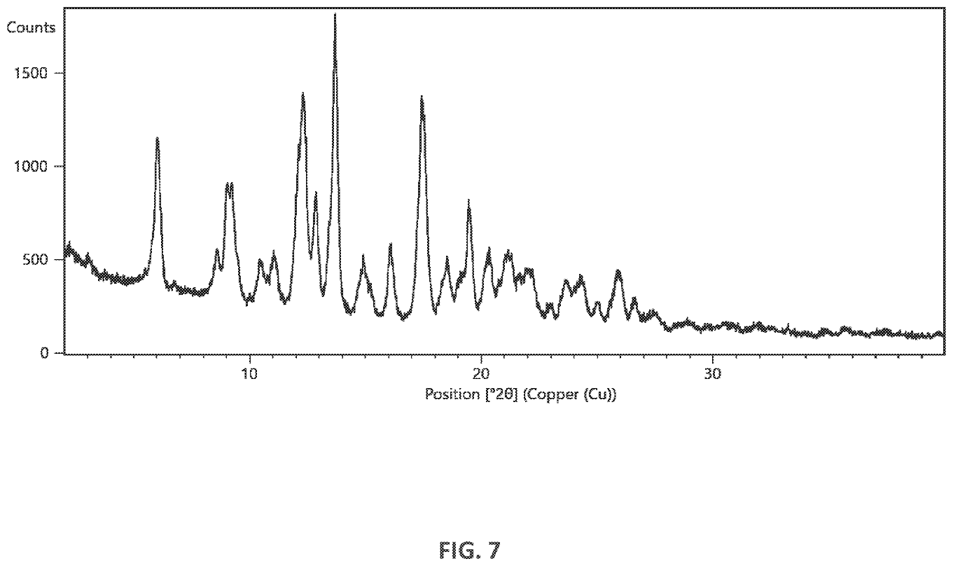

In some embodiments, the Compound A citrate salt is a crystalline polymorphic Form C characterized by XRPD pattern as set forth in FIG. 7.

In some embodiments, the Compound A citrate salt is a crystalline polymorphic Form C characterized XRPD after DVS substantially similar to that shown in FIG. 7.

In some embodiments, the Compound A citrate salt is a crystalline polymorphic Form C characterized by XRPD pattern that is about 95% similar to that shown in FIG. 7.

In some embodiments, the Compound A citrate salt is a crystalline polymorphic Form C characterized by XRPD pattern that is about 90% similar to that shown in FIG. 7.

In some embodiments, the Compound A citrate salt is a crystalline polymorphic Form C characterized by XRPD pattern that is about 85% similar to that shown in FIG. 7.

In some embodiments, the Compound A citrate salt is a crystalline polymorphic Form C characterized by one, two, three, four, five, six, seven, eight, nine, ten, eleven, twelve, thirteen, fourteen, fifteen, sixteen, seventeen, eighteen, or nineteen PXRD signals selected from those set forth in Table 4.

TABLE 4
Compound A salt is a crystalline polymorphic Form
C PXRD (±0.2 °2θ; ±0.1 °2θ;
or ±0.0 °2θ; Cu Kα1 radiation).

| Pos. [°2θ] | Rel. Int. [%] |
|---|---|
| 6.1 | 60.27 |
| 8.6 | 26.53 |
| 9.0 | 47.12 |
| 9.2 | 46.86 |
| 10.4 | 23.47 |
| 11.1 | 25.25 |
| 12.1 | 52.06 |
| 12.3 | 75.41 |
| 12.9 | 44.83 |
| 13.7 | 100 |
| 14.9 | 23.41 |
| 16.1 | 27.67 |
| 17.4 | 73.58 |
| 18.5 | 24.28 |
| 19.5 | 41.03 |
| 20.3 | 24.92 |
| 21.1 | 24.91 |
| 22.0 | 20.24 |
| 23.0 | 9.21 |
| 23.6 | 17.14 |
| 24.3 | 17.5 |
| 25.0 | 10.5 |
| 25.9 | 19.98 |
| 26.6 | 11.32 |
| 27.5 | 6.85 |

In some embodiments, the crystalline Compound A salt is a crystalline polymorphic Form C characterized by a PXRD signal at 13.7°2θ (±0.2°2θ; ±0.1°2θ; or ±0.0°2θ; Cu Kα1 radiation).

In some embodiments, the Compound A salt is a crystalline polymorphic Form C characterized by PXRD signals at 12.3°2θ and 13.7°2θ (±0.2°2θ; ±0.1°2θ; or ±0.0°2θ; Cu Kα1 radiation).

In some embodiments, the Compound A salt is a crystalline polymorphic Form C characterized by two, or three PXRD signals selected from the group consisting of 12.3°2θ, 13.7°2θ, and 17.4°2θ (±0.2°2θ; ±0.1°2θ; or ±0.0°2θ; Cu Kα1 radiation).

In some embodiments, the Compound A salt is a crystalline polymorphic Form C characterized by PXRD signals 12.3°2θ, 13.7°2θ, and 17.4°2θ (±0.2°2θ; ±0.1°2θ; or ±0.0°2θ; Cu Kα1 radiation).

In some embodiments, the Compound A salt is a crystalline polymorphic Form C characterized by two or more, or three or more PXRD signals selected from the group consisting of 6.1°2θ, 12.3°2θ, 13.7°2θ, and 17.4°2θ (±0.2°2θ; ±0.1°2θ; or ±0.0°2θ; Cu Kα1 radiation).

In some embodiments, the Compound A salt is a crystalline polymorphic Form C characterized by PXRD signals 6.1°2θ, 12.3°2θ, 13.7°2θ, and 17.4°2θ (±0.2°2θ; ±0.1°2θ; or ±0.0°2θ; Cu Kα1 radiation).

In some embodiments, the Compound A salt is a crystalline polymorphic Form C characterized by two or more, or three or more PXRD signals selected from the group consisting of 6.1°2θ, 12.1°2θ, 12.3°2θ, 13.7°2θ, and 17.4°2θ (±0.2°2θ; ±0.1°2θ; or ±0.0°2θ; Cu Kα1 radiation).

In some embodiments, the Compound A salt is a crystalline polymorphic Form C characterized by PXRD signals 6.1°2θ, 12.1°2θ, 12.3°2θ, 13.7°2θ, and 17.4°2θ (±0.2°2θ; ±0.1°2θ; or ±0.0°2θ; Cu Kα1 radiation).

In some embodiments, the Compound A salt is a crystalline polymorphic Form C characterized by two or more, or three or more PXRD signals selected from the group consisting of 6.1°2θ, 9.0°2θ, 12.1°2θ, 12.3°2θ, 13.7°2θ, and 17.4°2θ (±0.2°2θ; ±0.1°2θ; or ±0.0°2θ; Cu Kα1 radiation).

In some embodiments, the Compound A salt is a crystalline polymorphic Form C characterized by PXRD signals 6.1°2θ, 9.0°2θ, 12.1°2θ, 12.3°2θ, 13.7°2θ, and 17.4°2θ (±0.2°2θ; ±0.1°2θ; or ±0.0°2θ; Cu Kα1 radiation).

In some embodiments, the Compound A salt is a crystalline polymorphic Form C characterized by two or more, or three or more PXRD signals selected from the group consisting of 6.1°2θ, 9.0°2θ, 9.2°2θ, 12.1°2θ, 12.3°2θ, 13.7°2θ, and 17.4°2θ (±0.2°2θ; ±0.1°2θ; or ±0.0°2θ; Cu Kα1 radiation).

In some embodiments, the Compound A salt is a crystalline polymorphic Form C characterized by PXRD signals 6.1°2θ, 9.0°2θ, 9.2°2θ, 12.1°2θ, 12.3°2θ, 13.7°2θ, and 17.4°2θ (±0.2°2θ; ±0.1°2θ; or ±0.0°2θ; Cu Kα1 radiation).

In some embodiments, the Compound A salt is a crystalline polymorphic Form C characterized by two or more, or three or more PXRD signals selected from the group consisting of 6.1°2θ, 9.0°2θ, 9.2°2θ, 12.1°2θ, 12.3°2θ, 12.9°2θ, 13.7°2θ, and 17.4°2θ (±0.2°2θ; ±0.1°2θ; or ±0.0°2θ; Cu Kα1 radiation).

In some embodiments, the Compound A salt is a crystalline polymorphic Form C characterized by PXRD signals 6.1°2θ, 9.0°2θ, 9.2°2θ, 12.1°2θ, 12.3°2θ, 12.9°2θ, 13.7°2θ, and 17.4°2θ (±0.2°2θ; ±0.1°2θ; or ±0.0°2θ; Cu Kα1 radiation).

In some embodiments, the Compound A salt is a crystalline polymorphic Form C characterized by two or more, or three or more PXRD signals selected from the group consisting of 6.1°2θ, 9.0°2θ, 9.2°2θ, 12.1°2θ, 12.3°2θ, 12.9°2θ, 13.7°2θ, 17.4°2θ, and 19.5°2θ (±0.2°2θ; ±0.1°2θ; or ±0.0°2θ; Cu Kα1 radiation).

In some embodiments, the Compound A salt is a crystalline polymorphic Form C characterized by PXRD signals at 6.1°2θ, 9.0°2θ, 9.2°2θ, 12.1°2θ, 12.3°2θ, 12.9°2θ, 13.7°2θ, 17.4°2θ, and 19.5°2θ (±0.2°2θ; ±0.1°2θ; or ±0.0°2θ; Cu Kα1 radiation).

In some embodiments, the Compound A salt is a crystalline polymorphic Form C characterized by two or more, or three or more PXRD signals selected from the group consisting of 6.1°2θ, 9.0°2θ, 9.2°2θ, 12.1°2θ, 12.3°2θ, 12.9°2θ, 13.7°2θ, 16.1°2θ, 17.4°2θ, and 19.5°2θ (±0.2°2θ; ±0.1°2θ; or ±0.0°2θ; Cu Kα1 radiation).

In some embodiments, the Compound A salt is a crystalline polymorphic Form C characterized by PXRD signals at 6.1°2θ, 9.0°2θ, 9.2°2θ, 12.1°2θ, 12.3°2θ, 12.9°2θ, 13.7°2θ, 16.1°2θ, 17.4°2θ, and 19.5°2θ (±0.2°2θ; ±0.1°2θ; or ±0.0°2θ; Cu Kα1 radiation).

In some embodiments, the Compound A salt is a crystalline polymorphic Form C characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five XRPD signals selected from those set forth in Table 4.

In some embodiments, the Compound A salt is a crystalline polymorphic Form C characterized by one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four, or twenty-five XRPD signals selected from those set forth in Table 4.

In some embodiments, the Compound A salt is a crystalline polymorphic Form C characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five XRPD signals selected from 6.1°2θ, 8.6°2θ, 9.0°2θ, 9.2°2θ, 10.4°2θ, 11.1°2θ, 12.1°2θ, 12.3°2θ, 12.9°2θ, 13.7°2θ, 14.9°2θ, 16.1°2θ, 17.4°2θ, 18.5°2θ, 19.5°2θ, 20.3°2θ, 21.1°2θ, 22.0°2θ, 23.0°2θ, 23.6°2θ, 24.3°2θ, 25.0°2θ, 25.9°2θ, 26.6°2θ, and 27.5°2θ (±0.2°2θ; ±0.1°2θ; or ±0.0°2θ; Cu Kα1 radiation).

In some embodiments, the Compound A salt is a crystalline polymorphic Form C characterized by one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four, or twenty-five XRPD signals selected from 6.1°2θ, 8.6°2θ, 9.0°2θ, 9.2°2θ, 10.4°2θ, 11.1°2θ, 12.1°2θ, 12.3°2θ, 12.9°2θ, 13.7°2θ, 14.9°2θ, 16.1°2θ, 17.4°2θ, 18.5°2θ, 19.5°2θ, 20.3°2θ, 21.1°2θ, 22.0°2θ, 23.0°2θ, 23.6°2θ, 24.3°2θ, 25.0°2θ, 25.9°2θ, 26.6°2θ, and 27.5°2θ (±0.2°2θ; ±0.1°2θ; or ±0.0°2θ; Cu Kα1 radiation).

In some embodiments, the Compound A citrate salt is a crystalline Compound Form C and is characterized by an endothermic event with an onset temperature at approximately 112° C. as measured by DSC. In some embodiments, the Compound A citrate salt is a crystalline Compound Form C and is characterized by an endothermic event with an onset temperature at approximately 130° C. as measured by DSC. In some embodiments, the Compound A citrate salt is a crystalline Compound Form C and is characterized by an endothermic event with an onset temperature at approximately 156° C. as measured by DSC.

In some embodiments, the Compound A citrate salt is a crystalline Compound Form C and is characterized by an endothermic event with an onset temperature at approximately 112° C. with a peak temperature at approximately 117° C. as measured by DSC. In some embodiments, the Compound A citrate salt is a crystalline Compound Form C and is characterized by an endothermic event with an onset temperature at approximately 130° C. with a peak temperature at approximately 140° C. as measured by DSC. In some embodiments, the Compound A citrate salt is a crystalline Compound Form C and is characterized by an endothermic event with an onset temperature at approximately 156° C. with a peak temperature at approximately 180° C. as measured by DSC.

In some embodiments, the Compound A citrate salt is a crystalline Compound Form C and is characterized by an endothermic event with an onset temperature at approximately 27° C. as measured by DSC. In some embodiments, the Compound A citrate salt is a crystalline Compound Form C and is characterized by an endothermic event with an onset temperature at approximately 42° C. as measured by DSC. In some embodiments, the Compound A citrate salt is a crystalline Compound Form C and is characterized by an endothermic event with an onset temperature at approximately 109° C. as measured by DSC. In some embodiments, the Compound A citrate salt is a crystalline Compound Form C and is characterized by an endothermic event with an onset temperature at approximately 128° C. as measured by DSC. In some embodiments, the Compound A citrate salt is a crystalline Compound Form C and is characterized by an endothermic event with an onset temperature at approximately 154° C. as measured by DSC.

In some embodiments, the Compound A citrate salt is a crystalline Compound Form C and is characterized by an endothermic event with an onset temperature at approximately 27° C. with a peak temperature at approximately 31° C. as measured by DSC. In some embodiments, the Compound A citrate salt is a crystalline Compound Form C and is characterized by an endothermic event with an onset temperature at approximately 42° C. with a peak temperature at approximately 53° C. as measured by DSC. In some embodiments, the Compound A citrate salt is a crystalline Compound Form C and is characterized by an endothermic event with an onset temperature at approximately 109° C. with a peak temperature at approximately 114° C. as measured by DSC. In some embodiments, the Compound A citrate salt is a crystalline Compound Form C and is characterized by an endothermic event with an onset temperature at approximately 128° C. with a peak temperature at approximately 139° C. as measured by DSC. In some embodiments, the Compound A citrate salt is a crystalline Compound Form C and is characterized by an endothermic event with an onset temperature at approximately 154° C. with a peak temperature at approximately 179° C. as measured by DSC.

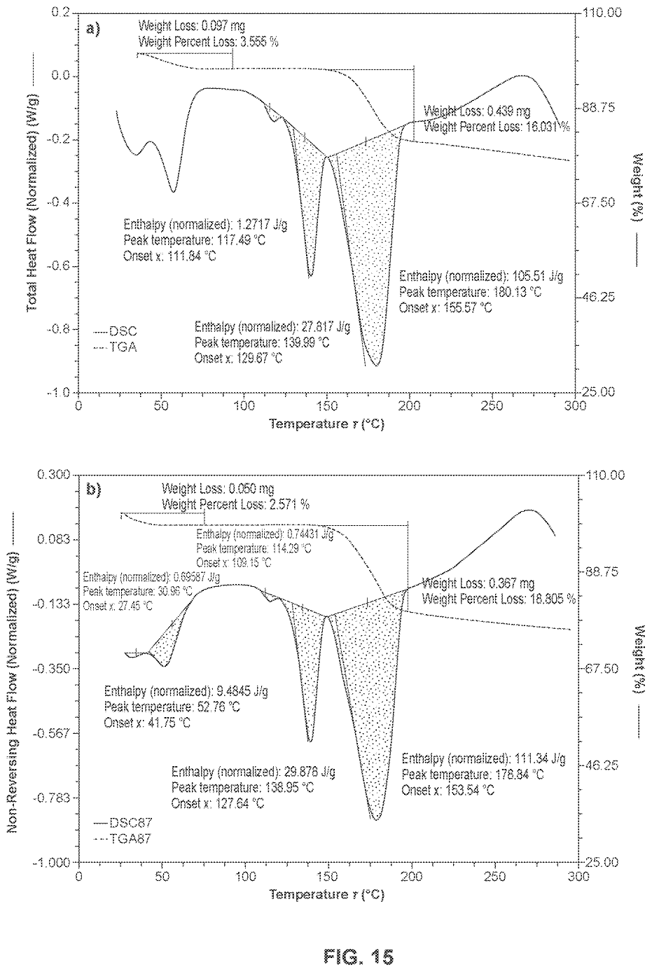

In some embodiments, the Compound A citrate salt is a crystalline polymorphic Form C characterized by DSC/TGA thermograms substantially similar to that shown in FIG. 15.

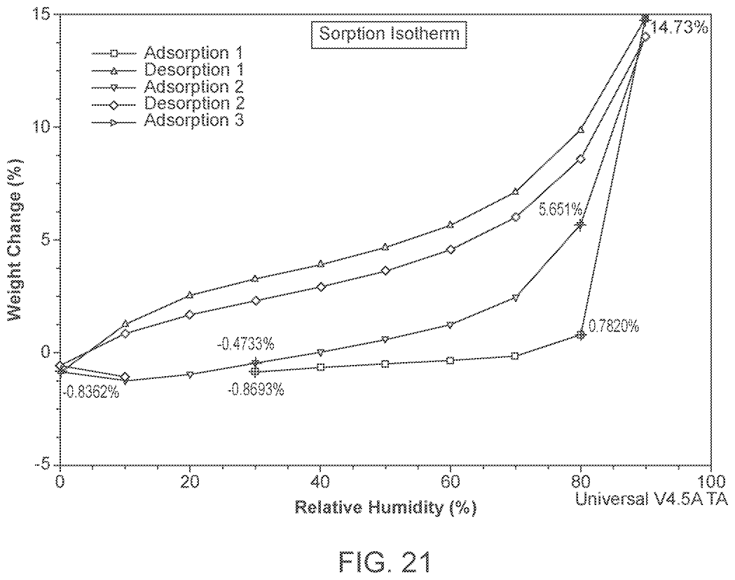

In some embodiments, the Compound A citrate salt is a crystalline polymorphic Form C characterized by sorption isotherm substantially similar to that shown in FIG. 21.

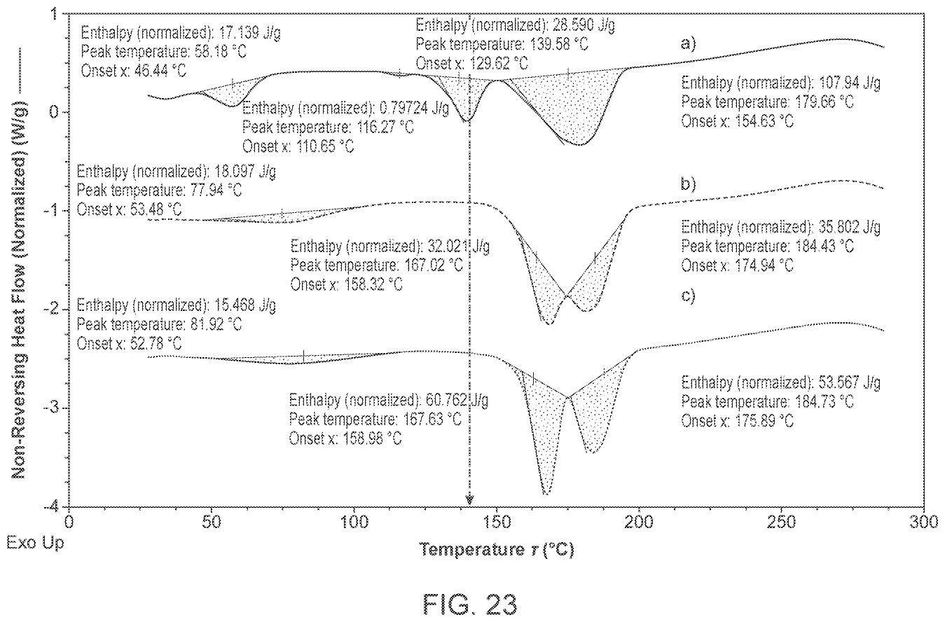

In some embodiments, the Compound A citrate salt is a crystalline polymorphic Form C characterized by DSC/TGA thermograms before DVS substantially similar to that shown in FIG. 23 (trace a).

In some embodiments, the Compound A citrate salt is a crystalline polymorphic Form C characterized by DSC/TGA thermograms after DVS substantially similar to that shown in FIG. 23 (trace b).

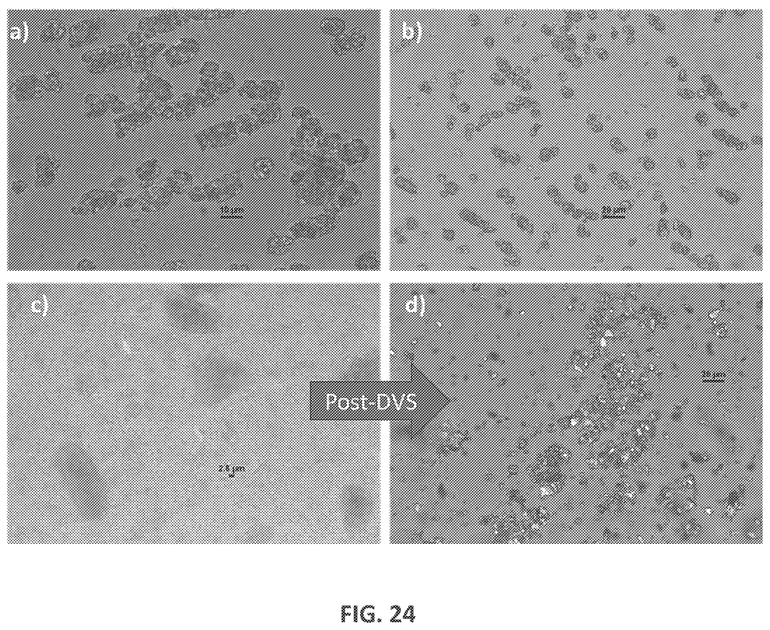

In some embodiments, the Compound A citrate salt is a crystalline polymorphic Form C characterized by PLM images substantially similar to that shown in FIG. 24.

In some embodiments, the Compound A citrate salt is a crystalline polymorphic Form C which is isolated from anhydrous acetonitrile and readily converts to Form A upon exposure to water/humidity.

### Preparation of Compound A Citrate Salt

Methods for synthesizing Compound A are known in the art, for example, the synthesis of Compound A is described in PCT Patent Appl. Publication No. WO 2017/214367.

The present disclosure provides detailed methods for preparing Compound A citrate salt of the present invention according to the following schemes and as shown in the Examples.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

The synthetic processes of the invention can tolerate a wide variety of functional groups, therefore various substituted starting materials can be used. The processes generally provide the desired final compound at or near the end of the overall process, although it may be desirable in certain instances to further convert the compound to a pharmaceutically acceptable salt, ester or prodrug thereof.

In some embodiments, Compound A monocitrate monohydrate salt is prepared according to Scheme 1 below.

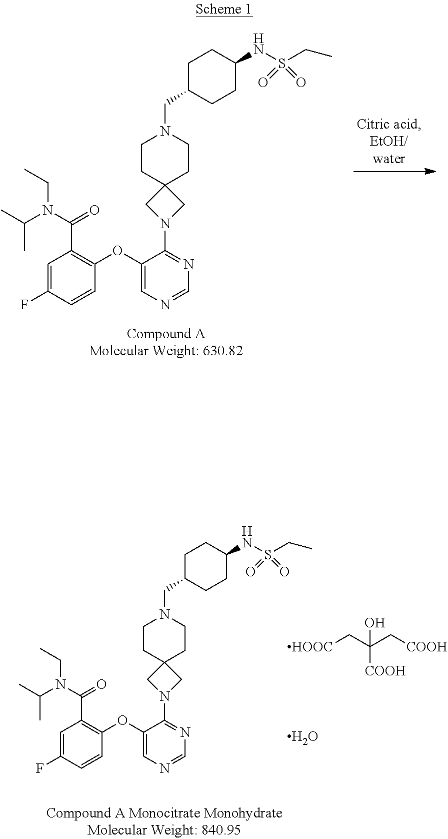

In some embodiments, Compound A monocitrate monohydrate tablets are prepared using steps comprising:
(a) blending Compound A monocitrate monohydrate with a glidant (e.g., hydrophobic colloidal silica) to form a pre-blend,
(b) blending the pre-blend with remaining excipients to form an intragranular blend.
(c) roller compacting and milling the intragranular blend to generate Compound A milled granules,
(d) blending the Compound A milled granules with a lubricant (e.g., magnesium stearate) to obtain Compound A final blend,
(e) compressing Compound A final blend into Compound A tablets,
(f) film coating Compound A tablets to form Compound A coated tablets, and
(g) packaging the Compound A coated tablets (e.g., into bottles or blisters).

### Formulations/Pharmaceutical Compositions

The formulations comprising Compound A, a salt thereof, or a polymorphic form thereof described herein can be administered orally, nasally, intranasally, transdermally, pulmonary, inhalationally, buccally, sublingually, intraperitoneally, subcutaneously, intramuscularly, intravenously, rectally, intrapleurally, intrathecally and parenterally. In some embodiments, the formulations comprising Compound A, a salt thereof, or a polymorphic form thereof are administered orally. One skilled in the art will recognize the advantages of certain routes of administration.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

Dosage forms for the topical or transdermal administration of the formulations comprising Compound A, a salt thereof, or a polymorphic form thereof described herein include powders, sprays, ointments, pastes, creams, lotions, gels, solutions, patches and inhalants. In some embodiments, the formulations comprising Compound A, a salt thereof, or a polymorphic form thereof described herein may be mixed under sterile conditions with a pharmaceutically acceptable carrier, and with any preservatives, buffers or propellants that are required.

A pharmaceutical composition of the disclosure is formulated to be compatible with its intended route of administration.

In some embodiments, the present disclosure is directed to a formulation comprising a Compound A, a salt thereof, or a polymorphic form thereof according to any of the embodiments described herein, and a pharmaceutically acceptable excipient.

In some embodiments, the present disclosure is directed to a formulation comprising a Compound A monocitrate monohydrate Form A salt according to any of the embodiments described herein, and a pharmaceutically acceptable excipient.

In some embodiments, the present disclosure is directed to a formulation comprising a Compound A citrate salt Form A salt according to any of the embodiments described herein, and a pharmaceutically acceptable excipient.

In some embodiments, the formulation of the present disclosure comprises a Compound A monocitrate monohydrate Form A salt, such that the formulation comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A monocitrate monohydrate Form A salt.

In some embodiments, the formulation of the present disclosure comprises a Compound A monocitrate monohydrate Form A salt, such that the formulation comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A monocitrate monohydrate Form A salt.

In some embodiments, the formulation of the present disclosure comprises a Compound A monocitrate monohydrate Form A salt, such that the formulation comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A.

In some embodiments, the formulation of the present disclosure comprises a Compound A monocitrate monohydrate Form A salt, such that the formulation comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, about 21%, about 22%, about 23%, about 23.8%, about 24%, about 24.4%, about 25%, about 26%, about 27%, about 28%, about 29%, or about 30% wt/wt of Compound A monocitrate monohydrate Form A salt.

In some embodiments, the formulation of the present disclosure comprises a Compound A monocitrate monohydrate Form A salt, such that the formulation comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, about 21%, about 22%, about 23%, about 23.8%, about 24%, about 24.4%, about 25%, about 26%, about 27%, about 28%, about 29%, or about 30% wt/wt of Compound A monocitrate monohydrate Form A salt calculated as free base equivalents.

In some embodiments, the formulation of the present disclosure comprises a Compound A monocitrate monohydrate Form A salt, such that the formulation comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, about 21%, about 22%, about 23%, about 23.8%, about 24%, about 24.4%, about 25%, about 26%, about 27%, about 28%, about 29%, or about 30% wt/wt of Compound A.

In some embodiments, the formulation comprises about 10 about 300 mg of Compound A.

In some embodiments, the formulation comprises about 10 about 300 mg of Compound A monocitrate monohydrate Form A salt.

In some embodiments, the formulation of the present disclosure comprises about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A.

In some embodiments, the formulation of the present disclosure comprises a Compound A citrate salt in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A citrate salt.

In some embodiments, the formulation of the present disclosure comprises a Compound A monocitrate monohydrate Form A salt in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A monocitrate monohydrate Form A salt.

In some embodiments, the formulation further comprises from about 1% to about 10% wt/wt of sodium bicarbonate.

In some embodiments, the formulation further comprises about 1%, about 2%, about 3%, about 4%, about 4.8%, about 5%, about 6%, about 7%, about 8%, about 9%, or about 10% wt/wt of sodium bicarbonate.

In some embodiments, the formulation further comprises from about 1 mg to about 100 mg of sodium bicarbonate.

In some embodiments, the formulation of the present disclosure comprises one or more fillers (such as microcrystalline cellulose, mannitol, and sorbitol), disintegrants (such as starch, croscarmellose sodium, and sodium starch glycolate), binders (such as low substituted hydroxypropyl cellulose, Povidone K30 (polyvinylpyrrolidone), gelatin, sodium alginate, methylcellulose, hydroxypropylmethylcellulose, lactose, and sucrose), glidants (such as talc, fumed silica, and magnesium carbonate), lubricants (such as stearic acid, maize starch, sodium lauryl sulfate, and calcium stearate), or film coatings.

In some embodiments, the formulation of the present disclosure comprises:
from about 15% to about 30% wt/wt of a Compound A monocitrate monohydrate Form A salt, and
from about 1% to about 10% wt/wt sodium bicarbonate.

In some embodiments, the formulation of the present disclosure comprises:
from about 15% to about 30% wt/wt of a Compound A monocitrate monohydrate Form A salt,
from about 30% to about 60% wt/wt of microcrystalline cellulose; and
from about 1% to about 10% wt/wt sodium bicarbonate.

In some embodiments, the formulation of the present disclosure comprises:
from about 15% to about 30% wt/wt of a Compound A monocitrate monohydrate Form A salt,
from about 30% to about 60% wt/wt of microcrystalline cellulose;
from about 1% to about 10% wt/wt sodium bicarbonate; and
from about 0.1% to about 2% wt/wt hydrophobic colloidal silica.

In some embodiments, the formulation of the present disclosure comprises:
from about 15% to about 30% wt/wt of a Compound A monocitrate monohydrate Form A salt,
from about 30% to about 60% wt/wt of microcrystalline cellulose;
from about 1% to about 10% wt/wt sodium bicarbonate;
from about 0.1% to about 2% wt/wt hydrophobic colloidal silica; and
from about 0.1% to about 7% wt/wt magnesium stearate.

In some embodiments, the formulation of the present disclosure comprises:
from about 15% to about 30% wt/wt of a Compound A monocitrate monohydrate Form A salt,
from about 30% to about 60% wt/wt of microcrystalline cellulose;
from about 5% to about 15% wt/wt crospovidone;
from about 1% to about 10% wt/wt sodium bicarbonate;
from about 0.1% to about 2% wt/wt hydrophobic colloidal silica; and
from about 0.1% to about 7% wt/wt magnesium stearate.

In some embodiments, the formulation of the present disclosure comprises:
from about 15% to about 30% wt/wt of a Compound A monocitrate monohydrate Form A salt,
from about 45% to about 60% wt/wt of microcrystalline cellulose; and
from about 1% to about 10% wt/wt sodium bicarbonate.

In some embodiments, the formulation of the present disclosure comprises:
from about 15% to about 30% wt/wt of a Compound A monocitrate monohydrate Form A salt,
from about 45% to about 60% wt/wt of microcrystalline cellulose;
from about 1% to about 10% wt/wt sodium bicarbonate; and
from about 0.1% to about 2% wt/wt hydrophobic colloidal silica.

In some embodiments, the formulation of the present disclosure comprises:
from about 15% to about 30% wt/wt of a Compound A monocitrate monohydrate Form A salt,
from about 45% to about 60% wt/wt of microcrystalline cellulose;
from about 1% to about 10% wt/wt sodium bicarbonate;
from about 0.1% to about 2% wt/wt hydrophobic colloidal silica; and
from about 0.1% to about 7% wt/wt magnesium stearate.

In some embodiments, the formulation of the present disclosure comprises:
from about 15% to about 30% wt/wt of a Compound A monocitrate monohydrate Form A salt,
from about 45% to about 60% wt/wt of microcrystalline cellulose;
from about 5% to about 15% wt/wt crospovidone;
from about 1% to about 10% wt/wt sodium bicarbonate;
from about 0.1% to about 2% wt/wt hydrophobic colloidal silica; and
from about 0.1% to about 7% wt/wt magnesium stearate.

In some embodiments, the formulation of the present disclosure comprises:
from about 15% to about 30% wt/wt of a Compound A monocitrate monohydrate Form A salt,
from about 45% to about 60% wt/wt of a mixture of microcrystalline cellulose and dicalcium phosphate; and
from about 1% to about 10% wt/wt sodium bicarbonate.

In some embodiments, the formulation of the present disclosure comprises:
from about 15% to about 30% wt/wt of a Compound A monocitrate monohydrate Form A salt,
from about 45% to about 60% wt/wt of a mixture of microcrystalline cellulose and dicalcium phosphate;
from about 1% to about 10% wt/wt sodium bicarbonate; and
from about 0.1% to about 2% wt/wt hydrophobic colloidal silica.

In some embodiments, the formulation of the present disclosure comprises:
from about 15% to about 30% wt/wt of a Compound A monocitrate monohydrate Form A salt,
from about 45% to about 60% wt/wt of a mixture of microcrystalline cellulose and dicalcium phosphate;
from about 1% to about 10% wt/wt sodium bicarbonate;
from about 0.1% to about 2% wt/wt hydrophobic colloidal silica; and
from about 0.1% to about 7% wt/wt magnesium stearate.

In some embodiments, the formulation of the present disclosure comprises:
from about 15% to about 30% wt/wt of a Compound A monocitrate monohydrate Form A salt,
from about 45% to about 60% wt/wt of a mixture of microcrystalline cellulose and dicalcium phosphate;
from about 5% to about 15% wt/wt crospovidone;
from about 1% to about 10% wt/wt sodium bicarbonate;
from about 0.1% to about 2% wt/wt hydrophobic colloidal silica; and
from about 0.1% to about 7% wt/wt magnesium stearate.

In some embodiments, the formulation of the present disclosure comprises Compound A monocitrate monohydrate Form A salt, such that the formulation comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A monocitrate monohydrate Form A salt.

In some embodiments, the formulation of the present disclosure comprises a Compound A monocitrate monohydrate Form A salt, such that the formulation comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A monocitrate monohydrate Form A salt calculated as free base equivalents.

In some embodiments, the formulation of the present disclosure comprises a Compound A monocitrate monohydrate Form A salt, such that the formulation comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A monocitrate monohydrate Form A salt.

In some embodiments, the formulation of the present disclosure comprises a Compound A monocitrate monohydrate Form A salt, such that the formulation comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A monocitrate monohydrate Form A salt calculated as free base equivalents.

In some embodiments, the formulation of the present disclosure comprises a Compound A monocitrate monohydrate Form A salt, such that the formulation comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, about 21%, about 22%, about 23%, about 23.8%, about 24%, about 24.4%, about 25%, about 26%, about 27%, about 28%, about 29%, or about 30% wt/wt of Compound A monocitrate monohydrate Form A salt.

In some embodiments, the formulation of the present disclosure comprises a Compound A monocitrate monohydrate Form A salt, such that the formulation comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, about 21%, about 22%, about 23%, about 23.8%, about 24%, about 24.4%, about 25%, about 26%, about 27%, about 28%, about 29%, or about 30% wt/wt of Compound A monocitrate monohydrate Form A salt calculated as free base equivalents.

In some embodiments, the formulation comprises about 10 about 300 mg of Compound A monocitrate monohydrate Form A salt calculated as free base equivalents.

In some embodiments, the formulation of the present disclosure comprises a Compound A monocitrate monohydrate Form A salt in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A monocitrate monohydrate Form A salt calculated as free base equivalents.

In some embodiments, the formulation comprises Compound A monocitrate monohydrate Form A salt in an amount of about 25 mg, about 33.4 mg, about 110 mg, about 135 mg, about 146.5 mg, about 160 mg, about 180.2 mg, about 213.2 mg, or about 250 mg calculated as free base equivalents.

In some embodiments, the formulation of the present disclosure comprises:
from about 15% to about 30% wt/wt of a Compound A monocitrate monohydrate Form A salt calculated as free base equivalents, and
from about 1% to about 10% wt/wt sodium bicarbonate.

In some embodiments, the formulation of the present disclosure comprises:
from about 15% to about 30% wt/wt of a Compound A monocitrate monohydrate Form A salt calculated as free base equivalents,
from about 30% to about 60% wt/wt of microcrystalline cellulose; and
from about 1% to about 10% wt/wt sodium bicarbonate.

In some embodiments, the formulation of the present disclosure comprises:
from about 15% to about 30% wt/wt of a Compound A monocitrate monohydrate Form A salt calculated as free base equivalents,
from about 30% to about 60% wt/wt of microcrystalline cellulose;
from about 1% to about 10% wt/wt sodium bicarbonate; and
from about 0.1% to about 2% wt/wt hydrophobic colloidal silica.

In some embodiments, the formulation of the present disclosure comprises:
from about 15% to about 30% wt/wt of a Compound A monocitrate monohydrate Form A salt calculated as free base equivalents,
from about 30% to about 60% wt/wt of microcrystalline cellulose;
from about 1% to about 10% wt/wt sodium bicarbonate;
from about 0.1% to about 2% wt/wt hydrophobic colloidal silica; and
from about 0.1% to about 7% wt/wt magnesium stearate.

In some embodiments, the formulation of the present disclosure comprises:
from about 15% to about 30% wt/wt of a Compound A monocitrate monohydrate Form A salt calculated as free base equivalents,
from about 30% to about 60% wt/wt of microcrystalline cellulose;
from about 5% to about 15% wt/wt crospovidone;
from about 1% to about 10% wt/wt sodium bicarbonate;
from about 0.1% to about 2% wt/wt hydrophobic colloidal silica; and
from about 0.1% to about 7% wt/wt magnesium stearate.

In some embodiments, the formulation of the present disclosure comprises:
from about 15% to about 30% wt/wt of a Compound A monocitrate monohydrate Form A salt calculated as free base equivalents,
from about 45% to about 60% wt/wt of microcrystalline cellulose; and
from about 1% to about 10% wt/wt sodium bicarbonate.

In some embodiments, the formulation of the present disclosure comprises:
from about 15% to about 30% wt/wt of a Compound A monocitrate monohydrate Form A salt calculated as free base equivalents,
from about 45% to about 60% wt/wt of microcrystalline cellulose;
from about 1% to about 10% wt/wt sodium bicarbonate; and
from about 0.1% to about 2% wt/wt hydrophobic colloidal silica.

In some embodiments, the formulation of the present disclosure comprises:
from about 15% to about 30% wt/wt of a Compound A monocitrate monohydrate Form A salt calculated as free base equivalents,
from about 45% to about 60% wt/wt of microcrystalline cellulose;
from about 1% to about 10% wt/wt sodium bicarbonate;
from about 0.1% to about 2% wt/wt hydrophobic colloidal silica; and
from about 0.1% to about 7% wt/wt magnesium stearate.

In some embodiments, the formulation of the present disclosure comprises:
from about 15% to about 30% wt/wt of a Compound A monocitrate monohydrate Form A salt calculated as free base equivalents,
from about 45% to about 60% wt/wt of microcrystalline cellulose;
from about 5% to about 15% wt/wt crospovidone;
from about 1% to about 10% wt/wt sodium bicarbonate;
from about 0.1% to about 2% wt/wt hydrophobic colloidal silica; and
from about 0.1% to about 7% wt/wt magnesium stearate.

In some embodiments, the formulation of the present disclosure comprises:
from about 25 mg to about 250 mg of a Compound A monocitrate monohydrate Form A salt, and
from about 5 mg to about 50 mg sodium bicarbonate.

In some embodiments, the formulation of the present disclosure comprises:
from about 25 mg to about 250 mg of a Compound A monocitrate monohydrate Form A salt,
from about 40 mg to about 500 mg of microcrystalline cellulose; and
from about 5 mg to about 50 mg sodium bicarbonate.

In some embodiments, the formulation of the present disclosure comprises:
from about 25 mg to about 250 mg of a Compound A monocitrate monohydrate Form A salt,
from about 40 mg to about 500 mg of microcrystalline cellulose;
from about 5 mg to about 50 mg sodium bicarbonate; and
from about 1 mg to about 10 mg hydrophobic colloidal silica.

In some embodiments, the formulation of the present disclosure comprises:
from about 25 mg to about 250 mg of a Compound A monocitrate monohydrate Form A salt,
from about 40 mg to about 500 mg of microcrystalline cellulose;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt,
from about 40 mg to about 500 mg of microcrystalline cellulose;
from about 10 mg to about 100 mg crospovidone;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt,
from about 40 mg to about 500 mg of microcrystalline cellulose;
from about 10 mg to about 100 mg hypromellose;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt,
from about 40 mg to about 500 mg of microcrystalline cellulose;
from about 10 mg to about 100 mg hypromellose;
from about 10 mg to about 100 mg crospovidone;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt calculated as free base equivalents,
from about 40 mg to about 500 mg of microcrystalline cellulose; and
from about 5 mg to about 50 mg sodium bicarbonate.

In some embodiments, the formulation of the present disclosure comprises:
from about 25 mg to about 250 mg of a Compound A monocitrate monohydrate Form A salt calculated as free base equivalents,
from about 40 mg to about 500 mg of microcrystalline cellulose;
from about 5 mg to about 50 mg sodium bicarbonate; and
from about 1 mg to about 10 mg hydrophobic colloidal silica.

In some embodiments, the formulation of the present disclosure comprises:
from about 25 mg to about 250 mg of a Compound A monocitrate monohydrate Form A salt calculated as free base equivalents,
from about 40 mg to about 500 mg of microcrystalline cellulose;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt calculated as free base equivalents,
from about 40 mg to about 500 mg of microcrystalline cellulose;
from about 10 mg to about 100 mg crospovidone;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt calculated as free base equivalents,
from about 40 mg to about 500 mg of microcrystalline cellulose;
from about 10 mg to about 100 mg hypromellose;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt calculated as free base equivalents,
from about 40 mg to about 500 mg of microcrystalline cellulose;
from about 10 mg to about 100 mg hypromellose;
from about 10 mg to about 100 mg crospovidone;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt,
from about 50 mg to about 500 mg of microcrystalline cellulose; and
from about 5 mg to about 50 mg sodium bicarbonate.

In some embodiments, the formulation of the present disclosure comprises:
from about 25 mg to about 250 mg of a Compound A monocitrate monohydrate Form A salt,
from about 50 mg to about 500 mg of microcrystalline cellulose;
from about 5 mg to about 50 mg sodium bicarbonate; and
from about 1 mg to about 10 mg hydrophobic colloidal silica.

In some embodiments, the formulation of the present disclosure comprises:
from about 25 mg to about 250 mg of a Compound A monocitrate monohydrate Form A salt,
from about 50 mg to about 500 mg of microcrystalline cellulose;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt,
from about 50 mg to about 500 mg of microcrystalline cellulose;
from about 10 mg to about 100 mg crospovidone;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt,
from about 50 mg to about 500 mg of microcrystalline cellulose;
from about 10 mg to about 100 mg hypromellose;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt,
from about 50 mg to about 500 mg of microcrystalline cellulose;
from about 10 mg to about 100 mg hypromellose;
from about 10 mg to about 100 mg crospovidone;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt calculated as free base equivalents, and
from about 5 mg to about 50 mg sodium bicarbonate.

In some embodiments, the formulation of the present disclosure comprises:
from about 25 mg to about 250 mg of a Compound A monocitrate monohydrate Form A salt calculated as free base equivalents,
from about 50 mg to about 500 mg of microcrystalline cellulose; and
from about 5 mg to about 50 mg sodium bicarbonate.

In some embodiments, the formulation of the present disclosure comprises:
from about 25 mg to about 250 mg of a Compound A monocitrate monohydrate Form A salt calculated as free base equivalents,
from about 50 mg to about 500 mg of microcrystalline cellulose;
from about 5 mg to about 50 mg sodium bicarbonate; and
from about 1 mg to about 10 mg hydrophobic colloidal silica.

In some embodiments, the formulation of the present disclosure comprises:
from about 25 mg to about 250 mg of a Compound A monocitrate monohydrate Form A salt calculated as free base equivalents,
from about 50 mg to about 500 mg of microcrystalline cellulose;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt calculated as free base equivalents,
from about 50 mg to about 500 mg of microcrystalline cellulose;
from about 10 mg to about 100 mg crospovidone;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt calculated as free base equivalents,
from about 50 mg to about 500 mg of microcrystalline cellulose;
from about 10 mg to about 100 mg hypromellose;
from about 5 mg to about 50 mg sodium bicarbonate;
from about 1 mg to about 10 mg hydrophobic colloidal silica; and
from about 0.5 mg to about 15 mg magnesium stearate.

In some embodiments, the formulation of the present disclosure comprises:
from about 25 mg to about 250 mg of a Compound A monocitrate monohydrate Form A salt calculated as free base equivalents,
from about 50 mg to about 500 mg of microcrystalline cellulose;
from about 10 mg to about 100 mg hypromellose;
from about 10 mg to about 100 mg crospovidone;
from about 5 mg to about 50 mg sodium bicarbonate;
from about 1 mg to about 10 mg hydrophobic colloidal silica; and
from about 0.5 mg to about 15 mg magnesium stearate.

### Tablet Formulation

In some embodiments, the present disclosure is directed to a tablet comprising a Compound A citrate salt according to any of the embodiments described herein, and a pharmaceutically acceptable excipient.

In some embodiments, the present disclosure is directed to a tablet comprising a Compound A citrate salt polymorphic form according to any of the embodiments described herein, and a pharmaceutically acceptable excipient.

In some embodiments, the present disclosure is directed to a tablet comprising a Compound A citrate salt Form A salt according to any of the embodiments described herein, and a pharmaceutically acceptable excipient.

In some embodiments, the present disclosure is directed to a tablet comprising the Compound A monocitrate monohydrate Form A salt and a pharmaceutically acceptable excipient.

In some embodiments, the tablet of the present disclosure further comprises a bicarbonate.

In some embodiments, the tablet of the present disclosure further comprises sodium or potassium bicarbonate.

In some embodiments, the tablet of the present disclosure further comprises sodium bicarbonate.

In some embodiments, the tablet of the present disclosure comprises an intragranular phase and an extragranular phase, wherein:
(a) the intragranular phase comprises Compound A citrate salt, or a polymorphic form thereof (e.g., Form A, B, and/or C) dispersed within a first pharmaceutically acceptable excipient; and
(b) the extragranular phase comprises a second pharmaceutically acceptable excipient.

In some embodiments, the tablet further comprises an intragranular phase and an extragranular phase, wherein:
(a) the intragranular phase comprises Compound A monocitrate monohydrate Form A salt dispersed within a first pharmaceutically acceptable excipient; and
(b) the extragranular phase comprises a second pharmaceutically acceptable excipient.

In some embodiments, the tablet of the present disclosure comprises a Compound A citrate salt, or a polymorphic form thereof (e.g., Form A, B, and/or C), such that the tablet comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A citrate salt, or a polymorphic form thereof.

In some embodiments, the tablet of the present disclosure comprises a Compound A citrate salt, or a polymorphic form thereof (e.g., Form A, B, and/or C), such that the tablet comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A citrate salt, or a polymorphic form thereof calculated as free base equivalents.

In some embodiments, the tablet of the present disclosure comprises a Compound A monocitrate monohydrate, or a polymorphic form thereof (e.g., Form A, B, and/or C), such that the tablet comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A monocitrate monohydrate, or a polymorphic form thereof.

In some embodiments, the tablet of the present disclosure comprises a Compound A monocitrate monohydrate, or a polymorphic form thereof (e.g., Form A, B, and/or C), such that the tablet comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A monocitrate monohydrate, or a polymorphic form thereof calculated as free base equivalents.

In some embodiments, the tablet comprises a Compound A citrate salt, or a polymorphic form thereof (e.g., Form A, B, and/or C), such that the tablet comprises from about 10% to about 40% wt/wt of Compound A citrate salt, or a polymorphic form thereof.

In some embodiments, the tablet comprises a Compound A citrate salt, or a polymorphic form thereof (e.g., Form A, B, and/or C), such that the tablet comprises from about 10% to about 30% wt/wt of Compound A citrate salt, or a polymorphic form thereof.

In some embodiments, the tablet comprises a Compound A citrate salt, or a polymorphic form thereof (e.g., Form A, B, and/or C), such that the tablet comprises from about 10% to about 25% wt/wt of Compound A citrate salt, or a polymorphic form thereof.

In some embodiments, the tablet comprises a Compound A citrate salt, or a polymorphic form thereof (e.g., Form A, B, and/or C), such that the tablet comprises from about 10% to about 20% wt/wt of Compound A citrate salt, or a polymorphic form thereof.

In some embodiments, the tablet comprises a Compound A citrate salt, or a polymorphic form thereof (e.g., Form A, B, and/or C), such that the tablet comprises from about 12% to about 30% wt/wt of Compound A citrate salt, or a polymorphic form thereof.

In some embodiments, the tablet comprises a Compound A citrate salt, or a polymorphic form thereof (e.g., Form A, B, and/or C), such that the tablet comprises from about 12% to about 25% wt/wt of Compound A citrate salt, or a polymorphic form thereof.

In some embodiments, the tablet comprises a Compound A citrate salt, or a polymorphic form thereof (e.g., Form A, B, and/or C), such that the tablet comprises from about 12% to about 20% wt/wt of Compound A citrate salt, or a polymorphic form thereof.

In some embodiments, the tablet comprises a Compound A citrate salt, or a polymorphic form thereof (e.g., Form A, B, and/or C), such that the tablet comprises from about 20% to about 25% wt/wt of Compound A citrate salt, or a polymorphic form thereof.

In some embodiments, the tablet comprises a Compound A citrate salt, or a polymorphic form thereof (e.g., Form A, B, and/or C), such that the tablet comprises from about 15% to about 30% wt/wt of Compound A citrate salt, or a polymorphic form thereof.

In some embodiments, the tablet comprises a Compound A citrate salt, or a polymorphic form thereof (e.g., Form A, B, and/or C), such that the tablet comprises from about 15% to about 25% wt/wt of Compound A citrate salt, or a polymorphic form thereof.

In some embodiments, the tablet comprises a Compound A citrate salt, or a polymorphic form thereof (e.g., Form A, B, and/or C), such that the tablet comprises from about 15% to about 20% wt/wt of Compound A citrate salt, or a polymorphic form thereof.

In some embodiments, the tablet comprises a Compound A citrate salt, or a polymorphic form thereof (e.g., Form A, B, and/or C), such that the tablet comprises from about 10% to about 40% wt/wt of Compound A citrate salt, or a polymorphic form thereof calculated as free base equivalents.

In some embodiments, the tablet comprises a Compound A citrate salt, or a polymorphic form thereof (e.g., Form A, B, and/or C), such that the tablet comprises from about 10% to about 30% wt/wt of Compound A citrate salt, or a polymorphic form thereof calculated as free base equivalents.

In some embodiments, the tablet comprises a Compound A citrate salt, or a polymorphic form thereof (e.g., Form A, B, and/or C), such that the tablet comprises from about 10% to about 25% wt/wt of Compound A citrate salt, or a polymorphic form thereof calculated as free base equivalents.

In some embodiments, the tablet comprises a Compound A citrate salt, or a polymorphic form thereof (e.g., Form A, B, and/or C), such that the tablet comprises from about 10% to about 20% wt/wt of Compound A citrate salt, or a polymorphic form thereof calculated as free base equivalents.

In some embodiments, the tablet comprises a Compound A citrate salt, or a polymorphic form thereof (e.g., Form A, B, and/or C), such that the tablet comprises from about 20% to about 25% wt/wt of Compound A citrate salt, or a polymorphic form thereof calculated as free base equivalents.

In some embodiments, the tablet comprises a Compound A citrate salt, or a polymorphic form thereof (e.g., Form A, B, and/or C), such that the tablet comprises from about 12% to about 30% wt/wt of Compound A citrate salt, or a polymorphic form thereof calculated as free base equivalents.

In some embodiments, the tablet comprises a Compound A citrate salt, or a polymorphic form thereof (e.g., Form A, B, and/or C), such that the tablet comprises from about 12% to about 25% wt/wt of Compound A citrate salt, or a polymorphic form thereof calculated as free base equivalents.

In some embodiments, the tablet comprises a Compound A citrate salt, or a polymorphic form thereof (e.g., Form A, B, and/or C), such that the tablet comprises from about 12% to about 20% wt/wt of Compound A citrate salt, or a polymorphic form thereof calculated as free base equivalents.

In some embodiments, the tablet comprises a Compound A citrate salt, or a polymorphic form thereof (e.g., Form A, B, and/or C), such that the tablet comprises from about 15% to about 30% wt/wt of Compound A citrate salt, or a polymorphic form thereof calculated as free base equivalents.

In some embodiments, the tablet comprises a Compound A citrate salt, or a polymorphic form thereof (e.g., Form A, B, and/or C), such that the tablet comprises from about 15% to about 25% wt/wt of Compound A citrate salt, or a polymorphic form thereof calculated as free base equivalents.

In some embodiments, the tablet comprises a Compound A citrate salt, or a polymorphic form thereof (e.g., Form A, B, and/or C), such that the tablet comprises from about 15% to about 20% wt/wt of Compound A citrate salt, or a polymorphic form thereof calculated as free base equivalents.

In some embodiments, the tablet comprises from about 12% to about 30% wt/wt of Compound A citrate salt, or a polymorphic form thereof (e.g., Form A, B, and/or C).

In some embodiments, the tablet comprises from about 12% to about 25% wt/wt of Compound A citrate salt, or a polymorphic form thereof (e.g., Form A, B, and/or C).

In some embodiments, the tablet comprises from about 15% to about 30% wt/wt. of Compound A citrate salt, or a polymorphic form thereof (e.g., Form A, B, and/or C).

In some embodiments, the tablet comprises from about 15% to about 25% wt/wt of Compound A citrate salt, or a polymorphic form thereof (e.g., Form A, B, and/or C).

In some embodiments, the tablet comprises from about 20% to about 30% wt/wt. of Compound A citrate salt, or a polymorphic form thereof (e.g., Form A, B, and/or C).

In some embodiments, the tablet comprises from about 20% to about 25% wt/wt of Compound A citrate salt, or a polymorphic form thereof (e.g., Form A, B, and/or C).

In some embodiments, the tablet comprises from about 12% to about 30% wt/wt of Compound A citrate salt, or a polymorphic form thereof (e.g., Form A, B, and/or C) calculated as free base equivalents.

In some embodiments, the tablet comprises from about 12% to about 25% wt/wt of Compound A citrate salt, or a polymorphic form thereof (e.g., Form A, B, and/or C) calculated as free base equivalents.

In some embodiments, the tablet comprises from about 15% to about 30% wt/wt. of Compound A citrate salt, or a polymorphic form thereof (e.g., Form A, B, and/or C) calculated as free base equivalents.

In some embodiments, the tablet comprises from about 15% to about 25% wt/wt of Compound A citrate salt, or a polymorphic form thereof (e.g., Form A, B, and/or C) calculated as free base equivalents.

In some embodiments, the tablet comprises from about 20% to about 30% wt/wt. of Compound A citrate salt, or a polymorphic form thereof (e.g., Form A, B, and/or C) calculated as free base equivalents.

In some embodiments, the tablet comprises from about 20% to about 25% wt/wt of Compound A citrate salt, or a polymorphic form thereof (e.g., Form A, B, and/or C) calculated as free base equivalents.

In some embodiments, the tablet comprises Compound A citrate Form A salt such that the tablet comprises from about 10% to about 30% wt/wt of Compound A.

In some embodiments, the tablet comprises Compound A citrate Form A salt such that the tablet comprises from about 10% to about 25% wt/wt of Compound A.

In some embodiments, the tablet comprises Compound A citrate Form A salt such that the tablet comprises from about 10% to about 20% wt/wt of Compound A.

In some embodiments, the tablet comprises Compound A citrate Form A salt such that the tablet comprises from about 12% to about 30% wt/wt of Compound A.

In some embodiments, the tablet comprises Compound A citrate Form A salt such that the tablet comprises from about 12% to about 25% wt/wt of Compound A.

In some embodiments, the tablet comprises Compound A citrate Form A salt such that the tablet comprises from about 12% to about 20% wt/wt of Compound A.

In some embodiments, the tablet comprises Compound A citrate Form A salt such that the tablet comprises from about 15% to about 30% wt/wt of Compound A.

In some embodiments, the tablet comprises Compound A citrate Form A salt such that the tablet comprises from about 15% to about 25% wt/wt of Compound A.

In some embodiments, the tablet comprises Compound A citrate Form A salt such that the tablet comprises from about 15% to about 20% wt/wt of Compound A.

In some embodiments, the tablet comprises Compound A citrate Form A salt such that the tablet comprises from about 10% to about 30% wt/wt of Compound A calculated as free base equivalents.

In some embodiments, the tablet comprises Compound A citrate Form A salt such that the tablet comprises from about 10% to about 25% wt/wt of Compound A calculated as free base equivalents.

In some embodiments, the tablet comprises Compound A citrate Form A salt such that the tablet comprises from about 10% to about 20% wt/wt of Compound A calculated as free base equivalents.

In some embodiments, the tablet comprises Compound A citrate Form A salt such that the tablet comprises from about 12% to about 30% wt/wt of Compound A calculated as free base equivalents.

In some embodiments, the tablet comprises Compound A citrate Form A salt such that the tablet comprises from about 12% to about 25% wt/wt of Compound A calculated as free base equivalents.

In some embodiments, the tablet comprises Compound A citrate Form A salt such that the tablet comprises from about 12% to about 20% wt/wt of Compound A calculated as free base equivalents.

In some embodiments, the tablet comprises Compound A citrate Form A salt such that the tablet comprises from about 15% to about 30% wt/wt of Compound A calculated as free base equivalents.

In some embodiments, the tablet comprises Compound A citrate Form A salt such that the tablet comprises from about 15% to about 25% wt/wt of Compound A calculated as free base equivalents.

In some embodiments, the tablet comprises Compound A citrate Form A salt such that the tablet comprises from about 15% to about 20% wt/wt of Compound A calculated as free base equivalents.

In some embodiments, the tablet comprises from about 12% to about 30% wt/wt of Compound A citrate Form A salt.

In some embodiments, the tablet comprises from about 12% to about 25% wt/wt of Compound A citrate Form A salt.

In some embodiments, the tablet comprises from about 15% to about 30% wt/wt of Compound A citrate Form A salt.

In some embodiments, the tablet comprises from about 15% to about 25% wt/wt of Compound A citrate Form A salt.

In some embodiments, the tablet comprises from about 20% to about 30% wt/wt of Compound A citrate Form A salt.

In some embodiments, the tablet comprises from about 20% to about 25% wt/wt of Compound A citrate Form A salt.

In some embodiments, the tablet comprises from about 12% to about 30% wt/wt of Compound A citrate Form A salt calculated as free base equivalents.

In some embodiments, the tablet comprises from about 12% to about 25% wt/wt of Compound A citrate Form A salt calculated as free base equivalents.

In some embodiments, the tablet comprises from about 15% to about 30% wt/wt of Compound A citrate Form A salt calculated as free base equivalents.

In some embodiments, the tablet comprises from about 15% to about 25% wt/wt of Compound A citrate Form A salt calculated as free base equivalents.

In some embodiments, the tablet comprises from about 20% to about 30% wt/wt of Compound A citrate Form A salt calculated as free base equivalents.

In some embodiments, the tablet comprises from about 20% to about 25% wt/wt of Compound A citrate Form A salt calculated as free base equivalents.

In some embodiments, the tablet of the present disclosure comprises a Compound A citrate salt, or a polymorphic form thereof (e.g., Form A, B, and/or C), such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, about 21%, about 22%, about 23%, about 23.8%, about 24%, about 24.4%, about 25%, about 26%, about 27%, about 28%, about 29%, or about 30% wt/wt of Compound A citrate salt, or a polymorphic form thereof.

In some embodiments, the tablet of the present disclosure comprises a Compound A citrate salt, or a polymorphic form thereof (e.g., Form A, B, and/or C), such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, about 21%, about 22%, about 23%, about 23.8%, about 24%, about 24.4%, about 25%, about 26%, about 27%, about 28%, about 29%, or about 30% wt/wt of Compound A citrate salt, or a polymorphic form thereof calculated as free base equivalents.

In some embodiments, the tablet of the present disclosure comprises a Compound A monocitrate monohydrate, or a polymorphic form thereof (e.g., Form A, B, and/or C), such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, about 21%, about 22%, about 23%, about 23.8%, about 24%, about 24.4%, about 25%, about 26%, about 27%, about 28%, about 29%, or about 30% wt/wt of Compound A monocitrate monohydrate, or a polymorphic form thereof.

In some embodiments, the tablet of the present disclosure comprises a Compound A monocitrate monohydrate, or a polymorphic form thereof (e.g., Form A, B, and/or C), such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, about 21%, about 22%, about 23%, about 23.8%, about 24%, about 24.4%, about 25%, about 26%, about 27%, about 28%, about 29%, or about 30% wt/wt of Compound A monocitrate monohydrate, or a polymorphic form thereof calculated as free base equivalents.

In some embodiments, the tablet of the present disclosure comprises a Compound A citrate Form A salt, such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, about 21%, about 22%, about 23%, about 23.8%, about 24%, about 24.4%, about 25%, about 26%, about 27%, about 28%, about 29%, or about 30% wt/wt of Compound A citrate Form A salt.

In some embodiments, the tablet of the present disclosure comprises a Compound A citrate Form A salt, such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, about 21%, about 22%, about 23%, about 23.8%, about 24%, about 24.4%, about 25%, about 26%, about 27%, about 28%, about 29%, or about 30% wt/wt of Compound A citrate Form A salt calculated as free base equivalents.

In some embodiments, the tablet of the present disclosure comprises a Compound A monocitrate monohydrate Form A salt, such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, about 21%, about 22%, about 23%, about 23.8%, about 24%, about 24.4%, about 25%, about 26%, about 27%, about 28%, about 29%, or about 30% wt/wt of Compound A monocitrate monohydrate Form A salt.

In some embodiments, the tablet of the present disclosure comprises a Compound A monocitrate monohydrate Form A salt, such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, about 21%, about 22%, about 23%, about 23.8%, about 24%, about 24.4%, about 25%, about 26%, about 27%, about 28%, about 29%, or about 30% wt/wt of Compound A monocitrate monohydrate Form A salt calculated as free base equivalents.

In some embodiments, the Compound A monocitrate monohydrate Form A salt in the tablet is from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt.

In some embodiments, the Compound A monocitrate monohydrate Form A salt is present in the tablet such that Compound A is from about 10% to about 30% wt/wt.

In some embodiments, the Compound A monocitrate monohydrate Form A salt in the tablet is from about 10% to about 25% wt/wt.

In some embodiments, the tablet comprises about 10 about 300 mg of Compound A.

In some embodiments, the tablet comprises about 10 about 300 mg of Compound A monocitrate monohydrate Form A salt.

In some embodiments, the tablet comprises about 10 about 300 mg of Compound A monocitrate monohydrate Form A salt and further comprises a bicarbonate.

In some embodiments, the tablet comprises about 10 about 300 mg of Compound A monocitrate monohydrate Form A salt and further comprises sodium bicarbonate.

In some embodiments, the tablet of the present disclosure comprises a Compound A citrate salt, or a polymorphic form thereof (e.g., Form A, B, and/or C) in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A citrate salt, or a polymorphic form thereof.

In some embodiments, the tablet of the present disclosure comprises a Compound A citrate salt, or a polymorphic form thereof (e.g., Form A, B, and/or C) in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A citrate salt, or a polymorphic form thereof calculated as free base equivalents.

In some embodiments, the tablet of the present disclosure comprises a Compound A monocitrate monohydrate, or a polymorphic form thereof (e.g., Form A, B, and/or C) in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A monocitrate monohydrate, or a polymorphic form thereof.

In some embodiments, the tablet of the present disclosure comprises a Compound A monocitrate monohydrate, or a polymorphic form thereof (e.g., Form A, B, and/or C) in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A monocitrate monohydrate, or a polymorphic form thereof calculated as free base equivalents.

In some embodiments, the tablet of the present disclosure comprises a Compound A citrate Form A salt in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A citrate Form A salt.

In some embodiments, the tablet of the present disclosure comprises a Compound A citrate Form A salt in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A citrate Form A salt calculated as free base equivalents.

In some embodiments, the tablet of the present disclosure comprises a Compound A monocitrate monohydrate Form A salt in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A monocitrate monohydrate Form A salt.

In some embodiments, the tablet of the present disclosure comprises a Compound A monocitrate monohydrate Form A salt in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A monocitrate monohydrate Form A salt calculated as free base equivalents.

In some embodiments, the tablet comprises Compound A citrate salt, or a polymorphic form thereof (e.g., Form A, B, and/or C) in an amount of about 25 mg, about 33.4 mg, about 110 mg, about 135 mg, about 146.5 mg, about 160 mg, about 180.2 mg, about 213.2 mg, or about 250 mg.

In some embodiments, the tablet comprises Compound A citrate salt, or a polymorphic form thereof (e.g., Form A, B, and/or C) in an amount of about 25 mg, about 33.4 mg, about 110 mg, about 135 mg, about 146.5 mg, about 160 mg, about 180.2 mg, about 213.2 mg, or about 250 mg calculated as free base equivalents.

In some embodiments, the tablet comprises Compound A monocitrate monohydrate in an amount of about 25 mg, about 33.4 mg, about 110 mg, about 135 mg, about 146.5 mg, about 160 mg, about 180.2 mg, about 213.2 mg, or about 250 mg.

In some embodiments, the tablet comprises Compound A monocitrate monohydrate in an amount of about 25 mg, about 33.4 mg, about 110 mg, about 135 mg, about 146.5 mg, about 160 mg, about 180.2 mg, about 213.2 mg, or about 250 mg calculated as free base equivalents.

In some embodiments, the tablet comprises Compound A monocitrate monohydrate such that the tablet comprises from about 10 mg to about 300 mg of Compound A.

In some embodiments, the tablet comprises Compound A monocitrate monohydrate such that the tablet comprises from about 10 mg to about 200 mg of Compound A.

In some embodiments, the tablet comprises Compound A monocitrate monohydrate such that the tablet comprises from about 25 mg to about 300 mg of Compound A.

In some embodiments, the tablet comprises Compound A monocitrate monohydrate such that the tablet comprises from about 25 mg to about 200 mg of Compound A.

In some embodiments, the tablet comprises Compound A monocitrate monohydrate such that the tablet comprises from about 25 mg to about 160 mg of Compound A.

In some embodiments, the tablet comprises Compound A monocitrate monohydrate such that the tablet comprises about 25 mg of Compound A.

In some embodiments, the tablet comprises Compound A monocitrate monohydrate such that the tablet comprises about 33.4 mg of Compound A.

In some embodiments, the tablet comprises Compound A monocitrate monohydrate such that the tablet comprises about 110 mg of Compound A.

In some embodiments, the tablet comprises Compound A monocitrate monohydrate such that the tablet comprises about 135 mg of Compound A.

In some embodiments, the tablet comprises Compound A monocitrate monohydrate such that the tablet comprises about 146.5 mg of Compound A.

In some embodiments, the tablet comprises Compound A monocitrate monohydrate such that the tablet comprises about 160 mg of Compound A.

In some embodiments, the tablet comprises Compound A monocitrate monohydrate such that the tablet comprises about 180.2 mg of Compound A.

In some embodiments, the tablet comprises Compound A monocitrate monohydrate such that the tablet comprises about 213.2 mg of Compound A calculated as free base equivalents.

In some embodiments, the tablet comprises Compound A monocitrate monohydrate such that the tablet comprises from about 10 mg to about 300 mg of Compound A calculated as free base equivalents.

In some embodiments, the tablet comprises Compound A monocitrate monohydrate such that the tablet comprises from about 10 mg to about 200 mg of Compound A calculated as free base equivalents.

In some embodiments, the tablet comprises Compound A monocitrate monohydrate such that the tablet comprises from about 25 mg to about 300 mg of Compound A calculated as free base equivalents.

In some embodiments, the tablet comprises Compound A monocitrate monohydrate such that the tablet comprises from about 25 mg to about 200 mg of Compound A calculated as free base equivalents.

In some embodiments, the tablet comprises Compound A monocitrate monohydrate such that the tablet comprises from about 25 mg to about 160 mg of Compound A free base.

In some embodiments, the tablet comprises Compound A monocitrate monohydrate such that the tablet comprises about 25 mg of Compound A calculated as free base equivalents.

In some embodiments, the tablet comprises Compound A monocitrate monohydrate such that the tablet comprises about 33.4 mg of Compound A calculated as free base equivalents.

In some embodiments, the tablet comprises Compound A monocitrate monohydrate such that the tablet comprises about 110 mg of Compound A calculated as free base equivalents.

In some embodiments, the tablet comprises Compound A monocitrate monohydrate such that the tablet comprises about 135 mg of Compound A calculated as free base equivalents.

In some embodiments, the tablet comprises Compound A monocitrate monohydrate such that the tablet comprises about 146.5 mg of Compound A calculated as free base equivalents.

In some embodiments, the tablet comprises Compound A monocitrate monohydrate such that the tablet comprises about 160 mg of Compound A calculated as free base equivalents.

In some embodiments, the tablet comprises Compound A monocitrate monohydrate such that the tablet comprises about 180.2 mg of Compound A calculated as free base equivalents.

In some embodiments, the tablet comprises Compound A monocitrate monohydrate such that the tablet comprises about 213.2 mg of Compound A calculated as free base equivalents.

In some embodiments, the tablet contains Compound A monocitrate monohydrate Form A salt so that the dosage strength is about 10 about 300 mg of Compound A.

In some embodiments, the tablet contains Compound A monocitrate monohydrate Form A salt such that the dosage is about 25 mg, about 110 mg, about 135 mg, or about 160 mg of Compound A.

In some embodiments, the tablet further comprises from about 0.1% to about 10% wt/wt of an alkalizer (e.g., sodium bicarbonate or anhydrous dibasic calcium phosphate).

In some embodiments, the tablet further comprises from about 0.1% to about 10% wt/wt of sodium bicarbonate.

In some embodiments, the tablet further comprises from about 1% to about 10% wt/wt of sodium bicarbonate.

In some embodiments, the tablet further comprises from about 1% to about 9% wt/wt of sodium bicarbonate.

In some embodiments, the tablet further comprises from about 1% to about 8% wt/wt of sodium bicarbonate.

In some embodiments, the tablet further comprises from about 1% to about 7% wt/wt of sodium bicarbonate.

In some embodiments, the tablet further comprises from about 1% to about 6% wt/wt of sodium bicarbonate.

In some embodiments, the tablet further comprises from about 2% to about 10% wt/wt of sodium bicarbonate.

In some embodiments, the tablet further comprises from about 2% to about 10% wt/wt of sodium bicarbonate.

In some embodiments, the tablet further comprises from about 2% to about 9% wt/wt of sodium bicarbonate.

In some embodiments, the tablet further comprises from about 2% to about 8% wt/wt of sodium bicarbonate.

In some embodiments, the tablet further comprises from about 2% to about 7% wt/wt of sodium bicarbonate.

In some embodiments, the tablet further comprises from about 2% to about 6% wt/wt of sodium bicarbonate.

In some embodiments, the tablet further comprises from about 3% to about 10% wt/wt of sodium bicarbonate.

In some embodiments, the tablet further comprises from about 3% to about 10% wt/wt of sodium bicarbonate.

In some embodiments, the tablet further comprises from about 3% to about 9% wt/wt of sodium bicarbonate.

In some embodiments, the tablet further comprises from about 3% to about 8% wt/wt of sodium bicarbonate.

In some embodiments, the tablet further comprises from about 3% to about 7% wt/wt of sodium bicarbonate.

In some embodiments, the tablet further comprises from about 3% to about 6% wt/wt of sodium bicarbonate.

In some embodiments, the tablet further comprises about 1%, about 2%, about 3%, about 4%, about 4.8%, about 5%, about 6%, about 7%, about 8%, about 9%, or about 10% wt/wt of sodium bicarbonate.

In some embodiments, the sodium bicarbonate in the tablet is about 5% wt/wt.

In some embodiments, the tablet further comprises from about 1 mg to about 100 mg of sodium bicarbonate.

In some embodiments, the tablet further comprises from about 1 mg to about 90 mg of sodium bicarbonate. In some embodiments, the tablet further comprises from about 1 mg to about 80 mg of sodium bicarbonate.

In some embodiments, the tablet further comprises from about 1 mg to about 70 mg of sodium bicarbonate.

In some embodiments, the tablet further comprises from about 1 mg to about 60 mg of sodium bicarbonate.

In some embodiments, the tablet further comprises from about 1 mg to about 50 mg of sodium bicarbonate.

In some embodiments, the tablet further comprises from about 1 mg to about 40 mg of sodium bicarbonate.

In some embodiments, the tablet further comprises from about 1 mg to about 30 mg of sodium bicarbonate.

In some embodiments, the tablet further comprises from about 1 mg to about 20 mg of sodium bicarbonate.

In some embodiments, the tablet further comprises from about 1 mg to about 10 mg of sodium bicarbonate.

In some embodiments, the tablet further comprises about 5 mg of sodium bicarbonate.

In some embodiments, the tablet further comprises about 6.7 mg of sodium bicarbonate.

In some embodiments, the tablet further comprises about 10 mg of sodium bicarbonate.

In some embodiments, the tablet further comprises about 15 mg of sodium bicarbonate.

In some embodiments, the tablet further comprises about 20 mg of sodium bicarbonate.

In some embodiments, the tablet further comprises about 25 mg of sodium bicarbonate.

In some embodiments, the tablet further comprises about 29.4 mg of sodium bicarbonate.

In some embodiments, the tablet further comprises about 30 mg of sodium bicarbonate.

In some embodiments, the tablet further comprises about 35 mg of sodium bicarbonate.

In some embodiments, the tablet further comprises about 36 mg of sodium bicarbonate.

In some embodiments, the tablet further comprises about 40 mg of sodium bicarbonate.

In some embodiments, the tablet further comprises about 42.7 mg of sodium bicarbonate.

In some embodiments, the tablet further comprises about 45 mg of sodium bicarbonate.

In some embodiments, the tablet further comprises about 50 mg of sodium bicarbonate.

In some embodiments, the tablet further comprises about 55 mg of sodium bicarbonate.

In some embodiments, the tablet further comprises about 60 mg of sodium bicarbonate.

In some embodiments, the tablet further comprises about 65 mg of sodium bicarbonate.

In some embodiments, the tablet further comprises about 70 mg of sodium bicarbonate.

In some embodiments, the tablet further comprises about 75 mg of sodium bicarbonate.

In some embodiments, the tablet further comprises about 80 mg of sodium bicarbonate.

In some embodiments, the tablet further comprises about 85 mg of sodium bicarbonate.

In some embodiments, the tablet further comprises about 90 mg of sodium bicarbonate.

In some embodiments, the tablet further comprises about 95 mg of sodium bicarbonate.

In some embodiments, the tablet further comprises about 100 mg of sodium bicarbonate.

Other excipients include: fillers (such as microcrystalline cellulose, mannitol, and sorbitol), disintegrants (such as starch, croscarmellose sodium, and sodium starch glycolate), binders (such as low substituted hydroxypropyl cellulose, Povidone K30 (polyvinylpyrrolidone), gelatin, sodium alginate, methylcellulose, hydroxypropylmethylcellulose, lactose, and sucrose), glidants (such as talc, fumed silica, and magnesium carbonate), lubricants (such as stearic acid, maize starch, sodium lauryl sulfate, and calcium stearate), and film coatings.

In some embodiments, the tablet comprises
Compound A monocitrate monohydrate;
bicarbonate; and
a pharmaceutically acceptable excipient.

In some embodiments, the tablet is prepared by a process which comprises combining Compound A monocitrate monohydrate Form A; bicarbonate; and a pharmaceutically acceptable excipient.

In some embodiments, the tablet comprises
10% to 40% wt/wt of a Compound A monocitrate monohydrate salt;
0.1% to 10% wt/wt of a bicarbonate;
20% to 70% wt/wt of a filler.

In some embodiments, the tablet comprises
10% to 40% wt/wt of a Compound A monocitrate monohydrate salt;
0.1% to 10% wt/wt of a bicarbonate;
20% to 70% wt/wt of a filler;
1% to 20% wt/wt of a disintegrant;
1% to 20% wt/wt of a binder; and
0.5% to 5% wt/wt of a lubricant.

In some embodiments, the tablet comprises
10% to 40% wt/wt of a Compound A monocitrate monohydrate salt;
0.1% to 10% wt/wt of a bicarbonate;
20% to 70% wt/wt of a filler;
1% to 20% wt/wt of a disintegrant;
1% to 20% wt/wt of a binder; and
0.5% to 5% wt/wt of a lubricant.

In some embodiments, the tablet comprises an intragranular phase and an extragranular phase, wherein:
(a) the intragranular phase comprises:
   10% to 40% wt/wt of a Compound A monocitrate monohydrate salt;
   20% to 70% wt/wt of a filler;
   1% to 20% wt/wt of a disintegrant;
   1% to 20% wt/wt of a binder; and
(b) the extragranular phase comprises:
   0.5% to 5% wt/wt of a lubricant.

In some embodiments, the tablet comprises an intragranular phase and an extragranular phase, wherein:
(a) the intragranular phase comprises:
   10% to 40% wt/wt of a Compound A monocitrate monohydrate salt;
   20% to 70% wt/wt of a filler;
   1% to 20% wt/wt of a disintegrant;
   1% to 20% wt/wt of a binder; and
(b) the extragranular phase comprises:
   0.5% to 5% wt/wt of a lubricant.

In some embodiments, the tablet comprises an intragranular phase and an extragranular phase, wherein:
(a) the intragranular phase comprises:
   15% to 30% wt/wt of a Compound A monocitrate monohydrate salt;
   40% to 60% wt/wt of a filler;
   5% to 15% wt/wt of a disintegrant;
   5% to 10% wt/wt of a binder; and
(b) the extragranular phase comprises:
   0.5% to 5% wt/wt of a lubricant.

In some embodiments, the tablet comprises an intragranular phase and an extragranular phase, wherein:
(a) the intragranular phase comprises:
   15% to 30% wt/wt of a Compound A monocitrate monohydrate salt;
   40% to 60% wt/wt of a filler;
   5% to 15% wt/wt or a disintegrant;
   5% to 10% wt/wt or a binder;
   2% to 7% wt/wt sodium bicarbonate;
   0.1% to 2% wt/wt of a lubricant; and
(b) the extragranular phase comprises:
   0.5% to 5% wt/wt a lubricant.

In some embodiments, the tablet comprises an intragranular phase and an extragranular phase, wherein:
(a) the intragranular phase comprises:
   15% to 30% wt/wt of a Compound A monocitrate monohydrate Form A salt;
   45% to 60% wt/wt of a filler (e.g., a filler comprising microcrystalline cellulose);
   5% to 15% wt/wt a disintegrant (e.g., crospovidone);
   5% to 10% wt/wt a binder (e.g., hypromellose);
   2% to 7% wt/wt sodium bicarbonate;
   0.1% to 2% wt/wt a glidant (e.g., hydrophobic colloidal silica);
   0.1% to 2% wt/wt a lubricant (e.g., magnesium stearate); and
(b) the extragranular phase comprises:
   0.5% to 5% wt/wt a lubricant (e.g., magnesium stearate).

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt), and
from about 1% to about 10% wt/wt sodium bicarbonate.

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt),
from about 45% to about 60% wt/wt of a filler (e.g., a filler comprising microcrystalline cellulose); and
from about 1% to about 10% wt/wt sodium bicarbonate.

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt),
from about 45% to about 60% wt/wt of a filler (e.g., a filler comprising microcrystalline cellulose);
from about 1% to about 10% wt/wt sodium bicarbonate; and
from about 0.1% to about 2% wt/wt a glidant (e.g., hydrophobic colloidal silica).

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt),
from about 45% to about 60% wt/wt of a filler (e.g., a filler comprising microcrystalline cellulose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate).

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt),
from about 45% to about 60% wt/wt of a filler (e.g., a filler comprising microcrystalline cellulose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate).

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt),
from about 45% to about 60% wt/wt of a filler (e.g., a filler comprising microcrystalline cellulose);
from about 5% to about 20% wt/wt a binder (e.g., hypromellose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate).

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt),
from about 45% to about 60% wt/wt of a filler (e.g., a filler comprising microcrystalline cellulose);
from about 5% to about 20% wt/wt a binder (e.g., hypromellose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate).

In some embodiments, the tablet of the present disclosure comprises an intragranular phase and an extragranular phase, wherein:
(a) the intragranular phase comprises:
   from about 15% to about 30% wt/wt of a Compound A citrate salt (e.g., Compound A monocitrate monohydrate salt) or polymorphic form thereof (e.g., Compound A monocitrate monohydrate Form A salt); and
(b) the extragranular phase comprises:
   from about 0.5% to about 5% wt/wt a lubricant (e.g., magnesium stearate).

In some embodiments, the tablet of the present disclosure comprises an intragranular phase and an extragranular phase, wherein:
(a) the intragranular phase comprises:
   from about 15% to about 30% wt/wt of a Compound A citrate salt (e.g., Compound A monocitrate monohydrate salt) or polymorphic form thereof (e.g., Compound A monocitrate monohydrate Form A salt);
   about 1% to about 10% wt/wt of sodium bicarbonate; and
(b) the extragranular phase comprises:
   from about 0.5% to about 5% wt/wt a lubricant (e.g., magnesium stearate).

In some embodiments, the tablet of the present disclosure further comprises an intragranular phase and an extragranular phase, wherein:
(a) the intragranular phase comprises:
   from about 15% to about 30% wt/wt of a Compound A citrate salt (e.g., Compound A monocitrate monohydrate salt) or polymorphic form thereof (e.g., Compound A monocitrate monohydrate Form A salt);
   from about 45% to about 60% wt/wt of a filler (e.g., a filler comprising microcrystalline cellulose);
   from about 5% to about 15% wt/wt a disintegrant (e.g., crospovidone);
   from about 5% to about 20% wt/wt a binder (e.g., hypromellose);
   from about 2% to about 7% wt/wt sodium bicarbonate;
   from about 0.1% to about 2% wt/wt a glidant (e.g., hydrophobic colloidal silica);
   from about 0.1% to about 2% wt/wt a lubricant (e.g., magnesium stearate); and
(b) the extragranular phase comprises:
   from about 0.5% to about 5% wt/wt a lubricant (e.g., magnesium stearate).

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt) calculated as free base equivalents, and
from about 1% to about 10% wt/wt sodium bicarbonate.

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt) calculated as free base equivalents,
from about 45% to about 60% wt/wt of a filler (e.g., a filler comprising microcrystalline cellulose); and
from about 1% to about 10% wt/wt sodium bicarbonate.

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt) calculated as free base equivalents,
from about 45% to about 60% wt/wt of a filler (e.g., a filler comprising microcrystalline cellulose);
from about 1% to about 10% wt/wt sodium bicarbonate; and
from about 0.1% to about 2% wt/wt a glidant (e.g., hydrophobic colloidal silica).

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt) calculated as free base equivalents,
from about 45% to about 60% wt/wt of a filler (e.g., a filler comprising microcrystalline cellulose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate).

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt) calculated as free base equivalents,
from about 45% to about 60% wt/wt of a filler (e.g., a filler comprising microcrystalline cellulose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate).

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt) calculated as free base equivalents,
from about 45% to about 60% wt/wt of a filler (e.g., a filler comprising microcrystalline cellulose);
from about 5% to about 20% wt/wt a binder (e.g., hypromellose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate).

In some embodiments, the tablet of the present disclosure comprises:
from about 15% to about 30% wt/wt of a Compound A citrate salt (e.g., Compound A monocitrate monohydrate salt) or polymorphic form thereof (e.g., Compound A monocitrate monohydrate Form A salt) calculated as free base equivalents,
from about 45% to about 60% wt/wt of a filler (e.g., a filler comprising microcrystalline cellulose);
from about 5% to about 20% wt/wt a binder (e.g., hypromellose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate).

In some embodiments, the tablet of the present disclosure comprises an intragranular phase and an extragranular phase, wherein:
(a) the intragranular phase comprises:
   from about 15% to about 30% wt/wt of a Compound A citrate salt (e.g., Compound A monocitrate monohydrate salt) or polymorphic form thereof (e.g., Compound A monocitrate monohydrate Form A salt) calculated as free base equivalents; and
(b) the extragranular phase comprises:
   from about 0.5% to about 5% wt/wt a lubricant (e.g., magnesium stearate).

In some embodiments, the tablet of the present disclosure comprises an intragranular phase and an extragranular phase, wherein:
(a) the intragranular phase comprises:
   from about 15% to about 30% wt/wt of a Compound A citrate salt (e.g., Compound A monocitrate monohydrate salt) or polymorphic form thereof (e.g., Compound A monocitrate monohydrate Form A salt) calculated as free base equivalents;
   about 1% to about 10% wt/wt of sodium bicarbonate; and
(b) the extragranular phase comprises:
   from about 0.5% to about 5% wt/wt a lubricant (e.g., magnesium stearate).

In some embodiments, the tablet of the present disclosure further comprises an intragranular phase and an extragranular phase, wherein:
(a) the intragranular phase comprises:
   from about 15% to about 30% wt/wt of a Compound A citrate salt (e.g., Compound A monocitrate monohydrate salt) or polymorphic form thereof (e.g., Compound A monocitrate monohydrate Form A salt) calculated as free base equivalents;
   from about 45% to about 60% wt/wt of a filler (e.g., a filler comprising microcrystalline cellulose);
   from about 5% to about 15% wt/wt a disintegrant (e.g., crospovidone);
   from about 5% to about 20% wt/wt a binder (e.g., hypromellose);
   from about 2% to about 7% wt/wt sodium bicarbonate;
   from about 0.1% to about 2% wt/wt a glidant (e.g., hydrophobic colloidal silica);
   from about 0.1% to about 2% wt/wt a lubricant (e.g., magnesium stearate); and
(b) the extragranular phase comprises:
   from about 0.5% to about 5% wt/wt a lubricant (e.g., magnesium stearate).

In some embodiments, the tablet of the present disclosure comprises:
from about 15% to about 30% wt/wt of a Compound A monocitrate monohydrate Form A salt calculated as free base equivalents, and
from about 1% to about 10% wt/wt sodium bicarbonate.

In some embodiments, the tablet of the present disclosure comprises:
from about 15% to about 30% wt/wt of a Compound A monocitrate monohydrate Form A salt calculated as free base equivalents,
from about 45% to about 60% wt/wt of a filler (e.g., a filler comprising microcrystalline cellulose); and
from about 1% to about 10% wt/wt sodium bicarbonate.

In some embodiments, the tablet of the present disclosure comprises:
from about 15% to about 30% wt/wt of a Compound A monocitrate monohydrate Form A salt calculated as free base equivalents,
from about 45% to about 60% wt/wt of a filler (e.g., a filler comprising microcrystalline cellulose);
from about 1% to about 10% wt/wt sodium bicarbonate; and
from about 0.1% to about 2% wt/wt a glidant (e.g., hydrophobic colloidal silica).

In some embodiments, the tablet of the present disclosure comprises:
from about 15% to about 30% wt/wt of a Compound A monocitrate monohydrate Form A salt calculated as free base equivalents,
from about 45% to about 60% wt/wt of a filler (e.g., a filler comprising microcrystalline cellulose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate).

In some embodiments, the tablet of the present disclosure comprises:
from about 15% to about 30% wt/wt of a Compound A monocitrate monohydrate Form A salt calculated as free base equivalents,
from about 45% to about 60% wt/wt of a filler (e.g., a filler comprising microcrystalline cellulose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate).

In some embodiments, the tablet of the present disclosure comprises:
from about 15% to about 30% wt/wt of a Compound A monocitrate monohydrate Form A salt calculated as free base equivalents,
from about 45% to about 60% wt/wt of a filler (e.g., a filler comprising microcrystalline cellulose);
from about 5% to about 20% wt/wt a binder (e.g., hypromellose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate).

In some embodiments, the tablet of the present disclosure comprises:
from about 15% to about 30% wt/wt of a Compound A monocitrate monohydrate Form A salt calculated as free base equivalents,
from about 45% to about 60% wt/wt of a filler (e.g., a filler comprising microcrystalline cellulose);
from about 5% to about 20% wt/wt a binder (e.g., hypromellose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate).

In some embodiments, the tablet of the present disclosure comprises an intragranular phase and an extragranular phase, wherein:
(a) the intragranular phase comprises:
   from about 15% to about 30% wt/wt of a Compound A monocitrate monohydrate Form A salt calculated as free base equivalents; and
(b) the extragranular phase comprises:
   from about 0.5% to about 5% wt/wt a lubricant (e.g., magnesium stearate).

In some embodiments, the tablet of the present disclosure comprises an intragranular phase and an extragranular phase, wherein:
(a) the intragranular phase comprises:
   from about 15% to about 30% wt/wt of a Compound A monocitrate monohydrate Form A salt calculated as free base equivalents;
   about 1% to about 10% wt/wt of sodium bicarbonate; and
(b) the extragranular phase comprises:
   from about 0.5% to about 5% wt/wt a lubricant (e.g., magnesium stearate).

In some embodiments, the tablet of the present disclosure further comprises an intragranular phase and an extragranular phase, wherein:
(a) the intragranular phase comprises:
   from about 15% to about 30% wt/wt of a Compound A monocitrate monohydrate Form A salt calculated as free base equivalents;
   from about 45% to about 60% wt/wt of a filler (e.g., a filler comprising microcrystalline cellulose);
   from about 5% to about 15% wt/wt a disintegrant (e.g., crospovidone);
   from about 5% to about 20% wt/wt a binder (e.g., hypromellose);
   from about 2% to about 7% wt/wt sodium bicarbonate;
   from about 0.1% to about 2% wt/wt a glidant (e.g., hydrophobic colloidal silica);
   from about 0.1% to about 2% wt/wt a lubricant (e.g., magnesium stearate); and
(b) the extragranular phase comprises:
   from about 0.5% to about 5% wt/wt a lubricant (e.g., magnesium stearate).

In some embodiments, the tablet comprises about 213.2 mg of Compound A monocitrate monohydrate Form A salt calculated as free base equivalents.

In some embodiments, the tablet of the present disclosure comprises:
from about 25 mg to about 250 mg of a Compound A citrate salt (e.g., Compound A monocitrate monohydrate salt) or polymorphic form thereof (e.g., Compound A monocitrate monohydrate Form A salt) calculated as free base equivalents, and
from about 5 mg to about 50 mg sodium bicarbonate.

In some embodiments, the tablet of the present disclosure comprises:
from about 25 mg to about 250 mg of a Compound A citrate salt (e.g., Compound A monocitrate monohydrate salt) or polymorphic form thereof (e.g., Compound A monocitrate monohydrate Form A salt) calculated as free base equivalents,
from about 50 mg to about 500 mg of a filler (e.g., a filler comprising microcrystalline cellulose); and
from about 5 mg to about 50 mg sodium bicarbonate.

In some embodiments, the tablet of the present disclosure comprises:
from about 25 mg to about 250 mg of a Compound A citrate salt (e.g., Compound A monocitrate monohydrate salt) or polymorphic form thereof (e.g., Compound A monocitrate monohydrate Form A salt) calculated as free base equivalents,
from about 50 mg to about 500 mg of a filler (e.g., a filler comprising microcrystalline cellulose);
from about 5 mg to about 50 mg sodium bicarbonate; and
from about 1 mg to about 10 mg a glidant (e.g., hydrophobic colloidal silica).

In some embodiments, the tablet of the present disclosure comprises:
from about 25 mg to about 250 mg of a Compound A citrate salt (e.g., Compound A monocitrate monohydrate salt) or polymorphic form thereof (e.g., Compound A monocitrate monohydrate Form A salt) calculated as free base equivalents,
from about 50 mg to about 500 mg of a filler (e.g., a filler comprising microcrystalline cellulose);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate).

In some embodiments, the tablet of the present disclosure comprises:
from about 25 mg to about 250 mg of a Compound A citrate salt (e.g., Compound A monocitrate monohydrate salt) or polymorphic form thereof (e.g., Compound A monocitrate monohydrate Form A salt) calculated as free base equivalents,
from about 50 mg to about 500 mg of a filler (e.g., a filler comprising microcrystalline cellulose);
from about 10 mg to about 100 mg a disintegrant (e.g., crospovidone);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate).

In some embodiments, the tablet of the present disclosure comprises:
from about 25 mg to about 250 mg of a Compound A citrate salt (e.g., Compound A monocitrate monohydrate salt) or polymorphic form thereof (e.g., Compound A monocitrate monohydrate Form A salt) calculated as free base equivalents,
from about 50 mg to about 500 mg of a filler (e.g., a filler comprising microcrystalline cellulose);
from about 10 mg to about 100 mg a binder (e.g., hypromellose);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate).

In some embodiments, the tablet of the present disclosure comprises:
from about 25 mg to about 250 mg of a Compound A citrate salt (e.g., Compound A monocitrate monohydrate salt) or polymorphic form thereof (e.g., Compound A monocitrate monohydrate Form A salt) calculated as free base equivalents,
from about 50 mg to about 500 mg of a filler (e.g., a filler comprising microcrystalline cellulose);
from about 10 mg to about 100 mg a binder (e.g., hypromellose);
from about 10 mg to about 100 mg a disintegrant (e.g., crospovidone);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate).

In some embodiments, the tablet of the present disclosure comprises an intragranular phase and an extragranular phase, wherein:
(a) the intragranular phase comprises:
   from about 25 mg to about 250 mg of a Compound A citrate salt (e.g., Compound A monocitrate monohydrate salt) or polymorphic form thereof (e.g., Compound A monocitrate monohydrate Form A salt) calculated as free base equivalents; and
(b) the extragranular phase comprises:
   from about 1 mg to about 10 mg a lubricant (e.g., magnesium stearate).

In some embodiments, the tablet of the present disclosure comprises an intragranular phase and an extragranular phase, wherein:
(a) the intragranular phase comprises:
   from about 25 mg to about 250 mg of a Compound A citrate salt (e.g., Compound A monocitrate monohydrate salt) or polymorphic form thereof (e.g., Compound A monocitrate monohydrate Form A salt) calculated as free base equivalents;
   about 5 mg to about 50 mg of sodium bicarbonate; and
(b) the extragranular phase comprises:
   from about 1 mg to about 10 mg a lubricant (e.g., magnesium stearate).

In some embodiments, the tablet of the present disclosure further comprises an intragranular phase and an extragranular phase, wherein:
(a) the intragranular phase comprises:
   from about 25 mg to about 250 mg of a Compound A citrate salt (e.g., Compound A monocitrate monohydrate salt) or polymorphic form thereof (e.g., Compound A monocitrate monohydrate Form A salt) calculated as free base equivalents;
   from about 50 mg to about 500 mg of a filler (e.g., a filler comprising microcrystalline cellulose);
   from about 10 mg to about 100 mg a disintegrant (e.g., crospovidone);
   from about 10 mg to about 100 mg a binder (e.g., hypromellose);
   from about 5 mg to about 50 mg sodium bicarbonate;
   from about 1 mg to about 10 mg a glidant (e.g., hydrophobic colloidal silica);
   from about 0.5 mg to about 15 mg a lubricant (e.g., magnesium stearate); and
(b) the extragranular phase comprises:
   from about 1 mg to about 10 mg a lubricant (e.g., magnesium stearate).

In some embodiments, the tablet of the present disclosure comprises:
from about 25 mg to about 250 mg of a Compound A monocitrate monohydrate Form A salt calculated as free base equivalents, and
from about 5 mg to about 50 mg sodium bicarbonate.

In some embodiments, the tablet of the present disclosure comprises:
from about 25 mg to about 250 mg of a Compound A monocitrate monohydrate Form A salt calculated as free base equivalents,
from about 50 mg to about 500 mg of a filler (e.g., a filler comprising microcrystalline cellulose); and
from about 5 mg to about 50 mg sodium bicarbonate.

In some embodiments, the tablet of the present disclosure comprises:
from about 25 mg to about 250 mg of a Compound A monocitrate monohydrate Form A salt calculated as free base equivalents,
from about 50 mg to about 500 mg of a filler (e.g., a filler comprising microcrystalline cellulose);
from about 5 mg to about 50 mg sodium bicarbonate; and
from about 1 mg to about 10 mg a glidant (e.g., hydrophobic colloidal silica).

In some embodiments, the tablet of the present disclosure comprises:
from about 25 mg to about 250 mg of a Compound A monocitrate monohydrate Form A salt calculated as free base equivalents,
from about 50 mg to about 500 mg of a filler (e.g., a filler comprising microcrystalline cellulose);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate).

In some embodiments, the tablet of the present disclosure comprises:
from about 25 mg to about 250 mg of a Compound A monocitrate monohydrate Form A salt calculated as free base equivalents,
from about 50 mg to about 500 mg of a filler (e.g., a filler comprising microcrystalline cellulose);
from about 10 mg to about 100 mg a disintegrant (e.g., crospovidone);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate).

In some embodiments, the tablet of the present disclosure comprises:
from about 25 mg to about 250 mg of a Compound A monocitrate monohydrate Form A salt calculated as free base equivalents,
from about 50 mg to about 500 mg of a filler (e.g., a filler comprising microcrystalline cellulose);
from about 10 mg to about 100 mg a binder (e.g., hypromellose);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate).

In some embodiments, the tablet of the present disclosure comprises:
from about 25 mg to about 250 mg of a Compound A monocitrate monohydrate Form A salt calculated as free base equivalents,
from about 50 mg to about 500 mg of a filler (e.g., a filler comprising microcrystalline cellulose);
from about 10 mg to about 100 mg a binder (e.g., hypromellose);
from about 10 mg to about 100 mg a disintegrant (e.g., crospovidone);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate).

In some embodiments, the tablet of the present disclosure comprises an intragranular phase and an extragranular phase, wherein:
(a) the intragranular phase comprises:
   from about 25 mg to about 250 mg of a Compound A monocitrate monohydrate Form A salt calculated as free base equivalents; and
(b) the extragranular phase comprises:
   from about 1 mg to about 10 mg a lubricant (e.g., magnesium stearate).

In some embodiments, the tablet of the present disclosure comprises an intragranular phase and an extragranular phase, wherein:
(a) the intragranular phase comprises:
   from about 25 mg to about 250 mg of a Compound A monocitrate monohydrate Form A salt calculated as free base equivalents;
   about 5 mg to about 50 mg of sodium bicarbonate; and
(b) the extragranular phase comprises:
   from about 1 mg to about 10 mg a lubricant (e.g., magnesium stearate).

In some embodiments, the tablet of the present disclosure further comprises an intragranular phase and an extragranular phase, wherein:
(a) the intragranular phase comprises:
   from about 25 mg to about 250 mg of a Compound A monocitrate monohydrate Form A salt calculated as free base equivalents;
   from about 50 mg to about 500 mg of a filler (e.g., a filler comprising microcrystalline cellulose);
   from about 10 mg to about 100 mg a disintegrant (e.g., crospovidone);
   from about 10 mg to about 100 mg a binder (e.g., hypromellose);
   from about 5 mg to about 50 mg sodium bicarbonate;
   from about 1 mg to about 10 mg a glidant (e.g., hydrophobic colloidal silica);
   from about 0.5 mg to about 15 mg a lubricant (e.g., magnesium stearate); and
(b) the extragranular phase comprises:
   from about 1 mg to about 10 mg a lubricant (e.g., magnesium stearate).

In some embodiments, the tablet of the present disclosure comprises:
about 33.4 mg of Compound A citrate salt (e.g., Compound A monocitrate monohydrate salt) or polymorphic form thereof (e.g., Compound A monocitrate monohydrate Form A salt), and
about 6.7 mg sodium bicarbonate.

In some particular embodiments, the tablet of the present disclosure comprises:

| Material | Concentration (% w/w) |
|---|---|
| Tablet Strength | 25 mg |

Intra-granular

| | |
|---|---|
| Compound A monocitrate | 18.7 (calculated as free base |
| monohydrate | equivalents |
| Avicel-DG ™ | 53.8 |
| Crospovidone | 10.0 |
| Hypromellose | 10.0 |
| Sodium bicarbonate | 5.0 |
| Hydrophobic colloidal silica | 1.0 |
| Magnesium stearate | 0.5 |
| Total Intragranular | 99 |

Extra-granular

| | |
|---|---|
| Magnesium stearate | 1.0 |
| Total core tablet | 100 |
| Opadry Film Coat | 4.8 (Opadry II Pink) |
| Purified Water | Removed during processing |

In some particular embodiments, the tablet of the present disclosure comprises:

| Material | Concentration (% w/w) |
|---|---|
| Tablet Strength | 110 mg |

Intra-granular

| | |
|---|---|
| Compound A monocitrate | 18.7 (calculated as free base |
| monohydrate | equivalents) |
| Avicel-DG ™ | 53.8 |
| Crospovidone | 10.0 |
| Hypromellose | 10.0 |
| Sodium bicarbonate | 5.0 |
| Hydrophobic colloidal silica | 1.0 |
| Magnesium stearate | 0.5 |
| Total Intragranular | 99 |

Extra-granular

| | |
|---|---|
| Magnesium stearate | 1.0 |
| Total core tablet | 100 |
| Opadry Film Coat | 4.8 (Opadry II Beige) |
| Purified Water | Removed during processing |

In some particular embodiments, the tablet of the present disclosure comprises:

| Material | Concentration (% w/w) |
|---|---|
| Tablet Strength | 160 mg |

Intra-granular

| | |
|---|---|
| Compound A monocitrate | 18.7 (calculated as free base |
| monohydrate | equivalents) |
| Avicel-DG ™ | 53.8 |
| Crospovidone | 10.0 |
| Hypromellose | 10.0 |
| Sodium bicarbonate | 5.0 |
| Hydrophobic colloidal silica | 1.0 |
| Magnesium stearate | 0.5 |
| Total Intragranular | 99 |

Extra-granular

| | |
|---|---|
| Magnesium stearate | 1.0 |
| Total core tablet | 100 |
| Opadry Film Coat | 4.8 (Opadry II Purple) |
| Purified Water | Removed during processing |

In some particular embodiments, the tablet of the present disclosure comprises:

| Material | Concentration (% w/w) |
|---|---|
| Tablet Strength | 25 mg |

Intra-granular

| | |
|---|---|
| Compound A monocitrate | 23.8 (Calculated as Compound A |
| monohydrate | monocitrate monohydrate salt and |
| | assuming an Assay of 100%) |
| Avicel-DG ® | 45.3 |
| Crospovidone | 9.5 |
| Hypromellose | 9.5 |
| Sodium bicarbonate | 4.8 |
| Hydrophobic colloidal silica | 1.0 |
| Magnesium stearate | 0.5 |
| Total Intragranular | 99.0 |

Extra-granular

| | |
|---|---|
| Magnesium stearate | 1.0 |
| Total core tablet | 95.2 |
| Opadry ® II Pink Film Coat | 4.8 |
| Opadry ® II Beige Film Coat | — |
| Opadry ® II Purple Film Coat | — |
| Purified Water | Removed during processing |

In some particular embodiments, the tablet of the present disclosure comprises:

| Material | Concentration (% w/w) |
|---|---|
| Tablet Strength | 110 mg |

Intra-granular

| | |
|---|---|
| Compound A monocitrate | 23.8 (Calculated as Compound A |
| monohydrate | monocitrate monohydrate salt and |
| | assuming an Assay of 100%) |
| Avicel-DG ® | 45.3 |
| Crospovidone | 9.5 |
| Hypromellose | 9.5 |
| Sodium bicarbonate | 4.8 |
| Hydrophobic colloidal silica | 1.0 |
| Magnesium stearate | 0.5 |
| Total Intragranular | 99.0 |

Extra-granular

| | |
|---|---|
| Magnesium stearate | 1.0 |
| Total core tablet | 95.2 |
| Opadry ® II Pink Film Coat | — |
| Opadry ® II Beige Film Coat | 4.8 |
| Opadry ® II Purple Film Coat | — |
| Purified Water | Removed during processing |

In some particular embodiments, the tablet of the present disclosure comprises:

| Material | Concentration (% w/w) |
|---|---|
| Tablet Strength | 160 mg |

Intra-granular

| | |
|---|---|
| Compound A monocitrate | 23.8 (Calculated as Compound A |
| monohydrate | monocitrate monohydrate salt and |
| | assuming an Assay of 100%) |
| Avicel-DG ® | 45.3 |
| Crospovidone | 9.5 |
| Hypromellose | 9.5 |
| Sodium bicarbonate | 4.8 |
| Hydrophobic colloidal silica, | 1.0 |
| NF/EP | |
| Magnesium stearate | 0.5 |
| Total Intragranular | 99.0 |

Extra-granular

| | |
|---|---|
| Magnesium stearate | 1.0 |
| Total core tablet | 95.2 |
| Opadry ® II Pink Film Coat | — |
| Opadry ® II Beige Film Coat | — |
| Opadry ® II Purple Film Coat | 4.8 |
| Purified Water | Removed during processing |

In some particular embodiments, the tablet of the present disclosure comprises:

| | |
|---|---|
| | Tablet Strength |
| Material | 25 mg |

Amount, mg

| | |
|---|---|
| Compound A monocitrate | 33.4 |
| monohydrate Form A salt | |
| Avicel ® DG | 63.7 |
| Crospovidone | 13.4 |
| Hypromellose | 13.4 |
| Sodium Bicarbonate | 6.7 |
| Hydrophobic Colloidal | 1.3 |
| Silica | |
| Magnesium Stearate | 0.7 |
| (Intra-granular) | |
| Magnesium Stearate | 1.3 |
| (Extra-granular) | |
| Total | 134 |
| Compound A Core Tablet | 134 |
| Opadry ® Film C | 6.7 (Opadry Pink) |
| Purified Water | Removed |

In some particular embodiments, the tablet of the present disclosure comprises:

| | |
|---|---|
| | Tablet Strength |
| Material | 110 mg |

Amount, mg

| | |
|---|---|
| Compound A monocitrate | 146.5 |
| monohydrate Form A salt | |
| Avicel ® DG | 279.0 |
| Crospovidone | 58.7 |
| Hypromellose | 58.7 |
| Sodium Bicarbonate | 29.4 |
| Hydrophobic Colloidal | 5.9 |
| Silica | |
| Magnesium Stearate | 2.9 |
| (Intra-granular) | |
| Magnesium Stearate | 5.9 |
| (Extra-granular) | |
| Total | 587 |
| Compound A Core Tablet | 587 |
| Opadry ® Film Co | 29.4 (Opadry Beige) |
| Purified Water | Removed |

In some particular embodiments, the tablet of the present disclosure comprises:

| | |
|---|---|
| | Tablet Strength |
| Material | 160 mg |

Amount, mg

| | |
|---|---|
| Compound A monocitrate | 213.2 |
| monohydrate Form A salt | |
| Avicel ® DG | 406.0 |
| Crospovidone | 85.4 |
| Hypromellose | 85.4 |
| Sodium Bicarbonate | 42.7 |
| Hydrophobic Colloidal Silica | 8.5 |
| Magnesium Stearate (Intra-granular) | 4.3 |
| Magnesium Stearate (Extra-granular) | 8.5 |
| Total | 854 |
| Compound A Core Tablet | 854 |
| Opadry ® Film Coat | 42.7 (Opadry Purple) |
| Purified Water | Removed |

In some particular embodiments, the tablet of the present disclosure comprises:

| | | 25 mg | 110 mg | 135 mg | 160 mg |
|---|---|---|---|---|---|
| | Composition | Strength | Strength | Strength | Strength |
| Component | % w/w | (mg/tablet) | (mg/tablet) | (mg/tablet) | (mg/tablet) |
| Compound A citrate | 23.8 | 33.4 | 146.5 | 180.2 | 213.2 |
| Avicel PH-101 | 22.6 | 31.8 | 139.1 | 170.9 | 202.4 |
| Avicel PH-102 | 11.3 | 15.9 | 69.9 | 85.8 | 101.6 |
| Anhydrous dibasic | 11.3 | 15.9 | 69.9 | 85.8 | 101.6 |
| calcium phosphate | | | | | |
| Crospovidone | 9.5 | 13.4 | 58.7 | 72.1 | 85.4 |
| Hypromellose | 9.5 | 13.4 | 58.7 | 72.1 | 85.4 |
| Sodium Bicarbonate | 4.8 | 6.7 | 29.4 | 36.0 | 42.7 |
| Hydrophobic | 1.0 | 1.3 | 5.9 | 7.2 | 8.5 |
| Colloidal Silica | | | | | |
| Magnesium Stearate | 0.5 | 0.7 | 2.9 | 3.6 | 4.3 |
| (Intra-granular) | | | | | |
| Magnesium Stearate | 1.0 | 1.3 | 5.9 | 7.2 | 8.5 |
| (Extra-granular) | | | | | |
| Compound A | — | 134 | 587 | 721 | 854 |
| Common Blend | | | | | |
| Opadry ® II Film | 4.8 | 6.7 | 29.4 | 36.1 | 42.7 |
| Coat | | (85F94190 | (85F97253- | (85F130040 | (85F90035 |
| | | Pink) | CN Beige) | Orange) | Purple) |

*Avicel PH-101 and PH-102 are both microcrystalline cellulose

In some embodiments, each 25 mg strength tablet contains 25 mg Compound A, equivalent to 33.4 mg Compound A citrate, and the following inactive ingredients: microcrystalline cellulose, dicalcium phosphate, crospovidone, hypromellose, sodium bicarbonate, hydrophobic colloidal silica, magnesium stearate, polyvinyl alcohol, titanium dioxide, polyethylene glycol, talc, and red iron oxide.

In some embodiments, each 110 mg strength tablet contains 110 mg Compound A, equivalent to 146.5 mg Compound A citrate, and the following inactive ingredients: microcrystalline cellulose, dicalcium phosphate, crospovidone, hypromellose, sodium bicarbonate, hydrophobic colloidal silica, magnesium stearate, polyvinyl alcohol, titanium dioxide, polyethylene glycol, talc, red iron oxide, and yellow iron oxide.

In some embodiments, each 135 mg strength tablet contains 135 mg Compound A, equivalent to 180.2 mg Compound A citrate, and the following inactive ingredients: microcrystalline cellulose, dicalcium phosphate, crospovidone, hypromellose, sodium bicarbonate, hydrophobic colloidal silica, magnesium stearate, and Opadry® II Film Coat.

In some embodiments, each 160 mg strength tablet contains 160 mg Compound A equivalent to 213.2 mg Compound A citrate, and the following inactive ingredients: microcrystalline cellulose, dicalcium phosphate, crospovidone, hypromellose, sodium bicarbonate, hydrophobic colloidal silica, magnesium stearate, polyvinyl alcohol, titanium dioxide, polyethylene glycol, talc, red iron oxide, and FD&C blue #2/indigo carmine aluminum lake.

In some embodiments, the dicalcium phosphate is anhydrous dibasic calcium phosphate.

In some embodiments, the tablet comprising Compound A citrate salt exhibits improved solubility compared to a tablet comprising Compound A sesquifumarate salt.

In some embodiments, the tablet comprising Compound A citrate salt and sodium bicarbonate exhibits improved solubility compared to a tablet comprising Compound A sesquifumarate salt.

In some embodiments, the tablet comprising Compound A citrate salt exhibits faster solubility compared to a tablet comprising Compound A sesquifumarate salt.

In some embodiments, the tablet comprising Compound A citrate salt and sodium bicarbonate exhibits faster solubility compared to a tablet comprising Compound A sesquifumarate salt.

In some embodiments, the tablet comprising Compound A citrate salt and sodium bicarbonate exhibits faster solubility compared to a capsule comprising Compound A sesquifumarate salt.

In some embodiments, the tablet comprising Compound A citrate salt exhibits higher stability compared to a tablet comprising Compound A sesquifumarate salt.

In some embodiments, the tablet comprising Compound A monocitrate monohydrate Form A salt exhibits higher stability compared to a tablet comprising Compound A sesquifumarate salt.

In some embodiments, the tablet comprising Compound A citrate salt and sodium bicarbonate exhibits higher stability compared to a tablet comprising Compound A sesquifumarate salt.

In some embodiments, the tablet comprising Compound A citrate salt and sodium bicarbonate exhibits higher stability compared to a capsule comprising Compound A sesquifumarate salt.

The tablets may include one or more pharmaceutically acceptable excipients, carriers, or diluents/fillers. Surfactants, diluents, sweeteners, disintegrants, binders, lubricants, glidants, colorants, flavors, stabilizing agents, mixtures thereof and the like can be used.

Glidants are, but not limited to, silicon dioxide, colloidal silicon dioxide, calcium silicate, hydrophobic colloidal silica, magnesium silicate, magnesium trisilicate, talc, starch, mixtures thereof or the like.

Lubricants are, but not limited to, calcium stearate, glyceryl monostearate, glyceryl behenate, glyceryl palmitostearate, hexagonal boron nitride, hydrogenated vegetable oil, light mineral oil, magnesium stearate, mineral oil, polyethylene glycol, poloxamer, sodium benzoate, sodium lauryl sulfate, sodium stearyl fumarate, stearic acid, talc, zinc stearate, mixtures thereof or the like.

Disintegrants are, but not limited to, sodium starch glycolate, sodium carboxymethyl cellulose, calcium carboxymethyl cellulose, croscarmellose sodium, crospovidone, chitosan, agar, alginic acid, calcium alginate, methyl cellulose, powdered cellulose, lower alkylsubstituted hydroxypropyl cellulose, hydroxylpropyl starch, low-substituted hydroxypropylcellulose, polacrilin potassium, starch, pregelatinized starch, sodium alginate, magnesium aluminum silicate, polacrilin potassium, povidone, sodium starch glycolate, mixtures thereof or the like.

### Liquid Formulation

In some embodiments, the present disclosure is directed to a liquid formulation prepared by combining a Compound A, a salt thereof, or a polymorphic form thereof according to any of the embodiments described herein, and a pharmaceutically acceptable excipient.

In some embodiments, the liquid formulation is a solution formulation or a suspension formulation.

In some embodiments, the liquid formulation of the present disclosure further comprises a bicarbonate.

In some embodiments, the liquid formulation of the present disclosure further comprises sodium or potassium bicarbonate.

In some embodiments, the liquid formulation of the present disclosure further comprises sodium bicarbonate.

In some embodiments, the liquid formulation of the present disclosure is prepared using Compound A (e.g., Compound A monocitrate monohydrate salt) or polymorphic form thereof (e.g., Compound A monocitrate monohydrate Form A salt) in an amount from about 1% to about 20%, from about 1% to about 19%, from about 1% to about 18%, from about 1% to about 17%, from about 1% to about 16%, from about 1% to about 15%, from about 1% to about 14%, from about 1% to about 13%, from about 1% to about 12%, from about 1% to about 11%, from about 1% to about 10%, from about 1% to about 9%, from about 1% to about 8%, from about 1% to about 7%, about 2% to about 18%, from about 2% to about 17%, from about 2% to about 16%, from about 2% to about 15%, from about 2% to about 14%, from about 2% to about 13%, from about 2% to about 12%, from about 2% to about 11%, from about 2% to about 10%, from about 2% to about 9%, from about 2% to about 8%, from about 2% to about 7%, about 3% to about 18%, from about 3% to about 17%, from about 3% to about 16%, from about 3% to about 15%, from about 3% to about 14%, from about 3% to about 13%, from about 3% to about 12%, from about 3% to about 11%, from about 3% to about 10%, from about 3% to about 9% from about 3% to about 8%, from about 3% to about 7%, about 4% to about 18%, from about 4% to about 17%, from about 4% to about 16%, from about 4% to about 15%, from about 4% to about 14%, from about 4% to about 13%, from about 4% to about 12%, from about 4% to about 11%, from about 4% to about 10%, from about 4% to about 9%, from about 4% to about 8%, from about 4% to about 7%, about 5% to about 18%, from about 5% to about 17%, from about 5% to about 16%, from about 5% to about 15%, from about 5% to about 14%, from about 5% to about 13%, from about 5% to about 12%, from about 5% to about 11%, from about 5% to about 10%, from about 5% to about 9%, from about 5% to about 8%, or from about 5% to about 7% wt/wt.

In some embodiments, the liquid formulation of the present disclosure is prepared using a Compound A citrate salt (e.g., Compound A monocitrate monohydrate salt) or polymorphic form thereof (e.g., Compound A monocitrate monohydrate Form A salt) in an amount from about 1% to about 20%, from about 1% to about 19%, from about 1% to about 18%, from about 1% to about 17%, from about 1% to about 16%, from about 1% to about 15%, from about 1% to about 14%, from about 1% to about 13%, from about 1% to about 12%, from about 1% to about 11%, from about 1% to about 10%, from about 1% to about 9% from about 1% to about 8%, from about 1% to about 7%, about 2% to about 18%, from about 2% to about 17%, from about 2% to about 16%, from about 2% to about 15%, from about 2% to about 14%, from about 2% to about 13%, from about 2% to about 12%, from about 2% to about 11%, from about 2% to about 10%, from about 2% to about 9%, from about 2% to about 8%, from about 2% to about 7%, about 3% to about 18%, from about 3% to about 17%, from about 3% to about 16%, from about 3% to about 15%, from about 3% to about 14%, from about 3% to about 13%, from about 3% to about 12%, from about 3% to about 11%, from about 3% to about 10%, from about 3% to about 9%, from about 3% to about 8%, from about 3% to about 7%, about 4% to about 18%, from about 4% to about 17%, from about 4% to about 16%, from about 4% to about 15%, from about 4% to about 14%, from about 4% to about 13%, from about 4% to about 12%, from about 4% to about 11%, from about 4% to about 10%, from about 4% to about 9%, from about 4% to about 8%, from about 4% to about 7%, about 5% to about 18%, from about 5% to about 17%, from about 5% to about 16%, from about 5% to about 15%, from about 5% to about 14%, from about 5% to about 13%, from about 5% to about 12%, from about 5% to about 11%, from about 5% to about 10%, from about 5% to about 9%, from about 5% to about 8%, or from about 5% to about 7% wt/wt calculated as free base equivalents.

In some embodiments, the liquid formulation of the present disclosure is prepared using a Compound A citrate salt (e.g., Compound A monocitrate monohydrate salt) or polymorphic form thereof (e.g., Compound A monocitrate monohydrate Form A salt) at a concentration from about 1 mg/mL to about 100 mg/mL, from about 1 mg/mL to about 90 mg/mL, from about 1 mg/mL to about 80 mg/mL, from about 1 mg/mL to about 70 mg/mL, from about 1 mg/mL to about 60 mg/mL, from about 1 mg/mL to about 50 mg/mL, from about 1 mg/mL to about 40 mg/mL, from about 10 mg/mL to about 100 mg/mL, from about 10 mg/mL to about 90 mg/mL, from about 10 mg/mL to about 80 mg/mL, from about 10 mg/mL to about 70 mg/mL, from about 10 mg/mL to about 60 mg/mL, from about 10 mg/mL to about 50 mg/mL, from about 10 mg/mL to about 40 mg/mL, from about 20 mg/mL to about 100 mg/mL, from about 20 mg/mL to about 90 mg/mL, from about 20 mg/mL to about 80 mg/mL, from about 20 mg/mL to about 70 mg/mL, from about 20 mg/mL to about 60 mg/mL, from about 20 mg/mL to about 50 mg/mL, from about 20 mg/mL to about 40 mg/mL, from about 30 mg/mL to about 100 mg/mL, from about 30 mg/mL to about 90 mg/mL, from about 30 mg/mL to about 80 mg/mL, from about 30 mg/mL to about 70 mg/mL, from about 30 mg/mL to about 60 mg/mL, from about 30 mg/mL to about 50 mg/mL, from about 30 mg/mL to about 40 mg/mL, from about 40 mg/mL to about 100 mg/mL, from about 40 mg/mL to about 90 mg/mL, from about 40 mg/mL to about 80 mg/mL, from about 40 mg/mL to about 70 mg/mL, from about 40 mg/mL to about 60 mg/mL, or from about 40 mg/mL to about 50 mg/mL calculated as free base equivalents.

In some embodiments, the liquid formulation of the present disclosure is prepared using a Compound A citrate salt (e.g., Compound A monocitrate monohydrate salt) or polymorphic form thereof (e.g., Compound A monocitrate monohydrate Form A salt) in an amount of about 1%, about 2%, about 3%, about 4%, about 5%, about 5.18%, about 6%, about 6.19%, about 7%, about 8%, about 9%, about 10%, about 11%, about 12%, about 13%, about 14%, about 15%, about 16%, about 17%, about 18%, about 19%, or about 20% wt/wt.

In some embodiments, the liquid formulation of the present disclosure is prepared using a Compound A citrate salt (e.g., Compound A monocitrate monohydrate salt) or polymorphic form thereof (e.g., Compound A monocitrate monohydrate Form A salt) in an amount of about 1%, about 2%, about 3%, about 4%, about 5%, about 5.18%, about 6%, about 6.19%, about 7%, about 8%, about 9%, about 10%, about 11%, about 12%, about 13%, about 14%, about 15%, about 16%, about 17%, about 18%, about 19%, or about 20% wt/wt calculated as free base equivalents.

In some embodiments, the liquid formulation of the present disclosure is prepared using a Compound A citrate salt (e.g., Compound A monocitrate monohydrate salt) or polymorphic form thereof (e.g., Compound A monocitrate monohydrate Form A salt) at a concentration of about 1 mg/mL, about 2 mg/mL, about 3 mg/mL, about 4 mg/mL, about 5 mg/mL, about 5.18 mg/mL, about 6 mg/mL, about 6.19 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, about 60 mg/mL, about 61 mg/mL, about 62 mg/mL, about 63 mg/mL, about 64 mg/mL, about 65 mg/mL, about 66 mg/mL, about 67 mg/mL, about 68 mg/mL, about 69 mg/mL, about 70 mg/mL, about 71 mg/mL, about 72 mg/mL, about 73 mg/mL, about 74 mg/mL, about 75 mg/mL, about 76 mg/mL, about 77 mg/mL, about 78 mg/mL, about 79 mg/mL, or about 80 mg/mL.

In some embodiments, the liquid formulation of the present disclosure is prepared using a Compound A citrate salt (e.g., Compound A monocitrate monohydrate salt) or polymorphic form thereof (e.g., Compound A monocitrate monohydrate Form A salt) at a concentration of about 1 mg/mL, about 2 mg/mL, about 3 mg/mL, about 4 mg/mL, about 5 mg/mL, about 5.18 mg/mL, about 6 mg/mL, about 6.19 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, about 60 mg/mL, about 61 mg/mL, about 62 mg/mL, about 63 mg/mL, about 64 mg/mL, about 65 mg/mL, about 66 mg/mL, about 67 mg/mL, about 68 mg/mL, about 69 mg/mL, about 70 mg/mL, about 71 mg/mL, about 72 mg/mL, about 73 mg/mL, about 74 mg/mL, about 75 mg/mL, about 76 mg/mL, about 77 mg/mL, about 78 mg/mL, about 79 mg/mL, or about 80 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) in an amount of about 5.2% wt/wt.

In some embodiments, the liquid formulation is prepared using a Compound A citrate salt (e.g., Compound A monocitrate monohydrate salt) or polymorphic form thereof (e.g., Compound A monocitrate monohydrate Form A salt) in an amount of about 6.2% wt/wt.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 10 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 20 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 30 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 40 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 50 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 60 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 70 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 80 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 10 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 20 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 30 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 40 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 50 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 60 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) at a concentration of about 70 mg/mL calculated as free base equivalents.

In some embodiments, the liquid formulation is prepared using a Compound A citrate salt (e.g., Compound A monocitrate monohydrate salt) or polymorphic form thereof (e.g., Compound A monocitrate monohydrate Form A salt) a concentration of about 80 mg/mL calculated as free base equivalents.

In some embodiments, the liquid formulation further comprises from about 0.1% to about 10% w/w of citric acid.

In some embodiments, the liquid formulation further comprises from about 0.1% to about 9% w/w of citric acid.

In some embodiments, the liquid formulation further comprises from about 0.1% to about 8% w/w of citric acid.

In some embodiments, the liquid formulation further comprises from about 0.1% to about 7% w/w of citric acid.

In some embodiments, the liquid formulation further comprises from about 0.1% to about 6% w/w of citric acid.

In some embodiments, the liquid formulation further comprises from about 0.1% to about 5% w/w of citric acid.

In some embodiments, the liquid formulation further comprises from about 0.1% to about 4% w/w of citric acid.

In some embodiments, the liquid formulation further comprises from about 0.1% to about 3% w/w of citric acid.

In some embodiments, the liquid formulation further comprises from about 0.1% to about 2% w/w of citric acid.

In some embodiments, the liquid formulation further comprises from about 0.1% to about 1% w/w of citric acid.

In some embodiments, the liquid formulation of the present disclosure further comprises of about 0.1%, about 0.2% about 0.3%, about 0.38%, about 0.4%, about 0.5%, about 0.6%, about 0.7% about 0.8% about 0.9% about 1%, about 1.1% about 1.2%, about 1.3%, about 1.4%, about 1.5%, about 1.6%, about 1.7%, about 1.8%, about 1.9%, about 2% about 3%, about 4%, or about 5% wt/wt of citric acid.

In some embodiments, the liquid formulation further comprises a sweetener (e.g., advantame or neotame).

In some embodiments, the liquid formulation further comprises a sweetener, wherein the sweetener is advantame or neotame.

In some embodiments, the liquid formulation further comprises advantame.

In some embodiments, the liquid formulation further comprises neotame.

In some embodiments, the liquid formulation further comprises from about 0.01% to about 1% w/w of a sweetener (e.g., advantame or neotame).

In some embodiments, the liquid formulation further comprises from about 0.01% to about 0.9% w/w of a sweetener (e.g., advantame or neotame).

In some embodiments, the liquid formulation further comprises from about 0.01% to about 0.8% w/w of a sweetener (e.g., advantame or neotame).

In some embodiments, the liquid formulation further comprises from about 0.01% to about 0.7% w/w of a sweetener (e.g., advantame or neotame).

In some embodiments, the liquid formulation further comprises from about 0.01% to about 0.6% w/w of a sweetener (e.g., advantame or neotame).

In some embodiments, the liquid formulation further comprises from about 0.01% to about 0.5% w/w of a sweetener (e.g., advantame or neotame).

In some embodiments, the liquid formulation further comprises from about 0.01% to about 0.4% w/w of a sweetener (e.g., advantame or neotame).

In some embodiments, the liquid formulation further comprises from about 0.01% to about 0.3% w/w of a sweetener (e.g., advantame or neotame).

In some embodiments, the liquid formulation further comprises from about 0.01% to about 0.2% w/w of a sweetener (e.g., advantame or neotame).

In some embodiments, the liquid formulation further comprises from about 0.01% to about 0.1% w/w of a sweetener (e.g., advantame or neotame).

In some embodiments, the liquid formulation of the present disclosure further comprises of about 0.01%, about 0.02%, about 0.03%, about 0.04%, about 0.05%, about 0.06%, about 0.07%, about 0.08%, about 0.09%, or about 0.1% wt/wt of a sweetener (e.g., advantame or neotame).

In some embodiments, the liquid formulation further comprises from about 0.1% to about 10% w/w of a taste masking agent (e.g., sodium chloride).

In some embodiments, the liquid formulation further comprises from about 0.1% to about 9% w/w of a taste masking agent (e.g., sodium chloride).

In some embodiments, the liquid formulation further comprises from about 0.1% to about 8% w/w of a taste masking agent (e.g., sodium chloride).

In some embodiments, the liquid formulation further comprises from about 0.1% to about 7% w/w of a taste masking agent (e.g., sodium chloride).

In some embodiments, the liquid formulation further comprises from about 0.1% to about 6% w/w of a taste masking agent (e.g., sodium chloride).

In some embodiments, the liquid formulation further comprises from about 0.1% to about 5% w/w of a taste masking agent (e.g., sodium chloride).

In some embodiments, the liquid formulation further comprises from about 0.1% to about 4% w/w of a taste masking agent (e.g., sodium chloride).

In some embodiments, the liquid formulation further comprises from about 0.1% to about 3% w/w of a taste masking agent (e.g., sodium chloride).

In some embodiments, the liquid formulation further comprises from about 0.1% to about 2% w/w of a taste masking agent (e.g., sodium chloride).

In some embodiments, the liquid formulation further comprises from about 0.1% to about 1% w/w of a taste masking agent (e.g., sodium chloride).

In some embodiments, the liquid formulation of the present disclosure further comprises of about 0.1%, about 0.2%, about 0.3%, about 0.38%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, or about 5% wt/wt of a taste masking agent (e.g., sodium chloride).

In some embodiments, the liquid formulation of the present disclosure further comprises of about 0.1%, about 0.2%, about 0.3%, about 0.38%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, or about 5% wt/wt of a flavor (e.g., grape flavor).

In some embodiments, the liquid formulation further comprises a bicarbonate.

In some embodiments, the liquid formulation further comprises from about 0.1% to about 10% w/w of sodium bicarbonate.

In some embodiments, the liquid formulation further comprises from about 0.1% to about 9% w/w of sodium bicarbonate.

In some embodiments, the liquid formulation further comprises from about 0.1% to about 8% w/w of sodium bicarbonate.

In some embodiments, the liquid formulation further comprises from about 0.1% to about 7% w/w of sodium bicarbonate.

In some embodiments, the liquid formulation further comprises from about 0.1% to about 6% w/w of sodium bicarbonate.

In some embodiments, the liquid formulation further comprises from about 0.1% to about 5% w/w of sodium bicarbonate.

In some embodiments, the liquid formulation further comprises from about 0.1% to about 4% w/w of sodium bicarbonate.

In some embodiments, the liquid formulation further comprises from about 0.1% to about 3% w/w of sodium bicarbonate.

In some embodiments, the liquid formulation further comprises from about 0.1% to about 2% w/w of sodium bicarbonate.

In some embodiments, the liquid formulation further comprises from about 0.1% to about 1% w/w of sodium bicarbonate.

In some embodiments, the liquid formulation of the present disclosure further comprises of about 0.1%, about 0.2% about 0.3%, about 0.4%, about 0.5%, about 0.6%, about 0.7%, about 0.8% about 0.9% about 1%, about 1.1%, about 1.2%, about 1.3%, about 1.4%, about 1.5%, about 1.6%, about 1.7%, about 1.8%, about 1.9%, about 2%, about 3%, about 4%, or about 5% wt/wt of sodium bicarbonate.

In some embodiments, the liquid formulation further comprises about 0.38% w/w of sodium bicarbonate.

In some embodiments, the liquid formulation further comprises about 6.7% w/w of sodium bicarbonate.

In some embodiments, the liquid formulation further comprises about 10% w/w of sodium bicarbonate.

In some embodiments, the liquid formulation further comprises about 15% w/w of sodium bicarbonate.

In some embodiments, the liquid formulation further comprises about 20% w/w of sodium bicarbonate.

In some embodiments, the liquid formulation further comprises about 25% w/w of sodium bicarbonate.

In some embodiments, the liquid formulation further comprises about 29.4% w/w of sodium bicarbonate.

In some embodiments, the liquid formulation further comprises about 30% w/w of sodium bicarbonate.

In some embodiments, the liquid formulation further comprises about 35% w/w of sodium bicarbonate.

In some embodiments, the liquid formulation further comprises about 40% w/w of sodium bicarbonate.

In some embodiments, the liquid formulation further comprises about 42.7% w/w of sodium bicarbonate.

In some embodiments, the liquid formulation further comprises about 45% w/w of sodium bicarbonate.

In some embodiments, the liquid formulation further comprises about 50% w/w of sodium bicarbonate.

In some embodiments, the liquid formulation of the present disclosure is prepared using: a Compound A citrate salt (e.g., Compound A monocitrate monohydrate salt) or polymorphic form thereof (e.g., Compound A monocitrate monohydrate Form A salt) in an amount from about 1% to about 10% wt/wt, and
from about 0.01% to about 0.1% w/w of a sweetener (e.g., advantame or neotame).

In some embodiments, the liquid formulation of the present disclosure is prepared using: a Compound A citrate salt (e.g., Compound A monocitrate monohydrate salt) or polymorphic form thereof (e.g., Compound A monocitrate monohydrate Form A salt) in an amount from about 1% to about 10% wt/wt,
from about 0.01% to about 0.1% w/w of a sweetener (e.g., advantame or neotame), and
from about 0.1% to about 2% w/w of citric acid.

In some embodiments, the liquid formulation of the present disclosure is prepared using: a Compound A citrate salt (e.g., Compound A monocitrate monohydrate salt) or polymorphic form thereof (e.g., Compound A monocitrate monohydrate Form A salt) in an amount from about 1% to about 10% wt/wt,
from about 0.01% to about 0.1% w/w of a sweetener (e.g., advantame or neotame),
from about 0.1% to about 2% w/w of citric acid, and
from about 0.1% to about 2% w/w of a taste masking agent (e.g., sodium chloride).

In some embodiments, the liquid formulation of the present disclosure is prepared using: a Compound A citrate salt (e.g., Compound A monocitrate monohydrate salt) or polymorphic form thereof (e.g., Compound A monocitrate monohydrate Form A salt) in an amount from about 1% to about 10% wt/wt,
from about 0.01% to about 0.1% w/w of a sweetener (e.g., advantame or neotame),
from about 0.1% to about 2% w/w of citric acid,
from about 0.1% to about 2% w/w of a taste masking agent (e.g., sodium chloride), and
from about 0.1% to about 5% w/w of sodium bicarbonate.

In some embodiments, the liquid formulation of the present disclosure has a pH of about 2.0, about 2.5, about 3.0, about 3.5, about 4.0, about 4.5, about 5.0, about 5.5, about 6.0, about 6.5, about 7.0, about 7.5, or about 8.0.

In some embodiments, the liquid formulation has a pH of about 3.0.

In some particular embodiments, the liquid formulation of the present disclosure is prepared by combining:

| | Component | Composition |
|---|---|---|
| Material | Function | Designation (F1) |
| Compound A salt form | — | Sesquifumarate |
| Compound A salt form (% w/w) | — | 6.19 (50 mg/mL |
| | | free-base |
| | | equivalents)^{a} |
| Citric acid, anhydrous (% w/w) | Buffering agent | 0.38 |
| Advantame (% w/w) | Sweetener | 0.07 |
| Methylparaben sodium (% w/w) | Preservative | 0.1 |
| Potassium sorbate (% w/w) | Preservative | 0.1 |
| Sodium chloride (% w/w) | Taste Masking | 1.0 |
| Sodium bicarbonate (% w/w) | Buffering Agent | 1.6 |
| Grape flavor (% w/w) | Flavor | 1.0 |
| Sodium hydroxide | pH adjuster^{c} | Adjusted to pH 6.5 |
| Hydrochloric acid | pH adjuster^{c} | |
| Purified water | Vehicle | Q.S. to 100% |

^{a}50 mg/mL free base of API is equivalent to 63.80 mg/mL of salt form of API. The specific gravity of the final formulation is 1.03 g/mL. A concentration of 50 mg/mL free base is therefore equivalent to 48.5 mg/g free base or 61.9 mg/g of sesquifumarate salt.
^{b}40 mg/mL free base of API is equivalent to 53.32 mg/mL of salt form of API. The specific gravity of the final formulation is 1.03. A concentration of 40 mg/mL free base is therefore equivalent to 38.8 mg/g (40 mg/mL/1.03) free base or 51.8 mg/g of citrate salt form.
^{c}For pH adjustment as needed.
API = Active Pharmaceutical Ingredient; N/A = Not Applicable; Q.S. = Quantum Sufficient.

In some particular embodiments, the liquid formulation of the present disclosure is prepared by combining:

| | Component | Composition |
|---|---|---|
| Material | Function | Designation (F2) |
| Compound A salt form | — | Sesquifumarate |
| Compound A salt form (% w/w) | — | 6.19 (50 mg/mL |
| | | free-base |
| | | equivalents)^{a} |
| Citric acid, anhydrous (% w/w) | Buffering agent | 0.38 |
| Advantame (% w/w) | Sweetener | 0.07 |
| Methylparaben sodium (% w/w) | Preservative | 0.18 |
| Potassium sorbate (% w/w) | Preservative | 0.20 |
| Sodium chloride (% w/w) | Taste Masking | 1.0 |
| Sodium bicarbonate (% w/w) | Buffering Agent | 1.6 |
| Grape flavor (% w/w) | Flavor | N/A |
| Sodium hydroxide | pH adjuster^{c} | Adjusted to pH 6.0 |
| Hydrochloric acid | pH adjuster^{c} | |
| Purified water | Vehicle | Q.S. to 100% |

^{a}50 mg/mL free base of API is equivalent to 63.80 mg/mL of salt form of API. The specific gravity of the final formulation is 1.03 g/mL. A concentration of 50 mg/mL free base is therefore equivalent to 48.5 mg/g free base or 61.9 mg/g of sesquifumarate salt.
^{b}40 mg/mL free base of API is equivalent to 53.32 mg/mL of salt form of API. The specific gravity of the final formulation is 1.03. A concentration of 40 mg/mL free base is therefore equivalent to 38.8 mg/g (40 mg/mL/1.03) free base or 51.8 mg/g of citrate salt form.
^{c}For pH adjustment as needed.
API = Active Pharmaceutical Ingredient; N/A= Not Applicable; Q.S. = Quantum Sufficient.

In some particular embodiments, the liquid formulation of the present disclosure is prepared by combining:

| | Component | Composition |
|---|---|---|
| Material | Function | Designation (F3) |
| Compound A salt form | — | Citrate |
| Compound A salt form | — | 5.18 (40 mg/mL |
| (% w/w) | | free-base |
| | | equivalents)^{b} |
| Citric acid, anhydrous | Buffering agent | 0.38 |
| (% w/w) | | |
| Advantame (% w/w) | Sweetener | 0.07 |
| Methylparaben sodium | Preservative | 0.10 |
| (% w/w) | | |
| Potassium sorbate (% w/w) | Preservative | 0.10 |
| Sodium chloride (% w/w) | Taste Masking | 1.0 |
| Sodium bicarbonate (% w/w) | Buffering Agent | N/A |
| Grape flavor (% w/w) | Flavor | 1.0 |
| Sodium hydroxide | pH adjuster^{c} | Adjusted to pH 3.0 |
| Hydrochloric acid | pH adjuster^{c} | |
| Purified water | Vehicle | Q.S. to 100% |

^{a} 50 mg/mL free base of API is equivalent to 63.80 mg/mL of salt form of API. The specific gravity of the final formulation is 1.03 g/mL. A concentration of 50 mg/mL free base is therefore equivalent to 48.5 mg/g free base or 61.9 mg/g of sesquifumarate salt.
^{b}40 mg/mL free base of API is equivalent to 53.32 mg/mL of salt form of API. The specific gravity of the final formulation is 1.03. A concentration of 40 mg/mL free base is therefore equivalent to 38.8 mg/g (40 mg/mL/1.03) free base or 51.8 mg/g of citrate salt form.
^{c}For pH adjustment as needed.
API = Active Pharmaceutical Ingredient; N/A = Not Applicable; Q.S. = Quantum Sufficient.

### Purity

In some embodiments, Compound A monocitrate monohydrate API (Form A) must meet acceptance criteria that demonstrate acceptable purity. In some embodiments, the acceptance criteria are selected from the group comprising: appearance, assay, total impurities, specified impurities, unspecified impurities, water content, residual solvents, and elemental impurities.

In some embodiments, tests for acceptance criteria are performed during the release of drug substance for use in the formulation i.e., the tests will be performed prior to use in the formulation preparation.

In some embodiments, tests for acceptance criteria are performed after to use in the formulation preparation.

In one embodiment, the assay of Compound A monocitrate monohydrate polymorphic API is at least about 90% w/w, at least about 91% w/w, at least about 92% w/w, at least about 93% w/w, at least about 94% w/w, at least about 95% w/w, at least about 96% w/w, at least about 97% w/w, at least about 98% w/w, or at least about 99% w/w.

In another embodiment, total impurities in Compound A monocitrate monohydrate polymorphic API are less than about 5.0% area, less than about 4.5% area, less than about 4.0% area, less than about 3.5% area, less than about 3.0% area, less than about 2.5% area, less than about 2.0% area, less than about 1.5% area, less than about 1.0% area, or less than about 0.5% area.

In another embodiment, Compound A monocitrate monohydrate polymorphic API has ≤0.5% w/w, ≤0.45% w/w, ≤0.40% w/w, ≤0.35% w/w, ≤0.30% w/w, ≤0.25% w/w, ≤0.2% w/w, ≤0.15% w/w, or ≤0.1% w/w of Impurity B.

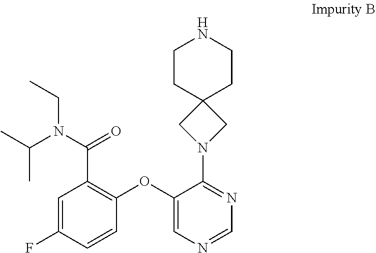 [SMILES: CCN(C(=O)c1cc(F)ccc1Oc1cncnc1N1CC2(CCNCC2)C1)C(C)C]

In other embodiments, Compound A monocitrate monohydrate polymorphic API has no more than about 1.0% area, no more than about 0.9% area, no more than about 0.8% area, no more than about 0.7% area, no more than about 0.6% area, no more than 0.5% area, no more than about 0.4% area, no more than about 0.3% area, no more than about 0.2% area, or no more than about 0.1% area of a single impurity.

In yet other embodiments, the impurities are measured by chromatography, nuclear magnetic resonance spectroscopy, mass spectrometry, powder X-ray diffraction or spectroscopy. In some embodiments, purity is confirmed by assay value and impurities (total, specified and unspecified).

In some embodiments, the residual solvents and elemental impurities in Compound A monocitrate monohydrate polymorphic API are as defined by ICH limits.

In some embodiments, the tablet of the present disclosure comprises an intragranular phase and an extragranular phase, wherein:
(a) the intragranular phase comprises Compound A citrate salt, or a polymorphic form thereof (e.g., Form A, B, and/or C) dispersed within a first pharmaceutically acceptable excipient; and
(b) the extragranular phase comprises a second pharmaceutically acceptable excipient, and wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure further comprises an intragranular phase and an extragranular phase, wherein:
(a) the intragranular phase comprises Compound A citrate salt Form A salt dispersed within a first pharmaceutically acceptable excipient; and
(b) the extragranular phase comprises a second pharmaceutically acceptable excipient, and wherein the Compound A citrate salt Form A salt is at least about 95% pure as measured by chromatography, nuclear magnetic resonance spectroscopy, mass spectrometry, powder X-ray diffraction or spectroscopy.

In some embodiments, the tablet of the present disclosure comprises a Compound A citrate salt, or a polymorphic form thereof (e.g., Form A, B, and/or C), such that the tablet comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A citrate salt, or a polymorphic form thereof and wherein the Compound A citrate Form A salt or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate, or a polymorphic form thereof (e.g., Form A, B, and/or C), such that the tablet comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A monocitrate monohydrate, or a polymorphic form thereof and wherein the Compound A monocitrate monohydrate, or a polymorphic form thereof is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate, or a polymorphic form thereof (e.g., Form A, B, and/or C), such that the tablet comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A monocitrate monohydrate, or a polymorphic form thereof calculated as free base equivalents and wherein the Compound A citrate Form A salt or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet of the present disclosure comprises a Compound A citrate salt, or a polymorphic form thereof (e.g., Form A, B, and/or C), such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, 21%, about 22%, about 23%, about 24%, about 24.4%, about 25%, about 26%, about 27%, about 28%, about 29%, or about 30% wt/wt of Compound A citrate salt, or a polymorphic form thereof and wherein the Compound A citrate Form A salt or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet of the present disclosure comprises a Compound A citrate salt, or a polymorphic form thereof (e.g., Form A, B, and/or C), such that the tablet comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A citrate salt, or a polymorphic form thereof calculated as free base equivalents and wherein the Compound A citrate Form A salt or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate, or a polymorphic form thereof (e.g., Form A, B, and/or C), such that the tablet comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A monocitrate monohydrate, or a polymorphic form thereof calculated as free base equivalents and wherein the Compound A monocitrate monohydrate, or a polymorphic form thereof is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate, or a polymorphic form thereof (e.g., Form A, B, and/or C), such that the tablet comprises from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt of Compound A monocitrate monohydrate, or a polymorphic form thereof and wherein the Compound A citrate Form A salt or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet of the present disclosure comprises a Compound A citrate salt, or a polymorphic form thereof (e.g., Form A, B, and/or C), such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, 21%, about 22%, about 23%, about 23.8%, about 24%, about 24.4%, about 25%, about 26%, about 27%, about 28%, about 29%, or about 30% wt/wt of Compound A citrate salt, or a polymorphic form thereof calculated as free base equivalents and wherein the Compound A citrate Form A salt or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate, or a polymorphic form thereof (e.g., Form A, B, and/or C), such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, 21%, about 22%, about 23%, about 23.8%, about 24%, about 24.4%, about 25%, about 26%, about 27%, about 28%, about 29%, or about 30% wt/wt of Compound A monocitrate monohydrate, or a polymorphic form thereof and wherein the Compound A monocitrate monohydrate, or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate, or a polymorphic form thereof (e.g., Form A, B, and/or C), such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, 21%, about 22%, about 23%, about 23.8%, about 24%, about 24.4%, about 25%, about 26%, about 27%, about 28%, about 29%, or about 30% wt/wt of Compound A monocitrate monohydrate, or a polymorphic form thereof calculated as free base equivalents and wherein the Compound A monocitrate monohydrate, or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet of the present disclosure comprises a Compound A citrate Form A salt, such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, 21%, about 22%, about 23%, about 23.8%, about 24%, about 24.4%, about 25%, about 26%, about 27%, about 28%, about 29%, or about 30% wt/wt of Compound A citrate Form A salt and wherein the Compound A citrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises a Compound A citrate Form A salt, such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, 21%, about 22%, about 23%, about 23.8%, about 24%, about 24.4%, about 25%, about 26%, about 27%, about 28%, about 29%, or about 30% wt/wt of Compound A citrate Form A salt calculated as free base equivalents and wherein the Compound A citrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate Form A salt, such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, 21%, about 22%, about 23%, about 23.8%, about 24%, about 24.4%, about 25%, about 26%, about 27%, about 28%, about 29%, or about 30% wt/wt of Compound A monocitrate monohydrate Form A salt and wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate Form A salt, such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, 21%, about 22%, about 23%, about 23.8%, about 24%, about 24.4%, about 25%, about 26%, about 27%, about 28%, about 29%, or about 30% wt/wt of Compound A monocitrate monohydrate Form A salt calculated as free base equivalents and wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate Form A salt, such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, 21%, about 22%, about 23%, about 23.8%, about 24%, about 24.4%, about 25%, about 26%, about 27%, about 28%, about 29%, or about 30% wt/wt of Compound A monocitrate monohydrate Form A salt and wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate Form A salt, such that the tablet comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 17.8%, about 18%, about 18.7%, about 19%, about 20%, 21%, about 22%, about 23%, about 23.8%, about 24%, about 24.4%, about 25%, about 26%, about 27%, about 28%, about 29%, or about 30% wt/wt of Compound A monocitrate monohydrate Form A salt calculated as free base equivalents and wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises a Compound A citrate salt, or a polymorphic form thereof (e.g., Form A, B, and/or C) in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 146.5 mg, about 120 mg, about 130 mg, about 135 mg, about 140 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A citrate salt, or a polymorphic form thereof and wherein the Compound A citrate salt, or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet of the present disclosure comprises a Compound A citrate salt, or a polymorphic form thereof (e.g., Form A, B, and/or C) in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A citrate salt, or a polymorphic form thereof calculated as free base equivalents and wherein the a Compound A citrate salt, or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate, or a polymorphic form thereof (e.g., Form A, B, and/or C) in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A monocitrate monohydrate, or a polymorphic form thereof and wherein the Compound A monocitrate monohydrate, or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate, or a polymorphic form thereof (e.g., Form A, B, and/or C) in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A monocitrate monohydrate, or a polymorphic form thereof calculated as free base equivalents and wherein the Compound A monocitrate monohydrate, or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet of the present disclosure comprises a Compound A citrate Form A salt in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A citrate Form A salt and wherein the Compound A citrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises a Compound A citrate Form A salt in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A citrate Form A salt calculated as free base equivalents and wherein the Compound A citrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate Form A salt in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A monocitrate monohydrate Form A salt and wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises a Compound A monocitrate monohydrate Form A salt in an amount of about 5 mg, about 10 mg, about 15 mg, about 20 mg, about 21 mg, about 22 mg, about 23 mg, about 24 mg, about 25 mg, about 26 mg, about 27 mg, about 28 mg, about 29 mg, about 30 mg, about 33.4 mg, about 35 mg, about 40 mg, about 50 mg, about 60 mg, about 70 mg, about 80 mg, about 90 mg, about 100 mg, about 110 mg, about 120 mg, about 130 mg, about 135 mg, about 140 mg, about 146.5 mg, about 150 mg, about 160 mg, about 170 mg, about 180 mg, about 180.2 mg, about 190 mg, about 200 mg, about 210 mg, about 213.2 mg, about 220 mg, about 230 mg, about 240 mg, about 250 mg, or about 300 mg of Compound A monocitrate monohydrate Form A salt calculated as free base equivalents and wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet comprises Compound A citrate salt, or a polymorphic form thereof (e.g., Form A, B, and/or C) in an amount of about 25 mg, about 33.4 mg, about 110 mg, about 135 mg, about 146.5 mg, about 160 mg, about 180.2 mg, about 213.2 mg or about 250 mg and wherein the Compound A citrate salt, or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet comprises Compound A citrate salt, or a polymorphic form thereof (e.g., Form A, B, and/or C) in an amount of about 25 mg, about 33.4 mg, about 110 mg, about 135 mg, about 146.5 mg, about 160 mg, about 180.2 mg, about 213.2 mg or about 250 mg calculated as free base equivalents and wherein the Compound A citrate salt, or a polymorphic form thereof (e.g., Form A, B, and/or C) is at least about 95% pure as measured by chromatography, nuclear magnetic resonance spectroscopy, mass spectrometry, powder X-ray diffraction or spectroscopy.

In some embodiments, the tablet comprises Compound A monocitrate monohydrate in an amount of about 25 mg, about 33.4 mg, about 110 mg, about 135 mg, about 146.5 mg, about 160 mg, about 180.2 mg, about 213.2 mg or about 250 mg and wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet comprises Compound A monocitrate monohydrate in an amount of about 25 mg, about 33.4 mg, about 110 mg, about 135 mg, about 146.5 mg, about 160 mg, about 180.2 mg, about 213.2 mg or about 250 mg calculated as free base equivalents and wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt, and
from about 1% to about 10% wt/wt sodium bicarbonate,
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt,
from about 45% to about 60% wt/wt of a filler (e.g., a filler comprising microcrystalline cellulose); and
from about 1% to about 10% wt/wt sodium bicarbonate,
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt,
from about 45% to about 60% wt/wt of a filler (e.g., a filler comprising microcrystalline cellulose);
from about 1% to about 10% wt/wt sodium bicarbonate; and
from about 0.1% to about 2% wt/wt a glidant (e.g., hydrophobic colloidal silica),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt,
from about 45% to about 60% wt/wt of a filler (e.g., a filler comprising microcrystalline cellulose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt,
from about 45% to about 60% wt/wt of a filler (e.g., a filler comprising microcrystalline cellulose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt,
from about 45% to about 60% wt/wt of a filler (e.g., a filler comprising microcrystalline cellulose);
from about 5% to about 20% wt/wt a binder (e.g., hypromellose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt,
from about 45% to about 60% wt/wt of a filler (e.g., a filler comprising microcrystalline cellulose);
from about 5% to about 20% wt/wt a binder (e.g., hypromellose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
(i) from about 15% to about 30% wt/wt of a Compound A citrate Form A salt, and
(ii) from about 1% to about 10% wt/wt of sodium bicarbonate,
wherein the Compound A citrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 15% to about 30% wt/wt of a Compound A citrate salt; and
(b) the extragranular phase comprises:
from about 0.5% to about 5% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 15% to about 30% wt/wt of a Compound A citrate salt;
about 1% to about 10% wt/wt of sodium bicarbonate; and
(b) the extragranular phase comprises:
from about 0.5% to about 5% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure further comprises an intragranular phase and an extragranular phase, wherein:
(a) the intragranular phase comprises:
   from about 15% to about 30% wt/wt of a Compound A citrate salt;
   from about 45% to about 60% wt/wt of a filler (e.g., a filler comprising microcrystalline cellulose);
   from about 5% to about 15% wt/wt a disintegrant (e.g., crospovidone);
   from about 5% to about 20% wt/wt a binder (e.g., hypromellose);
   from about 2% to about 7% wt/wt sodium bicarbonate;
   from about 0.1% to about 2% wt/wt a glidant (e.g., hydrophobic colloidal silica);
   from about 0.1% to about 2% wt/wt a lubricant (e.g., magnesium stearate); and
(b) the extragranular phase comprises:
from about 0.5% to about 5% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate, and
from about 1% to about 10% wt/wt sodium bicarbonate,
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate,
from about 45% to about 60% wt/wt of a filler (e.g., a filler comprising microcrystalline cellulose); and
from about 1% to about 10% wt/wt sodium bicarbonate,
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate salt calculated as free base equivalents,
from about 45% to about 60% wt/wt of a filler (e.g., a filler comprising microcrystalline cellulose); and
from about 1% to about 10% wt/wt sodium bicarbonate,
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate,
from about 45% to about 60% wt/wt of a filler (e.g., a filler comprising microcrystalline cellulose);
from about 1% to about 10% wt/wt sodium bicarbonate; and
from about 0.1% to about 2% wt/wt a glidant (e.g., hydrophobic colloidal silica),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate,
from about 45% to about 60% wt/wt of a filler (e.g., a filler comprising microcrystalline cellulose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate,
from about 45% to about 60% wt/wt of a filler (e.g., a filler comprising microcrystalline cellulose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate,
from about 45% to about 60% wt/wt of a filler (e.g., a filler comprising microcrystalline cellulose);
from about 5% to about 20% wt/wt a binder (e.g., hypromellose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate,
from about 45% to about 60% wt/wt of a filler (e.g., a filler comprising microcrystalline cellulose);
from about 5% to about 20% wt/wt a binder (e.g., hypromellose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
(i) from about 15% to about 30% wt/wt of a Compound A monocitrate monohydrate Form A salt, and
(ii) from about 1% to about 10% wt/wt of sodium bicarbonate,
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
(i) from about 15% to about 30% wt/wt of a Compound A monocitrate monohydrate Form A salt calculated as free base equivalents, and
(ii) from about 1% to about 10% wt/wt of sodium bicarbonate,
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 15% to about 30% wt/wt of a Compound A monocitrate monohydrate salt; and
(b) the extragranular phase comprises:
from about 0.5% to about 5% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 15% to about 30% wt/wt of a Compound A monocitrate monohydrate salt; about 1% to about 10% wt/wt of sodium bicarbonate; and
(b) the extragranular phase comprises:
from about 0.5% to about 5% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure further comprises an intragranular phase and an extragranular phase, wherein:
(a) the intragranular phase comprises:
   from about 15% to about 30% wt/wt of a Compound A monocitrate monohydrate salt;
   from about 45% to about 60% wt/wt of a filler (e.g., a filler comprising microcrystalline cellulose);
   from about 5% to about 15% wt/wt a disintegrant (e.g., crospovidone);
   from about 5% to about 20% wt/wt a binder (e.g., hypromellose);
   from about 2% to about 7% wt/wt sodium bicarbonate;
   from about 0.1% to about 2% wt/wt a glidant (e.g., hydrophobic colloidal silica);
   from about 0.1% to about 2% wt/wt a lubricant (e.g., magnesium stearate); and
(b) the extragranular phase comprises:
from about 0.5% to about 5% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate salt calculated as free base equivalents, and
from about 1% to about 10% wt/wt sodium bicarbonate,
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate salt calculated as free base equivalents,
from about 45% to about 60% wt/wt of a filler (e.g., a filler comprising microcrystalline cellulose); and
from about 1% to about 10% wt/wt sodium bicarbonate,
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate salt calculated as free base equivalents,
from about 45% to about 60% wt/wt of a filler (e.g., a filler comprising microcrystalline cellulose);
from about 1% to about 10% wt/wt sodium bicarbonate; and
from about 0.1% to about 2% wt/wt a glidant (e.g., hydrophobic colloidal silica),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate salt calculated as free base equivalents,
from about 45% to about 60% wt/wt of a filler (e.g., a filler comprising microcrystalline cellulose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate salt calculated as free base equivalents,
from about 45% to about 60% wt/wt of a filler (e.g., a filler comprising microcrystalline cellulose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate salt calculated as free base equivalents,
from about 45% to about 60% wt/wt of a filler (e.g., a filler comprising microcrystalline cellulose);
from about 5% to about 20% wt/wt a binder (e.g., hypromellose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate salt calculated as free base equivalents,
from about 45% to about 60% wt/wt of a filler (e.g., a filler comprising microcrystalline cellulose);
from about 5% to about 20% wt/wt a binder (e.g., hypromellose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
(i) from about 15% to about 30% wt/wt of a Compound A monocitrate monohydrate Form A salt calculated as free base equivalents, and
(ii) from about 1% to about 10% wt/wt of sodium bicarbonate,
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 15% to about 30% wt/wt of a Compound A monocitrate monohydrate salt calculated as free base equivalents; and
(b) the extragranular phase comprises:
from about 0.5% to about 5% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 15% to about 30% wt/wt of a Compound A monocitrate monohydrate salt calculated as free base equivalents;
about 1% to about 10% wt/wt of sodium bicarbonate; and
(b) the extragranular phase comprises:
from about 0.5% to about 5% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure further comprises an intragranular phase and an extragranular phase, wherein:
(a) the intragranular phase comprises:
   from about 15% to about 30% wt/wt of a Compound A monocitrate monohydrate salt calculated as free base equivalents;
from about 45% to about 60% wt/wt of a filler (e.g., a filler comprising microcrystalline cellulose);
from about 5% to about 15% wt/wt a disintegrant (e.g., crospovidone);
from about 5% to about 20% wt/wt a binder (e.g., hypromellose);
from about 2% to about 7% wt/wt sodium bicarbonate;
from about 0.1% to about 2% wt/wt a glidant (e.g., hydrophobic colloidal silica);
from about 0.1% to about 2% wt/wt a lubricant (e.g., magnesium stearate); and
(b) the extragranular phase comprises:
from about 0.5% to about 5% wt/wt a lubricant (e.g., magnesium stearate),
wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate Form A salt,
from about 45% to about 60% wt/wt of a filler (e.g., a filler comprising microcrystalline cellulose); and
from about 1% to about 10% wt/wt sodium bicarbonate,
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate Form A salt,
from about 45% to about 60% wt/wt of a filler (e.g., a filler comprising microcrystalline cellulose);
from about 1% to about 10% wt/wt sodium bicarbonate; and
from about 0.1% to about 2% wt/wt a glidant (e.g., hydrophobic colloidal silica),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate Form A salt,
from about 45% to about 60% wt/wt of a filler (e.g., a filler comprising microcrystalline cellulose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate Form A salt,
from about 45% to about 60% wt/wt of a filler (e.g., a filler comprising microcrystalline cellulose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate Form A salt,
from about 45% to about 60% wt/wt of a filler (e.g., a filler comprising microcrystalline cellulose);
from about 5% to about 20% wt/wt a binder (e.g., hypromellose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate Form A salt,
from about 45% to about 60% wt/wt of a filler (e.g., a filler comprising microcrystalline cellulose);
from about 5% to about 20% wt/wt a binder (e.g., hypromellose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 15% to about 30% wt/wt of a Compound A monocitrate monohydrate Form A salt; and
(b) the extragranular phase comprises:
from about 0.5% to about 5% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 15% to about 30% wt/wt of a Compound A monocitrate monohydrate Form A salt;
about 1% to about 10% wt/wt of sodium bicarbonate; and
(b) the extragranular phase comprises:
from about 0.5% to about 5% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure further comprises an intragranular phase and an extragranular phase, wherein:
(a) the intragranular phase comprises:
   from about 15% to about 30% wt/wt of a Compound A monocitrate monohydrate Form A salt;
from about 45% to about 60% wt/wt of a filler (e.g., a filler comprising microcrystalline cellulose);
from about 5% to about 15% wt/wt a disintegrant (e.g., crospovidone);
from about 5% to about 20% wt/wt a binder (e.g., hypromellose);
from about 2% to about 7% wt/wt sodium bicarbonate;
from about 0.1% to about 2% wt/wt a glidant (e.g., hydrophobic colloidal silica);
from about 0.1% to about 2% wt/wt a lubricant (e.g., magnesium stearate); and
(b) the extragranular phase comprises:
from about 0.5% to about 5% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate Form A salt calculated as free base equivalents, and
from about 1% to about 10% wt/wt sodium bicarbonate,
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate Form A salt calculated as free base equivalents,
from about 45% to about 60% wt/wt of a filler (e.g., a filler comprising microcrystalline cellulose); and
from about 1% to about 10% wt/wt sodium bicarbonate,
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate Form A salt calculated as free base equivalents,
from about 45% to about 60% wt/wt of a filler (e.g., a filler comprising microcrystalline cellulose);
from about 1% to about 10% wt/wt sodium bicarbonate; and
from about 0.1% to about 2% wt/wt a glidant (e.g., hydrophobic colloidal silica),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate Form A salt calculated as free base equivalents,
from about 45% to about 60% wt/wt of a filler (e.g., a filler comprising microcrystalline cellulose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate Form A salt calculated as free base equivalents,
from about 45% to about 60% wt/wt of a filler (e.g., a filler comprising microcrystalline cellulose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate Form A salt calculated as free base equivalents,
from about 45% to about 60% wt/wt of a filler (e.g., a filler comprising microcrystalline cellulose);
from about 5% to about 20% wt/wt a binder (e.g., hypromellose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A monocitrate monohydrate Form A salt calculated as free base equivalents,
from about 45% to about 60% wt/wt of a filler (e.g., a filler comprising microcrystalline cellulose);
from about 5% to about 20% wt/wt a binder (e.g., hypromellose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
(i) from about 15% to about 30% wt/wt of a Compound A monocitrate monohydrate Form A salt calculated as free base equivalents, and
(ii) from about 1% to about 10% wt/wt of sodium bicarbonate,
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 15% to about 30% wt/wt of a Compound A monocitrate monohydrate Form A salt calculated as free base equivalents; and
(b) the extragranular phase comprises:
from about 0.5% to about 5% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 15% to about 30% wt/wt of a Compound A monocitrate monohydrate Form A salt calculated as free base equivalents;
about 1% to about 10% wt/wt of sodium bicarbonate; and
(b) the extragranular phase comprises:
from about 0.5% to about 5% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure further comprises an intragranular phase and an extragranular phase, wherein:
(a) the intragranular phase comprises:
   from about 15% to about 30% wt/wt of a Compound A monocitrate monohydrate Form A salt calculated as free base equivalents;
from about 45% to about 60% wt/wt of a filler (e.g., a filler comprising microcrystalline cellulose);
from about 5% to about 15% wt/wt a disintegrant (e.g., crospovidone);
from about 5% to about 20% wt/wt a binder (e.g., hypromellose);
from about 2% to about 7% wt/wt sodium bicarbonate;
from about 0.1% to about 2% wt/wt a glidant (e.g., hydrophobic colloidal silica);
from about 0.1% to about 2% wt/wt a lubricant (e.g., magnesium stearate); and
(b) the extragranular phase comprises:
from about 0.5% to about 5% wt/wt a lubricant (e.g., magnesium stearate),
wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt calculated as free base equivalents, and
from about 1% to about 10% wt/wt sodium bicarbonate,
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt calculated as free base equivalents,
from about 45% to about 60% wt/wt of a filler (e.g., a filler comprising microcrystalline cellulose); and
from about 1% to about 10% wt/wt sodium bicarbonate,
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt calculated as free base equivalents,
from about 45% to about 60% wt/wt of a filler (e.g., a filler comprising microcrystalline cellulose);
from about 1% to about 10% wt/wt sodium bicarbonate; and
from about 0.1% to about 2% wt/wt a glidant (e.g., hydrophobic colloidal silica),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt calculated as free base equivalents,
from about 45% to about 60% wt/wt of a filler (e.g., a filler comprising microcrystalline cellulose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt calculated as free base equivalents,
from about 45% to about 60% wt/wt of a filler (e.g., a filler comprising microcrystalline cellulose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt calculated as free base equivalents,
from about 45% to about 60% wt/wt of a filler (e.g., a filler comprising microcrystalline cellulose);
from about 5% to about 20% wt/wt a binder (e.g., hypromellose);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 15% to about 30% wt/wt of a Compound A citrate salt calculated as free base equivalents,
from about 45% to about 60% wt/wt of a filler (e.g., a filler comprising microcrystalline cellulose);
from about 5% to about 20% wt/wt a binder (e.g., hypromellose);
from about 5% to about 15% wt/wt a disintegrant (e.g., crospovidone);
from about 1% to about 10% wt/wt sodium bicarbonate;
from about 0.1% to about 2% wt/wt a glidant (e.g., hydrophobic colloidal silica); and
from about 0.1% to about 7% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:

(i) from about 15% to about 30% wt/wt of a Compound A citrate Form A salt calculated as free base equivalents, and
(ii) from about 1% to about 10% wt/wt of sodium bicarbonate,
wherein the Compound A citrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 15% to about 30% wt/wt of a Compound A citrate salt calculated as free base equivalents; and
(b) the extragranular phase comprises:
from about 0.5% to about 5% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 15% to about 30% wt/wt of a Compound A citrate salt calculated as free base equivalents;
about 1% to about 10% wt/wt of sodium bicarbonate; and
(b) the extragranular phase comprises:
from about 0.5% to about 5% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure further comprises an intragranular phase and an extragranular phase, wherein:
(a) the intragranular phase comprises:
   from about 15% to about 30% wt/wt of a Compound A citrate salt calculated as free base equivalents;
from about 45% to about 60% wt/wt of a filler (e.g., a filler comprising microcrystalline cellulose);
from about 5% to about 15% wt/wt a disintegrant (e.g., crospovidone);
from about 5% to about 20% wt/wt a binder (e.g., hypromellose);
from about 2% to about 7% wt/wt sodium bicarbonate;
from about 0.1% to about 2% wt/wt a glidant (e.g., hydrophobic colloidal silica);
from about 0.1% to about 2% wt/wt a lubricant (e.g., magnesium stearate); and
(b) the extragranular phase comprises:
from about 0.5% to about 5% wt/wt a lubricant (e.g., magnesium stearate),
wherein the Compound A citrate salt is at least about 95% pure as measured by chromatography, nuclear magnetic resonance spectroscopy, mass spectrometry, powder X-ray diffraction or spectroscopy.

In some embodiments, the tablet comprises Compound A monocitrate monohydrate such that the tablet comprises about 213.2 mg of Compound A calculated as free base equivalents and wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A citrate salt calculated as free base equivalents, and
from about 5 mg to about 50 mg sodium bicarbonate
and wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A citrate salt calculated as free base equivalents,
from about 50 mg to about 500 mg of a filler (e.g., a filler comprising microcrystalline cellulose); and
from about 5 mg to about 50 mg sodium bicarbonate
and wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A citrate salt calculated as free base equivalents,
from about 50 mg to about 500 mg of a filler (e.g., a filler comprising microcrystalline cellulose);
from about 5 mg to about 50 mg sodium bicarbonate; and
from about 1 mg to about 10 mg a glidant (e.g., hydrophobic colloidal silica)
and wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A citrate salt calculated as free base equivalents,
from about 50 mg to about 500 mg of a filler (e.g., a filler comprising microcrystalline cellulose);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate)
and wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A citrate salt calculated as free base equivalents,
from about 50 mg to about 500 mg of a filler (e.g., a filler comprising microcrystalline cellulose);
from about 10 mg to about 100 mg a disintegrant (e.g., crospovidone);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate)
and wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A citrate salt calculated as free base equivalents,
from about 50 mg to about 500 mg of a filler (e.g., a filler comprising microcrystalline cellulose);
from about 10 mg to about 100 mg a binder (e.g., hypromellose);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate)
and wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A citrate salt calculated as free base equivalents,
from about 50 mg to about 500 mg of a filler (e.g., a filler comprising microcrystalline cellulose);
from about 10 mg to about 100 mg a binder (e.g., hypromellose);
from about 10 mg to about 100 mg a disintegrant (e.g., crospovidone);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate)
and wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
(i) from about 25 mg to about 250 mg of a Compound A citrate Form A salt calculated as free base equivalents, and
(ii) from about 10 mg to about 50 mg of sodium bicarbonate
and wherein the Compound A citrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 25 mg to about 250 mg of a Compound A citrate salt calculated as free base equivalents; and
(b) the extragranular phase comprises:
from about 1 mg to about 10 mg a lubricant (e.g., magnesium stearate)
and wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 25 mg to about 250 mg of a Compound A citrate salt calculated as free base equivalents;
about 5 mg to about 50 mg of sodium bicarbonate; and
(b) the extragranular phase comprises:
from about 1 mg to about 10 mg a lubricant (e.g., magnesium stearate)
and wherein the C Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure further comprises an intragranular phase and an extragranular phase, wherein:
(a) the intragranular phase comprises:
   from about 25 mg to about 250 mg of a Compound A citrate salt calculated as free base equivalents;
   from about 50 mg to about 500 mg of a filler (e.g., a filler comprising microcrystalline cellulose);
   from about 10 mg to about 100 mg a disintegrant (e.g., crospovidone);
   from about 10 mg to about 100 mg a binder (e.g., hypromellose);
   from about 5 mg to about 50 mg sodium bicarbonate;
   from about 1 mg to about 10 mg a glidant (e.g., hydrophobic colloidal silica);
   from about 0.5 mg to about 15 mg a lubricant (e.g., magnesium stearate); and
(b) the extragranular phase comprises:
from about 1 mg to about 10 mg a lubricant (e.g., magnesium stearate)
and wherein the Compound A citrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A monocitrate monohydrate salt calculated as free base equivalents, and
from about 5 mg to about 50 mg sodium bicarbonate
and wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A monocitrate monohydrate salt calculated as free base equivalents,
from about 50 mg to about 500 mg of a filler (e.g., a filler comprising microcrystalline cellulose); and
from about 5 mg to about 50 mg sodium bicarbonate
and wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A monocitrate monohydrate salt calculated as free base equivalents,
from about 50 mg to about 500 mg of a filler (e.g., a filler comprising microcrystalline cellulose);
from about 5 mg to about 50 mg sodium bicarbonate; and
from about 1 mg to about 10 mg a glidant (e.g., hydrophobic colloidal silica)
and wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A monocitrate monohydrate salt calculated as free base equivalents,
from about 50 mg to about 500 mg of a filler (e.g., a filler comprising microcrystalline cellulose);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate)
and wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A monocitrate monohydrate salt calculated as free base equivalents,
from about 50 mg to about 500 mg of a filler (e.g., a filler comprising microcrystalline cellulose);
from about 10 mg to about 100 mg a disintegrant (e.g., crospovidone);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate)
and wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A monocitrate monohydrate salt calculated as free base equivalents,
from about 50 mg to about 500 mg of a filler (e.g., a filler comprising microcrystalline cellulose);
from about 10 mg to about 100 mg a binder (e.g., hypromellose);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate).

and wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
from about 25 mg to about 250 mg of a Compound A monocitrate monohydrate salt calculated as free base equivalents,
from about 50 mg to about 500 mg of a filler (e.g., a filler comprising microcrystalline cellulose);
from about 10 mg to about 100 mg a binder (e.g., hypromellose);
from about 10 mg to about 100 mg a disintegrant (e.g., crospovidone);
from about 5 mg to about 50 mg sodium bicarbonate;
from about 1 mg to about 10 mg a glidant (e.g., hydrophobic colloidal silica); and
from about 0.5 mg to about 15 mg a lubricant (e.g., magnesium stearate)
and wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises:
(i) from about 25 mg to about 250 mg of a Compound A monocitrate monohydrate Form A salt calculated as free base equivalents, and
(ii) from about 10 mg to about 50 mg of sodium bicarbonate
and wherein the Compound A monocitrate monohydrate Form A salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 25 mg to about 250 mg of a Compound A monocitrate monohydrate salt calculated as free base equivalents; and
(b) the extragranular phase comprises:
from about 1 mg to about 10 mg a lubricant (e.g., magnesium stearate)
and wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure comprises an intragranular phase and an extragranular phase, wherein:
(a) the intragranular phase comprises:
   from about 25 mg to about 250 mg of a Compound A monocitrate monohydrate salt calculated as free base equivalents;
about 5 mg to about 50 mg of sodium bicarbonate; and
(b) the extragranular phase comprises:
from about 1 mg to about 10 mg a lubricant (e.g., magnesium stearate)
and wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, the tablet of the present disclosure further comprises an intragranular phase and an extragranular phase, wherein:
(a) the intragranular phase comprises:
   from about 25 mg to about 250 mg of a Compound A monocitrate monohydrate salt calculated as free base equivalents;
from about 50 mg to about 500 mg of a filler (e.g., a filler comprising microcrystalline cellulose);
from about 10 mg to about 100 mg a disintegrant (e.g., crospovidone);
from about 10 mg to about 100 mg a binder (e.g., hypromellose);
from about 5 mg to about 50 mg sodium bicarbonate;
from about 1 mg to about 10 mg a glidant (e.g., hydrophobic colloidal silica);
from about 0.5 mg to about 15 mg a lubricant (e.g., magnesium stearate); and
(b) the extragranular phase comprises:
from about 1 mg to about 10 mg a lubricant (e.g., magnesium stearate)
and wherein the Compound A monocitrate monohydrate salt is at least about 95% pure as measured by chromatography, nuclear magnetic resonance spectroscopy, mass spectrometry, powder X-ray diffraction or spectroscopy.

In some embodiments, purity of Compound A monocitrate monohydrate Form A salt is measured using High-performance liquid chromatography/Ultra-performance liquid chromatography (HPLC/UPLC) using ultraviolet (UV) detection.

In some embodiments, the tablet is stable at 25° C.±2° C./60% RH±5% RH for at least about 15 days, least about 1 month, at least about 3 months, at least about 6 months, at least about 9 months, at least about 11 months, at least about 12 months, at least about 18 months, or at least about 24 months.

In some embodiments, the tablet is stable at 40° C.±2° C./75% RH±5% RH for at least about 15 days, at least about 1 month, at least about 3 months, at least about 6 months, at least about 9 months, at least about 11 months, at least about 12 months, or at least about 18 months.

In some embodiments, the tablet is subjected to temperature cycling and is stable for at least about 1 month, at least about 3 months, at least about 6 months, at least about 9 months, at least about 11 months, at least about 12 months, at least about 18 months, or at least about 24 months after the temperature cycling; wherein each temperature cycle, Compound A monocitrate monohydrate tablet is stored at −20° C. for 3 days then allowed to equilibrate at 25° C./60% RH for 1 day followed by 3 days of storage at 50° C.

In some embodiments, the tablet is stable at 40° C.±2° C./75% RH±5% RH for at least about 1 month, at least about 3 months, at least about 6 months, at least about 9 months, at least about 11 months, at least about 12 months, or at least about 18 months.

### Methods of Use

In some embodiments, Compound A, a salt thereof, or a polymorphic form thereof (e.g., Compound A monocitrate monohydrate Form A salt), or a pharmaceutical composition (e.g. liquid formulation or solid formulation such as a tablet) thereof is used in treating or preventing a disease caused by, or associated with, menin expression, activity, and/or function.

In some embodiments, the Compound A, a salt thereof, or a polymorphic form thereof (e.g., Compound A monocitrate monohydrate Form A salt) is used to manufacture of a medicament for treating a cancer to a subject.

In some embodiments, Compound A, a salt thereof, or a polymorphic form thereof (e.g., Compound A monocitrate monohydrate Form A salt), or a pharmaceutical composition (e.g. liquid formulation or solid formulation such as a tablet) thereof is used in treating a cancer to a subject.

In some embodiments, the cancer is a hematological cancer. In some embodiments, the cancer is a leukemia. In some embodiments, the cancer is a lymphoma.

In some embodiments, the cancer is mixed lineage leukemia (MLL), MLL-related leukemia, MLL-associated leukemia, MLL-positive leukemia, MLL-induced leukemia, lysine methyltransferase 2A gene rearrangement (KMT2Ar or KMT2A) (also referred to as rearranged mixed lineage leukemia (MLL-r) or KMT2A translocation), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.

In some embodiments, the cancer is an abstract nucleophosmin (NPM1)-mutated acute myeloid leukemia (i.e., NPM1^{mut} acute myeloid leukemia).

In some embodiments, Compound A, a salt thereof, or a polymorphic form thereof (e.g., Compound A monocitrate monohydrate Form A salt), or a pharmaceutical composition (e.g. liquid formulation or solid formulation such as a tablet) thereof is used to treat cancer, wherein the cancer is a leukemia.

In some embodiments, the subject to be treated of acute leukemia with Compound A, a salt thereof, or a polymorphic form thereof (e.g., Compound A monocitrate monohydrate Form A salt), or a pharmaceutical composition (e.g. liquid formulation or solid formulation such as a tablet) thereof is selected based on the presence of lysine methyltransferase 2A gene rearrangement (KMT2Ar or KMT2A) in bone marrow cells.

### Disease Indications

In some embodiments is a method of treating a cancer in an individual in need thereof comprising administering Compound A, a salt thereof, or a polymorphic form thereof (e.g., Compound A monocitrate monohydrate Form A salt), or a pharmaceutical composition (e.g. liquid formulation or solid formulation such as a tablet) thereof. In some embodiments is a method of treating a cancer in an individual in need thereof comprising administering Compound A, a salt thereof, or a polymorphic form thereof (e.g., Compound A monocitrate monohydrate Form A salt), or a pharmaceutical composition (e.g. liquid formulation or solid formulation such as a tablet) thereof twice daily. Accordingly, the invention are believed to be effective against a broad range of cancers, including, but not limited to, hematological cancer (e.g., leukemia and lymphoma), bladder cancer, brain cancer (e.g., glioma, diffuse intrinsic pontine glioma (DIPG)), breast cancer (e.g., triple-negative breast cancer, estrogen-receptor-positive breast cancer (i.e., ER+ breast cancer)), colorectal cancer (e.g., refractory metastatic microsatellite stable (MSS) colorectal cancer (CRC)), cervical cancer, gastrointestinal cancer (e.g., colorectal carcinoma, gastric cancer), genitourinary cancer, head and neck cancer, liver cancer, lung cancer, melanoma, ovarian cancer, pancreatic cancer, prostate cancer (e.g., castration resistant prostate cancer), renal cancer (e.g., renal cell carcinoma), skin cancer, thyroid cancer (e.g., papillary thyroid carcinoma), testicular cancer, sarcoma (e.g., Ewing's sarcoma), and AIDS-related cancers. In some embodiments, the cancer is associated with a rearranged MLL gene. In some embodiments, the pathophysiology of the cancer is dependent on the MLL gene. In some embodiments, the MLL gene is MLL1. In some embodiments, the cancer is associated with mutant p53 gain-of-function.

In some embodiments, the specific cancers that may be treated by Compound A, a salt thereof, or a polymorphic form thereof (e.g., Compound A monocitrate monohydrate Form A salt), or a pharmaceutical composition (e.g. liquid formulation or solid formulation such as a tablet) thereof described herein include cardiac cancers, such as for example, sarcoma (e.g., angiosarcoma, fibrosarcoma, rhabdomyosarcoma, and liposarcoma), myxoma, rhabdomyoma, fibroma, lipoma and teratoma; lung cancers, including, for example, bronchogenic carcinoma (e.g., squamous cell, undifferentiated small cell, undifferentiated large cell, and adenocarcinoma), alveolar and bronchiolar carcinoma, bronchial adenoma, sarcoma, lymphoma, chondromatous hamartoma, mesothelioma, non-small cell lung cancer, small cell lung cancer, bronchial adenomas/carcinoids, and pleuropulmonary blastoma; gastrointestinal cancer, including, for example, cancers of the esophagus (e.g., squamous cell carcinoma, adenocarcinoma, leiomyosarcoma, and lymphoma), cancers of the stomach (e.g., carcinoma, lymphoma, and leiomyosarcoma), cancers of the pancreas (e.g., ductal adenocarcinoma, insulinoma, glucagonoma, gastrinoma, carcinoid tumors, and vipoma), cancers of the small bowel (e.g., adenocarcinoma, lymphoma, carcinoid tumors, Kaposi's sarcoma, leiomyoma, hemangioma, lipoma, neurofibroma, and fibroma), cancers of the large bowel or colon, (e.g., adenocarcinoma, tubular adenoma, villous adenoma, hamartoma, and leiomyoma), and other cancers of the digestive tract (e.g., anal cancer, anorectal cancer, appendix cancer, cancer of the anal canal, cancer of the tongue, gallbladder cancer, gastrointestinal stromal tumor (GIST), colon cancer, colorectal cancer (e.g., refractory metastatic microsatellite stable (MSS) colorectal cancer (CRC)), extrahepatic bile duct cancer, intrahepatic bile duct cancer, rectal cancer, and small intestine cancer); genitourinary tract cancers, including, for example, cancers of the kidney (e.g., adenocarcinoma, Wilm's tumor (nephroblastoma), lymphoma, and leukemia), cancers of the bladder and urethra (e.g., squamous cell carcinoma, transitional cell carcinoma, and adenocarcinoma), cancers of the prostate (e.g., adenocarcinoma and sarcoma), cancers of the testis, (e.g., seminoma, teratoma, embryonal carcinoma, teratocarcinoma, choriocarcinoma, sarcoma, interstitial cell carcinoma, fibroma, fibroadenoma, adenomatoid tumors, and lipoma), as well as transitional cell cancer, transitional cell cancer of the renal pelvis and ureter and other urinary organs, urethral cancer, and urinary bladder cancer; liver cancers, including, for example, hepatoma (e.g., hepatocellular carcinoma), cholangiocarcinoma, hepatoblastoma, angiosarcoma, hepatocellular adenoma, and hemangioma; bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; nervous system cancers, including, for example, cancers of the skull (e.g., osteoma, hemangioma, granuloma, xanthoma, and osteitis deformans); cancers of the meninges (e.g., meningioma, meningiosarcoma, and gliomatosis); cancers of the brain (e.g., astrocytoma, medulloblastoma, glioma, ependymoma, germinoma (pinealoma), glioblastoma multiforme, oligodendroglioma, schwannoma, retinoblastoma, and congenital tumors); cancers of the spinal cord (e.g., neurofibroma, meningioma, glioma, and sarcoma), and other nervous system cancers (e.g., brain stem glioma, diffuse intrinsic pontine glioma (DIPG), brain tumor, central nervous system cancer, cerebellar astrocytoma, cerebral astrocytoma/malignant glioma, childhood cerebellar astrocytoma, childhood cerebral astrocytoma, primary central nervous system lymphoma, visual pathway and hypothalamic glioma, nervous system lymphoma, supratentorial primitive neuroectodermal tumors, pineoblastoma and supratentorial primitive neuroectodermal tumors); gynecological cancers, including, for example, cancers of the uterus (e.g., endometrial carcinoma), cancers of the cervix (e.g., cervical carcinoma, and pre tumor cervical dysplasia), cancers of the ovaries (e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma), cancers of the vulva (e.g., squamous cell carcinoma, intraepithelial carcinoma, adenocarcinoma, fibrosarcoma, and melanoma), cancers of the vagina (e.g., clear cell carcinoma, squamous cell carcinoma, botryoid sarcoma, and embryonal rhabdomyosarcoma), and cancers of the fallopian tubes (e.g., carcinoma); other reproductive tract cancers, including, for example, endometrial cancer, endometrial uterine cancer, germ cell tumor, gestational trophoblastic tumor, gestational trophoblastic tumor glioma, ovarian epithelial cancer, ovarian germ cell tumor, ovarian low malignant potential tumor, penile cancer, vaginal cancer, vulvar cancer, extracranial germ cell tumor, extragonadal germ cell tumor, uterine cancer, uterine corpus cancer, uterine sarcoma; lymphatic and hematologic cancers, including, for example, cancers of the blood (e.g., acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, and myelodysplastic syndrome, Hodgkin's lymphoma, non Hodgkin's lymphoma (malignant lymphoma) and Waldenstrom's macroglobulinemia), and other lymphatic or hematologic cancers including, for example, childhood leukemia, plasma cell neoplasm/multiple myeloma, myelodysplasia, myelodysplastic syndrome, cutaneous T-cell lymphoma, lymphoid neoplasm, AIDS-related lymphoma, thymoma, thymoma and thymic carcinoma, mycosis fungoides, and Sézary Syndrome; skin cancers, including, for example, malignant melanoma, basal cell carcinoma, squamous cell carcinoma, Kaposi's sarcoma, moles dysplastic nevi, lipoma, angioma, dermatofibroma, keloids, psoriasis, merkel cell carcinoma, merkel cell skin carcinoma, melanoma, and carcinoid tumor; adrenal gland cancers, including, for example, neuroblastoma; other cancers associated with the endocrine system including, for example, adrenocortical carcinoma, multiple endocrine neoplasia (e.g., multiple endocrine neoplasia type I), multiple endocrine neoplasia syndrome, parathyroid cancer, pituitary tumor, pheochromocytoma, islet cell pancreatic cancer, and islet cell tumors); connective tissue cancer (e.g., bone cancer, bone and joint cancer, osteosarcoma and malignant fibrous histiocytoma); cancer associated with the head, neck, and mouth (e.g., head and neck cancer, paranasal sinus and nasal cavity cancer, metastatic squamous neck cancer, mouth cancer, throat cancer, esophageal cancer, laryngeal cancer, pharyngeal cancer, hypopharyngeal cancer, lip and oral cavity cancer, nasopharyngeal cancer, oral cancer, oropharyngeal cancer, and salivary gland cancer); and cancer associated with the eye (e.g., ocular cancer, intraocular melanoma). In some embodiments, the cancer is Ewing's sarcoma.

In some embodiments, the cancer is a hematological cancer such as leukemia or lymphoma. Example leukemia and lymphomas treatable by Compound A, a salt thereof, or a polymorphic form thereof (e.g., Compound A monocitrate monohydrate Form A salt), or a pharmaceutical composition (e.g. liquid formulation or solid formulation such as a tablet) thereof of the invention include mixed lineage leukemia (MLL), MLL-related leukemia, MLL-associated leukemia, MLL-positive leukemia, MLL-induced leukemia, lysine methyltransferase 2A gene rearrangement (KMT2Ar or KMT2A), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL) (also referred to as acute lymphoblastic leukemia or acute lymphoid leukemia), acute myeloid leukemia (AML) (also referred to as acute myelogenous leukemia or acute myeloblastic leukemia), acute granulocytic leukemia, acute nonlymphocytic leukemia, chronic lymphocytic leukemia (CLL) (also referred to as chronic lymphoblastic leukemia), chronic myelogenous leukemia (CML) (also referred to as chronic myeloid leukemia), therapy related leukemia, myelodysplastic syndrome (MDS), myeloproliferative neoplasia (MPN), plasma cell neoplasm, multiple myeloma, myelodysplasia, cutaneous T-cell lymphoma, nucleophosmin (NPM1) AML, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), and Waldenstrom's macroglobulinemia. In some embodiments, the acute myeloid leukemia (AML) is abstract nucleophosmin (NPM1)-mutated acute myeloid leukemia (i.e., NPM1^{mut} acute myeloid leukemia).

In particular embodiments, Compound A, a salt thereof, or a polymorphic form thereof (e.g., Compound A monocitrate monohydrate Form A salt), or a pharmaceutical composition (e.g. liquid formulation or solid formulation such as a tablet) thereof of the invention is used to treat leukemia associated with a MLL rearrangement, acute lymphocytic leukemia associated with a MLL rearrangement, acute lymphoblastic leukemia associated with a MLL rearrangement, acute lymphoid leukemia associated with a MLL rearrangement, acute myeloid leukemia associated with a MLL rearrangement, acute myelogenous leukemia associated with a MLL rearrangement, or acute myeloblastic leukemia associated with a MLL rearrangement. As used herein, “MLL rearrangement” means a rearrangement of the MLL gene.

Malignant lymphomas are neoplastic transformations of cells that reside predominantly within lymphoid tissues. Two groups of malignant lymphomas are Hodgkin's lymphoma and non-Hodgkin's lymphoma (NHL). Both types of lymphomas infiltrate reticuloendothelial tissues. However, they differ in the neoplastic cell of origin, site of disease, presence of systemic symptoms, and response to treatment (Freedman et al., “Non-Hodgkin's Lymphomas” Chapter 134, Cancer Medicine, (an approved publication of the American Cancer Society, B.C. Decker Inc., Hamilton, Ontario, 2003).

The acute leukemia population having a lysine methyltransferase 2A (KMT2A) gene rearrangement consists of both adult and pediatric patients. A pharmaceutical composition (e.g., liquid formulation, or a solid formulation such as a tablet) comprising Compound A, a salt thereof, or a polymorphic form thereof of the present invention is additionally useful for the treatment of leukemia patients with an MLL KMT2A gene rearrangement.

In some embodiments, the present disclosure is directed to a method of treating a cancer comprising administering the Compound A monocitrate monohydrate Form A salt, or tablet to a subject in need thereof.

In some embodiments, the present disclosure is directed to a method of treating a subject with relapsed or refractory acute leukemia with a lysine methyltransferase 2A gene (KMT2A) rearrangement, the method comprising administering the Compound A monocitrate monohydrate Form A salt, a tablet comprising Compound A monocitrate monohydrate Form A salt, or a liquid formulation comprising Compound A monocitrate monohydrate Form A salt to the subject.

In some embodiments, the present disclosure is directed to a pharmaceutical composition comprising a Compound A, a salt thereof, or a polymorphic form thereof (e.g., Compound A monocitrate monohydrate Form A salt), or a pharmaceutical composition (e.g. liquid formulation or solid formulation such as a tablet) thereof and a pharmaceutically acceptable excipient.

In some embodiments, the present disclosure is directed to a pharmaceutical composition comprising a Compound A, a salt thereof, or a polymorphic form thereof (e.g., Compound A monocitrate monohydrate Form A salt), or a pharmaceutical composition (e.g. liquid formulation or solid formulation such as a tablet) thereof for use in the treatment or prevention of a cancer in a subject.

In some embodiments, the present disclosure is directed to a pharmaceutical composition comprising a Compound A, a salt thereof, or a polymorphic form thereof (e.g., Compound A monocitrate monohydrate Form A salt), or a pharmaceutical composition (e.g. liquid formulation or solid formulation such as a tablet) thereof for use in the treatment of a cancer in a subject.

In some embodiments, present disclosure is directed to a method of treating a disease where menin-MLL interaction plays a role comprising administering Compound A monocitrate monohydrate Form A salt, tablet, or liquid formulation to a subject in need thereof.

In some embodiments, present disclosure is directed to a method of treating a disease where menin-MLL interaction plays a role comprising administering a combination comprising Compound A monocitrate monohydrate Form A salt and a CYP3A4 inhibitor to a subject in need thereof.

In some embodiments, present disclosure is directed to a method of treating a disease where menin-MLL interaction plays a role comprising administering a pharmaceutical composition comprising Compound A monocitrate monohydrate Form A salt to a subject in need thereof.

### Leukemia

Disclosed herein, in certain embodiments, is a method for treating a leukemia in an individual in need thereof, comprising: administering Compound A, a salt thereof, or a polymorphic form thereof (e.g., Compound A monocitrate monohydrate Form A salt), or a pharmaceutical composition (e.g. liquid formulation or solid formulation such as a tablet) thereof.

Leukemia is a cancer of the blood or bone marrow characterized by an abnormal increase of blood cells, usually leukocytes (white blood cells). Leukemia is a broad term covering a spectrum of diseases. The first division is between its acute and chronic forms: (i) acute leukemia is characterized by the rapid increase of immature blood cells. This crowding makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children; (ii) chronic leukemia is distinguished by the excessive build up of relatively mature, but still abnormal, white blood cells. Typically taking months or years to progress, the cells are produced at a much higher rate than normal cells, resulting in many abnormal white blood cells in the blood. Chronic leukemia mostly occurs in older people, but can theoretically occur in any age group. Additionally, the diseases are subdivided according to which kind of blood cell is affected. This split divides leukemias into lymphoblastic or lymphocytic leukemias and myeloid or myelogenous leukemias: (i) lymphoblastic or lymphocytic leukemias, the cancerous change takes place in a type of marrow cell that normally goes on to form lymphocytes, which are infection-fighting immune system cells; (ii) myeloid or myelogenous leukemias, the cancerous change takes place in a type of marrow cell that normally goes on to form red blood cells, some other types of white cells, and platelets.

Within these main categories, there are several subcategories including, but not limited to, Acute lymphoblastic leukemia (ALL), Acute myelogenous leukemia (AML), Chronic myelogenous leukemia (CML), and Hairy cell leukemia (HCL).

Symptoms, diagnostic tests, and prognostic tests for each of the above-mentioned conditions are known. See, e.g., “Harrison's Principles of Internal Medicine” 16th ed., 2004, The McGraw-Hill Companies, Inc. Dey et al. (2006), Cytojournal 3(24), and the “Revised European American Lymphoma” (REAL) classification system (see, e.g., the website maintained by the National Cancer Institute).

A number of animal models are useful for establishing a range of therapeutically effective doses of inhibitor compounds, such as the menin inhibitor (e.g., Compound A or a pharmaceutical acceptable salt thereof), for treating any of the foregoing diseases.

The therapeutic efficacy of menin inhibitor (e.g., Compound A or a pharmaceutical acceptable salt thereof) for any one of the foregoing diseases can be optimized during a course of treatment. For example, a subject being treated can undergo a diagnostic evaluation to correlate the relief of disease symptoms or pathologies to inhibition of in vivo menin activity achieved by administering a given dose of the menin inhibitor (e.g., Compound A or a pharmaceutical acceptable salt thereof). Cellular assays known in the art can be used to determine in vivo activity. Thus, the amount of the menin inhibitor (e.g., Compound A or a pharmaceutical acceptable salt thereof) that is administered to a subject can be increased or decreased as needed so as to maintain a level of menin inhibition optimal for treating the subject's disease state.

In some embodiments, Compound A, a salt thereof, or a polymorphic form thereof (e.g., Compound A monocitrate monohydrate Form A salt), or a pharmaceutical composition (e.g. liquid formulation or solid formulation such as a tablet) thereof is used for the manufacture of a medicament for treating any of the foregoing conditions. In some embodiments, Compound A, a salt thereof, or a polymorphic form thereof (e.g., Compound A monocitrate monohydrate Form A salt), or a pharmaceutical composition (e.g. liquid formulation or solid formulation such as a tablet) thereof of the present invention is directed to the treatment of leukemia. In some embodiments, the present invention is directed to the treatment of leukemia in a patient in need thereof comprising administering Compound A, a salt thereof, or a polymorphic form thereof (e.g., Compound A monocitrate monohydrate Form A salt), or a pharmaceutical composition (e.g. liquid formulation or solid formulation such as a tablet) thereof.

Acute leukemias generally result from acquired mutations in hematopoietic progenitor cells. Chromosomal abnormalities are often discrete mutational features in leukemia. Many of these chromosomal abnormalities are due to specific translocations that lead to the formation of fusion genes which become drivers for tumorigenesis and tumor development. A specific example involves the MLL1 gene. Translocations at the MLL1 locus (11q23) can lead to the formation of oncogenic gene fusions that characterize MLLr acute leukemias. The MLL1 protein is a key regulator of development and is the mammalian homologue of Drosophila trithorax. It is an important epigenetic regulator of HOX gene expression. Translocations at the MLL1 locus create chimeric proteins that fuse the N-terminus of MLL1 to variable C-terminal domains derived from different translocation partners. Currently, more than 90 different fusion partners are known. Expression of these fusions enables an aberrant transcription program characterized by overexpression of HOX and other developmental genes. This transcription program suppresses differentiation and enhances proliferation, leading to the MLLr acute leukemias. Translocations involving the MLL1 locus (11q23) are routinely diagnosed using fluorescence in situ hybridization (FISH). Depending on the progenitor cell of origin, MLLr can phenotypically appear as ALL, AML, or mixed phenotype acute leukemia (MPAL). These translocations are rare and MLLr has a combined annual incidence of ~4000 cases per year in the United States (US), Europe and Japan. Approximately 10% of all leukemias harbor MLL1 translocations.

Compound A, a salt thereof, or a polymorphic form thereof (e.g., Compound A monocitrate monohydrate Form A salt), or a pharmaceutical composition (e.g. liquid formulation or solid formulation such as a tablet) thereof is additionally useful for the treatment of leukemia patients with an MLL KMT2A gene rearrangement.

### Treatment of Relapsed or Refractory MLLr Acute Leukemias

The relapse risk for MLLr patients is high after conventional chemotherapy and stem cell transplantation, with an overall 5-year survival rate of only approximately 35%. No therapies are currently available that specifically target MLLr leukemia.

The interaction of MLL1 fusion proteins with menin is a key driver of MLLr acute leukemias. Both MLL1 and MLLr fusions bind to a well-characterized high affinity site on the chromatin-associated protein menin. The binding of MLL1 fusions to menin is mediated by amino acid residues 9-13 (FPARP) found at the N-terminus of MLL1. Binding to menin localizes these fusions to chromatin where they enable a leukemic transcription program, which includes upregulation of HOXA locus and MEIS1 genes. The interaction between the fusion protein and menin is required to maintain this transcription program.

The menin inhibitor (e.g., Compound A or a pharmaceutically acceptable salt thereof) binds with high affinity to the MLL1 binding pocket on menin and displays activity across a range of cells harboring MLLr fusions. Menin inhibitor (e.g., Compound A or a pharmaceutically acceptable salt thereof) disrupts the interaction between menin and the MLL1 fusion proteins which is required for leukemogenic activity, thus impairing expression of critical oncogenes, causing growth arrest and the inhibition of cellular proliferation. Small molecule inhibitors of the menin-MLL interaction have been reported. These inhibitors have demonstrated anti-proliferative activity against MLLr cell lines and have shown single agent survival benefit in mouse models of MLLr leukemia. These data indicate that pharmacologic inhibition of the menin-MLL interaction represents a potential targeted strategy for the treatment MLLr acute leukemias.

### NPM1c AML

AML is an acute leukemia characterized by accumulation of myeloid cells in the bone marrow as a result of impaired differentiation and proliferation. NPM1 is among the most frequently mutated genes in AML. Point mutations in the NPM1 gene lead to the aberrant cytoplasmic localization of the mutant proteins, which are termed NPM1c. The identification of NPM1c is an essential part of the diagnostic screening of AML. AML patients with mutations in NPM1 alone have a favorable prognosis with ~60% five-year overall survival (OS). However, most (>80%) NPM1c AML patients harbor multiple concurrent mutations which can adversely affect the prognosis. Co-mutations have been found in FLT3, DNMT3A, NRAS, TET2 and IDH1/2 genes, among others. NPM1c is generally not found in ALL.

Treatment of Patients with NPM1c AML

The American Cancer Society estimates there will be ~21,450 new cases of AML in the US in 2019. In adult AML, ~30% of patients have NPM1c.

NPM1 normally exists as a nucleolar protein that shuttles between the nucleus and cytoplasm. It has multiple functions, including ribosomal protein assembly and transport, control of centrosome duplication, and regulation of the tumor suppressor ARE. The cytosolic localization of mutant NPM1c leads to the aberrant partitioning of NPM1c-associated nuclear proteins into the cytoplasm, including several transcription factors. Among these is PU.1, a master driver of monocyte lineage differentiation. Loss of PU.1 from the nucleus in NPM1c AML leads to suppression of >500 terminal differentiation genes. The suppression of differentiation by NPM1c enables a leukemic transcription program that is highly dependent on the up-regulated expression of HOXA cluster and MEIS1 genes. Expression of these genes further blocks differentiation and induces long-term proliferation, leading to the leukemic phenotype.

In addition, the HOX/MEIS signatures of NPM1c AMLs overlap with those of MLLr leukemias and hematopoietic stem cells (HSC). Maintenance of this transcription signature in NPM1c cells depends directly on the menin-MLL1 interaction.

While little is known regarding how mutant NPM1c cells maintain aberrant gene expression, mutation of the menin binding motif in MLL1 has been shown to strongly inhibit the proliferative capacity of NPM1c cells due to loss of MLL1 binding to menin. In addition, the small molecule menin-MLL interaction inhibitor, MI-503, was shown to suppress the HOXA/MEIS1 transcription program in NPM1c cells, leading to growth arrest, terminal differentiation and cell death, confirming a critical role for the menin-MLL1 interaction in NPM1c cells. These findings were validated and extended in another report that demonstrated that the orally active menin inhibitor, KO-539, had robust anti-leukemic activity in NPM1c mutant AML patient-derived xenograft models. Overall, these results demonstrated that NPM1c harboring cells are very sensitive to menin-MLL interaction inhibitors.

In some embodiments, Compound A, a salt thereof, or a polymorphic form thereof (e.g., Compound A monocitrate monohydrate Form A salt), or a pharmaceutical composition (e.g. liquid formulation or solid formulation such as a tablet) thereof of the present invention is directed to the treatment of NMP1 AML. In some embodiments, the present invention is directed to the treatment of NMP1 AML in a patient in need thereof comprising administering Compound A, a salt thereof, or a polymorphic form thereof (e.g., Compound A monocitrate monohydrate Form A salt), or a pharmaceutical composition (e.g. liquid formulation or solid formulation such as a tablet) thereof.

### Pharmaceutical Compositions/Formulations

Disclosed herein, in certain embodiments, are pharmaceutical compositions comprising Compound A, a salt thereof, or a polymorphic form thereof (e.g., Compound A monocitrate monohydrate Form A salt), and a pharmaceutically-acceptable excipient, diluent, or carrier.

In some embodiments, the Compound A citrate salt is amorphous or crystalline.

Pharmaceutical compositions may be formulated in a conventional manner using one or more physiologically acceptable carriers including excipients and auxiliaries which facilitate processing of the active compounds into preparations which can be used pharmaceutically.

Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference in their entirety.

A pharmaceutical composition, as used herein, refers to Compound A, a salt thereof, or a polymorphic form thereof (e.g., Compound A monocitrate monohydrate Form A salt) and/or an additional therapeutic agent with other chemical components, such as carriers, stabilizers, diluents, dispersing agents, suspending agents, thickening agents, and/or excipients. A pharmaceutical composition, as used herein, refers to Compound A, a salt thereof, or a polymorphic form thereof (e.g., Compound A monocitrate monohydrate Form A salt) and one or more of the following excipients: microcrystalline cellulose, crospovidone, Hypromellose, sodium bicarbonate, hydrophobic colloidal silica, and magnesium stearate.

A pharmaceutical composition, as used herein, refers to crystalline Compound A citrate salt and/or an additional therapeutic agent with other chemical components, such as carriers, stabilizers, diluents, dispersing agents, suspending agents, thickening agents, and/or excipients. A pharmaceutical composition, as used herein, refers to crystalline Compound A citrate salt and one or more of the following excipients: microcrystalline cellulose, crospovidone, Hypromellose, sodium bicarbonate, hydrophobic colloidal silica, and magnesium stearate.

A pharmaceutical composition, as used herein, refers to crystalline Compound A monocitrate monohydrate salt and/or an additional therapeutic agent with other chemical components, such as carriers, stabilizers, diluents, dispersing agents, suspending agents, thickening agents, and/or excipients. A pharmaceutical composition, as used herein, refers to crystalline Compound A citrate salt and one or more of the following excipients: microcrystalline cellulose, crospovidone, Hypromellose, sodium bicarbonate, hydrophobic colloidal silica, and magnesium stearate.

A pharmaceutical composition, as used herein, refers to amorphous Compound A citrate salt and/or an additional therapeutic agent with other chemical components, such as carriers, stabilizers, diluents, dispersing agents, suspending agents, thickening agents, and/or excipients. A pharmaceutical composition, as used herein, refers to amorphous Compound A citrate salt and one or more of the following excipients: microcrystalline cellulose, crospovidone, hypromellose, sodium bicarbonate, hydrophobic colloidal silica, and magnesium stearate.

A pharmaceutical composition, as used herein, refers to amorphous Compound A monocitrate monohydrate salt and/or an additional therapeutic agent with other chemical components, such as carriers, stabilizers, diluents, dispersing agents, suspending agents, thickening agents, and/or excipients. A pharmaceutical composition, as used herein, refers to amorphous Compound A citrate salt and one or more of the following excipients: microcrystalline cellulose, crospovidone, hypromellose, sodium bicarbonate, hydrophobic colloidal silica, and magnesium stearate.

In practicing the methods of treatment or use provided herein, therapeutically effective amounts of Compound A, a salt thereof, or a polymorphic form thereof (e.g., Compound A monocitrate monohydrate Form A salt) disclosed herein are administered having a disease, disorder, or condition to be treated. In some embodiments, the mammal is a human. The therapeutically effective amounts of the compounds may vary depending on the severity of the disease, the age and relative health of the subject, and other factors.

Pharmaceutical compositions including Compound A, a salt thereof, or a polymorphic form thereof (e.g., Compound A monocitrate monohydrate Form A salt) described herein may be manufactured in a conventional manner, such as, by way of example only, by means of conventional mixing, dissolving, granulating, dragee-making, levigating, emulsifying, encapsulating, entrapping or compression processes.

### Dosage Forms

The pharmaceutical compositions of the present application comprise a Compound A, a salt thereof, or a polymorphic form thereof (e.g., Compound A monocitrate monohydrate Form A salt) of the present application or a polymorphic form thereof formulated together with one or more pharmaceutically acceptable excipients. Compounds of the application may be administered as pharmaceutical compositions by any conventional route, in particular enterally, e.g., orally, e.g., in the form of tablets or capsules, or parenterally, e.g., in the form of injectable solutions or suspensions, or topically, e.g., in the form of lotions, gels, ointments or creams, or in a nasal or suppository form.

Pharmaceutical compositions including the individual compounds of the combination of the present application in free form or in a pharmaceutically acceptable salt form in association with at least one pharmaceutically acceptable carrier or diluent may be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present application with a carrier. A carrier may include absorbable pharmacologically acceptable solvents to assist passage through the skin of the host. For example, transdermal devices may be in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives. The pharmaceutical compositions of the present application comprise a therapeutically effective amount of a compound of the present application formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which may serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylenepolyoxy propylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this application may be administered to humans and other animals orally, rectally, parenterally, intracisternally, intravaginally, intraperitoneally, topically (as by powders, ointments, or drops), buccally, or as an oral or nasal spray.

As used herein, the term “pharmaceutically acceptable carrier” means a non-toxic, inert solid, semi-solid or liquid filler, diluent, encapsulating material or formulation auxiliary of any type. Some examples of materials which may serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylenepolyoxy propylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration may include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous, or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this application with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds may also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this application include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this application.

The ointments, pastes, creams and gels may contain, in addition to an active compound of this application, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this application, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The pharmaceutical compositions described herein may be formulated for administration via any conventional means including, but not limited to, oral, parenteral (e.g., intravenous, subcutaneous, or intramuscular), buccal, intranasal, rectal or transdermal administration routes. As used herein, the terms “subject”, “individual” and “patient” are used interchangeably and mean an animal, preferably a mammal, including a human or non-human. None of the terms require the supervision (continuous or otherwise) of a medical professional. As used herein, the subject is an adult (age 18 or over) or a pediatric patient (age ranging from and includes 1 month (30 days) to 18 years). In some embodiments, the pediatric patient is 6 months and older.

The pharmaceutical compositions described herein are formulated into any suitable dosage form, including but not limited to, solid oral dosage forms, controlled release formulations, fast melt formulations, effervescent formulations, tablets, powders, pills, capsules, delayed release formulations, extended release formulations, pulsatile release formulations, multiparticulate formulations, and mixed immediate release and controlled release formulations.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al, The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical dosage forms described herein may include one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the pharmaceutical compositions. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this application include powders, sprays, ointments, pastes, creams, lotions, gels, solutions, patches and inhalants. In some embodiments, the active compound is mixed under sterile conditions with a pharmaceutically acceptable carrier, and with any preservatives, buffers or propellants that are required.

The pharmaceutical compositions containing active compounds of the present application may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Techniques for formulation and administration of the disclosed compounds of the application can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

### Dosing and Treatment Regimens

The dosage regimen utilizing the compounds of the disclosure is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

The dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day up to, and including about 1,000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 1,000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 950 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 900 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 850 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 800 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 750 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 700 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 650 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 600 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 550 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 500 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 450 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 400 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 350 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 300 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 250 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 200 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 150 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 100 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 25 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 50 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 100 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 150 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 200 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 250 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 300 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 350 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 400 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 450 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 500 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 550 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 600 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 650 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 700 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 750 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 800 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 850 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 900 mg/day to about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 20 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 25 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 50 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 100 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 110 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 150 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 160 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 200 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 220 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 250 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 270 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 300 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 320 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 350 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 400 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 440 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 450 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 500 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 540 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 550 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 600 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 650 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 700 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 750 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 800 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 850 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 900 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 950 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 1000 mg/day as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day up to, and including about 1,000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 1,000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 950 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 900 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 850 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 800 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 750 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 700 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 650 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 600 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 550 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 500 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 450 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 400 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 350 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 300 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 250 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 200 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 150 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/day to about 100 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 25 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 50 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 100 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 150 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 200 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 250 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 300 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 350 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 400 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 450 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 500 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 550 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 600 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 650 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 700 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 750 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 800 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 850 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 900 mg/day to about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 20 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 25 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 50 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 100 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 110 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 150 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 160 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 200 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 220 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 250 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 270 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 300 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 320 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 350 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 400 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 440 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 450 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 500 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 540 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 550 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 600 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 650 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 700 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 750 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 800 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 850 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 900 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 950 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 1000 mg/day.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg up to, and including about 1,000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 1,000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 25 mg to about 270 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 950 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 900 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 850 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 800 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 750 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 700 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 650 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 600 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 550 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 500 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 450 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 400 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 350 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 300 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 250 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 200 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 150 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 100 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 25 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 50 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 100 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 150 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 200 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 250 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 300 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 350 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 400 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 450 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 500 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 550 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 600 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 650 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 700 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 750 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 800 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 850 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 900 mg to about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 20 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 25 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 50 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 100 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 110 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 150 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 160 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 200 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 220 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 250 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 270 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 300 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 320 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 350 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 400 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 440 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 450 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 500 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 540 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 550 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 600 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 650 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 700 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 750 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 800 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 850 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 900 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 950 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 1000 mg as free base equivalents twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg up to, and including about 1,000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 1,000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 950 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 900 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 850 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 800 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 750 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 700 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 650 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 600 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 550 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 500 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 450 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 400 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 350 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 300 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 250 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 200 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 150 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg to about 100 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 25 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 50 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 100 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 150 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 200 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 250 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 300 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 350 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 400 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 450 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 500 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 550 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 600 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 650 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 700 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 750 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 800 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 850 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 900 mg to about 1000 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 20 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 25 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 50 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 100 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 110 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 150 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 160 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 200 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 220 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 250 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 270 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 300 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 320 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 350 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 400 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 440 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 450 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 500 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 540 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 550 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 600 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 650 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 700 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 750 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 800 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 850 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 900 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 950 mg twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 1000 mg twice daily.

In some embodiments, the amount of Compound A monocitrate monohydrate salt that is administered to a subject in need thereof is about 20 mg as free base equivalents orally twice daily.

In some embodiments, the amount of Compound A monocitrate monohydrate salt that is administered to a subject in need thereof is about 25 mg as free base equivalents orally twice daily.

In some embodiments, the amount of Compound A monocitrate monohydrate salt that is administered to a subject in need thereof is about 50 mg as free base equivalents orally twice daily.

In some embodiments, the amount of Compound A monocitrate monohydrate salt that is administered to a subject in need thereof is about 75 mg as free base equivalents orally twice daily.

In some embodiments, the amount of Compound A monocitrate monohydrate salt that is administered to a subject in need thereof is about 80 mg as free base equivalents orally twice daily.

In some embodiments, the amount of Compound A monocitrate monohydrate salt that is administered to a subject in need thereof is about 95 mg as free base equivalents orally twice daily.

In some embodiments, the amount of Compound A monocitrate monohydrate salt that is administered to a subject in need thereof is about 100 mg as free base equivalents orally twice daily.

In some embodiments, the amount of Compound A monocitrate monohydrate salt that is administered to a subject in need thereof is about 110 mg as free base equivalents orally twice daily.

In some embodiments, the amount of Compound A monocitrate monohydrate salt that is administered to a subject in need thereof is about 135 mg as free base equivalents orally twice daily.

In some embodiments, the amount of Compound A monocitrate monohydrate salt that is administered to a subject in need thereof is about 150 mg as free base equivalents orally twice daily.

In some embodiments, the amount of Compound A monocitrate monohydrate salt that is administered to a subject in need thereof is about 160 mg as free base equivalents orally twice daily.

In some embodiments, the amount of Compound A monocitrate monohydrate salt that is administered to a subject in need thereof is about 185 mg as free base equivalents orally twice daily.

In some embodiments, the amount of Compound A monocitrate monohydrate salt that is administered to a subject in need thereof is about 200 mg as free base equivalents orally twice daily.

In some embodiments, the amount of Compound A monocitrate monohydrate salt that is administered to a subject in need thereof is about 220 mg as free base equivalents orally twice daily.

In some embodiments, the amount of Compound A monocitrate monohydrate salt that is administered to a subject in need thereof is about 250 mg as free base equivalents orally twice daily.

In some embodiments, the amount of Compound A monocitrate monohydrate salt that is administered to a subject in need thereof is about 270 mg as free base equivalents orally twice daily.

In some embodiments, the amount of Compound A monocitrate monohydrate salt that is administered to a subject in need thereof is about 300 mg as free base equivalents orally twice daily.

In some embodiments, the amount of Compound A monocitrate monohydrate salt that is administered to a subject in need thereof is about 320 mg as free base equivalents orally twice daily.

In some embodiments, the amount of Compound A monocitrate monohydrate salt that is administered to a subject in need thereof is about 350 mg as free base equivalents orally twice daily.

In some embodiments, the amount of Compound A monocitrate monohydrate salt that is administered to a subject in need thereof is about 400 mg as free base equivalents orally twice daily.

In some embodiments, the amount of Compound A monocitrate monohydrate salt that is administered to a subject in need thereof is about 440 mg as free base equivalents orally twice daily.

In some embodiments, the amount of Compound A monocitrate monohydrate salt that is administered to a subject in need thereof is about 450 mg as free base equivalents orally twice daily.

In some embodiments, the amount of Compound A monocitrate monohydrate salt that is administered to a subject in need thereof is about 500 mg as free base equivalents orally twice daily.

In some embodiments, the amount of Compound A monocitrate monohydrate salt that is administered to a subject in need thereof is about 540 mg as free base equivalents orally twice daily.

In some embodiments, the amount of Compound A monocitrate monohydrate salt that is administered to a subject in need thereof is about 550 mg as free base equivalents orally twice daily.

In some embodiments, the amount of Compound A monocitrate monohydrate salt that is administered to a subject in need thereof is about 600 mg as free base equivalents orally twice daily.

In some embodiments, the amount of Compound A monocitrate monohydrate salt that is administered to a subject in need thereof is about 650 mg as free base equivalents orally twice daily.

In some embodiments, the amount of Compound A monocitrate monohydrate salt that is administered to a subject in need thereof is about 700 mg as free base equivalents orally twice daily.

In some embodiments, the amount of Compound A monocitrate monohydrate salt that is administered to a subject in need thereof is about 750 mg as free base equivalents orally twice daily.

In some embodiments, the amount of Compound A monocitrate monohydrate salt that is administered to a subject in need thereof is about 800 mg as free base equivalents orally twice daily.

In some embodiments, the amount of Compound A monocitrate monohydrate salt that is administered to a subject in need thereof is about 850 mg as free base equivalents orally twice daily.

In some embodiments, the amount of Compound A monocitrate monohydrate salt that is administered to a subject in need thereof is about 900 mg as free base equivalents orally twice daily.

In some embodiments, the amount of Compound A monocitrate monohydrate salt that is administered to a subject in need thereof is about 950 mg as free base equivalents orally twice daily.

In some embodiments, the amount of Compound A monocitrate monohydrate salt that is administered to a subject in need thereof is about 1000 mg as free base equivalents orally twice daily.

In some embodiments, the amount of Compound A monocitrate monohydrate salt that is administered to a subject in need thereof is from about 270 mg/day to about 160 mg/day.

In some embodiments, the amount of Compound A monocitrate monohydrate salt that is administered to a subject in need thereof is from about 160 mg/day to about 95 mg/day.

In some embodiments, the amount of Compound A monocitrate monohydrate salt that is administered to a subject in need thereof is about 25 mg orally twice daily.

In some embodiments, the amount of Compound A monocitrate monohydrate salt that is administered to a subject in need thereof is about 50 mg orally twice daily.

In some embodiments, the amount of Compound A monocitrate monohydrate salt that is administered to a subject in need thereof is about 75 mg orally twice daily.

In some embodiments, the amount of Compound A monocitrate monohydrate salt that is administered to a subject in need thereof is about 95 mg orally twice daily.

In some embodiments, the amount of Compound A monocitrate monohydrate salt that is administered to a subject in need thereof is about 100 mg orally twice daily.

In some embodiments, the amount of Compound A monocitrate monohydrate salt that is administered to a subject in need thereof is about 110 mg orally twice daily.

In some embodiments, the amount of Compound A monocitrate monohydrate salt that is administered to a subject in need thereof is about 135 mg orally twice daily.

In some embodiments, the amount of Compound A monocitrate monohydrate salt that is administered to a subject in need thereof is about 150 mg orally twice daily.

In some embodiments, the amount of Compound A monocitrate monohydrate salt that is administered to a subject in need thereof is about 160 mg orally twice daily.

In some embodiments, the amount of Compound A monocitrate monohydrate salt that is administered to a subject in need thereof is about 185 mg orally twice daily.

In some embodiments, the amount of Compound A monocitrate monohydrate salt that is administered to a subject in need thereof is about 200 mg orally twice daily.

In some embodiments, the amount of Compound A monocitrate monohydrate salt that is administered to a subject in need thereof is about 220 mg orally twice daily.

In some embodiments, the amount of Compound A monocitrate monohydrate salt that is administered to a subject in need thereof is about 250 mg orally twice daily.

In some embodiments, the amount of Compound A monocitrate monohydrate salt that is administered to a subject in need thereof is about 270 mg orally twice daily.

In some embodiments, the amount of Compound A monocitrate monohydrate salt that is administered to a subject in need thereof is about 300 mg orally twice daily.

In some embodiments, the amount of Compound A monocitrate monohydrate salt that is administered to a subject in need thereof is about 320 mg orally twice daily.

In some embodiments, the amount of Compound A monocitrate monohydrate salt that is administered to a subject in need thereof is about 350 mg orally twice daily.

In some embodiments, the amount of Compound A monocitrate monohydrate salt that is administered to a subject in need thereof is about 400 mg orally twice daily.

In some embodiments, the amount of Compound A monocitrate monohydrate salt that is administered to a subject in need thereof is about 440 mg orally twice daily.

In some embodiments, the amount of Compound A monocitrate monohydrate salt that is administered to a subject in need thereof is about 450 mg orally twice daily.

In some embodiments, the amount of Compound A monocitrate monohydrate salt that is administered to a subject in need thereof is about 500 mg orally twice daily.

In some embodiments, the amount of Compound A monocitrate monohydrate salt that is administered to a subject in need thereof is about 540 mg orally twice daily.

In some embodiments, the amount of Compound A monocitrate monohydrate salt that is administered to a subject in need thereof is about 550 mg orally twice daily.

In some embodiments, the amount of Compound A monocitrate monohydrate salt that is administered to a subject in need thereof is about 600 mg orally twice daily.

In some embodiments, the amount of Compound A monocitrate monohydrate salt that is administered to a subject in need thereof is about 650 mg orally twice daily.

In some embodiments, the amount of Compound A monocitrate monohydrate salt that is administered to a subject in need thereof is about 700 mg orally twice daily.

In some embodiments, the amount of Compound A monocitrate monohydrate salt that is administered to a subject in need thereof is about 750 mg orally twice daily.

In some embodiments, the amount of Compound A monocitrate monohydrate salt that is administered to a subject in need thereof is about 800 mg orally twice daily.

In some embodiments, the amount of Compound A monocitrate monohydrate salt that is administered to a subject in need thereof is about 850 mg orally twice daily.

In some embodiments, the amount of Compound A monocitrate monohydrate salt that is administered to a subject in need thereof is about 900 mg orally twice daily.

In some embodiments, the amount of Compound A monocitrate monohydrate salt that is administered to a subject in need thereof is about 950 mg orally twice daily.

In some embodiments, the amount of Compound A monocitrate monohydrate salt that is administered to a subject in need thereof is about 1000 mg orally twice daily.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² up to, and including about 1,000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 1,000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 950 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 900 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 850 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 800 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 750 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 700 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 650 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 600 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 550 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 500 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 450 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 400 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 350 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 300 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 250 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 200 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 150 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 100 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 95 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 25 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 50 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 95 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 95 mg/m² to about 160 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 100 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 150 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 200 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 250 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 300 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 350 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 400 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 450 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 500 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 550 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 600 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 650 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 700 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 750 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 800 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 850 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 900 mg/m² to about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 20 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 30 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 60 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 65 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 95 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 100 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 130 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 150 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 160 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 190 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 220 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 250 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 270 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 300 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 320 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 350 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 400 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 440 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 450 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 500 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 540 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 550 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 600 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 650 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 700 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 750 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 800 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 850 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 900 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 950 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 1000 mg/m².

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² up to, and including about 1,000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 1,000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 950 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 900 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 850 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 800 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 750 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 700 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 650 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 600 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 550 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 500 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 450 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 400 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 350 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 300 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 250 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 200 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 150 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 100 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 20 mg/m² to about 95 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 25 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 50 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 95 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 95 mg/m² to about 160 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 100 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 150 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 200 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 250 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 300 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 350 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 400 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 450 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 500 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 550 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 600 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 650 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 700 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 750 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 800 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 850 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is from about 900 mg/m² to about 1000 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 20 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 30 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 60 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 65 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 95 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 100 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 130 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 150 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 160 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 190 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 220 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 250 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 270 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 300 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 320 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 350 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 400 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 440 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 450 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 500 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 540 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 550 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 600 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 650 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 700 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 750 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 800 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 850 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 900 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 950 mg/m² as free base equivalents.

In some embodiments, the amount of Compound A, Compound A citrate salt (e.g., Compound A monocitrate monohydrate salt) or polymorphic form thereof (e.g., Compound A monocitrate monohydrate Form A salt) that is administered to a subject in need thereof is about 1000 mg/m² as free base equivalents.

In some embodiments, the amount of Compound A monocitrate monohydrate salt that is administered to a subject in need thereof is about 30 mg/m² orally twice daily.

In some embodiments, the amount of Compound A monocitrate monohydrate salt that is administered to a subject in need thereof is about 65 mg/m² orally twice daily.

In some embodiments, the amount of Compound A monocitrate monohydrate salt that is administered to a subject in need thereof is about 95 mg/m² orally twice daily.

In some embodiments, the amount of Compound A monocitrate monohydrate salt that is administered to a subject in need thereof is about 100 mg/m² orally twice daily.

In some embodiments, the amount of Compound A monocitrate monohydrate salt that is administered to a subject in need thereof is about 110 mg/m² orally twice daily.

In some embodiments, the amount of Compound A monocitrate monohydrate salt that is administered to a subject in need thereof is about 150 mg/m² orally twice daily.

In some embodiments, the amount of Compound A monocitrate monohydrate salt that is administered to a subject in need thereof is about 160 mg/m² orally twice daily.

In some embodiments, the amount of Compound A monocitrate monohydrate salt that is administered to a subject in need thereof is about 200 mg/m² orally twice daily.

In some embodiments, the amount of Compound A monocitrate monohydrate salt that is administered to a subject in need thereof is about 220 mg/m² orally twice daily.

In some embodiments, the amount of Compound A monocitrate monohydrate salt that is administered to a subject in need thereof is about 250 mg/m² orally twice daily.

In some embodiments, the amount of Compound A monocitrate monohydrate salt that is administered to a subject in need thereof is about 270 mg/m² orally twice daily.

In some embodiments, the amount of Compound A monocitrate monohydrate salt that is administered to a subject in need thereof is about 300 mg/m² orally twice daily.

In some embodiments, the amount of Compound A monocitrate monohydrate salt that is administered to a subject in need thereof is about 320 mg/m² orally twice daily.

In some embodiments, the amount of Compound A monocitrate monohydrate salt that is administered to a subject in need thereof is about 350 mg/m² orally twice daily.

In some embodiments, the amount of Compound A monocitrate monohydrate salt that is administered to a subject in need thereof is about 400 mg/m² orally twice daily.

In some embodiments, the amount of Compound A monocitrate monohydrate salt that is administered to a subject in need thereof is about 440 mg/m² orally twice daily.

In some embodiments, the amount of Compound A monocitrate monohydrate salt that is administered to a subject in need thereof is about 450 mg/m² orally twice daily.

In some embodiments, the amount of Compound A monocitrate monohydrate salt that is administered to a subject in need thereof is about 500 mg/m² orally twice daily.

In some embodiments, the amount of Compound A monocitrate monohydrate salt that is administered to a subject in need thereof is about 540 mg/m² orally twice daily.

In some embodiments, the amount of Compound A monocitrate monohydrate salt that is administered to a subject in need thereof is about 550 mg/m² orally twice daily.

In some embodiments, the amount of Compound A monocitrate monohydrate salt that is administered to a subject in need thereof is about 600 mg/m² orally twice daily.

In some embodiments, the amount of Compound A monocitrate monohydrate salt that is administered to a subject in need thereof is about 650 mg/m² orally twice daily.

In some embodiments, the amount of Compound A monocitrate monohydrate salt that is administered to a subject in need thereof is about 700 mg/m² orally twice daily.

In some embodiments, the amount of Compound A monocitrate monohydrate salt that is administered to a subject in need thereof is about 750 mg/m² orally twice daily.

In some embodiments, the amount of Compound A monocitrate monohydrate salt that is administered to a subject in need thereof is about 800 mg/m² orally twice daily.

In some embodiments, the amount of Compound A monocitrate monohydrate salt that is administered to a subject in need thereof is about 850 mg/m² orally twice daily.

In some embodiments, the amount of Compound A monocitrate monohydrate salt that is administered to a subject in need thereof is about 900 mg/m² orally twice daily.

In some embodiments, the amount of Compound A monocitrate monohydrate salt that is administered to a subject in need thereof is about 950 mg/m² orally twice daily.

In some embodiments, the amount of Compound A monocitrate monohydrate salt that is administered to a subject in need thereof is about 1000 mg/m² orally twice daily.

In some embodiments, the amount of Compound A monocitrate monohydrate salt that is administered to a subject in need thereof is from about 95 mg/m² to about 160 mg/m² orally twice daily.

In some embodiments, the dose is administered once daily.

In some embodiments, the dose is administered twice daily.

In some embodiments, the dose is administered three times daily.

In some embodiments, the dose is administered four times daily.

In some embodiments, for patients ≥40 kg the recommended starting dose of Compound A is 270 mg orally twice daily, with or without food, until disease progression or unacceptable toxicity, wherein Compound A is administered as the monocitrate monohydrate salt.

In some embodiments, for patients weighing <40 kg the dose of Compound A is 160 mg orally twice daily, with or without food, until disease progression or unacceptable toxicity, wherein Compound A is administered as the monocitrate monohydrate salt.

In some embodiments, for patients weighing <40 kg the dose of Compound A is 100 mg orally twice daily, with or without food, until disease progression or unacceptable toxicity, wherein Compound A is administered as the monocitrate monohydrate salt.

In some embodiments, for patients weighing <40 kg the dose of Compound A is 160 mg/m² orally twice daily, with or without food, until disease progression or unacceptable toxicity, wherein Compound A is administered as the monocitrate monohydrate salt.

In some embodiments, for patients weighing <40 kg the dose of Compound A is 95 mg/m² orally twice daily, with or without food, until disease progression or unacceptable toxicity, wherein Compound A is administered as the monocitrate monohydrate salt.

In some embodiments, for patients ≥40 kg the recommended starting dose of Compound A is 270 mg orally twice daily, with or without food, until disease progression or unacceptable toxicity.

In some embodiments, for patients ≥40 kg the recommended starting dose of Compound A is 270 mg orally twice daily, with or without food, until disease progression or unacceptable toxicity, wherein Compound A is administered as the monocitrate monohydrate salt.

In some embodiments, for patients ≥40 kg the recommended starting dose of Compound A is 160 mg orally twice daily, with or without food, until disease progression or unacceptable toxicity.

In some embodiments, for patients ≥40 kg the recommended starting dose of Compound A is 160 mg (as free base equivalents) orally twice daily, with or without food, until disease progression or unacceptable toxicity, wherein Compound A is administered as the monocitrate monohydrate salt.

In some embodiments, for patients ≥40 kg the recommended starting dose of Compound A is 100 mg (as free base equivalents) orally twice daily, with or without food, until disease progression or unacceptable toxicity.

In some embodiments, for patients ≥40 kg the recommended starting dose of Compound A is 100 mg (as free base equivalents) orally twice daily, with or without food, until disease progression or unacceptable toxicity, wherein Compound A is administered as the monocitrate monohydrate salt.

In some embodiments, for patients <40 kg the recommended starting dose of Compound A is 160 mg/m² (as free base equivalents) orally twice daily, with or without food, until disease progression or unacceptable toxicity.

In some embodiments, for patients <40 kg the recommended starting dose of Compound A is 160 mg/m² (as free base equivalents) orally twice daily, with or without food, until disease progression or unacceptable toxicity, wherein Compound A is administered as the monocitrate monohydrate salt.

In some embodiments, for patients <40 kg the recommended starting dose of Compound A is 95 mg/m² orally twice daily, with or without food, until disease progression or unacceptable toxicity.

In some embodiments, for patients <40 kg the recommended starting dose of Compound A is 95 mg/m² orally twice daily, with or without food, until disease progression or unacceptable toxicity, wherein Compound A is administered as the monocitrate monohydrate salt.

In some embodiments, Compound A is administered to a patient (6 months and older) in need thereof with dose as provided in Table 5A.

In some embodiments, Compound A is administered to a patient (6 months and older) in need thereof with dose as provided in Table 5A, wherein Compound A is administered as the monocitrate monohydrate salt.

TABLE 5A
Compound A Monocitrate Monohydrate Salt Recommended
Dosage for Patients 6 Months and Older

| | Without Strong CYP3A4 | With Strong CYP3A4 |
|---|---|---|
| | Inhibitors | Inhibitors |
| Patients Weighing | 270 mg orally | 160 mg orally |
| 40 kg or more | twice daily | twice daily |
| Patients Weighing | 160 mg/m² orally | 95 mg/m² orally |
| less than 40 kg | twice daily | twice daily |

In some embodiments, if a strong CYP3A4 inhibitor is co-administered, the dose of Compound A for patients ≥40 kg is reduced from 270 mg to 160 mg orally twice daily.

In some embodiments, if a strong CYP3A4 inhibitor is co-administered, the dose of Compound A monocitrate monohydrate salt for patients <40 kg is reduced from 160 mg/m² to 95 mg/m² orally twice daily (based on free base equivalents).

In some embodiments, if a strong CYP3A4 inhibitor is co-administered, the dose of Compound A monocitrate monohydrate salt for patients ≥40 kg is reduced from 270 mg to 160 mg orally twice daily (based on free base equivalents), with or without food, until disease progression or unacceptable toxicity, wherein the patient is 6 months and older.

In some embodiments, if a strong CYP3A4 inhibitor is co-administered, the dose of Compound A monocitrate monohydrate salt for patients <40 kg is reduced from 160 mg/m² to 95 mg/m² orally twice daily (based on free base equivalents), with or without food, until disease progression or unacceptable toxicity, wherein the patient is 6 months and older.

In some embodiments, Compound A monocitrate monohydrate salt is administered to a patient in need thereof with dose reduction as provided in Table 5B.

TABLE 5B
Starting Dose of Compound A Monocitrate
Monohydrate Salt and Dose Modifications

| Dose Level | Patients Weighing ≥ 40 kg | Patients Weighing < 40 kg |
|---|---|---|
| Starting Dose | 270 mg orally twice daily | 160 mg/m² orally |
| | | twice daily |
| First Dose | 160 mg orally twice daily | 95 mg/m² orally |
| Reduction | | twice daily |
| Second Dose | 110 mg orally twice daily | 65 mg/m² orally |
| Reduction | | twice daily |

In some embodiments, Compound A monocitrate monohydrate salt is administered to a patient 6 months and older not on strong CYP3A4 inhibitors with dose reduction as provided in Table 5C to accommodate for adverse reactions.

TABLE 5C
Starting Dosage of Compound A Monocitrate Monohydrate
Salt and Dosage Modifications for Patients 6 months
and Older not on Strong CYP3A4 Inhibitors

| | Patients Weighing 40 kg | Patients Weighing |
|---|---|---|
| Dose Level | or Greater | Less Than 40 kg |
| Starting Dose | 270 mg orally twice daily | 160 mg/m² orally |
| | | twice daily |
| Reduced Dose | 160 mg orally twice daily | 95 mg/m² orally |
| | | twice daily |

In some embodiments, if a strong CYP3A4 inhibitor is not co-administered, the dose of Compound A for patients 40 kg or greater is reduced from 270 mg to 160 mg orally twice daily.

In some embodiments, if a strong CYP3A4 inhibitor is not co-administered, the dose of Compound A monocitrate monohydrate salt for patients less than 40 kg is reduced from 160 mg/m² to 95 mg/m² orally twice daily (based on free base equivalents) to accommodate for adverse reactions.

In some embodiments, if a strong CYP3A4 inhibitor is not co-administered, the dose of Compound A monocitrate monohydrate salt for patients 40 kg or greater is reduced from 270 mg to 160 mg orally twice daily (based on free base equivalents) to accommodate for adverse reactions, with or without food, until disease progression or unacceptable toxicity, wherein the patient is 6 months and older.

In some embodiments, if a strong CYP3A4 inhibitor is not co-administered, the dose of Compound A monocitrate monohydrate salt for patients less than 40 kg is reduced from 160 mg/m² to 95 mg/m² orally twice daily (based on free base equivalents) to accommodate for adverse reactions, with or without food, until disease progression or unacceptable toxicity, wherein the patient is 6 months and older.

In some embodiments, Compound A monocitrate monohydrate salt is administered to a patient 6 months and older on strong CYP3A4 inhibitors with dose reduction as provided in Table 5D to accommodate for adverse reactions.

TABLE 5D
Starting Dosage of Compound A Monocitrate Monohydrate
Salt and Dosage Modifications for Patients 6 months
and Older on Strong CYP3A4 Inhibitors

| | Patients Weighing 40 kg | Patients Weighing |
|---|---|---|
| Dose Level | or Greater | Less Than 40 kg |
| Starting Dose | 160 mg orally twice daily | 95 mg/m² orally |
| | | twice daily |
| Reduced Dose | 110 mg orally twice daily | 65 mg/m² orally |
| | | twice daily |

In some embodiments, if a strong CYP3A4 inhibitor must be co-administered, the dose of Compound A monocitrate monohydrate salt for patients 40 kg or greater is reduced from 160 mg to 110 mg orally twice daily (based on free base equivalents) to accommodate for adverse reactions.

In some embodiments, if a strong CYP3A4 inhibitor must be co-administered, the dose of Compound A monocitrate monohydrate salt for patients less than 40 kg is reduced from 95 mg/m² to 65 mg/m² orally twice daily (based on free base equivalents) to accommodate for adverse reactions.

In some embodiments, if a strong CYP3A4 inhibitor must be co-administered, the dose of Compound A monocitrate monohydrate salt for patients 40 kg or greater is reduced from 160 mg to 110 mg orally twice daily (based on free base equivalents) to accommodate for adverse reactions, with or without food, until disease progression or unacceptable toxicity, wherein the patient is 6 months and older.

In some embodiments, if a strong CYP3A4 inhibitor must be co-administered, the dose of Compound A monocitrate monohydrate salt for patients less than 40 kg is reduced from 95 mg/m² to 65 mg/m² orally twice daily (based on free base equivalents) to accommodate for adverse reactions, with or without food, until disease progression or unacceptable toxicity, wherein the patient is 6 months and older.

In some embodiments, if a strong CYP3A4 inhibitor must be co-administered, the dose for patients ≥40 kg is reduced from 270 mg to 160 mg orally twice daily.

In some embodiments, for patients ≥40 kg the recommended starting dose of Compound A is 270 mg orally twice daily, with or without food, until disease progression or unacceptable toxicity. In some embodiments, for patients <40 kg the recommended starting dose of Compound A is 160 mg/m² orally twice daily, with or without food, until disease progression or unacceptable toxicity.

In some embodiments, for patients ≥40 kg the recommended starting dose of Compound A monocitrate monohydrate salt is 270 mg orally twice daily (based on free base equivalents), with or without food, until disease progression or unacceptable toxicity. In some embodiments, for patients <40 kg the recommended starting dose of Compound A monocitrate monohydrate salt is 160 mg/m² orally twice daily (based on free base equivalents), with or without food, until disease progression or unacceptable toxicity. In some embodiments, the dose for patients <40 kg is reduced from 160 mg/m² (based on free base equivalents) to 95 mg/m² (based on free base equivalents) orally twice daily, with or without food, until disease progression or unacceptable toxicity.

In some embodiments, Compound A monocitrate monohydrate salt is administered to a patient in need thereof (e.g., an infant at 1 to <2 months and 2-6 months) with dose adjustment as provided in Table 6A:

TABLE 6A
Dose Adjustment of Compound A Monocitrate Monohydrate
Salt to a patient in need thereof (e.g., an infant
at 1 to < 2 months and 2-6 months)

| | Dose adjustment without | Dose adjustment with Strong |
|---|---|---|
| Age Range | Strong CYP3A4i (mg/m²) | CYP3A4i (mg/m²) |
| 1-< 2 months | 70 | 40 |
| 2-< 6 months | 100 | 60 |

In some embodiments, the dose for patients of age range 1-<2 months, is 70 mg/m² once daily (if administered without Strong CYP3A4i).

In some embodiments, the dose for patients of age range 1-<2 months, is 40 mg/m² once daily (if administered with Strong CYP3A4i).

In some embodiments, the dose for patients of age range 2-<6 months, is 100 mg/m² once daily (if administered without Strong CYP3A4i).

In some embodiments, the dose for patients of age range 2-<6 months, is 60 mg/m² once daily (if administered with Strong CYP3A4i).

In some embodiments, the dose for patients of age range 1-<2 months, is 70 mg/m² twice daily (if administered without Strong CYP3A4i).

In some embodiments, the dose for patients of age range 1-<2 months, is 40 mg/m² twice daily (if administered with Strong CYP3A4i).

In some embodiments, the dose for patients of age range 2-<6 months, is 100 mg/m² twice daily (if administered without Strong CYP3A4i).

In some embodiments, the dose for patients of age range 2-<6 months, is 60 mg/m² twice daily (if administered with Strong CYP3A4i).

In some embodiments, Compound A monocitrate monohydrate salt is administered to a patient with body surface area (BSA) 1.4 m² or less and body weight less than 40 kg as provided in Table 6B.

TABLE 6B
Recommended Dose of Compound A Monocitrate
Monohydrate Salt for Patients with
BSA 1.4 m² or less and body weight less than 40 kg

| | Compound A | Compound A | Compound A |
|---|---|---|---|
| | Monocitrate | Monocitrate | Monocitrate |
| | Monohydrate | Monohydrate | Monohydrate |
| | Salt | Salt | Salt |
| BSA (m²) | 160 mg/m² | 95 mg/m² | 65 mg/m² |
| 1.4 | 220 mg | 135 mg | 100 mg |
| 1.3 | 220 mg | 135 mg | 75 mg |
| 1.2 | 185 mg | 110 mg | 75 mg |
| 1.1 | 185 mg | 110 mg | 75 mg |
| 1.0 | 160 mg | 100 mg | 50 mg |
| 0.9 | 135 mg | 75 mg | 50 mg |
| 0.8 | 135 mg | 75 mg | 50 mg |
| 0.7 | 110 mg | 50 mg | 50 mg |
| 0.6 | 100 mg | 50 mg | 25 mg |
| 0.5 | 75 mg | 50 mg | 25 mg |
| 0.4 | 50 mg | 25 mg | 25 mg |

In some embodiments, the dose of Compound A monocitrate monohydrate salt for a patient with BSA 1.4 m^{a} or less and body weight less than 40 kg is 160 mg/m² twice daily (based on free base equivalents).

In some embodiments, the dose of Compound A monocitrate monohydrate salt for a patient with BSA 1.4 m² and body weight less than 40 kg is 220 mg twice daily (based on free base equivalents).

In some embodiments, the dose of Compound A monocitrate monohydrate salt for a patient with BSA 1.3 m² and body weight less than 40 kg is 220 mg twice daily (based on free base equivalents).

In some embodiments, the dose of Compound A monocitrate monohydrate salt for a patient with BSA 1.2 m² and body weight less than 40 kg is 185 mg twice daily (based on free base equivalents).

In some embodiments, the dose of Compound A monocitrate monohydrate salt for a patient with BSA 1.1 m² and body weight less than 40 kg is 185 mg twice daily (based on free base equivalents).

In some embodiments, the dose of Compound A monocitrate monohydrate salt for a patient with BSA 1.0 m² and body weight less than 40 kg is 160 mg twice daily (based on free base equivalents).

In some embodiments, the dose of Compound A monocitrate monohydrate salt for a patient with BSA 0.9 m² and body weight less than 40 kg is 135 mg twice daily (based on free base equivalents).

In some embodiments, the dose of Compound A monocitrate monohydrate salt for a patient with BSA 0.8 m² and body weight less than 40 kg is 135 mg twice daily (based on free base equivalents).

In some embodiments, the dose of Compound A monocitrate monohydrate salt for a patient with BSA 0.7 m² and body weight less than 40 kg is 110 mg twice daily (based on free base equivalents).

In some embodiments, the dose of Compound A monocitrate monohydrate salt for a patient with BSA 0.6 m² and body weight less than 40 kg is 100 mg twice daily (based on free base equivalents).

In some embodiments, the dose of Compound A monocitrate monohydrate salt for a patient with BSA 0.5 m² and body weight less than 40 kg is 75 mg twice daily (based on free base equivalents).

In some embodiments, the dose of Compound A monocitrate monohydrate salt for a patient with BSA 0.4 m² and body weight less than 40 kg is 50 mg twice daily (based on free base equivalents).

In some embodiments, the dose of Compound A monocitrate monohydrate salt for a patient with BSA 1.4 m² or less and body weight less than 40 kg is 95 mg/m² twice daily (based on free base equivalents).

In some embodiments, the dose of Compound A monocitrate monohydrate salt for a patient with BSA 1.4 m² and body weight less than 40 kg is 135 mg twice daily (based on free base equivalents).

In some embodiments, the dose of Compound A monocitrate monohydrate salt for a patient with BSA 1.3 m² and body weight less than 40 kg is 135 mg twice daily (based on free base equivalents).

In some embodiments, the dose of Compound A monocitrate monohydrate salt for a patient with BSA 1.2 m² and body weight less than 40 kg is 110 mg twice daily (based on free base equivalents).

In some embodiments, the dose of Compound A monocitrate monohydrate salt for a patient with BSA 1.1 m² and body weight less than 40 kg is 110 mg twice daily (based on free base equivalents).

In some embodiments, the dose of Compound A monocitrate monohydrate salt for a patient with BSA 1.0 m² and body weight less than 40 kg is 100 mg twice daily (based on free base equivalents).

In some embodiments, the dose of Compound A monocitrate monohydrate salt for a patient with BSA 0.9 m² and body weight less than 40 kg is 75 mg twice daily (based on free base equivalents).

In some embodiments, the dose of Compound A monocitrate monohydrate salt for a patient with BSA 0.8 m² and body weight less than 40 kg is 75 mg twice daily (based on free base equivalents).

In some embodiments, the dose of Compound A monocitrate monohydrate salt for a patient with BSA 0.7 m² and body weight less than 40 kg is 50 mg twice daily (based on free base equivalents).

In some embodiments, the dose of Compound A monocitrate monohydrate salt for a patient with BSA 0.6 m² and body weight less than 40 kg is 50 mg twice daily (based on free base equivalents).

In some embodiments, the dose of Compound A monocitrate monohydrate salt for a patient with BSA 0.5 m² and body weight less than 40 kg is 50 mg twice daily (based on free base equivalents).

In some embodiments, the dose of Compound A monocitrate monohydrate salt for a patient with BSA 0.4 m² and body weight less than 40 kg is 25 mg twice daily (based on free base equivalents).

In some embodiments, the dose of Compound A monocitrate monohydrate salt for a patient with BSA 1.4 m² or less and body weight less than 40 kg is 65 mg/m² twice daily (based on free base equivalents).

In some embodiments, the dose of Compound A monocitrate monohydrate salt for a patient with BSA 1.4 m² and body weight less than 40 kg is 100 mg twice daily (based on free base equivalents).

In some embodiments, the dose of Compound A monocitrate monohydrate salt for a patient with BSA 1.3 m² and body weight less than 40 kg is 75 mg twice daily (based on free base equivalents).

In some embodiments, the dose of Compound A monocitrate monohydrate salt for a patient with BSA 1.2 m² and body weight less than 40 kg is 75 mg twice daily (based on free base equivalents).

In some embodiments, the dose of Compound A monocitrate monohydrate salt for a patient with BSA 1.1 m² and body weight less than 40 kg is 75 mg twice daily (based on free base equivalents).

In some embodiments, the dose of Compound A monocitrate monohydrate salt for a patient with BSA 1.0 m² and body weight less than 40 kg is 50 mg twice daily (based on free base equivalents).

In some embodiments, the dose of Compound A monocitrate monohydrate salt for a patient with BSA 0.9 m² and body weight less than 40 kg is 50 mg twice daily (based on free base equivalents).

In some embodiments, the dose of Compound A monocitrate monohydrate salt for a patient with BSA 0.8 m² and body weight less than 40 kg is 50 mg twice daily (based on free base equivalents).

In some embodiments, the dose of Compound A monocitrate monohydrate salt for a patient with BSA 0.7 m² and body weight less than 40 kg is 50 mg twice daily (based on free base equivalents).

In some embodiments, the dose of Compound A monocitrate monohydrate salt for a patient with BSA 0.6 m² and body weight less than 40 kg is 25 mg twice daily (based on free base equivalents).

In some embodiments, the dose of Compound A monocitrate monohydrate salt for a patient with BSA 0.5 m² and body weight less than 40 kg is 25 mg twice daily (based on free base equivalents).

In some embodiments, the dose of Compound A monocitrate monohydrate salt for a patient with BSA 0.4 m² and body weight less than 40 kg is 25 mg twice daily (based on free base equivalents).

In some embodiments, the method of treating, preventing, or reducing the severity of a cancer in a subject in need thereof, comprises administering to the subject a Compound A monocitrate monohydrate Form A salt, tablet, or liquid formulation thereof at a dosage of between about 25 mg and about 276 mg twice daily, and wherein upon administration, the area under plasma concentration versus time curve from time 0 to 12 hours (AUC(_{0-12 h})) of compound A is from about 5000 ng·h/mL to about 25000 ng·h/mL, from about 7500 ng·h/mL to about 24000 ng·h/mL, from about 8000 ng·h/mL to about 23000 ng·h/mL, from about 9000 ng·h/mL to about 22000 ng·h/mL, from about 10000 ng·h/mL to about 21000 ng·h/mL, from about 11000 ng·h/mL to about 20000 ng·h/mL, or from about 12000 ng·h/mL to about 19000 ng·h/mL.

In some embodiments, the method of treating, preventing, or reducing the severity of a cancer in a subject in need thereof, comprises administering to the subject a Compound A monocitrate monohydrate Form A salt, tablet, or liquid formulation thereof at a dosage of between about 25 mg and about 276 mg twice daily, and wherein upon administration, the area under plasma concentration versus time curve from time 0 to 12 hours (AUC(_{0-12 h})) of compound A is about 5000 ng·h/mL, about 6000 ng·h/mL, about 7000 ng·h/mL, about 8000 ng·h/mL, about 9000 ng·h/mL, about 10000 ng·h/mL, about 10150 ng·h/mL, about 11000 ng·h/mL, about 12000 ng·h/mL, about 13000 ng·h/mL, about 14000 ng·h/mL, about 15000 ng·h/mL, about 16000 ng·h/mL, about 17000 ng·h/mL, about 18000 ng·h/mL, about 19000 ng·h/mL, about 20000 ng·h/mL, about 21000 ng·h/mL, about 22000 ng·h/mL, about 23000 ng·h/mL, about 24000 ng·h/mL, or about 25000 ng·h/mL.

In some embodiments, the method of treating, preventing, or reducing the severity of a cancer in a subject in need thereof, comprises administering to the subject a Compound A monocitrate monohydrate Form A salt, tablet, or liquid formulation thereof at a dosage of about 276 mg twice daily, and wherein upon administration, the area under plasma concentration versus time curve from time 0 to 12 hours (AUC(_{0-12 h})) of compound A is about 10150 ng·h/mL.

In some embodiments, the method of treating, preventing, or reducing the severity of a cancer in a subject in need thereof, comprises administering to the subject a combination comprising a CYP3A4 inhibitor (e.g., a strong CYP3A4 inhibitor) and Compound A monocitrate monohydrate Form A salt, tablet, or liquid formulation thereof at a dosage of between about 25 mg and about 276 mg twice daily, and wherein upon administration, the area under plasma concentration versus time curve from time 0 to 12 hours (AUC(_{0-12 h})) of compound A is from about 5000 ng·h/mL to about 25000 ng·h/mL, from about 7500 ng·h/mL to about 24000 ng·h/mL, from about 8000 ng·h/mL to about 23000 ng·h/mL, from about 9000 ng·h/mL to about 22000 ng·h/mL, from about 10000 ng·h/mL to about 21000 ng·h/mL, from about 11000 ng·h/mL to about 20000 ng·h/mL, or from about 12000 ng·h/mL to about 19000 ng·h/mL.

In some embodiments, the method of treating, preventing, or reducing the severity of a cancer in a subject in need thereof, comprises administering to the subject a combination comprising a CYP3A4 inhibitor (e.g., a strong CYP3A4 inhibitor) and Compound A monocitrate monohydrate Form A salt, tablet, or liquid formulation thereof at a dosage of between about 25 mg and about 276 mg twice daily, and wherein upon administration, the area under plasma concentration versus time curve from time 0 to 12 hours (AUC(_{0-12 h})) of compound A is about 5000 ng·h/mL, about 6000 ng·h/mL, about 7000 ng·h/mL, about 8000 ng·h/mL, about 9000 ng·h/mL, about 10000 ng·h/mL, about 11000 ng·h/mL, about 12000 ng·h/mL, about 13000 ng·h/mL, about 14000 ng·h/mL, about 15000 ng·h/mL, about 16000 ng·h/mL, about 17000 ng·h/mL, about 18000 ng·h/mL, about 19000 ng·h/mL, about 20000 ng·h/mL, about 21000 ng·h/mL, about 22000 ng·h/mL, about 22610 ng·h/mL, about 23000 ng·h/mL, about 24000 ng·h/mL, or about 25000 ng·h/mL.

In some embodiments, the method of treating, preventing, or reducing the severity of a cancer in a subject in need thereof, comprises administering to the subject a combination comprising a CYP3A4 inhibitor (e.g., a strong CYP3A4 inhibitor) and Compound A monocitrate monohydrate Form A salt, tablet, or liquid formulation thereof at a dosage of about 163 mg twice daily, and wherein upon administration, the area under plasma concentration versus time curve from time 0 to 12 hours (AUC(_{0-12 h})) of compound A is about 22610 ng·h/mL.

In some embodiments, the method of treating, preventing, or reducing the severity of a cancer in a subject in need thereof, comprises administering to the subject a Compound A monocitrate monohydrate Form A salt, tablet, or liquid formulation thereof at a dosage of between about 25 mg and about 276 mg twice daily, and wherein upon administration, the maximum plasma concentration (Cₘₐₓ) of compound A is from about 500 ng/mL to about 5000 ng/mL, from about 750 ng/mL to about 4750 ng/mL, from about 1000 ng/mL to about 4500 ng/mL, from about 1250 ng/mL to about 4250 ng/mL, from about 1500 ng/mL to about 4000 ng/mL, from about 1750 ng/mL to about 3750 ng/mL, or from about 2000 ng/mL to about 3500 ng/mL.

In some embodiments, the method of treating, preventing, or reducing the severity of a cancer in a subject in need thereof, comprises administering to the subject a Compound A monocitrate monohydrate Form A salt, tablet, or liquid formulation thereof at a dosage of between about 25 mg and about 276 mg twice daily, and wherein upon administration, the maximum plasma concentration (Cₘₐₓ) of compound A is about 500 ng/mL, about 750 ng/mL, about 1000 ng/mL, about 1250 ng/mL, about 1500 ng/mL, about 1750 ng/mL, about 2000 ng/mL, about 2052 ng/mL, about 2250 ng/mL, about 2500 ng/mL, about 2750 ng/mL, about 3000 ng/mL, about 3250 ng/mL, about 3500 ng/mL, about 3750 ng/mL, about 4000 ng/mL, about 4250 ng/mL, about 4500 ng/mL, about 4750 ng/mL, or about 5000 ng/mL.

In some embodiments, the method of treating, preventing, or reducing the severity of a cancer in a subject in need thereof, comprises administering to the subject a Compound A monocitrate monohydrate Form A salt, tablet, or liquid formulation thereof at a dosage of about 276 mg twice daily, and wherein upon administration, the maximum plasma concentration (Cₘₐₓ) of compound A is about 2052 ng/mL.

In some embodiments, the method of treating, preventing, or reducing the severity of a cancer in a subject in need thereof, comprises administering to the subject a combination comprising a CYP3A4 inhibitor (e.g., a strong CYP3A4 inhibitor) and Compound A monocitrate monohydrate Form A salt, tablet, or liquid formulation thereof at a dosage of between about 25 mg and about 276 mg twice daily, and wherein upon administration, the maximum plasma concentration (Cₘₐₓ) of compound A is from about 500 ng/mL to about 5000 ng/mL, from about 750 ng/mL to about 4750 ng/mL, from about 1000 ng/mL to about 4500 ng/mL, from about 1250 ng/mL to about 4250 ng/mL, from about 1500 ng/mL to about 4000 ng/mL, from about 1750 ng/mL to about 3750 ng/mL, or from about 2000 ng/mL to about 3500 ng/mL.

In some embodiments, the method of treating, preventing, or reducing the severity of a cancer in a subject in need thereof, comprises administering to the subject a combination comprising a CYP3A4 inhibitor (e.g., a strong CYP3A4 inhibitor) and Compound A monocitrate monohydrate Form A salt, tablet, or liquid formulation thereof at a dosage of between about 25 mg and about 276 mg twice daily, and wherein upon administration, the maximum plasma concentration (Cₘₐₓ) of compound A is about 500 ng/mL, about 750 ng/mL, about 1000 ng/mL, about 1250 ng/mL, about 1500 ng/mL, about 1750 ng/mL, about 2000 ng/mL, about 2250 ng/mL, about 2500 ng/mL, about 2750 ng/mL, about 3000 ng/mL, about 3220 ng/mL, about 3250 ng/mL, about 3500 ng/mL, about 3750 ng/mL, about 4000 ng/mL, about 4250 ng/mL, about 4500 ng/mL, about 4750 ng/mL, or about 5000 ng/mL.

In some embodiments, the method of treating, preventing, or reducing the severity of a cancer in a subject in need thereof, comprises administering to the subject a combination comprising a CYP3A4 inhibitor (e.g., a strong CYP3A4 inhibitor) and Compound A monocitrate monohydrate Form A salt, tablet, or liquid formulation thereof at a dosage of about 163 mg twice daily, and wherein upon administration, the maximum plasma concentration (Cₘₐₓ) of compound A is about 3220 ng/mL.

### Combination Therapy

The disclosure further relates to a combination therapy for treating a disease or a disorder described herein. In some embodiments, the combination therapy comprises administering at least one compound of the present disclosure in combination with one or more other pharmaceutically active agents for treating cancer or other disorders mediated by menin/MLL. In some embodiments, the combination therapy comprises administering at least one compound of the present disclosure in combination with one or more other pharmaceutically active agents, such as for the treatment of cancer. The pharmaceutically active agents can be combined with a compound of the disclosure in a single dosage form, or the therapeutics can be administered simultaneously or sequentially as separate dosage forms.

In some embodiments, the invention provides a combination therapy comprising a menin inhibitor of the present disclosure (e.g., Compound A citrate salt or polymorphic form thereof) and a CYP3A4 inhibitor. In certain embodiments, the invention provides for a pharmaceutical composition comprising: (a) a menin inhibitor of the present disclosure (e.g., Compound A citrate salt or polymorphic form thereof), and (b) a CYP3A4 inhibitor. In some embodiments, the invention is directed to a method for treating a patient comprising (a) administering a menin inhibitor of the present disclosure (e.g., Compound A citrate salt or polymorphic form thereof), and (b) administering a CYP3A4 inhibitor.

In some embodiments, the invention provides a combination therapy comprising Compound A citrate salt or polymorphic form thereof and a CYP3A4 inhibitor. In certain embodiments, the invention provides for a pharmaceutical composition comprising: (a) Compound A citrate salt (e.g., Compound A monocitrate monohydrate salt) or polymorphic form thereof (e.g., Compound A monocitrate monohydrate Form A salt), and (b) a CYP3A4 inhibitor. In some embodiments, the invention is directed to a method for treating a patient comprising (a) administering Compound A citrate salt (e.g., Compound A monocitrate monohydrate salt) or polymorphic form thereof (e.g., Compound A monocitrate monohydrate Form A salt), and (b) administering a CYP3A4 inhibitor.

Some embodiments of this invention are directed to combination therapies designed to treat or manage cancer in a subject, wherein the combination therapies comprise administering a menin inhibitor of the present disclosure (e.g., Compound A citrate salt or polymorphic form thereof) in combination with a CYP3A4 inhibitor. In particular, some embodiments of this invention are directed to methods of treating or managing cancer in a subject, comprising administering a menin inhibitor in combination with a therapeutically effective amount of a CYP3A4 inhibitor administered simultaneously, separately or sequentially.

Some embodiments of this invention are directed to combination therapies designed to treat or manage cancer in a subject, wherein the combination therapies comprise administering Compound A citrate salt (e.g., Compound A monocitrate monohydrate salt) or polymorphic form thereof (e.g., Compound A monocitrate monohydrate Form A salt) in combination with a CYP3A4 inhibitor.

In some embodiments, the CYP3A inhibitor is: an antiarrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A inhibitor is: posaconazole, alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; verapamil; telaprevir; vincristine; voriconazole; or any combinations thereof.

In some embodiments, the CYP3A4 inhibitor is posaconazole, cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiments, the menin inhibitor and the CYP3A4 inhibitor are in separate dosage forms. In some embodiments, the pharmaceutical composition is in a combined dosage form. In some embodiments, the CYP3A4 inhibitor is posaconazole.

In some embodiments, the CYP3A4 inhibitor is a strong CYP3A4 inhibitor, a moderate CYP3A4 inhibitor, or a weak CYP3A4 inhibitor.

In some embodiments, the CYP3A4 inhibitor is posaconazole, itraconazole, voriconazole, cobicistat, fluconazole and isavuconazole, cimetidine, rifampin, or efavirenz.

In some embodiments, the CYP3A4 inhibitor is ketoconazole. In some embodiments, the CYP3A4 inhibitor is ritonavir. In some embodiments, the menin inhibitor and the CYP3A4 inhibitor are in separate dosage forms. In some embodiments, the pharmaceutical composition is in a combined dosage form. In some embodiments, the CYP3A4 inhibitor is posaconazole.

In some embodiments, the pharmaceutical composition comprises an amount of the CYP3A4 inhibitor that is effective to increase the oral bioavailability of the menin inhibitor. The compounds according to the disclosure may also be used in combination with immunotherapies, including but not limited to cell-based therapies, antibody therapies and cytokine therapies, for the treatment of a disease or disorder disclosed herein.

In certain embodiments, compounds according to the disclosure are used in combination with one or more passive immunotherapies, including but not limited to naked monoclonal antibody drugs and conjugated monoclonal antibody drugs. Examples of naked monoclonal antibody drugs that can be used include, but are not limited to, rituximab (Rituxan®), an antibody against the CD20 antigen; trastuzumab (Herceptin®), an antibody against the HER2 protein; alemtuzumab (Lemtrada®, Campath®), an antibody against the CD52 antigen; cetuximab (Erbitux®), an antibody against the EGFR protein; and bevacizumab (Avastin®) which is an anti-angiogenesis inhibitor of VEGF protein.

Examples of conjugated monoclonal antibodies that can be used include, but are not limited to, radiolabeled antibody ibritumomab tiuxetan (Zevalin®); radiolabeled antibody tositumomab (Bexxar®); and immunotoxin gemtuzumab ozogamicin (Mylotarg®) which contains calicheamicin; BL22, an anti-CD22 monoclonal antibody-immunotoxin conjugate; radiolabeled antibodies such as OncoScint® and ProstaScint®; brentuximab vedotin (Adcetris®); ado-trastuzumab emtansine (Kadcyla®, also called TDM-1).

Further examples of therapeutic antibodies that can be used include, but are not limited to, REOPRO® (abciximab), an antibody against the glycoprotein IIb/IIIa receptor on platelets; ZENAPAX® (daclizumab) an immunosuppressive, humanized anti-CD25 monoclonal antibody; PANOREX™, a murine anti-17-IA cell surface antigen IgG2a antibody; BEC2, a murine anti-idiotype (GD3 epitope) IgG antibody; IMC-C225, a chimeric anti-EGFR IgG antibody; VITAXIN™ a humanized anti-αVβ3 integrin antibody; Campath 1H/LDP-03, a humanized anti CD52 IgG1 antibody; Smart M195, a humanized anti-CD33 IgG antibody; LYMPHOCIDE™, a humanized anti-CD22 IgG antibody; LYMPHOCIDE™ Y-90; Lymphoscan; Nuvion® (against CD3; CM3, a humanized anti-ICAM3 antibody; IDEC-114 a primatized anti-CD80 antibody; IDEC-131 a humanized anti-CD40L antibody; IDEC-151 a primatized anti-CD4 antibody; IDEC-152 a primatized anti-CD23 antibody; SMART anti-CD3, a humanized anti-CD3 IgG; 5G1.1, a humanized anti-complement factor 5 (C5) antibody; D2E7, a humanized anti-TNF-α antibody; CDP870, a humanized anti-TNF-α Fab fragment; IDEC-151, a primatized anti-CD4 IgG1 antibody; MDX-CD4, a human anti-CD4 IgG antibody; CD20-streptdavidin (+biotin-yttrium 90); CDP571, a humanized anti-TNF-α IgG4 antibody; LDP-02, a humanized anti-α4β7 antibody; OrthoClone OKT4A, a humanized anti-CD4 IgG antibody; ANTOVA™, a humanized anti-CD40L IgG antibody; ANTEGREN™, a humanized anti-VLA-4 IgG antibody; and CAT-152, a human anti-TGF-β₂ antibody.

In certain embodiments, compounds according to the disclosure are used in combination with one or more targeted immunotherapies containing toxins but not an antibody, including but not limited to denileukin diftitox (Ontak®), IL-2 linked to diphtheria toxin.

The compounds according to the disclosure may also be used in combination with adjuvant immunotherapies for the treatment of a disease or disorder disclosed herein. Such adjuvant immunotherapies include, but are not limited to, cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guérin (BCG); Keyhole limpet hemocyanin (KLH); Incomplete Freund's adjuvant (IFA); QS-21; DETOX; Levamisole; and Dinitrophenyl (DNP), and combinations thereof, such as, for example, combinations of interleukins, for example IL-2, with other cytokines, such as IFN-alpha.

In certain embodiments, compounds according to the disclosure are used in combination with vaccine therapy, including but not limited to autologous and allogeneic tumor cell vaccines, antigen vaccines (including polyvalent antigen vaccines), dendritic cell vaccines, and viral vaccines.

In another embodiment, the present disclosure comprises administering to a subject with cancer an effective amount of a compound of the disclosure and one or more additional anti-cancer therapies selected from: surgery, anti-cancer agents/drugs, biological therapy, radiation therapy, anti-angiogenesis therapy, immunotherapy, adoptive transfer of effector cells, gene therapy or hormonal therapy. Examples of anti-cancer agents/drugs are described below.

In some embodiments, the anti-cancer agents/drug is, for example, adriamycin, actinomycin, bleomycin, vinblastine, cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; palbociclib; Yervoy® (ipilimumab); Mekinist™ (trametinib); peginterferon alfa-2b, recombinant interferon alfa-2b; Sylatron™ (peginterferon alfa-2b); Tafinlar® (dabrafenib); Zelboraf® (vemurafenib); or nivolumab.

The compounds according to the present disclosure can be administered in combination with existing methods of treating cancers, for example by chemotherapy, irradiation, or surgery. Thus, there is further provided a method of treating cancer comprising administering an effective amount of a compound of the disclosure, or a pharmaceutically acceptable salt form thereof, to a subject in need of such treatment, wherein an effective amount of at least one additional cancer chemotherapeutic agent is administered to the subject. Examples of suitable cancer chemotherapeutic agents include any of: abarelix, ado-trastuzumab emtansine, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, emtansine, epirubicin, eribulin, erlotinib, estramustine, etoposide phosphate, etoposide, everolimus, exemestane, fentanyl citrate, filgrastim, floxuridine, fludarabine, fluorouracil, fruquintinib, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, ixabepilone, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, paclitaxel albumin-stabilized nanoparticle formulation, pamidronate, panitumumab, pegaspargase, pegfilgrastim, pemetrexed disodium, pentostatin, pertuzuma, pipobroman, plicamycin, procarbazine, quinacrine, rasburicase, rituximab, sorafenib, streptozocin, sulfatinib, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, volitinib, vorinostat, and zoledronate.

In particular embodiments, compounds according to the disclosure are used in combination with one or more anti-cancer agent selected from methotrexate, paclitaxel albumin-stabilized nanoparticle formulation, ado-trastuzumab emtansine, eribulin, doxorubicin, fluorouracil, everolimus, anastrozole, pamidronate disodium, exemestane, capecitabine, cyclophosphamide, docetaxel, epirubicin, toremifene, fulvestrant, letrozole, gemcitabine, gemcitabine hydrochloride, goserelin acetate, trastuzumab, ixabepilone, lapatinib ditosylate, megestrol acetate, tamoxifen citrate, pamidronate disodium, palbociclib, and pertuzumab for the treatment of breast cancer.

Other anti-cancer agents/drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; andrographolide; angiogenesis inhibitors; antagonist D; antagonist G; antarelix; anti-dorsalizing morphogenetic protein-1; antiandrogen; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors; castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclin-dependent kinase inhibitors; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitors; microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; zanoterone; zilascorb; zinostatin stimalamer; 5-fluorouracil; and leucovorin.

In some embodiments, the anti-cancer agent/drug is an agent that stabilizes microtubules. As used herein, a “microtubulin stabilizer” means an anti-cancer agent/drug which acts by arresting cells in the G2-M phases due to stabilization of microtubules. Examples of microtubulin stabilizers include ACLITAXEL® and Taxol® analogues. Additional examples of microtubulin stabilizers include without limitation the following marketed drugs and drugs in development: Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof.

In another embodiment, the anti-cancer agent/drug is an agent that inhibits microtubules. As used herein, a “microtubulin inhibitor” means an anti-cancer agent which acts by inhibiting tubulin polymerization or microtubule assembly. Examples of microtubulin inhibitors include without limitation the following marketed drugs and drugs in development: Erbulozole (also known as R-55104); Dolastatin 10 (also known as DLS-10 and NSC-376128); Mivobulin isethionate (also known as CI-980); Vincristine; NSC-639829; ABT-751 (Abbott, also known as E-7010); Altorhyrtins (such as Altorhyrtin A and Altorhyrtin C); Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9); Cemadotin hydrochloride (also known as LU-103793 and NSC-D-669356); Auristatin PE (also known as NSC-654663); Soblidotin (also known as TZT-1027), LS-4559-P (Pharmacia, also known as LS-4577); LS-4578 (Pharmacia, also known as LS-477-P); LS-4477 (Pharmacia), LS-4559 (Pharmacia); RPR-112378 (Aventis); Vincristine sulfate; DZ-3358 (Daiichi); GS-164 (Takeda); GS-198 (Takeda); KAR-2 (Hungarian Academy of Sciences); SAH-49960 (Lilly/Novartis); SDZ-268970 (Lilly/Novartis); AM-97 (Armad/Kyowa Hakko); AM-132 (Armad); AM-138 (Armad/Kyowa Hakko); IDN-5005 (Indena); Cryptophycin 52 (also known as LY-355703); Vitilevuamide; Tubulysin A; Canadensol; Centaureidin (also known as NSC-106969); T-138067 (Tularik, also known as T-67, TL-138067 and TI-138067); COBRA-1 (Parker Hughes Institute, also known as DDE-261 and WHI-261); H10 (Kansas State University); H16 (Kansas State University); Oncocidin A1 (also known as BTO-956 and DIME); DDE-313 (Parker Hughes Institute); SPA-2 (Parker Hughes Institute); SPA-1 (Parker Hughes Institute, also known as SPIKET-P); 3-IAABU (Cytoskeleton/Mt. Sinai School of Medicine, also known as MF-569); Narcosine (also known as NSC-5366); Nascapine, D-24851 (Asta Medica), A-105972 (Abbott); Hemiasterlin; 3-BAABU (Cytoskeleton/Mt. Sinai School of Medicine, also known as MF-191); TMPN (Arizona State University); Vanadocene acetylacetonate; T-138026 (Tularik); Monsatrol; Inanocine (also known as NSC-698666); 3-IAABE (Cytoskeleton/Mt. Sinai School of Medicine); A-204197 (Abbott); T-607 (Tularik, also known as T-900607); RPR-115781 (Aventis); Eleutherobins (such as Desmethyleleutherobin, Desaetyleleutherobin, Isoeleutherobin A, and Z-Eleutherobin); Halichondrin B; D-64131 (Asta Medica); D-68144 (Asta Medica); Diazonamide A; A-293620 (Abbott); NPI-2350 (Nereus); TUB-245 (Aventis); A-259754 (Abbott); Diozostatin; (−)-Phenylahistin (also known as NSCL-96F037); D-68838 (Asta Medica); D-68836 (Asta Medica); Myoseverin B; D-43411 (Zentaris, also known as D-81862); A-289099 (Abbott); A-318315 (Abbott); HTI-286 (also known as SPA-110, trifluoroacetate salt) (Wyeth); D-82317 (Zentaris); D-82318 (Zentaris); SC-12983 (NCI); Resverastatin phosphate sodium; BPR-0Y-007 (National Health Research Institutes); SSR-250411 (Sanofi); Combretastatin A4; eribulin (Halaven®); and analogs and derivatives thereof.

In further embodiments, compounds according to the disclosure are used in combination with one or more alkylating agents, antimetabolites, natural products, or hormones.

Examples of alkylating agents useful in the methods of the disclosure include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.).

Examples of antimetabolites useful in the methods of the disclosure include but are not limited to folic acid analog (e.g., methotrexate), or pyrimidine analogs (e.g., fluorouracil, floxouridine, cytarabine), and purine analogs (e.g., mercaptopurine, thioguanine, pentostatin). Examples of natural products useful in the methods of the disclosure include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin) or enzymes (e.g., L-asparaginase).

Examples of hormones and antagonists useful for the treatment of cancer include but are not limited to adrenocorticosteroids (e.g., prednisone), progestins (e.g., hydroxyprogesterone caproate, megestrol acetate, medroxyprogesterone acetate), estrogens (e.g., diethylstilbestrol, ethinyl estradiol), antiestrogen (e.g., tamoxifen), androgens (e.g., testosterone propionate, fluoxymesterone), antiandrogen (e.g., flutamide), and gonadotropin releasing hormone analog (e.g., leuprolide).

Other agents that can be used in combination with the compounds of the disclosure for the treatment of cancer include platinum coordination complexes (e.g., cisplatin, carboplatin), anthracenedione (e.g., mitoxantrone), substituted urea (e.g., hydroxyurea), methyl hydrazine derivative (e.g., procarbazine), and adrenocortical suppressant (e.g., mitotane, aminoglutethimide). Other anti-cancer agents/drugs that can be used in combination with the compounds of the disclosure include, but are not limited to, liver X receptor (LXR) modulators, including LXR agonists and LXR beta-selective agonists; aryl hydrocarbon receptor (AhR) inhibitors; inhibitors of the enzyme poly ADP ribose polymerase (PARP), including olaparib, iniparib, rucaparib, veliparib; inhibitors of vascular endothelial growth factor (VEGF) receptor tyrosine kinases, including cediranib; programmed cell death protein 1 (PD-1) inhibitors, including nivolumab (Bristol-Myers Squibb Co.) and pembrolizumab (Merck & Co., Inc.; MK-3475); MEK inhibitors, including cobimetinib; B-Raf enzyme inhibitors, including vemurafenib; cytotoxic T lymphocyte antigen (CTLA-4) inhibitors, including tremelimumab; programmed death-ligand 1 (PD-L1) inhibitors, including MEDI4736 (AstraZeneca); inhibitors of the Wnt pathway; inhibitors of epidermal growth factor receptor (EGFR) including AZD9291 (AstraZeneca), erlotinib, gefitinib, panitumumab, and cetuximab; adenosine A2A receptor inhibitors; adenosine A2B receptor inhibitors; colony-stimulating factor-1 receptor (CSF1R) inhibitors, including PLX3397 (Plexxikon), and inhibitors of CD73.

The compounds of the disclosure can be used in combination with one or more therapeutic strategies including immune checkpoint inhibitors, including inhibitors of PD-1, PD-L1, and CTLA-4.

The compounds of the disclosure can be used in combination with one or more anti-cancer agents selected from MCL-1 inhibitors, e.g., homoharringtonin (HHT) and omacetaxine; BCL-2 inhibitors, e.g., venetoclax (ABT-199), navitoclax (ABT-263), ABT-737, gossypol (AT-101), apogossypolone (ApoG2) and obatoclax; selective inhibitors of nuclear export (SINEs), e.g., selinexor (KPT-330).

In particular embodiments, the compounds of the disclosure are used in combination with one or more anti-cancer agents selected from methotrexate (Abitrexate®; Folex®; Folex PFS®; Mexate®; Mexate-AQ®); nelarabine (Arranon®); blinatumomab (Blincyto®); rubidomycin hydrochloride or daunorubicin hydrochloride (Cerubidine®); cyclophosphamide (Clafen®; Cytoxan®; Neosar®); clofarabine (Clofarex®; Clolar®); cytarabine (Cytosar-U®; Tarabine PFS®); dasatinib (Sprycel®); doxorubicin hydrochloride; asparaginase Erwinia chrysanthemi (Erwinaze); imatinib mesylate (Gleevec®); ponatinib hydrochloride (Iclusig®); mercaptopurine (Purinethol; Purixan); pegaspargase (Oncaspar®); prednisone; vincristine sulfate (Oncovin®, Vincasar PFS®, Vincrex®); vincristine sulfate liposome (Marqibo®); hyper-CVAD (fractionated cyclophosphamide, vincristine, adriamycin, and dexamethasone); arsenic trioxide (Trisenox®); idarubicin hydrochloride (Idamycin®); mitoxantrone hydrochloride; thioguanine (Tabloid®); ADE (cytarabine, daunorubicin, and etoposide); alemtuzumab (Lemtrada®, Campath®); chlorambucil (Ambochlorin®, Amboclorin®, Leukeran®, Linfolizin®); ofatumumab (Arzerra®); bendamustine hydrochloride (Treanda®); fludarabine phosphate (Fludara®); obinutuzumab (Gazyva®); ibrutinib (Imbruvica®); idelalisib (Zydelig®); mechlorethamine hydrochloride (Mustargen®); rituximab (Rituxan®); chlorambucil-prednisone; CVP (cyclophosphamide, vincristine, and prednisone); bosutinib (Bosulif®); busulfan (Busulfex®; Myleran®); omacetaxine mepesuccinate (Synribo®); nilotinib (Tasigna®); Intron® A (recombinant interferon Alfa-2b); DOT1L inhibitors, including EPZ-5676 (Epizyme, Inc.); and inhibitors of bromodomain and extra-terminal motif (BET) proteins (BET inhibitors), including MS417, JQ1, I-BET 762, and I-BET 151 for the treatment of leukemia.

Compounds of the disclosure can be used in combination with one or more other agents or therapies for the treatment of insulin resistance, pre-diabetes, diabetes (e.g., Type 2 diabetes or Type 1 diabetes), and risk of diabetes, including but not limited to insulins and insulin analogues, such as Humulin® (Eli Lilly), Lantus® (Sanofi Aventis), Novolin® (Novo Nordisk), and Exubera® (Pfizer); Avandamet® (metformin HCI and rosiglitazone maleate, GSK); Avandaryl® (glimepiride and rosiglitazone maleate, GSK); Metaglip® (glipizide and metformin HCI, Bristol Myers Squibb); Glucovance® (glyburide and metformin HCI, Bristol Myers Squibb); PPAR gamma agonists, such as Avandia® (rosiglitizone maleate, GSK) and Actos® (pioglitazone hydrochloride, Takeda/Eli Lilly); sulfonylureas, such as Amaryl® (glimepiride, Sanofi Aventis), Diabeta® (glyburide, Sanofi Aventis), Micronase®/Glynase® (glyburide, Pfizer), and Glucotrol®/Glucotrol XL® (glipizide, Pfizer); meglitinides, such as Prandin®/NovoNorm® (repaglinide, Novo Nordisk), Starlix® (nateglinide, Novartis), and Glufast® (mitiglinide, Takeda); biguanides, such as Glucophase®/Glucophase XR® (metformin HCI, Bristol Myers Squibb) and Glumetza® (metformin HCI, Depomed); thiazolidinediones; amylin analogs; GLP-1 analogs; DPP-IV inhibitors such as Januvia® (sitagliptin, Merck) and Galvus® (vildagliptin, Novartis); PTB-1 B inhibitors; protein kinase inhibitors (including AMP-activated protein kinase inhibitors); glucagon antagonists, glycogen synthase kinase-3 beta inhibitors; glucose-6-phosphatase inhibitors; glycogen phosphorylase inhibitors; sodium glucose co-transporter inhibitors; and alpha-glucosidase inhibitors, such as Glycet® (miglitol, Pfizer); statins, fibrates, and Zetia® (ezetimibe); alpha-blockers; beta-blockers; calcium channel blockers; diuretics; angiotensin converting enzyme (ACE) inhibitors; dual ACE and neutral endopeptidase (NEP) inhibitors; angiotensin-receptor blockers (ARBs); aldosterone synthase inhibitors; aldosterone-receptor antagonists; endothelin receptor antagonists; orlistat; phentermine; sibutramine; Acomplia® (rimonabant); thiazolidinediones (e.g., rosiglitazone, pioglitazone); SGLT 2 inhibitors (e.g., dapagliflozin, remogliflozin etabonate, sergliflozin, canagliflozin, and 1-chloro-4-(β-D-glucopyranos-1-yl)-2-[4-((‘S)-tetrahydrofuran-3-yloxy)-benzyl]-benzene); PPAR-gamma-agonists (e.g., Gl 262570) and antagonists; PPAR-gamma/alpha modulators (e.g., KRP 297); alpha-glucosidase inhibitors (e.g., acarbose, voglibose); DPPIV inhibitors (e.g., Januvia® (sitagliptin), Galvus®/Zomelis® (vildagliptin), Onglyza® (saxagliptin), Nesina®/Vipidia® (alogliptin), and Tradjenta®/Trajenta® (linagliptin)); alpha2-antagonists; glucagon-like protein-1 (GLP-1) receptor agonists and analogues (e.g., exendin-4); amylin; inhibitors of protein tyrosinephosphatase 1; substances that affect deregulated glucose production in the liver, e.g., inhibitors of glucose-6-phosphatase, or fructose-1,6-bisphosphatase, glycogen phosphorylase; glucagon receptor antagonists; inhibitors of phosphoenol pyruvate carboxykinase; glycogen synthase kinase and glucokinase activators; lipid lowering agents such as HMG-CoA-reductase inhibitors (e.g., simvastatin, atorvastatin); fibrates (e.g., bezafibrate, fenofibrate), nicotinic acid and the derivatives thereof, PPAR-alpha agonists, PPAR-delta agonists; ACAT inhibitors (e.g., avasimibe); cholesterol absorption inhibitors such as ezetimibe; bile acid-binding substances such as cholestyramine; inhibitors of ileac bile acid transport; HDL-raising compounds such as CETP inhibitors and ABC1 regulators; active substances for treating obesity such as sibutramine and tetrahydrolipostatin; SDRIs; axokine; leptin; leptin mimetics; antagonists of the cannabinoid 1 receptor; and MCH-1 receptor antagonists; MC4 receptor agonists; NPY5 and NPY2 antagonists; beta3 adrenergic agonists such as SB-418790 and AD-9677; agonists of the 5HT2c receptor; GABA-receptor antagonists; Na-channel blockers; topiramate; protein-kinase C inhibitors; advanced glycation end product inhibitors; and aldose reductase inhibitors.

In some embodiments, a combination of Compound A citrate salt (e.g., Compound A monocitrate monohydrate salt) or polymorphic form thereof (e.g., Compound A monocitrate monohydrate Form A salt) and systemic corticosteroids (e.g., dexamethasone 10 mg IV every 12 hours in adults or dexamethasone 0.25 mg/kg/dose IV every 12 hours in pediatric patients weighing less than 40 kg) is administered to a subject in need thereof.

In some embodiments, a combination of Compound A citrate salt (e.g., Compound A monocitrate monohydrate salt) or polymorphic form thereof (e.g., Compound A monocitrate monohydrate Form A salt) and hydroxyurea is administered to a subject in need thereof.

In some embodiments, the present disclosure is directed to Compound A, Compound A citrate salt (e.g., Compound A monocitrate monohydrate salt) or polymorphic form thereof (e.g., Compound A monocitrate monohydrate Form A salt) administered to a subject in need thereof is about 270 mg as free base equivalents twice daily, wherein the subject in need thereof weighs greater than or equal to 40 kg.

In some embodiments, the present disclosure is directed to Compound A, Compound A citrate salt (e.g., Compound A monocitrate monohydrate salt) or polymorphic form thereof (e.g., Compound A monocitrate monohydrate Form A salt) administered to a subject in need thereof is about 160 mg as free base equivalents twice daily, wherein the subject in need thereof weighs greater than or equal to 40 kg.

In some embodiments, the present disclosure is directed to Compound A, Compound A citrate salt (e.g., Compound A monocitrate monohydrate salt) or polymorphic form thereof (e.g., Compound A monocitrate monohydrate Form A salt) administered to a subject in need thereof is about 110 mg as free base equivalents twice daily, wherein the subject in need thereof weighs greater than or equal to 40 kg.

In some embodiments, the present disclosure is directed to Compound A, Compound A citrate salt (e.g., Compound A monocitrate monohydrate salt) or polymorphic form thereof (e.g., Compound A monocitrate monohydrate Form A salt) administered to a subject in need thereof is about 95 mg/m² as free base equivalents twice daily, wherein the subject in need thereof weighs less than 40 kg.

In some embodiments, the present disclosure is directed to Compound A, Compound A citrate salt (e.g., Compound A monocitrate monohydrate salt) or polymorphic form thereof (e.g., Compound A monocitrate monohydrate Form A salt) administered to a subject in need thereof is about 65 mg/m² as free base equivalents twice daily, wherein the subject in need thereof weighs less than 40 kg.

In some embodiments, the present disclosure is directed to a combination of Compound A monocitrate monohydrate Form A salt and a CYP3A4 inhibitor administered to a subject in need thereof, wherein the amount of Compound A monocitrate monohydrate Form A salt administered is about 95 mg/m² as free base equivalents twice daily.

In some embodiments, the present disclosure is directed to a combination of Compound A monocitrate monohydrate Form A salt and a CYP3A4 inhibitor administered to a subject in need thereof, wherein the amount of Compound A monocitrate monohydrate Form A salt administered is about 65 mg/m² as free base equivalents twice daily.

In some embodiments, the present disclosure is directed to a combination of Compound A monocitrate monohydrate Form A salt and a CYP3A4 inhibitor administered to a subject in need thereof, wherein the amount of Compound A monocitrate monohydrate Form A salt administered is about 30 mg/m² as free base equivalents twice daily.

In some embodiments, the present disclosure is directed to a combination of Compound A monocitrate monohydrate Form A salt and a CYP3A4 inhibitor administered to a subject in need thereof, wherein the amount of Compound A monocitrate monohydrate Form A salt administered is about 95 mg/m² as free base equivalents twice daily, wherein the subject in need thereof weighs less than 40 kg.

In some embodiments, the present disclosure is directed to a combination of Compound A monocitrate monohydrate Form A salt and a CYP3A4 inhibitor administered to a subject in need thereof, wherein the amount of Compound A monocitrate monohydrate Form A salt administered is about 65 mg/m² as free base equivalents twice daily, wherein the subject in need thereof weighs less than 40 kg.

In some embodiments, the present disclosure is directed to a combination of Compound A monocitrate monohydrate Form A salt and a CYP3A4 inhibitor administered to a subject in need thereof, wherein the amount of Compound A monocitrate monohydrate Form A salt administered is about 30 mg/m² as free base equivalents twice daily, wherein the subject in need thereof weighs less than 40 kg.

In some embodiments, the present disclosure is directed to a combination of Compound A monocitrate monohydrate Form A salt and a CYP3A4 inhibitor administered to a subject in need thereof, wherein the subject in need thereof weighs less than 40 kg and wherein the amount of Compound A monocitrate monohydrate Form A salt administered is adjusted by body surface area of the subject in need thereof.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 50 mg as free base equivalents twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 110 mg as free base equivalents twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 160 mg as free base equivalents twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 270 mg as free base equivalents twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 220 mg as free base equivalents twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 50 mg twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 110 mg twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 160 mg twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 270 mg twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 220 mg twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the subject in need thereof weighs less than 40 kg and wherein the amount of Compound A, Compound A citrate salt (e.g., Compound A monocitrate monohydrate salt) or polymorphic form thereof (e.g., Compound A monocitrate monohydrate Form A salt) administered is adjusted by body surface area of the subject in need thereof.

In some embodiments, the present disclosure is directed to a combination of Compound A monocitrate monohydrate Form A salt and a CYP3A4 inhibitor administered to a subject in need thereof, wherein the amount of Compound A monocitrate monohydrate Form A salt administered is about 160 mg as free base equivalents twice daily, wherein the subject in need thereof weighs great than or equal to 40 kg.

In some embodiments, the present disclosure is directed to a combination of Compound A monocitrate monohydrate Form A salt and a CYP3A4 inhibitor administered to a subject in need thereof, wherein the amount of Compound A monocitrate monohydrate Form A salt administered is about 110 mg as free base equivalents twice daily, wherein the subject in need thereof weighs great than or equal to 40 kg.

In some embodiments, the present disclosure is directed to a combination of Compound A monocitrate monohydrate Form A salt and a CYP3A4 inhibitor administered to a subject in need thereof, wherein the amount of Compound A monocitrate monohydrate Form A salt administered is about 50 mg as free base equivalents twice daily, wherein the subject in need thereof weighs great than or equal to 40 kg.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein:
(a) the subject in need thereof weighs less than 40 kg,
(b) the amount of Compound A, Compound A citrate salt (e.g., Compound A monocitrate monohydrate salt) or polymorphic form thereof (e.g., Compound A monocitrate monohydrate Form A salt) administered is adjusted by body surface area of the subject in need thereof, and
(c) the amount of Compound A, Compound A citrate salt (e.g., Compound A monocitrate monohydrate salt) or polymorphic form thereof (e.g., Compound A monocitrate monohydrate Form A salt) is administered in 25 mg (as free base equivalents) tablets.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein:
(a) the subject in need thereof weighs less than 40 kg,
(b) the amount of Compound A, Compound A citrate salt (e.g., Compound A monocitrate monohydrate salt) or polymorphic form thereof (e.g., Compound A monocitrate monohydrate Form A salt) administered is adjusted by body surface area of the subject in need thereof, and
(c) the amount of Compound A, Compound A citrate salt (e.g., Compound A monocitrate monohydrate salt) or polymorphic form thereof (e.g., Compound A monocitrate monohydrate Form A salt) is administered in 25 mg tablets.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein:
(a) the subject in need thereof weighs less than 40 kg,
(b) the amount of Compound A, Compound A citrate salt (e.g., Compound A monocitrate monohydrate salt) or polymorphic form thereof (e.g., Compound A monocitrate monohydrate Form A salt) administered is adjusted by body surface area of the subject in need thereof, and
(c) the amount of Compound A, Compound A citrate salt (e.g., Compound A monocitrate monohydrate salt) or polymorphic form thereof (e.g., Compound A monocitrate monohydrate Form A salt) is administered in oral solution.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 50 mg twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 110 mg twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 160 mg twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 270 mg twice daily.

In some embodiments, a combination of Compound A, Compound A citrate salt (e.g., Compound A monocitrate monohydrate salt) or polymorphic form thereof (e.g., Compound A monocitrate monohydrate Form A salt) and a CYP3A4 inhibitor is administered to a subject in need thereof, wherein the amount of Compound A, Compound A citrate salt (e.g., Compound A monocitrate monohydrate salt) or polymorphic form thereof (e.g., Compound A monocitrate monohydrate Form A salt) administered is about 220 mg twice daily.

In some embodiments, a combination of Compound A monocitrate monohydrate and a CYP3A4 inhibitor is administered to a subject in need thereof, wherein the amount of Compound A monocitrate monohydrate administered is from about 100 mg to about 500 mg as free base equivalents twice daily.

In some embodiments, a combination of Compound A monocitrate monohydrate and a CYP3A4 inhibitor is administered to a subject in need thereof, wherein the amount of Compound A monocitrate monohydrate administered is from about 110 mg to about 450 mg as free base equivalents twice daily.

In some embodiments, a combination of Compound A monocitrate monohydrate and a CYP3A4 inhibitor is administered to a subject in need thereof, wherein the amount of Compound A monocitrate monohydrate administered is from about 120 mg to about 400 mg as free base equivalents twice daily.

In some embodiments, a combination of Compound A monocitrate monohydrate and a CYP3A4 inhibitor is administered to a subject in need thereof, wherein the amount of Compound A monocitrate monohydrate administered is from about 130 mg to about 350 mg as free base equivalents twice daily.

In some embodiments, a combination of Compound A monocitrate monohydrate and a CYP3A4 inhibitor is administered to a subject in need thereof, wherein the amount of Compound A monocitrate monohydrate administered is from about 140 mg to about 300 mg as free base equivalents twice daily.

In some embodiments, a combination of Compound A monocitrate monohydrate and a CYP3A4 inhibitor is administered to a subject in need thereof, wherein the amount of Compound A monocitrate monohydrate administered is from about 150 mg to about 290 mg as free base equivalents twice daily.

In some embodiments, a combination of Compound A monocitrate monohydrate and a CYP3A4 inhibitor is administered to a subject in need thereof, wherein the amount of Compound A monocitrate monohydrate administered is from about 165 mg to about 280 mg as free base equivalents twice daily.

In some embodiments, a combination of Compound A monocitrate monohydrate and a CYP3A4 inhibitor is administered to a subject in need thereof, wherein the amount of Compound A monocitrate monohydrate administered is from about 50 mg/m² to about 300 mg/m² as free base equivalents twice daily.

In some embodiments, a combination of Compound A monocitrate monohydrate and a CYP3A4 inhibitor is administered to a subject in need thereof, wherein the amount of Compound A monocitrate monohydrate administered is from about 60 mg/m² to about 250 mg/m² as free base equivalents twice daily.

In some embodiments, a combination of Compound A monocitrate monohydrate and a CYP3A4 inhibitor is administered to a subject in need thereof, wherein the amount of Compound A monocitrate monohydrate administered is from about 70 mg/m² to about 200 mg/m² as free base equivalents twice daily.

In some embodiments, a combination of Compound A monocitrate monohydrate and a CYP3A4 inhibitor is administered to a subject in need thereof, wherein the amount of Compound A monocitrate monohydrate administered is from about 80 mg/m² to about 180 mg/m² as free base equivalents twice daily.

In some embodiments, a combination of Compound A monocitrate monohydrate and a CYP3A4 inhibitor is administered to a subject in need thereof, wherein the amount of Compound A monocitrate monohydrate administered is from about 90 mg/m² to about 170 mg/m² as free base equivalents twice daily.

In some embodiments, a combination of Compound A monocitrate monohydrate and a CYP3A4 inhibitor is administered to a subject in need thereof, wherein the amount of Compound A monocitrate monohydrate administered is from about 95 mg/m² to about 160 mg/m² as free base equivalents twice daily.

In some embodiments, a combination of Compound A monocitrate monohydrate and a CYP3A4 inhibitor is administered to a subject in need thereof, wherein the subject in need thereof weighs less than 40 kg and wherein the amount of Compound A monocitrate monohydrate administered is adjusted by body surface area of the subject in need thereof.

In some embodiments, a combination of Compound A monocitrate monohydrate and a CYP3A4 inhibitor is administered to a subject in need thereof, wherein:
(a) the subject in need thereof weighs less than 40 kg,
(b) the amount of Compound A monocitrate monohydrate administered is adjusted by body surface area of the subject in need thereof, and
(c) the amount of Compound A monocitrate monohydrate salt is administered in 25 mg (as free base equivalents) tablets.

In some embodiments, a combination of Compound A monocitrate monohydrate and a CYP3A4 inhibitor is administered to a subject in need thereof, wherein:
(a) the subject in need thereof weighs less than 40 kg,
(b) the amount of Compound A monocitrate monohydrate administered is adjusted by body surface area of the subject in need thereof, and
(c) the amount of Compound A monocitrate monohydrate salt is administered in 25 mg tablets.

In some embodiments, a combination of Compound A monocitrate monohydrate and a CYP3A4 inhibitor is administered to a subject in need thereof, wherein:
(a) the subject in need thereof weighs less than 40 kg,
(b) the amount of Compound A monocitrate monohydrate administered is adjusted by body surface area of the subject in need thereof, and
(c) the amount of Compound A monocitrate monohydrate salt is administered in oral solution.

In some embodiments, a combination of Compound A monocitrate monohydrate and a CYP3A4 inhibitor is administered to a subject in need thereof, wherein the amount of Compound A monocitrate monohydrate administered is about 160 mg twice daily.

In some embodiments, a combination of Compound A monocitrate monohydrate and a CYP3A4 inhibitor is administered to a subject in need thereof, wherein the amount of Compound A monocitrate monohydrate administered is about 110 mg twice daily.

In some embodiments, a combination of Compound A monocitrate monohydrate and a CYP3A4 inhibitor is administered to a subject in need thereof, wherein the amount of Compound A monocitrate monohydrate administered is about 270 mg twice daily.

In some embodiments, a combination of Compound A monocitrate monohydrate and a CYP3A4 inhibitor is administered to a subject in need thereof, wherein the amount of Compound A monocitrate monohydrate administered is about 220 mg twice daily.

If a strong CYP3A4 inhibitor must be co-administered, the dose for patients ≥40 kg is reduced from 270 mg to 160 mg orally twice daily. The dose for patients <40 kg is reduced from 160 mg/m² to 95 mg/m² orally twice daily, with or without food, until disease progression or unacceptable toxicity.

A list of strong CYP3A4 inhibitors is provided in Table 7.

TABLE 7
Strong CYP3A4 inhibitors
Strong inhibitors of CYP3A4^{a}

| | |
|---|---|
| boceprevir | nefazodone |
| clarithromycin | nelfinavir |
| conivapran | posaconazole |
| grapefruit juice | ritonavir |
| indinavir | saquinavir |
| itraconazole | telaprevir |
| ketoconazole | telithromycin |
| lopinavir | voriconazole |
| mibefradil | |

^{a}Increases the AUC of the substrate by ≥5-fold.

In some embodiments, if a strong CYP3A4 inhibitor must be co-administered, the dose for patients ≥40 kg is reduced from 270 mg to 160 mg orally twice daily.

If a strong CYP3A4 inhibitor must be co-administered, the dose for patients ≥40 kg is reduced from 270 mg to 160 mg orally twice daily (based on free base equivalents).

In some embodiments, if a strong CYP3A4 inhibitor must be co-administered, for patients ≥40 kg the recommended starting dose of Compound A monocitrate monohydrate salt is 160 mg orally twice daily, with or without food, until disease progression or unacceptable toxicity.

In some embodiments, if a strong CYP3A4 inhibitor must be co-administered, for patients ≥40 kg the recommended starting dose of Compound A monocitrate monohydrate salt is 110 mg orally twice daily, with or without food, until disease progression or unacceptable toxicity.

In some embodiments, if a strong CYP3A4 inhibitor must be co-administered, for patients ≥40 kg the recommended starting dose of Compound A monocitrate monohydrate salt is 50 mg orally twice daily, with or without food, until disease progression or unacceptable toxicity.

In some embodiments, if a strong CYP3A4 inhibitor must be co-administered, for patients <40 kg the recommended starting dose of Compound A monocitrate monohydrate salt is 95 mg/m² orally twice daily, with or without food, until disease progression or unacceptable toxicity.

In some embodiments, if a strong CYP3A4 inhibitor must be co-administered, for patients <40 kg the recommended starting dose of Compound A monocitrate monohydrate salt is 65 mg/m² orally twice daily, with or without food, until disease progression or unacceptable toxicity.

In some embodiments, if a strong CYP3A4 inhibitor must be co-administered, for patients <40 kg the recommended starting dose of Compound A monocitrate monohydrate salt is 30 mg/m² orally twice daily, with or without food, until disease progression or unacceptable toxicity.

In some embodiments, if a strong CYP3A4 inhibitor must be co-administered, for patients ≥40 kg the recommended starting dose of Compound A monocitrate monohydrate salt is 160 mg (as free base equivalents) orally twice daily, with or without food, until disease progression or unacceptable toxicity.

In some embodiments, if a strong CYP3A4 inhibitor must be co-administered, for patients ≥40 kg the recommended starting dose of Compound A monocitrate monohydrate salt is 110 mg (as free base equivalents) orally twice daily, with or without food, until disease progression or unacceptable toxicity.

In some embodiments, if a strong CYP3A4 inhibitor must be co-administered, for patients ≥40 kg the recommended starting dose of Compound A monocitrate monohydrate salt is 50 mg (as free base equivalents) orally twice daily, with or without food, until disease progression or unacceptable toxicity.

In some embodiments, if a strong CYP3A4 inhibitor must be co-administered, for patients <40 kg the recommended starting dose of Compound A monocitrate monohydrate salt is 95 mg/m² (as free base equivalents) orally twice daily, with or without food, until disease progression or unacceptable toxicity.

In some embodiments, if a strong CYP3A4 inhibitor must be co-administered, for patients <40 kg the recommended starting dose of Compound A monocitrate monohydrate salt is 65 mg/m² (as free base equivalents) orally twice daily, with or without food, until disease progression or unacceptable toxicity.

In some embodiments, if a strong CYP3A4 inhibitor must be co-administered, for patients <40 kg the recommended starting dose of Compound A monocitrate monohydrate salt is 30 mg/m² (as free base equivalents) orally twice daily, with or without food, until disease progression or unacceptable toxicity.

In some embodiments, Compound A monocitrate monohydrate salt is administered to a patient in need thereof and on Strong CYP3A4 inhibitors with dose reduction as provided in Table 8:

TABLE 8
Starting Dose of Compound A monocitrate monohydrate salt and
Dose Modifications for patients on Strong CYP3A4 Inhibitors

| Dose Level | Patients Weighing ≥ 40 kg | Patients Weighing < 40 kg |
|---|---|---|
| Starting Dose | 160 mg orally twice daily | 95 mg/m² orally |
| | | twice daily |
| First Dose | 110 mg orally twice daily | 65 mg/m² orally |
| Reduction | | twice daily |
| Second Dose | 50 mg orally twice daily | 30 mg/m² orally |
| Reduction | | twice daily |

The amount of a given agent that will correspond to such an amount will vary depending upon factors such as the particular compound, the severity of the disease, the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

It will be understood that data present in the Examples is considered to be part of the present invention. In some embodiments, the data located in the Examples are considered as inventive entities individually, are referred to by embodiments in the detailed description and are considered part of the detailed description.

### LISTING OF NUMBERED EMBODIMENTS

1. A Compound A monocitrate monohydrate Form A salt, wherein Compound A is of the following formula:

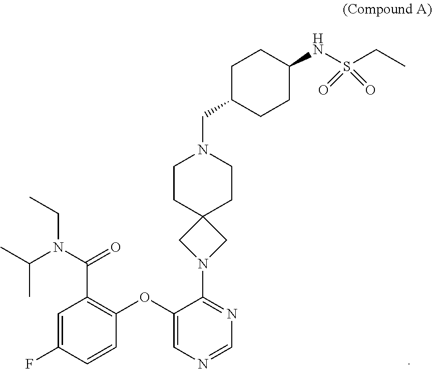 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

2. The Compound A monocitrate monohydrate Form A salt of embodiment 1, wherein the Compound A monocitrate monohydrate Form A salt is crystalline.
3. The Compound A monocitrate monohydrate Form A salt of embodiment 1, wherein the Compound A monocitrate salt is amorphous.
4. The Compound A monocitrate monohydrate Form A salt of embodiment 1 or 2, wherein the Compound A monocitrate monohydrate Form A salt is a crystalline salt characterized a PXRD signal at 17.0°2θ (±0.2°2θ Cu Kα1 radiation).
5. The Compound A monocitrate monohydrate Form A salt of embodiment 1 or 2, wherein the Compound A monocitrate monohydrate Form A salt is a crystalline salt characterized by two, or three PXRD signals selected from 5.2°2θ, 11.8°2θ, and 16.9°2θ (±0.2°2θ; ±0.1°2θ; or ±0.0°2θ; Cu Kα1 radiation).
6. The Compound A monocitrate monohydrate Form A salt of embodiment 1, 2, or 5, wherein the Compound A monocitrate monohydrate Form A salt is a salt characterized by PXRD signals at 5.2°2θ, 11.8°2θ, and 16.9°2θ (±0.2°2θ; ±0.1°2θ; or ±0.0°2θ; Cu Kα1 radiation).
7. The Compound A monocitrate monohydrate Form A salt of any one of embodiments 1-2 and 5-6, wherein the Compound A monocitrate monohydrate Form A salt is a crystalline salt characterized by two or more, or three or more PXRD signals selected from the group consisting of 5.2°2θ, 11.8°2θ, 16.2°2θ, and 16.9°2θ (±0.2°2θ; ±0.1°2θ; or ±0.0°2θ; Cu Kα1 radiation).
8. The Compound A monocitrate monohydrate Form A salt of any one of embodiments 1-2 and 5-7, wherein the Compound A monocitrate monohydrate Form A salt is a crystalline salt characterized by PXRD signals at 5.2°2θ, 11.8°2θ, 16.2°2θ, and 16.9°2θ (±0.2°2θ; ±0.1°2θ; or ±0.0°2θ; Cu Kα1 radiation).
9. The Compound A monocitrate monohydrate Form A salt of any one of embodiments 1-2 and 5-8, wherein the Compound A monocitrate monohydrate Form A salt is a crystalline salt characterized by two or more, or three or more PXRD signals selected from the group consisting of 5.2°2θ, 11.8°2θ, 16.2°2θ, 16.9°2θ, and 19.2°2θ (±0.2°2θ; ±0.1°2θ; or ±0.0°2θ; Cu Kα1 radiation).
10. The Compound A monocitrate monohydrate Form A salt of any one of embodiments 1-2 and 5-9, wherein the Compound A monocitrate monohydrate Form A salt is a crystalline salt characterized by PXRD signals at 5.2°2θ, 11.8°2θ, 16.2°2θ, 16.9°2θ, and 19.2°2θ (±0.2°2θ; ±0.1°2θ; or ±0.0°2θ; Cu Kα1 radiation).
11. The Compound A monocitrate monohydrate Form A salt of any one of embodiments 1-2 and 5-10, wherein the Compound A monocitrate monohydrate Form A salt is a crystalline salt characterized by two or more, or three or more PXRD signals selected from the group consisting of 5.2°2θ, 11.8°2θ, 16.2°2θ, 16.9°2θ, 19.2°2θ, and 20.9°2θ (±0.2°2θ; ±0.1°2θ; or ±0.0°2θ; Cu Kα1 radiation).
12. The Compound A monocitrate monohydrate Form A salt of any one of embodiments 1-2 and 5-11, wherein the Compound A monocitrate monohydrate Form A salt is a crystalline salt characterized by PXRD signals at 5.2°2θ, 11.8°2θ, 16.2°2θ, 16.9°2θ, 19.2°2θ, and 20.9°2θ (±0.2°2θ; ±0.1°2θ; or ±0.0°2θ; Cu Kα1 radiation).
13. The Compound A monocitrate monohydrate Form A salt of any one of embodiments 1-2 and 5-12, wherein the Compound A monocitrate monohydrate Form A salt is a crystalline salt characterized by two or more, or three or more PXRD signals selected from the group consisting of 5.2°2θ, 11.8°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).
14. The Compound A monocitrate monohydrate Form A salt of any one of embodiments 1-2 and 5-13, wherein the Compound A monocitrate monohydrate Form A salt is a crystalline salt characterized by PXRD signals at 5.2°2θ, 11.8°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).
15. The Compound A monocitrate monohydrate Form A salt of any one of embodiments 1-2 and 5-14, wherein the Compound A monocitrate monohydrate Form A salt is a crystalline salt characterized by two or more, or three or more PXRD signals selected from the group consisting of 5.2°2θ, 11.8°2θ, 12.6°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).
16. The Compound A monocitrate monohydrate Form A salt of any one of embodiments 1-2 and 5-15, wherein the Compound A monocitrate monohydrate Form A salt is a crystalline salt characterized by PXRD signals at 5.2°2θ, 11.8°2θ, 12.6°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).
17. The Compound A monocitrate monohydrate Form A salt of any one of embodiments 1-2 and 5-16, wherein the Compound A monocitrate monohydrate Form A salt is a crystalline salt characterized by two or more, or three or more PXRD signals selected from the group consisting of 5.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).
18. The Compound A monocitrate monohydrate Form A salt of any one of embodiments 1-2 and 5-17, wherein the Compound A monocitrate monohydrate Form A salt is a crystalline salt characterized by PXRD signals at 5.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).
19. The Compound A monocitrate monohydrate Form A salt of any one of embodiments 1-2 and 5-18, wherein the Compound A monocitrate monohydrate Form A salt is a crystalline salt characterized by two or more, or three or more PXRD signals selected from the group consisting of 5.2°2θ, 11.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).
20. The Compound A monocitrate monohydrate Form A salt of any one of embodiments 1-2 and 5-19, wherein the Compound A monocitrate monohydrate Form A salt is a crystalline salt characterized by PXRD signals at 5.2°2θ, 11.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).
21. The Compound A monocitrate monohydrate Form A salt of any one of embodiments 1-2 and 5-20, wherein the Compound A monocitrate monohydrate Form A salt is a crystalline salt characterized by one, two, three, four, five, six, seven, eight, nine, ten, eleven, twelve, thirteen, fourteen, fifteen, sixteen, seventeen, eighteen, or nineteen PXRD signals selected from those set forth in Table 1A.
22. The Compound A monocitrate monohydrate Form A salt of any one of embodiments 1-2 and 5-21, wherein the Compound A monocitrate monohydrate Form A salt is a crystalline salt characterized by a PXRD spectrum substantially similar to that shown in FIG. 1A.
23. The Compound A monocitrate monohydrate Form A salt of embodiment 1 or 2, wherein the Compound A monocitrate monohydrate Form A salt is a crystalline salt characterized by two, or three PXRD signals selected from 11.8°2θ, 12.7°2θ, and 17.0°2θ (±0.2°2θ; ±0.1°2θ; or ±0.0°2θ; Cu Kα1 radiation).
24. The Compound A monocitrate monohydrate Form A salt of embodiment 1, 2, or 23, wherein the Compound A monocitrate monohydrate Form A salt is a salt characterized by PXRD signals at 11.8°2θ, 12.7°2θ, and 17.0°2θ (±0.2°2θ; ±0.1°2θ; or ±0.0°2θ; Cu Kα1 radiation).
25. The Compound A monocitrate monohydrate Form A salt of any one of embodiments 1-2 and 23-24, wherein the Compound A monocitrate monohydrate Form A salt is a crystalline salt characterized by two or more, or three or more PXRD signals selected from the group consisting of 11.8°2θ, 12.7°2θ, 17.0°2θ, and 20.5°2θ (±0.2°2θ; ±0.1°2θ; or ±0.0°2θ; Cu Kα1 radiation).
26. The Compound A monocitrate monohydrate Form A salt of any one of embodiments 1-2 and 23-25, wherein the Compound A monocitrate monohydrate Form A salt is a crystalline salt characterized by PXRD signals at 11.8°2θ, 12.7°2θ, 17.0°2θ, and 20.5°2θ (±0.2°2θ; ±0.1°2θ; or ±0.0°2θ; Cu Kα1 radiation).
27. The Compound A monocitrate monohydrate Form A salt of any one of embodiments 1-2 and 23-26, wherein the Compound A monocitrate monohydrate Form A salt is a crystalline salt characterized by two or more, or three or more PXRD signals selected from the group consisting of 11.3°2θ, 11.8°2θ, 12.7°2θ, 17.0°2θ, and 20.5°2θ (±0.2°2θ; ±0.1°2θ; or ±0.0°2θ; Cu Kα1 radiation).
28. The Compound A monocitrate monohydrate Form A salt of any one of embodiments 1-2 and 23-27, wherein the Compound A monocitrate monohydrate Form A salt is a crystalline salt characterized by PXRD signals at 11.3°2θ, 11.8°2θ, 12.7°2θ, 17.0°2θ, and 20.5°2θ (±0.2°2θ; ±0.1°2θ; or ±0.0°2θ; Cu Kα1 radiation).
29. The Compound A monocitrate monohydrate Form A salt of any one of embodiments 1-2 and 23-28, wherein the Compound A monocitrate monohydrate Form A salt is a crystalline salt characterized by two or more, or three or more PXRD signals selected from the group consisting of 11.3°2θ, 11.8°2θ, 12.7°2θ, 16.3°2θ, 17.0°2θ, and 20.5°2θ (±0.2°2θ; ±0.1°2θ; or ±0.0°2θ; Cu Kα1 radiation).
30. The Compound A monocitrate monohydrate Form A salt of any one of embodiments 1-2 and 23-29, wherein the Compound A monocitrate monohydrate Form A salt is a crystalline salt characterized by PXRD signals at 11.3°2θ, 11.8°2θ, 12.7°2θ, 16.3°2θ, 17.0°2θ, and 20.5°2θ (±0.2°2θ; ±0.1°2θ; or ±0.0°2θ; Cu Kα1 radiation).
31. The Compound A monocitrate monohydrate Form A salt of any one of embodiments 1-2 and 23-30, wherein the Compound A monocitrate monohydrate Form A salt is a crystalline salt characterized by two or more, or three or more PXRD signals selected from the group consisting of 11.3°2θ, 11.8°2θ, 12.7°2θ, 16.3°2θ, 17.0°2θ, 17.3°2θ, and 20.5°2θ (±0.2°2θ; ±0.1°2θ; or ±0.0°2θ; Cu Kα1 radiation).
32. The Compound A monocitrate monohydrate Form A salt of any one of embodiments 1-2 and 23-31, wherein the Compound A monocitrate monohydrate Form A salt is a crystalline salt characterized by PXRD signals at 11.3°2θ, 11.8°2θ, 12.7°2θ, 16.3°2θ, 17.0°2θ, 17.3°2θ, and 20.5°2θ (±0.2°2θ; ±0.1°2θ; or ±0.0°2θ; Cu Kα1 radiation).
33. The Compound A monocitrate monohydrate Form A salt of any one of embodiments 1-2 and 23-32, wherein the Compound A monocitrate monohydrate Form A salt is a crystalline salt characterized by two or more, or three or more PXRD signals selected from the group consisting of 11.3°2θ, 11.8°2θ, 12.7°2θ, 16.3°2θ, 17.0°2θ, 17.3°2θ, 19.3°2θ, and 20.5°2θ (±0.2°2θ; ±0.1°2θ; or ±0.0°2θ; Cu Kα1 radiation).
34. The Compound A monocitrate monohydrate Form A salt of any one of embodiments 1-2 and 23-33, wherein the Compound A monocitrate monohydrate Form A salt is a crystalline salt characterized by PXRD signals at 11.3°2θ, 11.8°2θ, 12.7°2θ, 16.3°2θ, 17.0°2θ, 17.3°2θ, 19.3°2θ, and 20.5°2θ (±0.2°2θ; ±0.1°2θ; or ±0.0°2θ; Cu Kα1 radiation).
35. The Compound A monocitrate monohydrate Form A salt of any one of embodiments 1-2 and 23-34, wherein the Compound A monocitrate monohydrate Form A salt is a crystalline salt characterized by two or more, or three or more PXRD signals selected from the group consisting of 11.3°2θ, 11.8°2θ, 12.7°2θ, 16.3°2θ, 17.0°2θ, 17.3°2θ, 19.3°2θ, 20.3°2θ, and 20.5°2θ (±0.2°2θ; ±0.1°2θ; or ±0.0°2θ; Cu Kα1 radiation).
36. The Compound A monocitrate monohydrate Form A salt of any one of embodiments 1-2 and 23-35, wherein the Compound A monocitrate monohydrate Form A salt is a crystalline salt characterized by PXRD signals at 11.3°2θ, 11.8°2θ, 12.7°2θ, 16.3°2θ, 17.0°2θ, 17.3°2θ, 19.3°2θ, 20.3°2θ, and 20.5°2θ (±0.2°2θ; ±0.1°2θ; or ±0.0°2θ; Cu Kα1 radiation).
37. The Compound A monocitrate monohydrate Form A salt of any one of embodiments 1-2 and 23-36, wherein the Compound A monocitrate monohydrate Form A salt is a crystalline salt characterized by two or more, or three or more PXRD signals selected from the group consisting of 11.3°2θ, 11.8°2θ, 12.7°2θ, 16.3°2θ, 17.0°2θ, 17.3°2θ, 18.3°2θ, 19.3°2θ, 20.3°2θ, and 20.5°2θ (±0.2°2θ; ±0.1°2θ; or ±0.0°2θ; Cu Kα1 radiation).
38. The Compound A monocitrate monohydrate Form A salt of any one of embodiments 1-2 and 23-37, wherein the Compound A monocitrate monohydrate Form A salt is a crystalline salt characterized by PXRD signals at 11.3°2θ, 11.8°2θ, 12.7°2θ, 16.3°2θ, 17.0°2θ, 17.3°2θ, 18.3°2θ, 19.3°2θ, 20.3°2θ, and 20.5°2θ (±0.2°2θ; ±0.1°2θ; or ±0.0°2θ; Cu Kα1 radiation).
39. The Compound A monocitrate monohydrate Form A salt of any one of embodiments 1-2 and 23-38, wherein the Compound A monocitrate monohydrate Form A salt is a crystalline salt characterized by one, two, three, four, five, six, seven, eight, nine, ten, eleven, twelve, thirteen, fourteen, fifteen, sixteen, seventeen, eighteen, or nineteen PXRD signals selected from those set forth in Table 1B.
40. The Compound A monocitrate monohydrate Form A salt of any one of embodiments 1-2 and 23-39, wherein the Compound A monocitrate monohydrate Form A salt is a crystalline salt characterized by a PXRD spectrum substantially similar to that shown in FIG. 1B.
41. The Compound A monocitrate monohydrate Form A salt of any one of embodiments 1-2 and 4-40, wherein the Compound A monocitrate monohydrate Form A salt is a 1:1 Compound A:citrate salt.
42. The Compound A monocitrate monohydrate Form A salt of any one of embodiments 1-2 and 4-41, wherein the Compound A monocitrate monohydrate Form A salt has a particle size Dₓ(90) of about 15 μm to about 25 μm.
43. The Compound A monocitrate monohydrate Form A salt of any one of embodiments 1-2 and 4-42, wherein the Compound A monocitrate monohydrate Form A salt has a median particle size of about 8 μm to about 25 μm.
44. The Compound A monocitrate monohydrate Form A salt of any one of embodiments 1-2 and 4-43, wherein the Compound A monocitrate monohydrate Form A salt has an average particle size of about 8 μm to about 25 μm.
45. The Compound A monocitrate monohydrate Form A salt of any one of embodiments 1-2 and 4-44, wherein the Compound A monocitrate monohydrate Form A salt has a particle size Dₓ(50) of about 5 μm to about 10 μm.
46. The Compound A monocitrate monohydrate Form A salt of any one of embodiments 1-2 and 4-45, wherein the Compound A monocitrate monohydrate Form A salt has a particle size Dₓ(10) of about 1 μm to about 5 μm.
47. A pharmaceutical composition comprising the Compound A monocitrate monohydrate Form A salt according to any one of embodiments 1-46 and a pharmaceutically acceptable excipient.
48. A pharmaceutical composition comprising the Compound A monocitrate monohydrate Form A salt according to any one of embodiments 1-46 for use in the treatment or prevention of a cancer in a subject.
49. A tablet comprising the Compound A monocitrate monohydrate Form A salt according to any one of embodiments 1-46, and a pharmaceutically acceptable excipient.
50. The tablet of embodiment 49, wherein the tablet further comprises a bicarbonate.
51. The tablet of embodiment 49, wherein the tablet further comprises sodium bicarbonate.
52. The tablet of any one of embodiments 49-51, further comprising an intragranular phase and an extragranular phase, wherein:
(a) the intragranular phase comprises Compound A monocitrate monohydrate Form A salt dispersed within a first pharmaceutically acceptable excipient; and
(b) the extragranular phase comprises a second pharmaceutically acceptable excipient.
53. The tablet of any one of embodiments 49-52, wherein the Compound A monocitrate monohydrate Form A salt in the tablet is from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt.
54. The tablet of any one of embodiments 49-53, wherein the Compound A monocitrate monohydrate Form A salt is present in the tablet such that Compound A is from about 10% to about 30% wt/wt.
55. The tablet of any one of embodiments 49-54, wherein the Compound A monocitrate monohydrate Form A salt in the tablet is from about 10% to about 30% wt/wt.
56. The tablet of any one of embodiments 49-55, wherein the bicarbonate in the tablet is from about 0.1% to about 10% wt/wt.
57. The tablet of any one of embodiments 49-56, wherein the bicarbonate in the tablet is from about 3% to about 8% wt/wt.
58. The tablet of any one of embodiments 49-57, wherein the bicarbonate in the tablet is about 5% wt/wt.
59. The tablet of any one of embodiments 49-58, wherein the tablet contains Compound A monocitrate monohydrate Form A salt so that the dosage strength is from about 10 to about 300 mg of Compound A.
60. The tablet of embodiment 49, wherein the tablet contains Compound A monocitrate monohydrate Form A salt such that the dosage is about 25 mg, about 110 mg, about 135 mg, or about 160 mg of Compound A.
61. The tablet of embodiment 52, further comprises an intragranular phase and an extragranular phase, wherein:
(a) the intragranular phase comprises:
15% to 30% wt/wt of a Compound A monocitrate monohydrate Form A salt;
30% to 60% wt/wt of microcrystalline cellulose;
5% to 15% wt/wt crospovidone;
5% to 10% wt/wt hypromellose;
0.1% to 7% wt/wt sodium bicarbonate;
0.1% to 2% wt/wt hydrophobic colloidal silica;
0.1% to 2% wt/wt magnesium stearate; and
(b) the extragranular phase comprises:
0.5% to 5% wt/wt magnesium stearate.
62. A liquid formulation prepared by combining the Compound A monocitrate monohydrate Form A salt according to any one of embodiments 1-46, and a pharmaceutically acceptable excipient.
63. The liquid formulation of embodiment 62, further comprising advantame or neotame.
64. The liquid formulation of embodiment 62, further comprising a flavor.
65. The liquid formulation of embodiment 62, further comprising sodium bicarbonate.
66. The liquid formulation of any one of embodiments 62-65, wherein the Compound A monocitrate monohydrate Form A salt used in preparing the liquid formulation is from about 1% to about 10%, from about 1% to about 9%, from about 1% to about 8%, from about 1% to about 7%, from about 2% to about 10%, from about 2% to about 9%, from about 2% to about 8%, from about 2% to about 7%, from about 3% to about 10%, from about 3% to about 9%, from about 3% to about 8%, from about 3% to about 7%, from about 4% to about 10%, from about 4% to about 9%, from about 4% to about 8%, from about 4% to about 7%, from about 5% to about 10%, from about 5% to about 9%, from about 5% to about 8%, or from about 5% to about 7% wt/wt.
67. The liquid formulation of any one of embodiments 62-66, wherein the Compound A monocitrate monohydrate Form A salt used in preparing the liquid formulation from about 1% to about 10% wt/wt.
68. The liquid formulation of embodiment 62, wherein the liquid formulation is prepared by combining:
   from about 1% to about 10% wt/wt of Compound A monocitrate monohydrate Form A salt, and
   from about 0.01% to about 0.1% w/w of advantame or neotame.
69. The liquid formulation of any one of embodiments 62, 68 and 68, wherein the liquid formulation is prepared by combining:
   from about 1% to about 10% wt/wt of Compound A monocitrate monohydrate Form A salt,
   from about 0.01% to about 0.1% w/w of advantame or neotame, and
   from about 0.1% to about 2% w/w of citric acid.
70. The liquid formulation of any one of embodiments 62 and 68-69, wherein the liquid formulation is prepared by combining:
   from about 1% to about 10% wt/wt of Compound A monocitrate monohydrate Form A salt,
   from about 0.01% to about 0.1% w/w of advantame or neotame,
   from about 0.1% to about 2% w/w of citric acid, and
   from about 0.1% to about 2% w/w of sodium chloride.
71. The liquid formulation of any one of embodiments 62 and 68-70, wherein the liquid formulation is prepared by combining:
   from about 1% to about 10% wt/wt of Compound A monocitrate monohydrate Form A salt,
   from about 0.01% to about 0.1% w/w of advantame or neotame,
   from about 0.1% to about 2% w/w of citric acid,
   from about 0.1% to about 2% w/w of sodium chloride, and
   from about 0.1% to about 5% w/w of sodium bicarbonate.
72. The liquid formulation of any one of embodiments 62-71, wherein the liquid formulation has a pH of about 2.0, about 2.5, about 3.0, about 3.5, about 4.0, about 4.5, about 5.0, about 5.5, about 6.0, about 6.5, about 7.0, about 7.5, or about 8.0.
73. The liquid formulation of any one of embodiments 62-72, wherein the liquid formulation has a pH of about 3.0.
74. A method of treating a cancer comprising administering the Compound A monocitrate monohydrate Form A salt, tablet, or liquid formulation of any one of the preceding embodiments to a subject in need thereof.
75. The method of embodiment 74, wherein the cancer is a hematological cancer.
76. The method of either embodiment 74 or 75, wherein the cancer is a leukemia.
77. The method of either embodiment 74 or 75, wherein the cancer is a lymphoma.
78. The method of either embodiment 74 or 75, wherein the cancer is mixed lineage leukemia (MLL), MLL-related leukemia, MLL-associated leukemia, MLL-positive leukemia, MLL-induced leukemia, lysine methyltransferase 2A gene rearrangement (KMT2Ar or KMT2A), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.
79. The method of either embodiment 74 or 75, wherein the cancer is an abstract nucleophosmin (NPM1)-mutated acute myeloid leukemia (i.e., NPM1^{mut} acute myeloid leukemia).
80. The method of either embodiment 74 or 75, wherein the cancer is a lysine methyltransferase 2A gene rearrangement (KMT2Ar or KMT2A).
81. The method of any one of embodiments 74-80, wherein Compound A monocitrate monohydrate Form A salt, tablet, or liquid formulation is administered at a dosage from about 25 to about 300 mg as free base equivalents twice daily.
82. The method of any one of embodiments 74-80, wherein Compound A monocitrate monohydrate Form A salt, tablet, or liquid formulation is administered at a dosage from about 25 mg, 50 mg, 75 mg, 100 mg, 110 mg, 135 mg, 160 mg, 185 mg, 220 mg, or about 270 mg as free base equivalents twice daily.
83. The method of any one of embodiments 74-80, wherein Compound A monocitrate monohydrate Form A salt, tablet, or liquid formulation is administered at a dosage from about 65 mg/m², 95 mg/m², or 160 mg/m² as free base equivalents twice daily.
84. The method of any one of embodiments 74-83, wherein Compound A monocitrate monohydrate Form A salt, tablet, or liquid formulation is administered with a CYP3A4 Inhibitor.
85. The method of any one of embodiments 74-83, wherein Compound A monocitrate monohydrate Form A salt, tablet, or liquid formulation is administered with a strong CYP3A4 Inhibitor.
86. The Compound A monocitrate monohydrate Form A salt, tablet, or formulation of any one of the preceding embodiments for use in treating or preventing a disease caused by, or associated with, menin expression, activity, and/or function.
87. Use of the Compound A monocitrate monohydrate Form A salt of any one of the preceding embodiments for the manufacture of a medicament for treating a cancer to a subject.
88. The Compound A monocitrate monohydrate Form A salt, pharmaceutical composition, or tablet of any one of the preceding embodiments for use in treating a cancer to a subject.
89. The method or use of Compound A monocitrate monohydrate Form A salt, pharmaceutical composition, or tablet of any one of the preceding embodiments, wherein the cancer is a leukemia.
90. A combination of Compound A monocitrate monohydrate Form A salt and a CYP3A4 inhibitor is administered to a subject in need thereof, wherein the amount of Compound A monocitrate monohydrate Form A salt administered is about 25 mg as free base equivalents twice daily.
91. A combination of Compound A monocitrate monohydrate Form A salt and a CYP3A4 inhibitor is administered to a subject in need thereof, wherein the amount of Compound A monocitrate monohydrate Form A salt administered is about 135 mg as free base equivalents twice daily.
92. A combination of Compound A monocitrate monohydrate Form A salt and a CYP3A4 inhibitor is administered to a subject in need thereof, wherein the amount of Compound A monocitrate monohydrate Form A salt administered is about 160 mg as free base equivalents twice daily.
93. A combination of Compound A monocitrate monohydrate Form A salt and a CYP3A4 inhibitor is administered to a subject in need thereof, wherein the amount of Compound A monocitrate monohydrate Form A salt administered is about 110 mg as free base equivalents twice daily.
94. A combination of Compound A monocitrate monohydrate Form A salt and a CYP3A4 inhibitor is administered to a subject in need thereof, wherein the amount of Compound A monocitrate monohydrate Form A salt administered is about 270 mg as free base equivalents twice daily.
95. A combination of Compound A monocitrate monohydrate Form A salt and a CYP3A4 inhibitor is administered to a subject in need thereof, wherein the amount of Compound A monocitrate monohydrate Form A salt administered is about 220 mg as free base equivalents twice daily.
96. A combination of Compound A monocitrate monohydrate Form A salt and a CYP3A4 inhibitor is administered to a subject in need thereof, wherein the dosage of Compound A monocitrate monohydrate Form A salt administered is about 65 mg/m², 95 mg/m², or 160 mg/m² as free base equivalents twice daily.
97. The combination of any one of embodiments 90-96, wherein the subject in need thereof weighs less than 40 kg and wherein the amount of Compound A monocitrate monohydrate Form A salt administered is adjusted by body surface area of the subject in need thereof.
98. A combination of any one of embodiments 90-96, wherein the subject in need thereof is 1.4 m² and weighs less than 40 kg and wherein the amount of Compound A monocitrate monohydrate Form A salt administered is adjusted by body surface area of the subject in need thereof.
99. A combination of Compound A monocitrate monohydrate Form A salt thereof and a CYP3A4 inhibitor is administered to a subject in need thereof, wherein:
(a) the subject in need thereof weighs less than 40 kg,
(b) the amount of Compound A monocitrate monohydrate Form A salt administered is adjusted by body surface area of the subject in need thereof, and
(c) the amount of Compound A monocitrate monohydrate Form A salt is administered in 25 mg (as free base equivalents) tablets.
100. A combination of Compound A monocitrate monohydrate Form A salt thereof and a CYP3A4 inhibitor is administered to a subject in need thereof, wherein:
(a) the subject in need thereof weighs less than 40 kg,
(b) the amount of Compound A monocitrate monohydrate Form A salt administered is adjusted by body surface area of the subject in need thereof, and
(c) the amount of Compound A monocitrate monohydrate Form A salt thereof is administered in 25 mg tablets.
101. A combination of Compound A monocitrate monohydrate Form A salt and a CYP3A4 inhibitor is administered to a subject in need thereof, wherein:
(a) the subject in need thereof weighs less than 40 kg,
(b) the amount of Compound A monocitrate monohydrate Form A salt administered is adjusted by body surface area of the subject in need thereof, and
(c) the amount of Compound A monocitrate monohydrate Form A salt is administered in oral solution.
102. A combination of Compound A monocitrate monohydrate Form A salt and a CYP3A4 inhibitor is administered to a subject in need thereof, wherein the amount of Compound A monocitrate monohydrate Form A salt administered is about 160 mg twice daily.
103. A combination of Compound A monocitrate monohydrate Form A salt and a CYP3A4 inhibitor is administered to a subject in need thereof, wherein the amount of Compound A monocitrate monohydrate Form A salt administered is about 110 mg twice daily.
104. A combination of Compound A monocitrate monohydrate Form A salt and a CYP3A4 inhibitor is administered to a subject in need thereof, wherein the amount of Compound A monocitrate monohydrate Form A salt administered is about 270 mg twice daily.
105. A combination of Compound A monocitrate monohydrate Form A salt and a CYP3A4 inhibitor is administered to a subject in need thereof, wherein the amount of Compound A monocitrate monohydrate Form A salt administered is about 220 mg twice daily.
106. A combination of Compound A monocitrate monohydrate Form A salt thereof and a CYP3A4 inhibitor is administered to a subject in need thereof, wherein:
(a) the subject in need thereof weighs less than 40 kg,
(b) the amount of Compound A monocitrate monohydrate Form A salt administered is adjusted by body surface area of the subject in need thereof, and
(c) the amount of Compound A monocitrate monohydrate Form A salt is administered in 25 mg tablets.
107. A pharmaceutical composition comprising:
from about 15% to about 30% wt/wt of a Compound A monocitrate monohydrate Form A salt

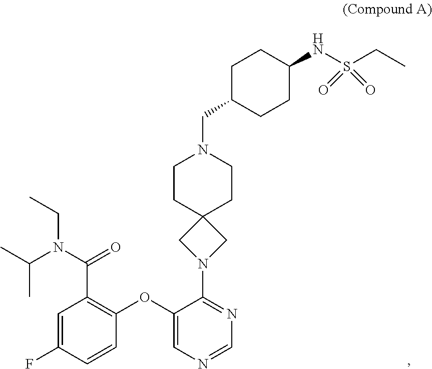 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

and
from about 0.1% to about 10% wt/wt sodium bicarbonate.
108. The pharmaceutical composition of embodiment 107, further comprising from about 30% to about 60% wt/wt of microcrystalline cellulose.
109. The pharmaceutical composition of embodiment 108, further comprising from about 1% to about 10% wt/wt sodium bicarbonate.
110. The pharmaceutical composition of embodiment 109, further comprising from about 0.1% to about 2% wt/wt hydrophobic colloidal silica.
111. The pharmaceutical composition of embodiment 110, further comprising from about 5% to about 15% wt/wt crospovidone.
112. The pharmaceutical composition of embodiment 111, further comprising from about 5% to about 20% wt/wt hypromellose.
113. The pharmaceutical composition of embodiment 112, further comprising from about 0.1% to about 7% wt/wt magnesium stearate.
114. A pharmaceutical composition comprising:
from about 15% to about 30% wt/wt of a Compound A monocitrate monohydrate Form A salt

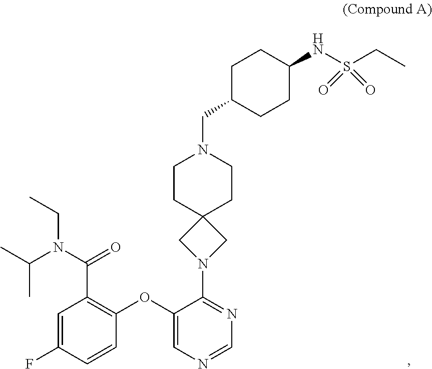 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

from about 30% to about 60% wt/wt of microcrystalline cellulose,
from about 5% to about 15% wt/wt crospovidone,
from about 1% to about 10% wt/wt sodium bicarbonate,
from about 0.1% to about 2% wt/wt hydrophobic colloidal silica, and
from about 0.1% to about 7% wt/wt magnesium stearate.
115. The pharmaceutical composition of embodiment 114, further comprising from about 5% to about 20% wt/wt hypromellose.
116. A pharmaceutical composition comprising an intragranular phase and an extragranular phase, wherein:
(a) the intragranular phase comprises:
from about 15% to about 30% wt/wt of a Compound A monocitrate monohydrate Form A salt

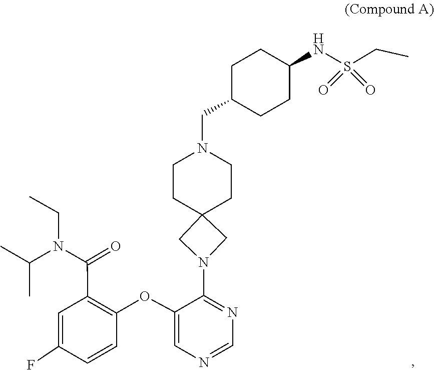 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

and
(b) the extragranular phase comprises at least one pharmaceutically acceptable excipient.
117. The pharmaceutical composition of embodiment 116, wherein the intragranular phase further comprises about 1% to about 10% wt/wt of sodium bicarbonate.
118. The pharmaceutical composition of embodiment 117, wherein the intragranular phase further comprises:
from about 30% to about 60% wt/wt of microcrystalline cellulose;
from about 5% to about 15% wt/wt crospovidone;
from about 5% to about 20% wt/wt hypromellose;
from about 0.1% to about 2% wt/wt hydrophobic colloidal silica;
from about 0.1% to about 2% wt/wt magnesium stearate.
119. A pharmaceutical composition comprising:
from about 15% to about 30% wt/wt of a Compound A monocitrate monohydrate Form A salt calculated as free base equivalents

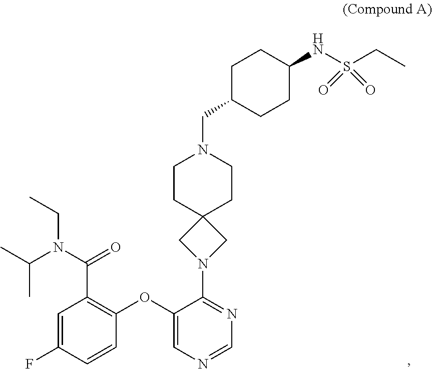 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

and
from about 1% to about 10% wt/wt sodium bicarbonate.
120. The pharmaceutical composition of embodiment 119, further comprising from about 30% to about 60% wt/wt of microcrystalline cellulose.
121. The pharmaceutical composition of embodiment 120, further comprising from about 1% to about 8% wt/wt sodium bicarbonate.
122. The pharmaceutical composition of any one of embodiments 119-121, further comprising from about 0.1% to about 2% wt/wt hydrophobic colloidal silica.
123. The pharmaceutical composition of any one of embodiments 119-122, further comprising from about 5% to about 15% wt/wt crospovidone.
124. The pharmaceutical composition of any one of embodiments 119-123, further comprising from about 5% to about 20% wt/wt hypromellose.
125. The pharmaceutical composition of any one of embodiments 119-124, further comprising from about 0.1% to about 7% wt/wt magnesium stearate.
126. A pharmaceutical composition comprising:
from about 15% to about 30% wt/wt of a Compound A monocitrate monohydrate Form A salt calculated as free base equivalents

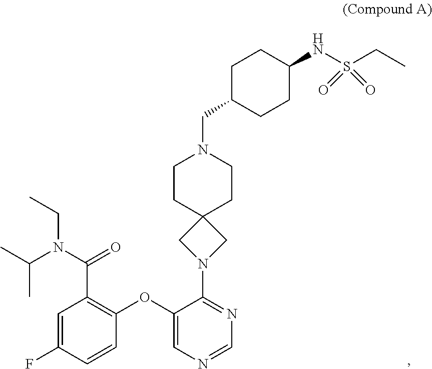 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

from about 30% to about 60% wt/wt of microcrystalline cellulose,
from about 5% to about 15% wt/wt crospovidone,
from about 1% to about 10% wt/wt sodium bicarbonate,
from about 0.1% to about 2% wt/wt hydrophobic colloidal silica, and
from about 0.1% to about 7% wt/wt magnesium stearate.
127. The pharmaceutical composition of embodiment 126, further comprising from about 5% to about 20% wt/wt hypromellose.
128. A pharmaceutical composition comprising an intragranular phase and an extragranular phase, wherein:
(a) the intragranular phase comprises:
from about 15% to about 30% wt/wt of a Compound A monocitrate monohydrate Form A salt calculated as free base equivalents

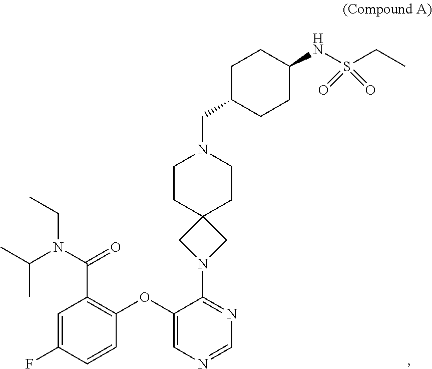 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]

and
(b) the extragranular phase comprises:
from about 0.5% to about 5% wt/wt magnesium stearate.
129. The pharmaceutical composition of embodiment 128, wherein the intragranular phase further comprises about 1% to about 10% wt/wt of sodium bicarbonate.
130. The pharmaceutical composition of embodiment 128 or embodiment 129, wherein the intragranular phase further comprises at least one of the following
from about 30% to about 60% wt/wt of microcrystalline cellulose;
from about 5% to about 15% wt/wt crospovidone;
from about 5% to about 20% wt/wt hypromellose;
from about 0.1% to about 2% wt/wt hydrophobic colloidal silica; and/or
from about 0.1% to about 2% wt/wt magnesium stearate.
131. A method of treating a disease where menin-MLL interaction plays a role comprising administering the Compound A monocitrate monohydrate Form A salt, tablet, or liquid formulation of any one of embodiments 1-73 to a subject in need thereof.
132. A method of treating a disease where menin-MLL interaction plays a role comprising administering the combination of any one of embodiments 90-106 to a subject in need thereof.
133. A method of treating a disease where menin-MLL interaction plays a role comprising administering the pharmaceutical composition of any one of embodiments 107-130 to a subject in need thereof.
134. A method of treating a cancer comprising administering the compound, composition, or combination of any one of the preceding embodiments to a subject in need thereof.
135. A method of treating a leukemia comprising administering the compound, composition, or combination of any one of the preceding embodiments to a subject in need thereof.
136. The method of embodiment 135, wherein the leukemia is mixed lineage leukemia (MLL), MLL-related leukemia, MLL-associated leukemia, MLL-positive leukemia, MLL-induced leukemia, lysine methyltransferase 2A gene rearrangement (KMT2Ar or KMT2A) (also referred to as rearranged mixed lineage leukemia (MLL-r) or KMT2A translocation), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non-Hodgkin's lymphoma (malignant lymphoma), Waldenstrom's macroglobulinemia or abstract nucleophosmin (NPM1)-mutated acute myeloid leukemia (i.e., NPM1mut acute myeloid leukemia).
137. A method for preparing tablets comprising Compound A monocitrate monohydrate Form A salt, the method comprising:
(a) blending Compound A monocitrate monohydrate Form A salt and a pharmaceutically acceptable excipient;
(b) adding a carbonate;
(c) blending; and
(d) compressing the blend into tablets.
138. A method for preparing tablets comprising Compound A monocitrate monohydrate Form A salt, the method comprising:
(a) blending Compound A monocitrate monohydrate Form A salt and a pharmaceutically acceptable excipient to form a pre-blend,
(b) combining the pre-blend with one or more excipients, wherein the one or more excipients comprises a carbonate to form an intragranular blend.
(c) generating milled granules; and
(d) compressing the blend into tablets.

### SECOND LISTING OF NUMBERED EMBODIMENTS

1. A Compound A monocitrate monohydrate Form A salt characterized by PXRD peaks at 11.8°2θ, and 16.9°2θ (±0.2°2θ; ±0.1°2θ; or ±0.0°2θ; Cu Kα1 radiation).
2. The Compound A monocitrate monohydrate Form A salt of embodiment, wherein the Compound A monocitrate monohydrate Form A salt is a salt characterized by PXRD signals at 5.2°2θ, 11.8°2θ, and 16.9°2θ (±0.2°2θ; ±0.1°2θ; or ±0.0°2θ; Cu Kα1 radiation).
3. The Compound A monocitrate monohydrate Form A salt of embodiment 1, wherein the Compound A monocitrate monohydrate Form A salt is a crystalline salt characterized by two or more, or three or more PXRD signals selected from the group consisting of 5.2°2θ, 11.8°2θ, 16.2°2θ, and 16.9°2θ (±0.2°2θ; ±0.1°2θ; or ±0.0°2θ; Cu Kα1 radiation).
4. The Compound A monocitrate monohydrate Form A salt of any one of the preceding embodiments, wherein the Compound A monocitrate monohydrate Form A salt is a crystalline salt characterized by PXRD signals at 5.2°2θ, 11.8°2θ, 16.2°2θ, and 16.9°2θ (±0.2°2θ; ±0.1°2θ; or ±0.0°2θ; Cu Kα1 radiation).
5. The Compound A monocitrate monohydrate Form A salt of any one of the preceding embodiments, wherein the Compound A monocitrate monohydrate Form A salt is a crystalline salt characterized by two or more, or three or more PXRD signals selected from the group consisting of 5.2°2θ, 11.8°2θ, 16.2°2θ, 16.9°2θ, and 19.2°2θ (±0.2°2θ; ±0.1°2θ; or ±0.0°2θ; Cu Kα1 radiation).
6. The Compound A monocitrate monohydrate Form A salt of any one of the preceding embodiments, wherein the Compound A monocitrate monohydrate Form A salt is a crystalline salt characterized by PXRD signals at 5.2°2θ, 11.8°2θ, 16.2°2θ, 16.9°2θ, and 19.2°2θ (±0.2°2θ; ±0.1°2θ; or ±0.0°2θ; Cu Kα1 radiation).
7. The Compound A monocitrate monohydrate Form A salt of any one of the preceding embodiments, wherein the Compound A monocitrate monohydrate Form A salt is a crystalline salt characterized by two or more, or three or more PXRD signals selected from the group consisting of 5.2°2θ, 11.8°2θ, 16.2°2θ, 16.9°2θ, 19.2°2θ, and 20.9°2θ (±0.2°2θ; ±0.1°2θ; or ±0.0°2θ; Cu Kα1 radiation).
8. The Compound A monocitrate monohydrate Form A salt of any one of the preceding embodiments, wherein the Compound A monocitrate monohydrate Form A salt is a crystalline salt characterized by PXRD signals at 5.2°2θ, 11.8°2θ, 16.2°2θ, 16.9°2θ, 19.2°2θ, and 20.9°2θ (±0.2°2θ; ±0.1°2θ; or ±0.0°2θ; Cu Kα1 radiation).
9. The Compound A monocitrate monohydrate Form A salt of any one of the preceding embodiments, wherein the Compound A monocitrate monohydrate Form A salt is a crystalline salt characterized by two or more, or three or more PXRD signals selected from the group consisting of 5.2°2θ, 11.8°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).
10. The Compound A monocitrate monohydrate Form A salt of any one of the preceding embodiments, wherein the Compound A monocitrate monohydrate Form A salt is a crystalline salt characterized by PXRD signals at 5.2°2θ, 11.8°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).
11. The Compound A monocitrate monohydrate Form A salt of any one of the preceding embodiments, wherein the Compound A monocitrate monohydrate Form A salt is a crystalline salt characterized by two or more, or three or more PXRD signals selected from the group consisting of 5.2°2θ, 11.8°2θ, 12.6°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).
12. The Compound A monocitrate monohydrate Form A salt of any one of the preceding embodiments, wherein the Compound A monocitrate monohydrate Form A salt is a crystalline salt characterized by PXRD signals at 5.2°2θ, 11.8°2θ, 12.6°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).
13. The Compound A monocitrate monohydrate Form A salt of any one of the preceding embodiments, wherein the Compound A monocitrate monohydrate Form A salt is a crystalline salt characterized by two or more, or three or more PXRD signals selected from the group consisting of 5.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).
14. The Compound A monocitrate monohydrate Form A salt of any one of the preceding embodiments, wherein the Compound A monocitrate monohydrate Form A salt is a crystalline salt characterized by PXRD signals at 5.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).
15. The Compound A monocitrate monohydrate Form A salt of any one of the preceding embodiments, wherein the Compound A monocitrate monohydrate Form A salt is a crystalline salt characterized by two or more, or three or more PXRD signals selected from the group consisting of 5.2°2θ, 11.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).
16. The Compound A monocitrate monohydrate Form A salt of any one of the preceding embodiments, wherein the Compound A monocitrate monohydrate Form A salt is a crystalline salt characterized by PXRD signals at 5.2°2θ, 11.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).
17. The Compound A monocitrate monohydrate Form A salt of any one of the preceding embodiments, wherein the Compound A monocitrate monohydrate Form A salt is a crystalline salt characterized by one, two, three, four, five, six, seven, eight, nine, ten, eleven, twelve, thirteen, fourteen, fifteen, sixteen, seventeen, eighteen, or nineteen PXRD signals selected from those set forth in Table 1A.
18. The Compound A monocitrate monohydrate Form A salt of any one of the preceding embodiments, wherein the Compound A monocitrate monohydrate Form A salt is a crystalline salt characterized by a PXRD spectrum substantially similar to that shown in FIG. 1A.
19. The Compound A monocitrate monohydrate Form A salt of any one of the preceding embodiments, wherein the Compound A monocitrate monohydrate Form A salt is a 1:1 Compound A:citrate salt.
20. The Compound A monocitrate monohydrate Form A salt of any one of the preceding embodiments, wherein the Compound A monocitrate monohydrate Form A salt has a particle size Dx(90) of about 15 μm to about 25 μm.
21. The Compound A monocitrate monohydrate Form A salt of any one of the preceding embodiments, wherein the Compound A monocitrate monohydrate Form A salt has a median particle size of about 8 μm to about 25 μm.
22. The Compound A monocitrate monohydrate Form A salt of any one of the preceding embodiments, wherein the Compound A monocitrate monohydrate Form A salt has an average particle size of about 8 μm to about 25 μm.
23. The Compound A monocitrate monohydrate Form A salt of any one of the preceding embodiments, wherein the Compound A monocitrate monohydrate Form A salt has a particle size Dx(50) of about 5 μm to about 10 μm.
24. The Compound A monocitrate monohydrate Form A salt of any one of the preceding embodiments, wherein the Compound A monocitrate monohydrate Form A salt has a particle size Dx(10) of about 1 μm to about 5 μm.
25. A pharmaceutical composition comprising the Compound A monocitrate monohydrate Form A salt of any one of the preceding embodiments and a pharmaceutically acceptable excipient.
26. A pharmaceutical composition comprising:
from about 15% to about 30% wt/wt of Compound A as the Compound A monocitrate monohydrate Form A salt of any one of embodiments 1-24, and
from about 0.1% to about 10% wt/wt sodium bicarbonate.
27. The pharmaceutical composition of embodiment 25 or embodiment 26, comprising from about 30% to about 60% wt/wt of microcrystalline cellulose.
28. The pharmaceutical composition of any one of embodiments 25-27, comprising from about 1% to about 10% wt/wt sodium bicarbonate.
29. The pharmaceutical composition any one of embodiments 25-28, comprising from about 0.1% to about 2% wt/wt hydrophobic colloidal silica.
30. The pharmaceutical composition any one of embodiments 25-29, comprising from about 5% to about 15% wt/wt crospovidone.
31. The pharmaceutical composition of any one of embodiments 25-30, comprising from about 5% to about 20% wt/wt hypromellose.
32. The pharmaceutical composition any one of embodiments 25-31, comprising from about 0.1% to about 7% wt/wt magnesium stearate.
33. A pharmaceutical composition comprising an intragranular phase and an extragranular phase, wherein:
(a) the intragranular phase comprises:
from about 15% to about 30% wt/wt of Compound A as the Compound A monocitrate monohydrate Form A salt of any one of embodiments 1-24, and
(b) the extragranular phase comprises at least one pharmaceutically acceptable excipient.
34. The pharmaceutical composition any one of embodiments 25-33, wherein the intragranular phase further comprises about 1% to about 10% wt/wt of sodium bicarbonate.
35. The pharmaceutical composition of any one of embodiments 25-34, comprising from about 1% to about 8% wt/wt sodium bicarbonate.
36. The pharmaceutical composition of any one of embodiments 25-35, comprising from about 1% to about 6% wt/wt sodium bicarbonate.
37. The pharmaceutical composition of any one of embodiments 25-36, wherein the intragranular phase further comprises at least one of the following:
from about 30% to about 60% wt/wt of microcrystalline cellulose;
from about 5% to about 15% wt/wt crospovidone;
from about 5% to about 20% wt/wt hypromellose;
from about 0.1% to about 2% wt/wt hydrophobic colloidal silica; and/or
from about 0.1% to about 2% wt/wt magnesium stearate.
38. The pharmaceutical composition of any one of embodiments 25-37, comprising from about 0.1% to about 2% wt/wt hydrophobic colloidal silica.
39. The pharmaceutical composition of any one of embodiments 25-38, comprising from about 5% to about 15% wt/wt crospovidone.
40. The pharmaceutical composition of any one of embodiments 25-39, comprising from about 5% to about 20% wt/wt hypromellose.
41. The pharmaceutical composition of any one of embodiments 25-40, further comprising from about 0.1% to about 7% wt/wt magnesium stearate.
42. A pharmaceutical composition comprising:
from about 15% to about 30% wt/wt of Compound A as the Compound A monocitrate monohydrate Form A salt (calculated as free base equivalents)
from about 30% to about 60% wt/wt of microcrystalline cellulose,
from about 5% to about 15% wt/wt crospovidone,
from about 1% to about 10% wt/wt sodium bicarbonate,
from about 0.1% to about 2% wt/wt hydrophobic colloidal silica, and
from about 0.1% to about 7% wt/wt magnesium stearate.
43. The pharmaceutical composition of embodiment 42, further comprising from about 5% to about 20% wt/wt hypromellose.
44. A pharmaceutical composition comprising an intragranular phase and an extragranular phase, wherein:
(a) the intragranular phase comprises:
from about 15% to about 30% wt/wt of Compound A as the Compound A monocitrate monohydrate Form A salt; and
(b) the extragranular phase comprises:
from about 0.5% to about 5% wt/wt magnesium stearate.
45. The pharmaceutical composition of embodiment 44, wherein the intragranular phase further comprises about 1% to about 10% wt/wt of sodium bicarbonate.
46. The pharmaceutical composition of embodiment 44 or embodiment 45, wherein the intragranular phase further comprises at least one of the following
from about 30% to about 60% wt/wt of microcrystalline cellulose;
from about 5% to about 15% wt/wt crospovidone;
from about 5% to about 20% wt/wt hypromellose;
from about 0.1% to about 2% wt/wt hydrophobic colloidal silica; and/or
from about 0.1% to about 2% wt/wt magnesium stearate.
47. The pharmaceutical composition of any one of the previous embodiments, wherein the composition is a tablet.
48. The tablet of embodiment 47, wherein the tablet is orally administered.
49. The tablet of embodiment 47 or 48, wherein the Compound A in the tablet is from about 1% to about 5%, from about 1% to about 10%, from about 1% to about 15%, from about 1% to about 20%, from about 1% to about 25%, from about 1% to about 30%, from about 1% to about 35%, or from about 1% to about 40% wt/wt, wherein the Compound A is present as the Compound A monocitrate monohydrate Form A salt.
50. The tablet of any one of embodiments 47-49, wherein the Compound A monocitrate monohydrate Form A salt is present in the tablet such that Compound A is from about 10% to about 30% wt/wt.
51. The tablet of any one of embodiments 47-50, wherein the Compound A monocitrate monohydrate Form A salt in the tablet is from about 10% to about 30% wt/wt.
52. The tablet of any one of embodiments 47-51, wherein the sodium bicarbonate in the tablet is from about 0.1% to about 10% wt/wt.
53. The tablet of any one of embodiments 47-52, wherein the sodium bicarbonate in the tablet is from about 1% to about 8% wt/wt.
54. The tablet of any one of embodiments 47-53, wherein the sodium bicarbonate in the tablet is about 2% to about 6% wt/wt.
55. The tablet of any one of embodiments 47-54, wherein the tablet contains Compound A monocitrate monohydrate Form A salt so that the dosage strength is from about 10 to about 300 mg of Compound A.
56. The tablet of any one of embodiments 47-55, wherein the tablet contains Compound A monocitrate monohydrate Form A salt such that the dosage is about 25 mg, about 110 mg, about 135 mg, or about 160 mg of Compound A.
57. The tablet of any one of embodiments 47-56, wherein
(a) the intragranular phase comprises:
   15% to 30% wt/wt of a Compound A monocitrate monohydrate Form A salt and at least one of the following:
10% to 60% wt/wt of microcrystalline cellulose;
5% to 10% wt/wt hypromellose;
0.1% to 7% wt/wt sodium bicarbonate;
0.1% to 2% wt/wt magnesium stearate; and
(b) the extragranular phase comprises:
0.1% to 5% wt/wt magnesium stearate.
58. A liquid formulation comprising Compound A, and a pharmaceutically acceptable carrier.
59. The liquid formulation of embodiment 58, further comprising advantame or neotame.
60. The liquid formulation of embodiment 58 or 59, further comprising an alkalizer.
61. The liquid formulation of any one of embodiments 58-60, wherein the Compound A in the liquid formulation is from about 1% to about 50%, from about 1% to about 45%, from about 1% to about 40%, from about 10% to about 35%, from about 10% to about 30%, from about 10% to about 25%, from about 1% to about 20%, from about 1% to about 10%, from about 1% to about 9%, from about 1% to about 8%, from about 1% to about 7%, from about 2% to about 10%, from about 2% to about 9%, from about 2% to about 8%, from about 2% to about 7%, from about 3% to about 10%, from about 3% to about 9%, from about 3% to about 8%, from about 3% to about 7%, from about 4% to about 10%, from about 4% to about 9%, from about 4% to about 8%, from about 4% to about 7%, from about 5% to about 10%, from about 5% to about 9%, from about 5% to about 8%, or from about 5% to about 7% wt/wt.
62. The liquid formulation of any one of embodiments 58-61, wherein the liquid formulation is prepared using the Compound A monocitrate monohydrate Form A salt.
63. The liquid formulation of any one of embodiments 58-62, wherein the liquid formulation comprises from about 0.01% to about 0.1% w/w of advantame or neotame.
64. The liquid formulation of any one of embodiments 58-63, wherein the liquid formulation comprises from about 0.1% to about 5% w/w of citric acid.
65. The liquid formulation of any one of embodiments 58-64, wherein the liquid formulation comprises from about 0.1% to about 2% w/w of sodium chloride.
66. The liquid formulation of any one of embodiments 58-65, wherein the liquid formulation comprises from about 0.1% to about 5% w/w of sodium bicarbonate.
67. The liquid formulation of any one of embodiments 58-66, wherein the liquid formulation has a pH of about 2.0 to about 8.0; about 2.0 to about 7.0; about 2.0 to about 6.0; about 2.0 to about 5.0; about 2.0 to about 4.5; about 2.0 to about 4.0; about 2.0 to about 3.5; about 2.0 to about 3.0; about 2.5 to about 8.0; about 2.5 to about 7.0; about 2.5 to about 6.0; about 2.5 to about 5.0; about 2.0 to about 4.5; about 2.5 to about 4.0; about 2.5 to about 3.5; or about 2.5 to about 3.0.
68. The liquid formulation of any one of embodiments 58-67, wherein the liquid formulation has a pH of about 2.0, about 2.5, about 3.0, about 3.5, about 4.0, about 4.5, about 5.0, about 5.5, about 6.0, about 6.5, about 7.0, about 7.5, or about 8.0.
69. The liquid formulation of any one of embodiments 58-68, wherein the liquid formulation has a pH of about 3.0.
70. A method of treating a disease where menin-MLL interaction plays a role comprising administering the Compound A monocitrate monohydrate Form A salt, tablet, or liquid formulation of any one of the previous embodiments to a subject in need thereof.
71. A method of treating a disease where menin-MLL interaction plays a role comprising administering the pharmaceutical composition of any one of the previous embodiments to a subject in need thereof.
72. A method of treating a cancer comprising administering the Compound A monocitrate monohydrate Form A salt, pharmaceutical composition, tablet, or liquid formulation of any one of the preceding embodiments to a subject in need thereof.
73. The method of embodiment 72, wherein the cancer is a hematological cancer.
74. The method of either embodiment 72 or embodiment 73, wherein the cancer is a leukemia.
75. The method of either embodiment 72 or embodiment 73, wherein the cancer is a lymphoma.
76. The method of any one of embodiments 72-74, wherein the cancer is mixed lineage leukemia (MLL), MLL-related leukemia, MLL-associated leukemia, MLL-positive leukemia, MLL-induced leukemia, lysine methyltransferase 2A gene rearrangement (KMT2Ar or KMT2A), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.
77. The method of any one of embodiments 72-74, wherein the cancer is an abstract nucleophosmin (NPM1)-mutated acute myeloid leukemia (i.e., NPM1mut acute myeloid leukemia).
78. The method of any one of embodiments 72-74, wherein the cancer is a lysine methyltransferase 2A gene rearrangement (KMT2Ar or KMT2A).
79. The method of any one of embodiments 70-78, wherein Compound A monocitrate monohydrate Form A salt, pharmaceutical composition, tablet, or liquid formulation is administered at a dosage of Compound A from about 25 to about 300 mg as free base equivalents twice daily.
80. The method of any one of embodiments 70-79, wherein Compound A monocitrate monohydrate Form A salt, pharmaceutical composition, tablet, or liquid formulation is administered at a dosage of Compound A of about 25 mg, 50 mg, 75 mg, 100 mg, 110 mg, 135 mg, 160 mg, 185 mg, 220 mg, or about 270 mg as free base equivalents twice daily.
81. The method of any one of embodiments 70-80, wherein Compound A monocitrate monohydrate Form A salt, pharmaceutical composition, tablet, or liquid formulation is administered at a dosage from about 65 mg/m2, 95 mg/m2, or 160 mg/m2 as free base equivalents twice daily.
82. The method of any one of embodiments 70-81, wherein Compound A monocitrate monohydrate Form A salt, pharmaceutical composition, tablet, or liquid formulation is administered without regard to food.
83. A method for preparing tablets comprising Compound A monocitrate monohydrate Form A salt, the method comprising:
(a) blending Compound A monocitrate monohydrate Form A salt and a pharmaceutically acceptable excipient;
(b) adding a carbonate;
(c) blending; and
(d) compressing the blend into tablets.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort can be had to various other embodiments, modifications, and equivalents thereof which can suggest themselves to those skilled in the art without departing from the spirit of the present disclosure and/or scope of the appended claims.

### Example 1. Synthesis of Compound A Monocitrate Monohydrate

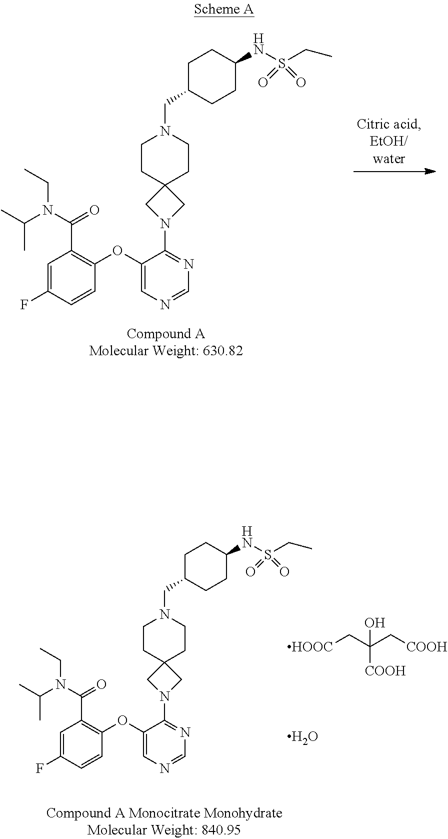

Methods for synthesizing Compound A are known in the art, for example, the synthesis of Compound A is described in PCT Patent Appl. Publication No. WO 2017/214367.

Charge Compound A and ethanol (9 L/Kg). Adjust the batch temp to 50-60° C. Polish filter the solution to another reactor to remove any extraneous materials. Charge (via polish filter) a solution of citric acid monohydrate (1.2 equiv) in water (1 L/Kg) at 50-60° C. Rinse the polish filter with ethanol (1 L/Kg). Maintain the batch at 50-60° C. for at least 0.5 h. Cool the batch to 46-56° C. and add seeds of Compound A monocitrate monohydrate (1.0-5.0% w/w). Stir the thin slurry at 46-56° C. for at least 0.5 h. Cool the batch to 20° C. at 0.1 C/min rate. Add ethanol (9 L/Kg) over a period of 8-10 h at 15-20° C. Stir the slurry for at least 4 h at 20° C. Cool the batch to 5 C (0-10° C.) at 0.1° C./min rate. Age the slurry at 0-10° C. for at least 18 h. Collect the solids by filtration and wash the cake with cold (0-10° C.) 99:1 EtOH/water (2 L/Kg). Dry the solids at 35-45° C. (target 40° C.). IPC: EtOH ≤2500 ppm. Dried solids are Compound A monocitrate monohydrate. Typical yields 65-75%.

### Example 2. Single Crystal Structures of Compound A Monocitrate Salt

Two crystal structures of Compound A monocitrate, one a monomethanolate and the other a monohydrate, were solved. They are isomorphous.

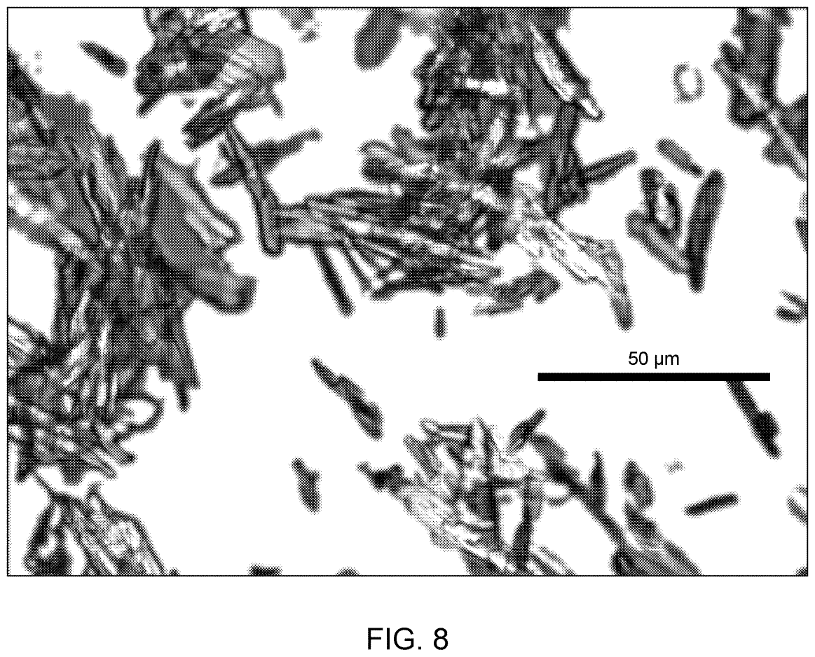

A PLM image of Compound A monocitrate monohydrate Form A salt is depicted in FIG. 8.

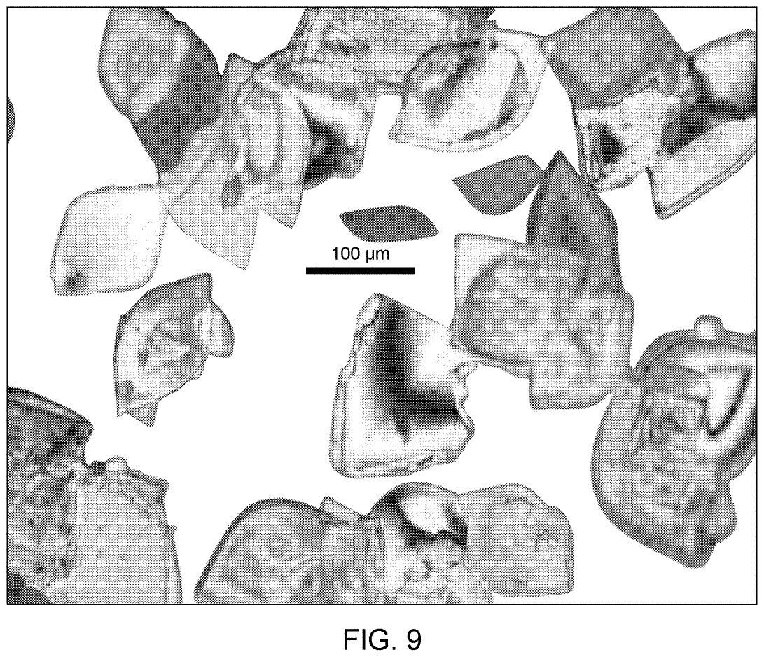

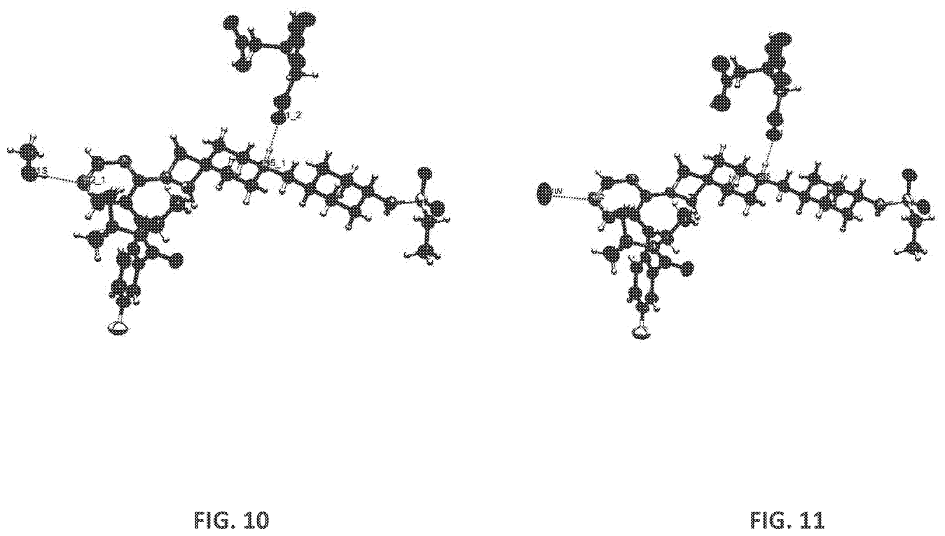

Plate-like crystals (FIG. 9) were grown by recrystallization from methanol of Compound A monocitrate monomethanolate by evaporation (prepared from methanol/water 90:10 v/v). X-ray diffraction data were collected from a freshly isolated crystal which was cooled at 223 K. The crystal structure solved from the data revealed a monocitrate monomethanolate (FIG. 10). The same crystal was analyzed again at 273 K after readily exposure to ambient conditions, and the solution showed that the original methanolate had transformed into a monohydrate (FIG. 11).

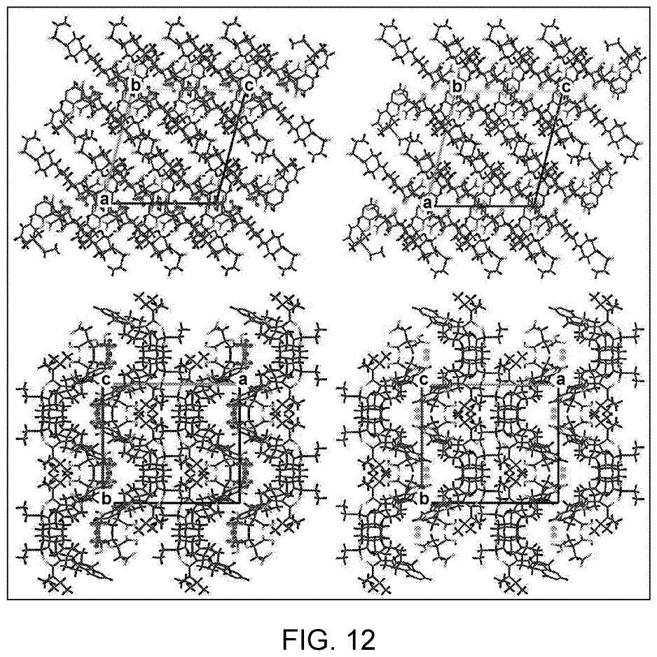

The two isomorphic structures contained solvent channels parallel to crystallographic b- and c-axes, occupied by methanol and water, respectively (FIG. 12). Methanol is readily replaced by water under ambient conditions.

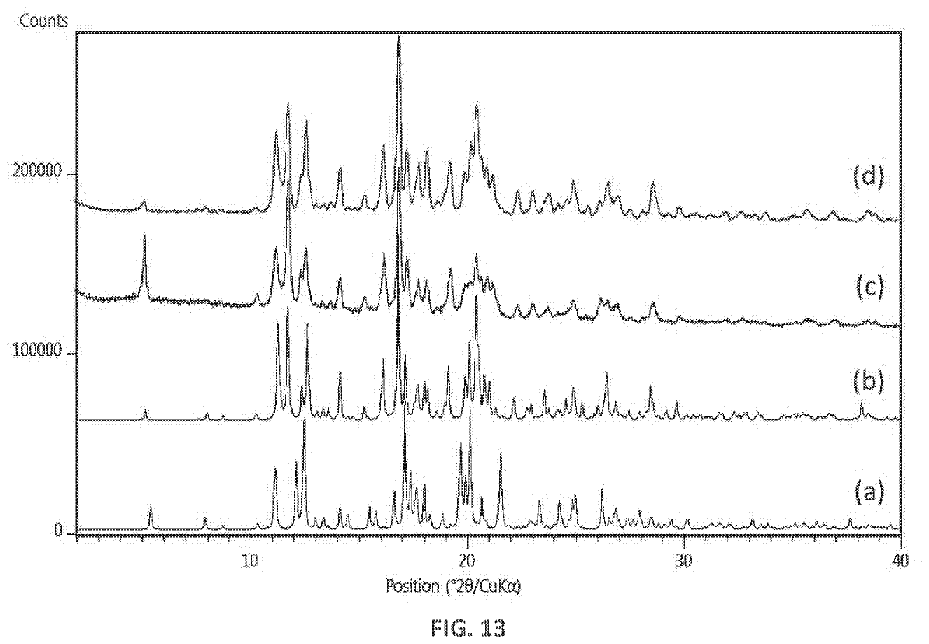

The simulated XRPD pattern of this monohydrate matched the bulk solid (air-dried) from which the single crystal was isolated, as well as the (oven-dried) bulk solids prepared from acetone-water (FIG. 13).

### Example 3. Polymorphs of Compound A Citrate Salt

Two additional forms were discovered: Form B from slurrying in methanol, and Form C from slurrying in ACN. As described in the below Form B converts to Form A upon drying. Form C can be isolated as a dried solid but converts to Form A upon exposure to humidity.

### Polymorph Analysis of Compound A Monocitrate Monohydrate Form A Salt

Based on the approximate solubility data of Compound A citrate salt, 18 polymorph experiments were started at RT with stirring (Table 9). Wet cakes from the slurry experiments were analyzed after 5 days of stirring. Wet cake XRPD patterns were checked and materials showing no pattern change were heated from RT to 50° C.

### Procedure

1) Slurry at RT: ~35 mg citrate salt was slurried in different solvents in 4 mL vials using a magnetic stirrer at RT. The solids in slurry were characterized by XRPD after 3-5 days.
2) Slurry at 50° C.: Slurries with no change in wet cake XRPD pattern when compared to starting material were heated from RT to 50° C. Samples were analyzed after 2-3 days of stirring.

Wet cake samples were isolated and kept at 25° C. for 2 h for drying. After drying, samples were analyzed by XRPD. Two new patterns were observed, one from MeCN designated as Form C and one from MeOH designated as Form B. Upon drying, the MeOH slurry sample changed pattern from Form B to Form A, as expected based on previously established knowledge from single-crystal analysis. The DSC-TGA data for the solid obtained from MeCN are shown in FIG. 15.

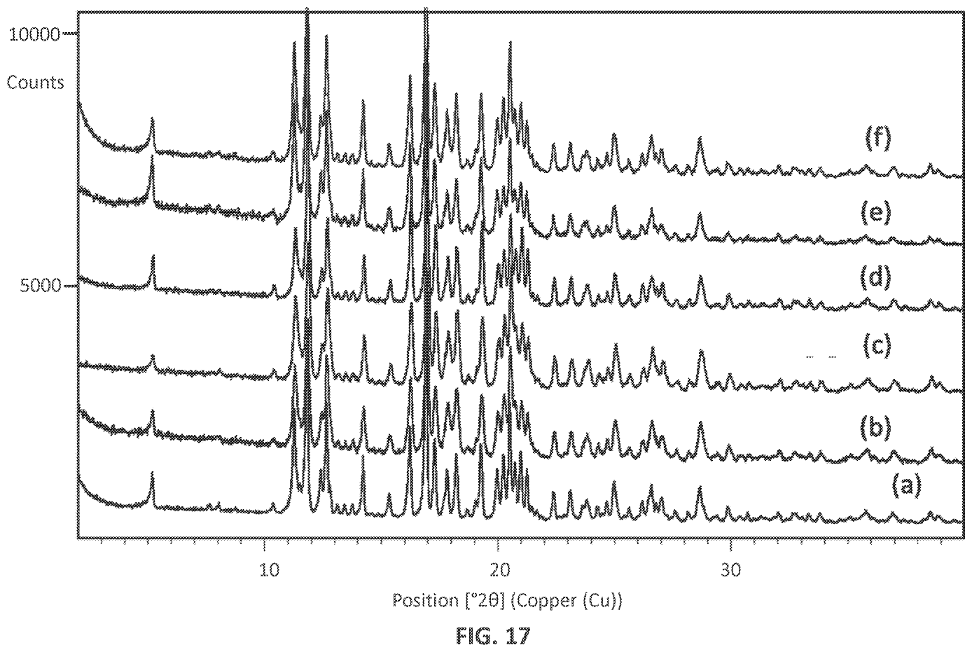

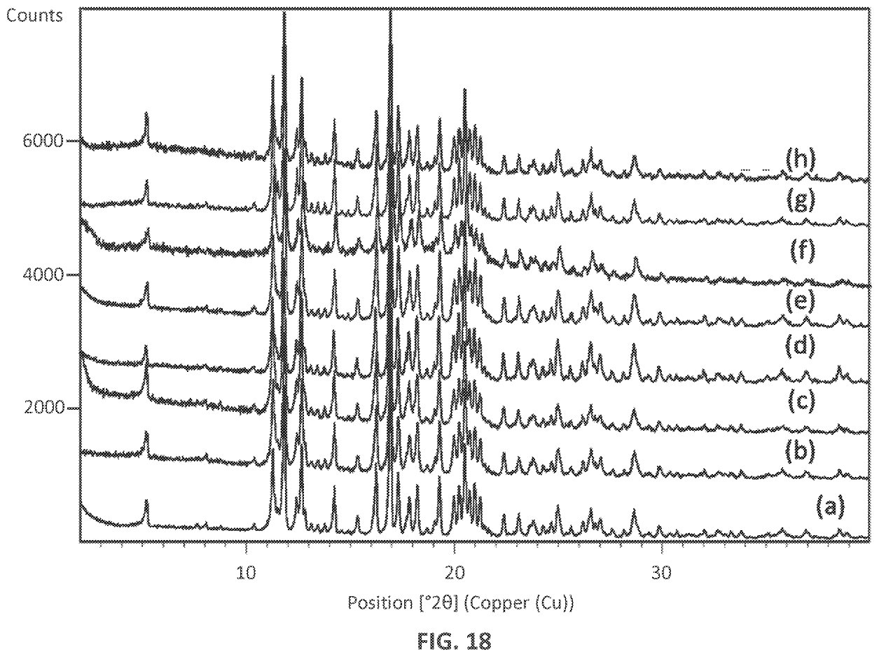

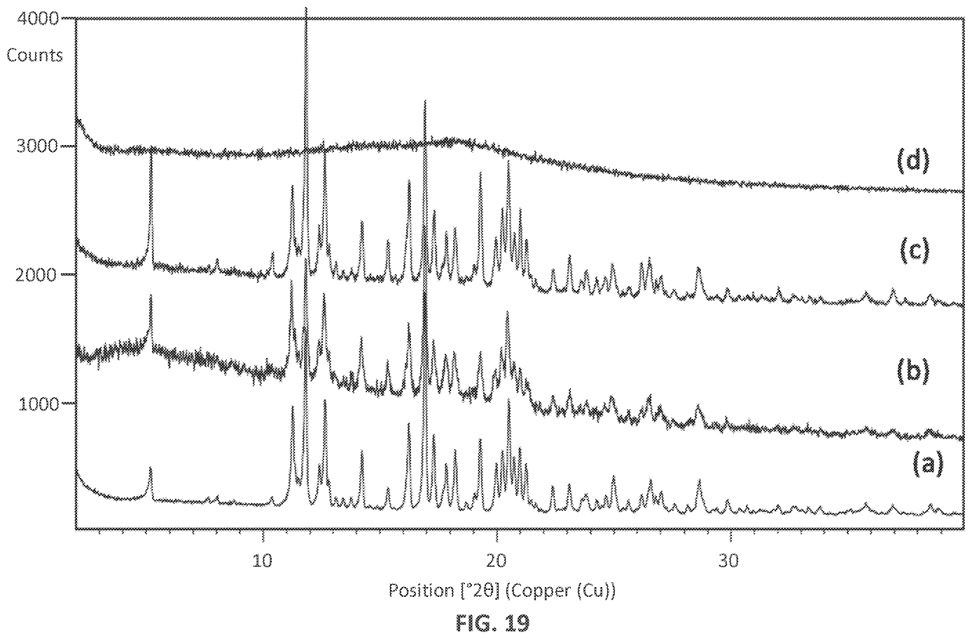

TABLE 9
Eighteen slurry and solvent evaporation experiments
of Compound A monocitrate monohydrate Form A salt

| Experiment No. | Solvent | Form |
|---|---|---|
| 1 | MeCN | Form C (FIG. 14) |
| 2 | EtOAc | Form A (FIG. 17) |
| 3 | MeOH or EtOH | Form B (Wet cake)→ air |
| | | dry→Form A (FIG. 16) |
| 4 | MEK | Form A (FIG. 17) |
| 5 | THF | Form A (FIG. 17) |
| 6 | DCM | Form A (FIG. 17) |
| 7 | Toluene | Form A (FIG. 17) |
| 8 | IPAc | Form A (FIG. 18) |
| 9 | MTBE | Form A (FIG. 18) |
| 10 | H₂O | Form A (FIG. 18) |
| 11 | 2-Me-THF* | Form A (FIG. 18) |
| 12 | DMF | CL |
| | DMF (SE) | CL |
| 13 | MeOH/H₂O (7:3) | CL |
| 14 | THF/H₂O (7:3) | CL |
| | THF/H₂O (7:3) (SE) | Amorphous (FIG. 19) |
| 15 | Acetone/H₂O (7:3) | Form A (FIG. 18) |
| 16 | Propanol/H₂O (7:3) | Form A (FIG. 18) |
| | Propanol/H₂O (7:3) (SE) | Form A (FIG. 19) |
| 17 | EtOAc/DMF (1:1) | CL |
| 18 | 1,4-dioxane/H₂O (7:3) | Form A (FIG. 18) |
| | 1,4-dioxane/H₂O (7:3) (SE) | Form A (FIG. 19) |

CL: clear solution; SE: Solvent evaporation;
*Starting material used was 1:1 mixture of Compound A monocitrate monohydrate Form A salt.

### Further Characterization of Form C

An experiment preparing Form C at 300-mg scale was completed; isolated material was dried under vacuum at 40° C. with a N₂ bleed and analyzed using XRPD (FIG. 20, FIG. 22), DSC and TGA (FIG. 15, FIG. 23), DVS (FIG. 21), PLM (FIG. 24), GC for residual solvent, and NMR for stoichiometry (Table 10).

### Procedure

Approximately 300 mg of Form A was weighed into a 4 mL glass vial and 2.5 mL of MeCN was added slowly to make a slurry. The sample vial was kept at 50° C. with stirring for 3 days. After 3 days, slurry material was isolated and dried at 40° C. with a N₂ bleed.

TABLE 10
Results of scale-up preparation of Compound A citrate Form C

| | | | DVS (weight | |
|---|---|---|---|---|
| Starting | % | XRPD/DSC/ | gain between | |
| material | Yield | TGA/NMR | 30%-80% RH) | Comment |
| Form A | 95.5 | Matched | 1. 1^{st} adsorption | 1. Free Base: Acid = |
| | | Form C data | cycle 1.6% | 1:1 by ¹H NMR; |
| | | | 2. 2^{nd} adsorption | 2. H₂O content by |
| | | | cycle 6.1% | oven evaporator KF = |
| | | | | 4.95% |
| | | | 3. Post DVS form | 3. Acetone content |
| | | | changed to Form- | by GC: 106 ppm |
| | | | A | 4. MeCN content by |
| | | | | GC: 410 ppm |

### Example 4. Compound A Monocitrate Monohydrate

Compound A monocitrate monohydrate has at least two advantageously and unexpected characteristics. First, the fumarate counterion reacts with the pyrimidine ring of Compound A resulting in degradation; whereas the citrate counterion does not react with the pyrimidine ring of Compound A. Solution stability studies at accelerated conditions confirmed the absence of this type of degradation. Second, in the manufacturing process, Compound A fumarate requires a separate slurrying step which results in a solvate with an organic solvent. Compound A monocitrate monohydrate salt is isolated directly from ethanol:water, no slurrying step is required. Accordingly, this manufacturing process does not require multiple steps and results in increased yields (no losses during the slurrying step). Additionally, the Compound A monocitrate forms a monohydrate instead of a solvate with one or more organic molecules.

### Example 5. Compound A Sesquifumarate Capsule Formulations

The acute leukemia population with a KMT2A rearrangement consists of both adult and pediatric patients. Solid oral dose forms are more convenient for patients thus a solid oral dose was developed for adults and children able to swallow. Both capsule and tablet solid oral dose forms were generated.

The capsule formulations utilized the sesquifumarate salt form of Compound A and were supplied as either active pharmaceutical ingredient in capsule (AIC) or blend in capsule (BIC) product.

The development work began using the sesquifumarate salt form of Compound A. The composition of Compound A capsules is provided in Table 11.

TABLE 11
Composition of Compound A Clinical Capsule Formulation
(25 mg AIC, 156 mg AIC, and 113 mg AIC and BIC)

| | | | Amount per Capsule (mg) | | | |
|---|---|---|---|---|---|---|
| | Quality | Component | 25 mg | 113 mg | 156 mg | 113 mg |
| Component | Standard | Function | AIC | AIC | AIC | BIC |
| Compound A | In-house | Active | 25.0^{a} | 113.0^{a} | 156.0^{a} | 113.00^{a} |
| sesquifumarate | | | | | | |
| Microcrystalline | NF, Ph. | Filler | N/A | N/A | N/A | 70.92 |
| cellulose | Eur, JP | | | | | |
| Magnesium | NF, Ph. | Lubricant | N/A | N/A | N/A | 1.08 |
| stearate | Eur. | | | | | |
| HPMC Capsule | N/A^{b} | Encapsulation | N/A | Each | N/A | Each |
| Size 1, Swedish | | | | | | |
| Orange | | | | | | |
| HPMC Capsule | N/A^{b} | Encapsulation | N/A | N/A | Each | N/A |
| Size 0, White | | | | | | |
| Opaque | | | | | | |
| HPMC Capsule, | N/A^{b} | Encapsulation | Each | N/A | N/A | NA |
| Size 0, Natural | | | | | | |
| Transparent | | | | | | |

^{a}Value expressed as free base. 25 mg is equivalent to 32 mg sesquifumarate salt, 113 mg is equivalent to 145 mg sesquifumarate salt, and 156 mg is equivalent to 200 mg sesquifumarate salt.
^{b}The HPMC capsule is non-compendial; however, the capsule components comply with compendial requirements.
AIC = API-in-capsule;
BIC = blend-in-capsule;
HPMC = hydroxypropyl methylcellulose;
JP = Japanese Pharmacopoeia;
N/A = Not applicable;
NF = National Formulary;
Ph. Eur. = European Pharmacopoeia.

### 25 mg API in Capsule

Compound A sesquifumarate salt meets the Biopharmaceutical Classification System definition of a highly soluble drug substance. Given the high solubility of the drug substance, the preferred formulation approach for initial clinical dosing was to encapsulate neat Compound A sesquifumarate in a capsule.

Compound A sesquifumarate was filled into size 0 capsules using Xcelodose® 120 encapsulation equipment to assess the maximum amount of drug that a Size 0 capsule could hold. Size 0 capsules were selected as the maximum acceptable size for dosing multiple units. Based on the feasibility evaluation, 25 mg (equivalent to 32 mg Compound A sesquifumarate salt) was the maximum amount of Compound A sesquifumarate capable of being filled into a Size 0 capsule using an Xcelodose®. In addition, drug substance poor flow properties meant that larger batch sizes of 25 mg capsules were not efficient to manufacture. Therefore, although 25 mg Compound A capsules were manufactured using Xcelodose® 120 equipment, another manufacturing process was required for higher dose strength.

The composition of 25 mg AIC is shown in Table 11. The 25 mg AIC drug product is stored at 2° C. to 8° C.

### 113 mg and 156 mg Active in Capsule

Higher capsule dose strengths required the ability to tamp down solids in the capsule and remove void spaces. Operators fill trays of capsules by manually brushing solids across the capsule openings, then tamp down the solids and repeat the process until the capsules are filled to the maximum capacity. A single batch of 113 mg drug product in Size 1 capsules and a single batch of 156 mg drug product in Size 0 capsules were manufactured from neat Compound A sesquifumarate drug substance using the MF-30 equipment. The composition of 113 mg and 156 mg AIC is shown in Table 11. The 113 mg and 156 mg AIC drug products are stored at 2° C. to 8° C.

### 113 mg Blend in Capsule

Additional batches of Compound A sesquifumarate drug substance were manufactured and it was observed that different drug substance batches had some variability in bulk density, which required development of a BIC formulation for volumetric encapsulation. The formulation was expected to require a filler and a lubricant. An excipient compatibility study was performed evaluating different fillers and lubricants in binary mixtures over 8 weeks at two accelerated conditions: 25° C./60% relative humidity (RH) or 40° C./75% RH. Compound A sesquifumarate capsule drug product is stored at 2° C. to 8° C., therefore both storage conditions for the excipient compatibility study represent thermal stress conditions.

Microcrystalline cellulose (Avicel PH-113) and magnesium stearate were chosen as excipients based on chemical and physical test results. A formulation for manual encapsulation process using an MF-30 encapsulator was developed using these two excipients. The final blend formula was Compound A sesquifumarate: 66.82%, microcrystalline cellulose: 32.68% and magnesium stearate: 0.50%.

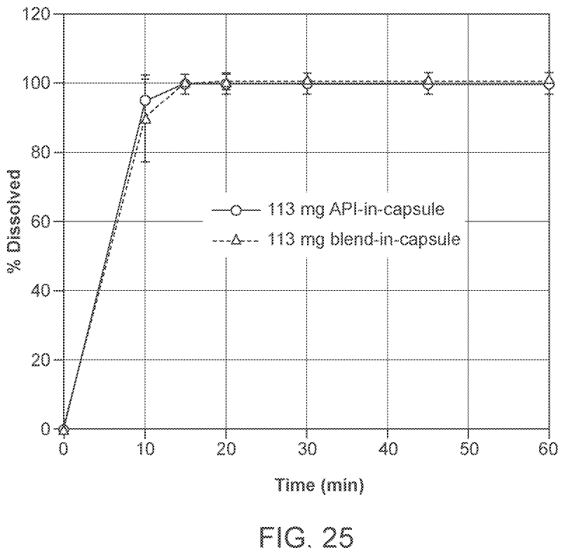

Dissolution profiles for capsules made with this blend were compared to capsules containing neat drug substance, shown in FIG. 25. Dissolution profiles were similar and extremely fast for both formulations, due to Compound A's high solubility.

In conclusion, a capsule formulation for Compound A sesquifumarate salt form was developed containing Avicel PH-113 and magnesium stearate based on an excipient compatibility study. Capsules manufactured using this formulation have similar performance to capsules containing neat drug substance, as evidenced by dissolution profiles. The composition of 113 mg BIC drug product is shown in Table 11. The 113 mg BIC drug product is stored at 2° C. to 8° C.

### Example 6. Compound A Monocitrate Monohydrate Tablets

The goal of this study was to develop a film coated immediate release tablets with similarly rapid dissolution profiles as the capsule drug product. The formulation was developed with three key design requirements:
Dissolution: Maintain comparable dissolution profile and pharmacokinetic properties with the capsule formulation to ensure equivalent performance.
Manufacturability: The required high drug loading combined with poor flowability of drug substance prompted development of a tablet formulation to ensure a robust, scalable manufacturing process.
Stability: The final drug product must be chemically and physically stable during bulk storage, distribution, and the duration of the product's shelf life.

Compound A monocitrate monohydrate tablets are prepared in 3 strengths—160 mg, 110 mg, and 25 mg (reported as free base equivalents). Tablet composition is listed in Tables 12 and 13.

TABLE 12
Formulation Composition (% w/w) for 25 mg,
110 mg and 160 mg Compound A Tablets

| | Concentration | | |
|---|---|---|---|
| Material | (% w/w) | | |
| Tablet Strength | 25 mg | 110 mg | 160 mg |

Intra-granular

| | | | |
|---|---|---|---|
| Compound A monocitrate | 23.8 | 23.8 | 23.8 |
| monohydrate¹ | | | |
| Avicel-DG ®* | 45.2 | 45.2 | 45.2 |
| Crospovidone, NF/EP | 9.5 | 9.5 | 9.5 |
| Hypromellose, USP | 9.5 | 9.5 | 9.5 |
| Sodium bicarbonate, NF/EP | 4.8 | 4.8 | 4.8 |
| Hydrophobic colloidal | 1.0 | 1.0 | 1.0 |
| silica, NF/EP | | | |
| Magnesium stearate, NF, | 0.5 | 0.5 | 0.5 |
| vegetable source | | | |
| Total Intragranular | 99.0 | 99.0 | 99.0 |

Extra-granular

| | | | |
|---|---|---|---|
| Magnesium stearate, NF, | 1.0 | 1.0 | 1.0 |
| vegetable source | | | |
| Total core tablet | 95.2 | 95.2 | 95.2 |
| Opadry ® II Pink Film Coat | 4.8 | — | — |
| Opadry ® II Beige Film Coat | — | 4.8 | — |
| Opadry ® II Purple Film Coat | — | — | 4.8 |
| Purified Water | Removed | Removed | Removed |
| | during | during | during |
| | processing | processing | processing |

¹Calculated as Compound A monocitrate monohydrate salt and assuming an Assay of 100%.
*Avicel DG is co-processed microcrystalline cellulose and anhydrous dibasic calcium phosphate

TABLE 13
Components and Composition of Compound A Tablet Formulations

| | Quality | Component | Tablet Strength | | |
|---|---|---|---|---|---|
| Material | Standard | Function | 25 mg¹ | 110 mg¹ | 160 mg¹ |
| Core Tablet | | | Amount, mg | | |
| Compound A | In-house | Active | 33.4 | 146.5 | 213.2 |
| monocitrate | | | | | |
| monohydrate | | | | | |
| (Compound A | | | | | |
| citrate) | | | | | |
| Avicel ® DG* | N/A² | Filler | 63.7 | 279.0 | 406.0 |
| Crospovidone | NF, Ph. Eur. | Disintegrant | 13.4 | 58.7 | 85.4 |
| Hypromellose | USP, Ph. Eur. | Binder | 13.4 | 58.7 | 85.4 |
| Sodium | USP, Ph. Eur. | Disintegrant | 6.7 | 29.4 | 42.7 |
| Bicarbonate | | | | | |
| Hydrophobic | NF, Ph. Eur. | Glidant | 1.3 | 5.9 | 8.5 |
| Colloidal Silica | | | | | |
| Magnesium | NF, Ph. Eur. | Lubricant | 0.7 | 2.9 | 4.3 |
| Stearate (Intra- | | | | | |
| granular) | | | | | |
| Magnesium | NF, Ph. Eur. | Lubricant | 1.3 | 5.9 | 8.5 |
| Stearate (Extra- | | | | | |
| granular) | | | | | |
| Total | | | 134 | 587 | 854 |
| Coated Tablet | | | Amount, mg | | |
| Compound A | In-house | Active | 134 | 587 | 854 |
| Citrate Core | | | | | |
| Tablet | | | | | |
| Opadry ® II | N/A² | Film Coating | 6.7³ | 29.4³ | 42.7³ |
| complete film | | | (Opadry ® II | (Opadry ® II | (Opadry ® II |
| coating systems | | | 85F94190 | 85F97253- | 85F90035 |
| | | | Pink) | CN Beige) | Purple) |
| Purified Water | USP⁴ | Dispersing | Removed⁵ | Removed⁵ | Removed⁵ |
| | | medium for | | | |
| | | coating agent | | | |

¹Reported as free base equivalents.
²Avicel ® DG and Opadry ® II Complete Film Coating Systems are not compendial, however the components comply with compendial requirements.
³Represents a 5.0% weight gain. The coating suspension is prepared as 15% w/w solids.
⁴Meets USP requirements for Purified Water and in-house specifications.
⁵Removed during processing.
*Avicel ® DG is co-processed microcrystalline cellulose and anhydrous dibasic calcium phosphate.
N/A = Not applicable; NF = National Formulary; Ph. Eur. = European Pharmacopoeia; USP = United States Pharmacopeia.

### Example 7. Excipient Compatibility Study

The first activity in developing a tablet formulation was selection of excipients. The main purpose of the excipient compatibility study was to evaluate changes in related substances, specifically Impurity A (relative retention time [RRT]~0.98). Previous drug substance and drug product stability studies showed the major change during stability was the formation of this degradant. However, formation of this degradant is not possible in product containing Compound A citrate, as it is formed by reaction of the sesquifumarate drug substance with the double bond in fumaric acid. A secondary purpose of the compatibility study was to assess changes in Compound A assay and physical properties. Even though formation of Impurity A is not possible for Compound A citrate, chemical and physical results of this study were still used to guide tablet excipient selection.

Common excipients were evaluated as part of the excipient compatibility study. The list of excipients and ratios of Compound A sesquifumarate to the excipients are shown in Table 14. Samples were incubated at 25° C./60% RH long-term condition as well as a 40° C./75% RH accelerated condition. Individual vials were removed for analysis at 0, 4, and 8 weeks. Samples were tested for appearance, Compound A sesquifumarate assay and related substances, X-ray powder diffraction (XRPD), and differential scanning calorimetry (DSC) at T=0 and 4 weeks for both long-term and accelerated conditions, and 8 weeks for the long-term condition. At 8 weeks, the accelerated condition samples were tested for Compound A sesquifumarate assay and related substances only. Assay and related substances were tested by reversed-phase ultra-performance liquid chromatography (RP-UPLC), with adjusted sample preparation to accommodate small sample amounts. Vials of Compound A sesquifumarate alone were used as controls.

TABLE 14
Excipient Compatibility Study Design

| | | | Mixture |
|---|---|---|---|
| Function | Excipient | Trade Name | Ratios |
| Filler | Microcrystalline cellulose | Avicel ® PH-113 | 1:10 |
| | Microcrystalline cellulose | Avicel ® PH-101 | 1:10 |
| | Microcrystalline | Avicel ® DG | 1:10 |
| | cellulose/Dicalcium | | |
| | phosphate | | |
| | Mannitol | Pearlitol ® 200 | 1:10 |
| | Sorbitol | Neosorb ® P300DC | 1:10 |
| Binder | Hypromellose | Methocel ™ E5 | 1:1 |
| | | Premium LV | |
| | Povidone K30 | Kollidon ® 30 | 1:1 |
| | Hydroxypropyl cellulose, | L-HPC LH-B1 | 1:1 |
| | low substituted | | |
| Disin- | Sodium starch glycolate | Explotab | 1:1 |
| tegrant | Crospovidone XL | Polyplasdone ™ XL | 1:1 |
| | Croscarmellose sodium | Ac-Di-Sol ® | 1:1 |
| | Pregelatinized starch | Starch 1500 | 1:1 |
| Alkalizer | Sodium bicarbonate^{a} | N/A | 10:1 |
| | Anhydrous dibasic | Emcompress ® | 10:1 |
| | calcium phosphate | Anhydrous | |
| Glidant | Colloidal silicon dioxide | Cab-O-Sil | 10:1 |
| Lubricant | Magnesium stearate | Hyqual ® | 10:1 |
| | Stearic acid, Grade 50 | Stearic Acid, Grade 50 | 10:1 |

^{a}Sodium bicarbonate was initially evaluated as an alkalizer but once the salt form was changed to the monocitrate monohydrate, it was retained in the formulation to aid tablet dissolution.
N/A = not applicable.

### i. Filler

Five different fillers were evaluated in the excipient compatibility study. No change was seen in appearance, XRPD or DSC over 8 weeks at 25° C./60% RH or 4 weeks at 40° C./75% RH. The Compound A sesquifumarate control sample had six impurities present at low levels at the start of the study. Five of these did not substantially change during the study, either in control sample or excipient mixtures. Impurity A increased in both control sample and excipient mixtures. This was expected as it is a degradant for the sesquifumarate salt form as well as a process impurity. The presence of polyols such as mannitol and sorbitol were found to accelerate the degradant formation, and these excipients were eliminated from consideration. Microcrystalline cellulose excipients such as Avicel® PH 101, Avicel® PH113, and Avicel® DG did not increase formation of Impurity A.

Microcrystalline cellulose-based fillers were identified as suitable candidates for the drug product formulation.

### ii. Binders

Three different binders were evaluated in the excipient compatibility study. No change was seen in appearance, XRPD or DSC over 8 weeks at 25° C./60% RH or 4 weeks at 40° C./75% RH. No trends were observed in Compound A sesquifumarate assay or related substances. All three binders were suitable candidates for the drug product formulation.

### iii. Disintegrants

Four different disintegrants were evaluated in the excipient compatibility study. No change was seen in appearance, XRPD or DSC over 8 weeks at 25° C./60% RH or 4 weeks at 40° C./75% RH. No trends were observed in Compound A sesquifumarate assay or related substances.

Pregelatinized starch was eliminated from consideration due to its high loss on drying (LOD) specification of 14%, which was considered a risk to the drug product stability due to potential formation of Impurity A with sesquifumarate salt.

Sodium starch glycolate, crospovidone XL and croscarmellose sodium were suitable candidates for the drug product formulation.

### iv. Alkalizers

Two different alkalizers were evaluated in the excipient compatibility study. No change was seen in appearance, XRPD or DSC over 8 weeks at 25° C./60% RH or 4 weeks at 40° C./75% RH. No trends were observed in Compound A sesquifumarate assay or related substances.

Both alkalizers were suitable candidates for the drug product formulation for the sesquifumarate salt form. Once the salt form was switched to the monocitrate monohydrate, sodium bicarbonate was retained as an additional disintegrant to facilitate tablet dissolution. Including sodium bicarbonate in the tablet formulation allowed the tablet dissolution profile to closely match the capsule dissolution profile.

### v. Glidant and Lubricants

Three different glidants and lubricants were evaluated in the excipient compatibility study. No change was seen in appearance, XRPD or DSC over 8 weeks at 25° C./60% RH or 4 weeks at 40° C./75% RH. No trends were observed in Compound A sesquifumarate assay or related substances.

Colloidal silicon dioxide and both lubricants were suitable candidates for the drug product formulation.

In summary, an excipient compatibility study was performed evaluating different fillers, binders, disintegrants, alkalizers, a glidant and lubricants in binary mixtures over 8 weeks at long-term and accelerated conditions. Several excipients were identified as suitable candidates for the Compound A drug product formulation. Although the drug substance salt form changed from sesquifumarate to citrate during tablet development, the results from excipient compatibility study guided excipient selection for Compound A citrate tablets as discussed below.

### Compound A Tablet Formulation Composition

### i. Filler

The initial formulation approach was to dry granulate (roller compact) a blend of Compound A drug substance, microcrystalline cellulose, a binder, disintegrants and a lubricant, followed by extragranular lubrication and compression. Initial attempts to roller compact using only microcrystalline cellulose were unsuccessful, with the blend sticking extensively to the rollers. Avicel® DG was therefore explored to reduce ribbons adhering to the rollers and improve powder flow. Avicel® DG is a combination of 75% microcrystalline cellulose and 25% dicalcium phosphate, produced using a spray-dried, co-processing technology. Avicel® DG is designed specifically to assist in roller compaction processes. Dicalcium phosphate is used in tablets and capsules as a diluent. Initial attempts to roller compact Compound A drug substance with Avicel® DG were successful in obtaining ribbons of acceptable quality. Avicel® DG was therefore selected as the filler/granulation aid for the Compound A drug product.

The amount of Avicel® DG was determined to be 47.5% w/w of the blend. Being a filler, the amount was based on a final drug substance concentration of 25% (free base equivalents) and total remaining excipient concentrations of 27.5% in the blend.

### ii. Binder

Povidone K30, hypromellose, and low-substituted hydroxypropyl cellulose were evaluated as binders for the Compound A citrate tablet formulation. Excipient compatibility studies showed compatibility with all three of the selected binders. Both povidone and hypromellose produced acceptable ribbons and granules in combination with Avicel® DG. Hypromellose was selected as the final binder as it produced denser roller compacted ribbons that resulted in better flow of the blend on the tablet press. It is available in several grades that vary in viscosity and extent of substitution. Low viscosity hypromellose was used in the Compound A tablet formulation as a binder.

Initial experiments evaluated hypromellose concentration of 15% w/w in the blend. The manufactured tablets had a disintegration time of >30 minutes. This was attributed to strong binding property of hypromellose and therefore the concentration was decreased. A final concentration of 10% w/w of hypromellose in the blend was selected based on ability to dry granulate (roller compact) Compound A drug substance and obtain ribbons of acceptable quality. Tablets manufactured from blend containing 10% w/w hypromellose had a disintegration time of <5 minutes.

### iii. Disintegrant

Crospovidone was selected as the disintegrant based on the excipient compatibility study. A range of concentrations from 7 to 15% w/w were studied, before selecting 10%. A formulation containing Compound A sesquifumarate, Avicel® DG as filler, hypromellose as binder, and crospovidone as disintegrant had slower than desired release profile. The tablets were only approximately 40% dissolved after 45 minutes in 900 mL of 0.1N HCl at 75 rpm paddle speed.

An important goal in developing the tablet formulation was to achieve a comparable dissolution profile in vitro and similar exposure in vivo to the fast-dissolving capsule drug product that was used in earlier clinical trials. Sodium bicarbonate was introduced as an additional disintegrant to facilitate tablet dissolution. Sodium bicarbonate is generally used in pharmaceutical formulations as a source of carbon dioxide in effervescent tablets and granules, and to facilitate disintegration.

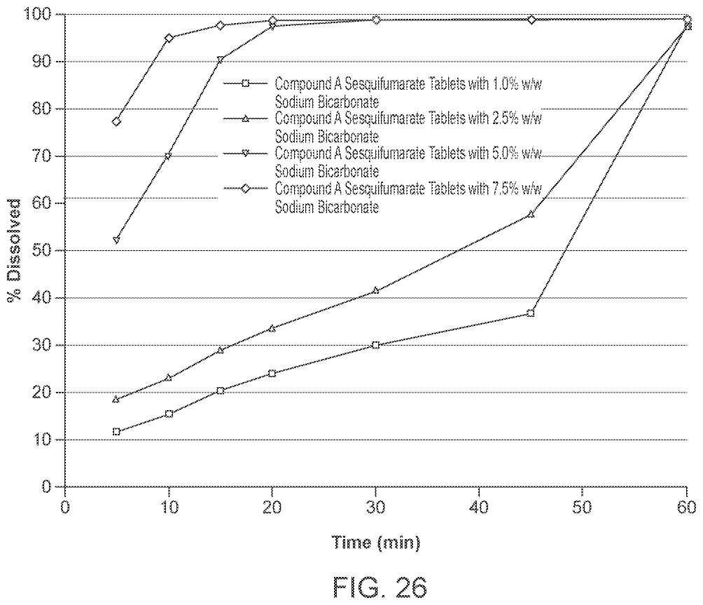

The tablet formulation with 10% w/w crospovidone and 10% sodium bicarbonate intragranular showed 100% release in 10 minutes. Various levels of sodium bicarbonate (1.0%, 2.5%, 5.0%, and 7.5%) were evaluated while keeping crospovidone level constant at 10% w/w in the blend. FIG. 26 shows the comparative dissolution profiles of formulations with varying levels of sodium bicarbonate. A concentration of 5.0% w/w in the blend was finalized as it was the minimal amount required to obtain an immediate release profile. The initial evaluation of disintegrants and their levels was performed with Compound A sesquifumarate tablets. The choice of disintegrants and their levels was leveraged for Compound A citrate tablets.

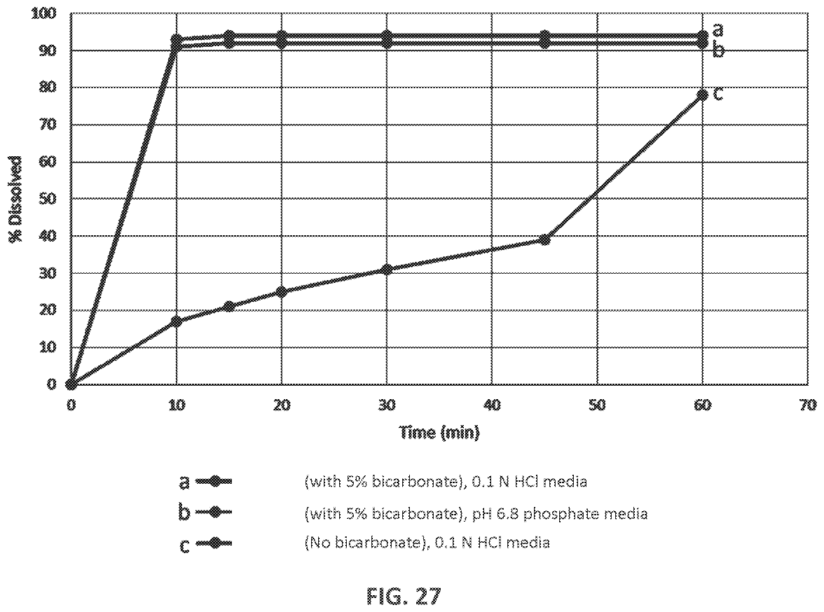

Once the drug substance salt form was changed to citrate salt, the addition of sodium bicarbonate was challenged by completely removing it from the formulation. FIG. 27 shows comparative release profile of tablets with and without sodium bicarbonate. The different salt forms showed similar dissolution profiles (1% sodium bicarbonate in Compound A sesquifumarate tablets had similar dissolution profile to 0% sodium bicarbonate in Compound A citrate tablets over 5 through 45 min).

These tablets were at 250 mg strength and the amount of Avicel DG was adjusted to compensate for the absence of sodium bicarbonate. Tablets with bicarbonate dissolved equally quickly in 0.1 N HCl or pH 6.8 phosphate media. But tablets without bicarbonate had a significantly slower dissolution profile. Note that in each dissolution experiment the paddle speed was increased to 250 rpm after 45 minutes. This caused the large increase in percent dissolved at 60 minutes for the formulation without bicarbonate.

As evidenced, removing sodium bicarbonate from the formulation significantly slowed down the drug release rate. Addition of sodium bicarbonate was therefore deemed necessary for Compound A tablet formulation to have the required immediate release characteristics.

### iv. Glidant

Hydrophobic colloidal silicon dioxide is commonly used as an anti-caking agent or glidant. The hydrophobic grade absorbs less moisture and helps improve powder flow properties.

Hydrophobic colloidal silica was added to the formulation to improve flow. Blending and milling Compound A citrate with 1% hydrophobic colloidal silica improved flow and compression process without adversely impacting any tablet characteristics. The 1% w/w concentration selection was based on previous experience with drug substances that had poor flow properties.

### v. Lubricant

Tablets typically require lubricant to avoid sticking to surfaces such as rollers and punches. Magnesium stearate was evaluated, being added both intra- and extra-granularly. The addition of intra-granular magnesium stearate (0.5%) was deemed necessary to facilitate roller compaction and avoid damage to the compactor rollers. A higher amount of intra-granular magnesium stearate (1.0%) was evaluated but it decreased drug release rate from the tablets. A concentration of 0.5% intra-granular magnesium stearate was therefore finalized. Magnesium stearate was also added extra-granularly (1.0%) to provide lubrication and facilitate blend flow and compression.

### vi. Film Coat

Compound A tablets were film coated for taste masking and to allow product differentiation. Opadry® II Complete Film Coating Systems are platforms of aqueous polyvinyl alcohol (PVA)-based immediate release film coating systems. These are water soluble, pH-independent film coatings, which allow immediate disintegration for fast release of the active ingredient. Opadry® II Complete Film Coating Systems were not included in the excipient compatibility study. However, stability studies on multiple development and Good Manufacturing Practice (GMP) batches demonstrate stability of tablets manufactured using Opadry® II film coatings and establish shelf-life of the drug product in multiple storage conditions.

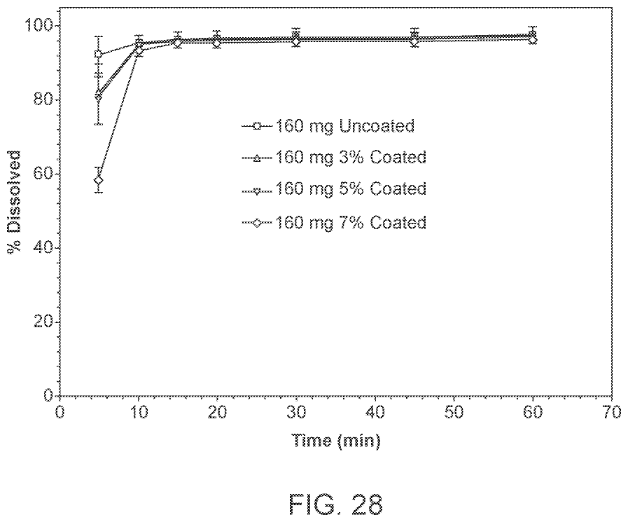

Opadry® II with pink, beige and purple colors were used for the 25 mg, 110 mg, and 160 mg tablets, respectively. The tablets were coated at 3%, 5%, and 7% weight gain to assess the effect of coating level on drug release. The tablets were also inspected for visual appearance and coat uniformity. At 3% coat level, the tablet surface showed visible defects and was not smooth. At 5% and 7% the tablets appeared to have uniform color distribution, fewer visible defects, and an overall smooth surface. The 5% vs. 7% coat level did not have any significant effect on drug release with 100% release by 10 minutes for 3%, 5%, or 7% weight gain coating levels. FIG. 28 shows comparative release profile from 160 mg core tablets (uncoated) and tablets coated at 3%, 5%, and 7% coat level. Similar observations were noted with 110 mg and 25 mg strengths. Based on this data, 5% weight gain was selected for film coating Compound A tablets.

In summary, a tablet formulation containing Avicel® DG, hydrophobic colloidal silica, hypromellose, crospovidone, sodium bicarbonate, and magnesium stearate was developed. The tablets were film coated with Opadry® II coating system. The composition of Compound A tablets manufactured using the Compound A citrate salt form, is provided in Table 12 and 13.

### CONCLUSION

The impact of salt and dose form change on drug product dissolution was evaluated. Compound A sesquifumarate capsule drug product, 25 mg and 113 mg are immediate-release capsules. The 25 mg capsules were manufactured using Xcelodose® 120 equipment. The 113 mg capsules were manufactured using a semi-manual process on MF-30 equipment. Compound A citrate tablet drug product is manufactured at strengths of 25 mg, 110 mg, and 160 mg. The process and equipment used for blending, roller compaction, compression, and film coating of the engineering (non-GMP) and GMP batches are representative of the proposed commercial tablet manufacture.

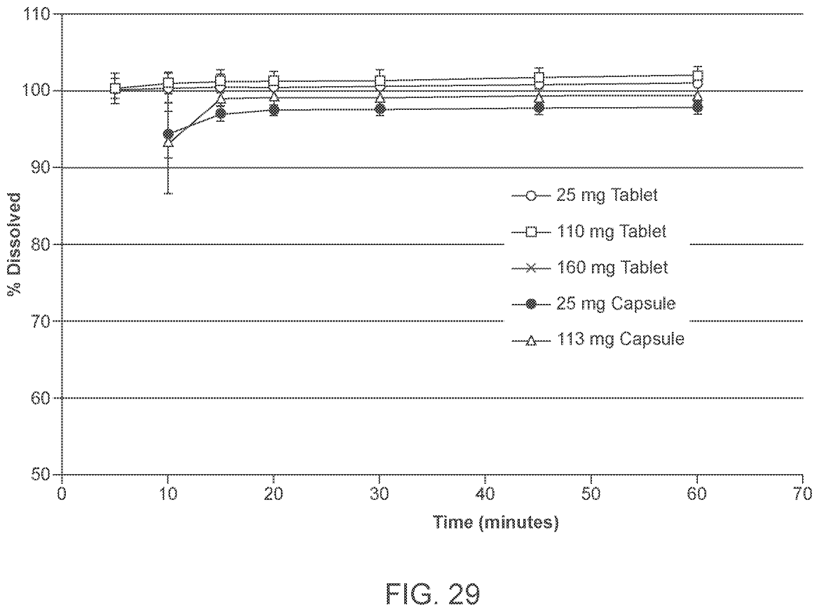

Dissolution profiles were collected for Compound A citrate immediate release tablets and Compound A sesquifumarate immediate release capsules. Both salt forms of Compound A were fast with all drug dissolved by 15 minutes, as shown in FIG. 29. This data is consistent with the high solubility of both drug substance salt forms.

The exposure of Compound A sesquifumarate capsules was compared to Compound A citrate tablets and to Compound A sesquifumarate oral solution in a crossover dog pharmacokinetic study. Assessment of the pharmacokinetic properties (Cmax, AUC0-t, and AUCinf) of the tablet compared to the capsule showed all three parameters were comparable.

Capsule, tablet, and oral solution drug product forms were demonstrated to have similar pharmacokinetics in humans based on Compound A's high solubility, similar in vitro dissolution profiles, and bioequivalence in dog pharmacokinetic study. Human exposure data collected demonstrates similar exposure from Compound A capsules and tablets.

### Example 8. Tablet Manufacturing Process

Compound A tablets are manufactured using the common pharmaceutical ingredients hydrophobic colloidal silica, microcrystalline cellulose, dicalcium phosphate, hypromellose, crospovidone, sodium bicarbonate and magnesium stearate. The process consists of six steps: preblending, intra-granular blending and lubrication, roller compaction and milling, extra-granular lubrication, compression, and coating. The manufacturing process has consistently produced a tablet product that meets all product quality attributes.

Batches were packaged in high-density polyethylene (HDPE) bottles containing 30 tablets, silica desiccant and polyester coil. Stability results are consistent with meeting the QTPP criteria.

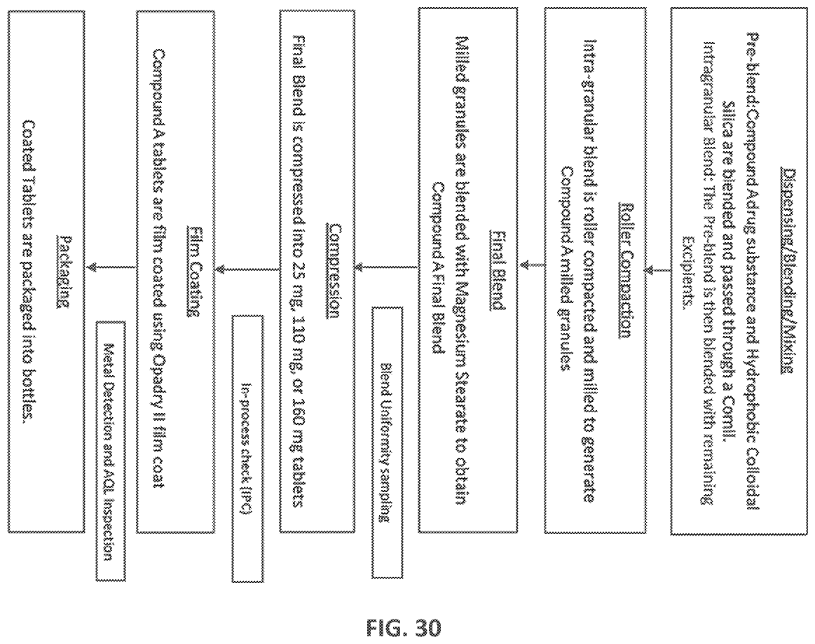

The tablet manufacturing process is shown in FIG. 30.

The process for preparing the tablet has five steps. The purpose of each step is described below.
Step 1: API is blended with hydrophobic colloidal silica to partly coat it and provide some improvement in flow properties.
Step 2: Roller compaction is required to increase bulk density. API is low density and increase is needed to allow blend to fit in the tablet dies.
Step 3: Magnesium stearate is added extragranularly as a lubricant.
Step 4: Compression to achieve the required strengths.
Step 5: A cosmetic film coat is applied for color differentiation purposes.

### Dispensing, Milling, and Intra-Granular Blending

Compound A drug substance (citrate salt, e.g. Form A) and hydrophobic colloidal silica are mixed together in a blender as a pre-blend and passed through a comil. The pre-blend is then mixed with Avicel DG, hypromellose, crospovidone, and sodium bicarbonate in a blender. Intra-granular magnesium stearate is then added and mixed to generate the intragranular blend.

### Roller Compaction, Milling, and Final Blending

The intragranular blend is processed through a roller compactor and milled. The resulting milled granules are blended with the extra-granular magnesium stearate in a blender to generate the final blend. Samples are taken and tested for blend uniformity according to established procedures.

### Compression

The final blend is compressed into tablets using a Fette tablet press equipped with the appropriate tablet punches. Target tablet weights and hardness are shown in Table 15.

TABLE 15
Tablet Target Weight and Hardness

| | | | Target Hardness |
|---|---|---|---|
| | Individual Target | Average Target | (kP), individual |
| Strength (mg) | Weight (mg) | Weight (mg) | and average |
| 25 | 134.0 mg ± 7% | 134.0 mg ± 5% | 8.0 |
| 110 | 587.0 mg ± 7% | 587.0 mg ± 5% | 15.0 |
| 160 | 854.0 mg ± 7% | 854.0 mg ± 5% | 20.0 |

Tablets are sampled at intervals and tested for appearance, weight, thickness, and hardness. Tablets are sampled at beginning, middle, and end of the compression process for disintegration and friability.

Tablets are dedusted after compression and passed through a metal detector. Tablets are sampled for AQL inspection and tested per established procedure.

### Film Coating

Tablets are coated on an O'Hara coater using the appropriate Opadry II materials listed in Tables 8-10. Tablets are coated to an approximate 5.0% weight gain. Coated tablets are sampled for AQL inspection and tested per established procedure.

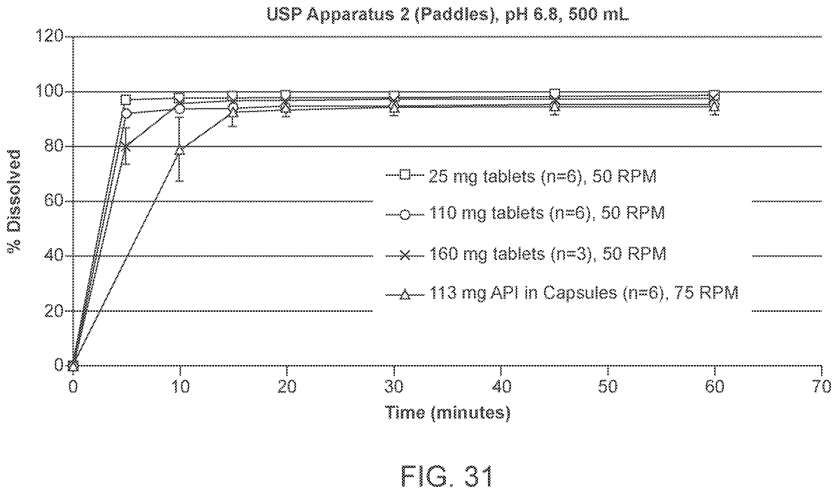

Dissolution was a key characteristic since tablets were designed to replace capsules. The tablet dissolution method uses pH 6.8 media, where drug has lowest solubility. It also uses the slowest recommended paddle speed of 50 rpm, and the lowest media volume of 500 mL. FIG. 31 presents an overlay of 25, 110, and 160 mg coated Compound A monocitrate monohydrate tablet dissolution profiles compared to 113 mg Compound A sesquifumarate capsules. The tablets were from engineering batches, manufactured at 30,000 unit scale using GMP equipment. Tablet dissolution was performed using the intended method conditions of pH 6.8 media and 50 rpm paddle speed. Capsule dissolution was tested in the same media but using a slightly different paddle speed. Both tablets and capsules meet the definition of “very rapidly dissolving”, i.e., more than 85% dissolved in 15 minutes.

### Example 9. Compound A Monocitrate Monohydrate Superior Characteristics vs. Sesquifumarate Form

Compound A sesquifumarate is subject to a chemical degradation where fumarate reacts with the pyrimidine ring forming impurity A. The structure of impurity A is:

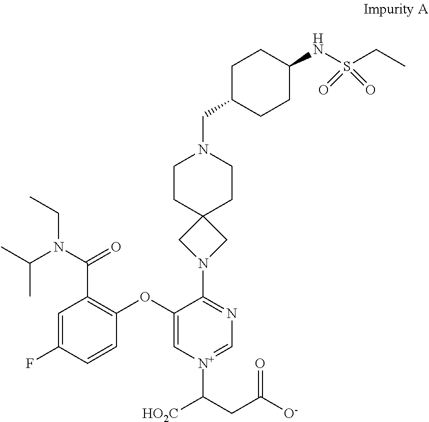 [SMILES: CCN(C(=O)c1cc(F)ccc1Oc1c[n+](C(CC(=O)[O-])C(=O)O)cnc1N1CC2(CCN(C[C@H]3CC[C@H](NS(=O)(=O)CC)CC3)CC2)C1)C(C)C]

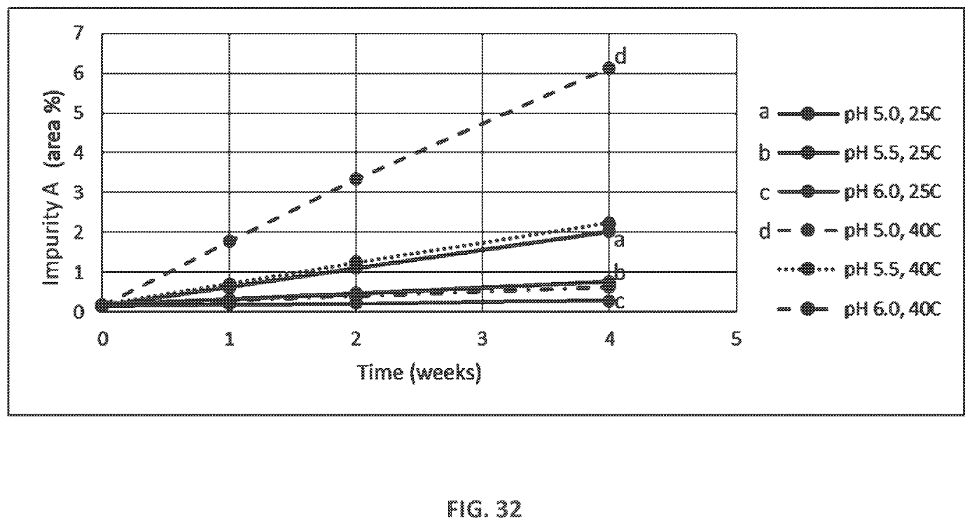

Although this degradation pathway is more accelerated in solution it was also observed in the solid API. Formation of Impurity A was the reason why Compound A sesquifumarate API and drug product were stored at refrigerated temperatures. Citrate counterion, due to lack of a carbon-carbon double bond, cannot form this type of adduct with Compound A. Solution stability studies at accelerated conditions confirmed the absence of this type of degradation (FIG. 32).

Furthermore, Compound A sesquifumarate has a large propensity to form solvates with organic solvents. The relatively high daily dose and pediatric component of the patient population meant that a solvated form would not be acceptable. In order to isolate a non-solvated form of the sesquifumarate salt the manufacturing process required first precipitating the sesquifumarate ethanolate salt from ethanol:water mixture and then slurrying in acetone. Acetone was the only organic solvent which could be used in a manufacturing setting that could be removed during drying to give the non-solvated sesquifumarate form. This manufacturing process required multiple steps and resulted in decreased yields (losses during the acetone slurry). The monocitrate monohydrate salt is isolated directly from ethanol:water, no slurrying step is required.

### Example 10. Effect of Adding Bicarbonate to the Formulation

In this study, the effect of leaving out the bicarbonate in the formulation is assessed. Formulations having the compositions in Table 16 are prepared. Additional Avicel DG is added to the formulation without bicarbonate to make up the difference.

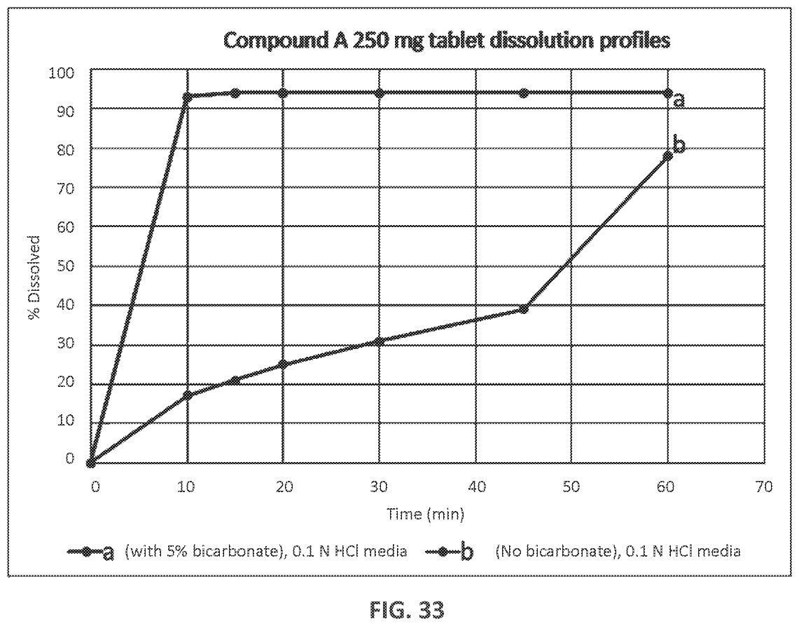

The comparison of the dissolution profiles for the formulations with and without bicarbonate is provided in FIG. 33. Removing bicarbonate results in significantly slower dissolution.

TABLE 16
Formulations of 250 mg Tablets Manufactured
with and without Bicarbonate

| | % w/w or mg/tab | | % w/w or mg/tab | |
|---|---|---|---|---|
| | for formulation | | for formulation, | |
| Ingredients | WITH bicarbonate | | NO bicarbonate | |
| Compound A monocitrate | 33.8% | 334.22¹ | 33.8% | 334.22¹ |
| monohydrate | | | | |
| Avicel DG | 39.8% | 390.78 | 44.5% | 440.78 |
| (Microcrystalline | | | | |
| cellulose & dibasic | | | | |
| calcium phosphate) | | | | |
| Crospovidone, USP, EP, | 10.0% | 100.00 | 10.0% | 100.00 |
| JP | | | | |
| (Polyplasdone ™XL-10) | | | | |
| Hydroxypropyl | 10.0% | 100.00 | 10.0% | 100.00 |
| Methycellulose USP, EP, | | | | |
| JP | | | | |
| (Methocel E50 Premium | | | | |
| LV) | | | | |
| Sodium Bicarbonate BP, | 5.0% | 50.00 | None | None |
| JP, USP, Ph. Eur. | | | | |
| Silicon Dioxide, USP/NF, | 1.0% | 10.00 | 1.0% | 10.00 |
| Ph. Eur (Aerosil R972) | | | | |
| Magnesium Stearate, | 0.5% | 5.00 | 0.5% | 5.00 |
| vegetable source, NF- | | | | |
| GenAR ®, BP, EP, JP | | | | |
| (Hyqual ®) | | | | |
| Magnesium Stearate, | 1.00% | 10.00 | 1.00% | 10.00 |
| vegetable source, NF- | | | | |
| GenAR ®, BP, EP, JP | | | | |
| (Hyqual ®) | | | | |

¹The free-base correction factor is = Weight percent assay * (MW of free-base)/(MW of monocitrate²) = 97.6% * 630.82/822.93 = 0.748
(250 mg base/0.748 correction factor) = 334.22 mg salt

Table 17 compares the formulation used for assessing effect of bicarbonate with the final Compound A core tablet formulation. The final formulation required less active ingredient in order to achieve suitable flow properties. The amount of Avicel DG was increased accordingly.

TABLE 17
Comparison of 250 mg Formulation to Final
Compound A Core Tablet Formulation

| | % w/w for | % w/w for | Final |
|---|---|---|---|
| | 250 mg tablet | 250 mg tablet | Compound A |
| | formulation | formulation, | Core Tablet |
| | WITH | NO | Formulation, |
| Ingredients | bicarbonate | bicarbonate | % w/w |
| Compound A | 33.8% | 33.8% | 25.0% |
| monocitrate | | | |
| monohydrate | | | |
| Avicel DG | 39.8% | 44.5% | 47.5% |
| (Microcrystalline | | | |
| cellulose & dibasic | | | |
| calcium phosphate) | | | |
| Crospovidone, USP, | 10.0% | 10.0% | 10.0% |
| EP, JP | | | |
| (Polyplasdone ™XL- | | | |
| 10) | | | |
| Hydroxypropyl | 10.0% | 10.0% | 10.0% |
| Methycellulose | | | |
| USP, EP, JP | | | |
| (Methocel E50 | | | |
| Premium LV) | | | |
| Sodium Bicarbonate | 5.0% | None | 5.0% |
| BP, JP, USP, Ph. | | | |
| Eur. | | | |
| Silicon Dioxide, | 1.0% | 1.0% | 1.0% |
| USP/NF, Ph. Eur | | | |
| (Aerosil R972) | | | |
| Intragranular | 0.5% | 0.5% | 0.5% |
| Magnesium | | | |
| Stearate, vegetable | | | |
| source, NF- | | | |
| GenAR ®, BP, EP, | | | |
| JP (Hyqual ®) | | | |
| Extragranular | 1.00% | 1.00% | 1.00% |
| Magnesium | | | |
| Stearate, vegetable | | | |
| source, NF- | | | |
| GenAR ®, BP, EP, | | | |
| JP (Hyqual ®) | | | |

Note:
(columns may not add up to exactly 100.0% due to rounding)

### Example 11. Liquid Formulations (i.e., Solution Formulations) Development

An alternate dosage form, an oral solution, was developed for adults and children unable to swallow a solid dosage form. The target patient population encompasses very young children and would require weight- or body-surface area-based dosing. Based on this requirement and Compound A's high solubility across the physiological pH range, an oral solution was selected as the alternate dose form. The composition of three liquid formulations is shown in Table 18.

### Formulation Development

The first oral solution formulation developed used sesquifumarate salt form was designated F1. The formulation composition was then modified by removing grape flavor and adjusting preservative concentrations. This formulation was designated as F2. During clinical development, the drug substance salt form was changed from the sesquifumarate salt to the monocitrate monohydrate salt. This change was made to ensure a scalable, more robust drug product, with greater stability. This formulation was designated as F3.

TABLE 18
Composition of the Oral Solution Formulations Comprising Compound A

| | Quality | Component | Composition Designation | | |
|---|---|---|---|---|---|
| Material | Standard | Function | F1 | F2 | F3 |
| Compound A | — | — | Sesquifumarate | Sesquifumarate | Citrate |
| salt form | | | | | |
| Compound A | — | — | 6.19 (50 | 6.19 (50 | 5.18 (40 |
| salt form | | | mg/mL | mg/mL | mg/mL |
| (% w/w) | | | free-base | free-base | free-base |
| | | | equivalents)^{a} | equivalents)^{a} | equivalents)^{b} |
| Citric acid, | USP, Ph. Eur. | Buffering | 0.38 | 0.38 | 0.38 |
| anhydrous | | agent | | | |
| (% w/w) | | | | | |
| Advantame | Manufacturer's | Sweetener | 0.07 | 0.07 | 0.07 |
| (% w/w) | grade | | | | |
| Methylparaben | NF, Ph. Eur. | Preservative | 0.1 | 0.18 | 0.10 |
| sodium | | | | | |
| (% w/w) | | | | | |
| Potassium | NF, Ph. Eur. | Preservative | 0.1 | 0.20 | 0.10 |
| sorbate | | | | | |
| (% w/w) | | | | | |
| Sodium | USP, Ph. Eur. | Taste | 1.0 | 1.0 | 1.0 |
| chloride | | Masking | | | |
| (% w/w) | | | | | |
| Sodium | USP, Ph. Eur. | Buffering | 1.6 | 1.6 | N/A |
| bicarbonate | | Agent | | | |
| (% w/w) | | | | | |
| Grape flavor | GRAS^{c,d} | Flavor | 1.0 | N/A | 1.0 |
| (% w/w) | | | | | |
| Sodium | NF, Ph. Eur. | pH | Adjusted to | Adjusted to | Adjusted to |
| hydroxide | | adjuster^{e} | pH 6.5 | pH 6.0 | pH 3.0 |
| Hydrochloric | NF, Ph. Eur. | pH | | | |
| acid | | adjuster^{e} | | | |
| Purified | USP, Ph. Eur. | Vehicle | Q.S. to 100% | Q.S. to 100% | Q.S. to 100% |
| water | | | | | |

^{a}50 mg/mL free base of API is equivalent to 63.80 mg/mL of salt form of API. The specific gravity of the final formulation is 1.03 g/mL. A concentration of 50 mg/mL free base is therefore equivalent to 48.5 mg/g free base or 61.9 mg/g of sesquifumarate salt.
^{b}40 mg/mL free base of API is equivalent to 53.32 mg/mL of salt form of API. The specific gravity of the final formulation is 1.03. A concentration of 40 mg/mL free base is therefore equivalent to 38.8 mg/g (40 mg/mL/1.03) free base or 51.8 mg/g of citrate salt form.
^{c}All flavor ingredients contained in FONA's artificial grape flavor 856.0196U are approved for use in a regulation of the Food and Drug Administration or are listed as being generally recognized as safe on the FEMA GRAS list. Propylene glycol is used as the solvent.
^{d}All flavor ingredients contained in Kerry Grape Nat/Art Flavor 20591717 are approved for use in a regulation of the Food and Drug Administration or are listed as being generally recognized as safe on the FEMA GRAS list.
^{e}For pH adjustment as needed.
API = Active Pharmaceutical Ingredient; FEMA = Flavor and Extract Manufacturers Association; GRAS = Generally Recognized as Safe; N/A = Not Applicable; NF = National Formulary; Ph. Eur. = European Pharmacopoeia; Q.S. = Quantum Sufficient; USP = United States Pharmacopeia.

### i. Quality Target Product Profile

The Quality Target Product Profile (QTPP) of Compound A oral solution is shown in Table 19. Key requirements include:
(a) Compound A concentration: the volume of administration must be not more than (NMT) 5 mL per dose for pediatric patients,
(b) organoleptic acceptance: the composition must mask the bitter drug substance taste sufficiently to ensure acceptance,
(c) manufacturing process: ensure a scalable manufacturing process to maintain adequate commercial supply, and
(d) stability: the final drug product must be chemically and physically compatible and stable during bulk storage, distribution, and the duration of the product's shelf life.

TABLE 19
QTPP for Compound A Oral Solution

| QTPP Elements | Target |
|---|---|
| Route of administration | Oral |
| Active ingredient | Compound A citrate |
| Dosage form | Oral solution |
| Dosage strength | Strength to allow dose volume NMT 5 mL |
| Specifications (comply | Appearance, pH, identification, assay, |
| with criteria) | impurity, preservative content, antimicrobial |
| | effectiveness testing |
| Stability | Shelf-life of at least 2 years at refrigerated |
| | condition (2° C.-8° C.) |
| Packaging | Bottles |

### Selection of Sweetener

Compound A drug substance has a strong bitter taste with lingering aftertaste. To achieve the likely dose levels needed the solution concentration would be tens of mg/mL. It was necessary to add sweetener to make a palatable oral solution, especially for children. During formulation development of the F1 formulation, solutions of Compound A sesquifumarate drug substance in water with multiple naturally occurring sweeteners were evaluated (Table 20), but they were not palatable.

TABLE 20
Initial Sweetener Evaluation

| Sample | Sweetener (% w/v) | Observation |
|---|---|---|
| 1 | 65% sucrose | Bitter taste |
| 2 | 20% fructose, 30% sucrose | Bitter taste |
| 3 | 20% glycerin, 0.05% sucralose | Bitter taste |
| 4 | 20% sorbitol, 30% sucrose | Bitter taste |
| 5 | 40% sucrose, 0.05% sucralose | Bitter taste |
| 6 | 20% sorbitol, 0.05% sucralose | Bitter taste |

Since naturally occurring sweeteners and sucralose were not successful in masking the bitter taste, high intensity sweeteners advantame and neotame were evaluated. This evaluation was done simultaneously with flavor selection. Advantame at 0.050% w/v or 0.090% w/v in a formulation containing grape flavor was able to mask the bitter taste. A final advantame concentration of 0.07% w/v was chosen for F1 formulation and maintained for the F2 and F3 formulations. The maximum aqueous solubility of advantame is 0.09% w/v. An advantame concentration of 0.07% w/v was chosen to ensure adequate taste masking while staying below its solubility limit.

Table 21 presents calculated advantame exposures for various age children and doses against FDA acceptable daily intake (ADI) limits of 32.8 mg/kg.

TABLE 21
Calculated Advantame Exposures With Respect to Age and Dose

| | | | | | Advantame | | % of |
|---|---|---|---|---|---|---|---|
| | | | | Single | Exposure | | FDA |
| | Estimated | Estimated | | Dose | per | FDA | ADI for |
| Age | Weight | BSA | Dose | Volume | Dose | ADI | BID |
| (years) | (kg) | (m²) | (mg/m²) | (mL) | (mg) | (mg) | Dosing |
| 0.08 | 3 | 0.25 | 65 | 0.41 | 0.28 | 98.40 | 0.58 |
| 0.08 | 3 | 0.25 | 95 | 0.59 | 0.42 | 98.40 | 0.84 |
| 0.08 | 3 | 0.25 | 160 | 1.00 | 0.70 | 98.40 | 1.42 |
| 1 | 9.5 | 0.47 | 65 | 0.76 | 0.53 | 311.60 | 0.34 |
| 1 | 9.5 | 0.47 | 95 | 1.12 | 0.78 | 311.60 | 0.50 |
| 1 | 9.5 | 0.47 | 160 | 1.88 | 1.32 | 311.60 | 0.84 |
| 6 | 20 | 0.79 | 65 | 1.28 | 0.90 | 656.00 | 0.27 |
| 6 | 20 | 0.79 | 95 | 1.88 | 1.31 | 656.00 | 0.40 |
| 6 | 20 | 0.79 | 160 | 3.16 | 2.21 | 656.00 | 0.67 |
| 12 | 42 | 1.3 | 65 | 2.11 | 1.48 | 1377.60 | 0.21 |
| 12 | 42 | 1.3 | 95 | 3.09 | 2.16 | 1377.60 | 0.31 |
| 12 | 42 | 1.3 | 160 | 5.20 | 3.64 | 1377.60 | 0.53 |
| 18 | 56 | 1.6 | 65 | 2.60 | 1.82 | 1836.80 | 0.20 |
| 18 | 56 | 1.6 | 95 | 3.80 | 2.66 | 1836.80 | 0.29 |
| 18 | 56 | 1.6 | 160 | 6.40 | 4.48 | 1836.80 | 0.49 |

Estimated weight from CDC, National Center for Health Statistics, Growth Charts. Female weight was used to provide maximum exposure calculations. Estimated BSA from Children's Cancer and Leukemia Group (www.cclg.org.uk).
ADI = Acceptable Daily Intake;
BID = Twice a day;
BSA = Body Surface Area;
CDC = Center for Disease Control and Prevention;
FDA = Food and Drug Administration.

### iii. Selection of Flavorant

During formulation development of the F1 formulation, in addition to sweetener, multiple flavoring agents were evaluated for their compatibility and for their ability to mask bitter taste. Samples were formulated at 50 mg/mL Compound A using 20% sorbitol and 0.05% sucralose. At the time, this was the sweetener combination identified as best able to mask taste. Formulations were prepared by the addition of sorbitol and sucralose, followed by flavoring agents. Compound A sesquifumarate was then added, and the pH adjusted to 6.0 by slow addition of sodium hydroxide. Finally, formulations were adjusted with water to the appropriate volume. Table 22 shows that all the flavorants had some incompatibility with formulation or preparation method. However, grape flavor was identified as the best for taste masking.

TABLE 22
First Flavoring Study

| Sam- | Sweet- | | Amount | |
|---|---|---|---|---|
| ple | eners^{a} | Flavorants | (% w/v) | Observations |
| 1 | A | Bubble gum, | 0.5, 0.5 | Flavorants formed slight |
| | | Mint | | emulsion, |
| | | | | bitter taste |
| 2 | A | Chocolate, | 0.5, 0.5 | Phase separation after |
| | | Mint | | adjustment to |
| | | | | pH 6.0, bitter taste |
| 3 | A | Bubble gum, | 0.5, 1.0, | Vanillin crystallized upon |
| | | Vanillin, | 1.0 | standing, |
| | | Glycine | | bitter taste |
| 4 | A | Chocolate, | 1.0, 1.0 | Phase separation after |
| | | Vanillin | | adjustment to |
| | | | | pH 6.0, bitter taste |
| 5 | A | Chocolate, | 1.0, 1.0, | Phase separation after |
| | | Vanillin, | 0.5 | adjustment to |
| | | Mint | | pH 6.0, bitter taste |
| 6 | A | Chocolate, | 1.0, 1.0, | Phase separation after |
| | | Vanillin, | 0.04 | adjustment to |
| | | Menthol | | pH 6.0, bitter taste |
| 7 | A | Artificial | 1.0 | Some precipitation of grape |
| | | Grape | | flavor |
| | | (Spectrum | | during pH adjustment to 6.0 |
| | | Chemical | | (black particulate). |
| | | G1427, purple | | Grape successful at lessening |
| | | liquid) | | initial taste, but bitter |
| | | | | aftertaste remained. |

^{a}A = 20% w/w sorbitol and 0.05% w/v sucralose.

A second flavor evaluation study was performed. This study focused on grape and chocolate/vanillin flavors and used the principle of taste balancing to mask bitterness. The hypothesis was that a combination of sweet, sour, and salty flavors could help reduce perception of bitterness. The sesquifumarate salt of Compound A was being used at the time, and fumarate is reported to have a sour taste. Formulations evaluated in the second study used advantame as a sweetener, and sodium chloride for a salty taste (Table 23). Formulations were prepared by dissolving sweetener and other excipients in water. Compound A sesquifumarate was then added and the pH adjusted to 6.0 by slow addition of sodium hydroxide. Finally, formulations were adjusted with water to the appropriate volume.

A formulation using advantame as a sweetener, grape as a flavor, and salty taste provided by sodium chloride was selected based on this study. An advantame concentration of 0.07% w/v was chosen, to ensure adequate palatability even in young children. Sodium chloride at 1.0% w/w was also added to the F1 formulation to help mask the bitter drug substance taste, and the same concentration was kept in the F2 and F3 formulations.

TABLE 23
Second Flavoring Study

| Sam- | Sweet- | | Amount | |
|---|---|---|---|---|
| ple | eners | Flavorants^{c} | (% w/v) | Observations |
| 9 | B^{a} | Grape^{b} | 1.0 | Masked initial taste, aftertaste |
| | | | | remained |
| 10 | B^{a} | Grape, NaCl | 1.0, 1.0 | Acceptable masking of both |
| | | | | initial and aftertaste |
| 11 | B^{a} | Chocolate, | 0.8, 0.8 | Less effective than grape for |
| | | Vanillin | | taste masking |
| 12 | B^{a} | Chocolate, | 0.8, 0.8, | Some phase separation observed |
| | | Vanillin, | 1.0 | during pH adjustment. Less |
| | | NaCl | | effective than grape for taste |
| | | | | masking |
| 13 | C^{c} | Grape, NaCl | 1.0, 1.0 | Acceptable masking of both |
| | | | | initial and aftertaste |

^{a}0.05% w/v advantame.
^{b}Grape flavor from Spectrum Chemicals (G1427), dark purple liquid.
^{c}0.09% w/v advantame.

The purple grape flavoring used was observed to precipitate during pH adjustment. Therefore, the Spectrum Chemicals grape flavor was replaced by a colorless artificial grape flavor, with minimal to no ultraviolet (UV) absorbance, from FONA International (product 856.0196U). Minimal or lack of UV absorbance was desirable since it would not interfere with assessing whether any degradants formed during storage. The FONA grape flavor did not show any precipitation during preparation and provided equally good taste masking. A combination of advantame as a sweetener, FONA grape flavor (product 856.0196U), and sodium chloride providing salty taste was selected for taste masking Compound A in the F1 formulation.

A formulation containing advantame and sodium chloride, but no grape flavor was also assessed and was considered acceptable, which is the basis for the F2 oral solution. The F1 formulation met United States Pharmacopeia (USP)<51> criteria for preservative efficacy testing, but did not meet European Pharmacopoeia (Ph. Eur.) 5.1.3 Day 14 criteria. An oral formulation suitable for use at European clinical trial sites was desired and was achieved by the minimal change of decreasing the pH from 6.5 to 6.0 and removing grape flavor. This second formulation was designated F2.

After the Compound A salt form was changed from sesquifumarate to citrate, reformulation efforts further evaluated taste and organoleptic acceptance. Compound A citrate also had a bitter taste, similar to the sesquifumarate salt. A solid and liquid grape flavor as well as a mixed berry flavor were evaluated for the F3 formulation. Table 24 shows key observations from the flavor evaluation study. All pH 3.0 formulations were clear and had ≥98% recoveries for preservatives as well as Compound A. Both grape flavors were equally effective in helping taste mask. Solid grape flavor was selected to allow ease of handling and dispensing during manufacturing.

TABLE 24
Third Flavoring Study

| Sam- | Sweet- | | Amount | | |
|---|---|---|---|---|---|
| ple | ener^{a} | Flavor | (% w/v) | pH | Observations |
| 1 | A | Powder | 1.0 | 3.0, 5.5 | pH 3.0 formulations |
| | | Grape^{b} | | | were clear while pH 5.5 |
| | | | | | formulations showed few |
| | | | | | small particles. |
| | | | | | Acceptable masking of both |
| | | | | | initial and aftertaste |
| 2 | A | Liquid | 1.0 | 3.0, 5.5 | pH 3.0 formulations |
| | | Grape^{c} | | | were clear while pH 5.5 |
| | | | | | formulations showed few |
| | | | | | small particles. |
| | | | | | Acceptable masking of both |
| | | | | | initial and aftertaste |
| 3 | A | Mixed | 1.0 | 3.0, 5.5 | pH 3.0 formulations |
| | | Berry^{d} | | | were clear while pH 5.5 |
| | | | | | formulations showed few |
| | | | | | small particles. |
| | | | | | Less effective than grape |
| | | | | | for taste masking. |

^{a}A = 0.07% w/v advantame.
^{b}Kerry Grape Flavor 20591717.
^{c}Sensient Grape Flavor 1116859.
^{d}Sensient Mixed Berry Flavor SN2000015236.

A pH 3.0 formulation using advantame as a sweetener, Kerry grape as a flavor, and salty taste provided by sodium chloride was selected for Compound A oral solution.

### iv. Selection of Buffering Agent

During F1 formulation development, the optimum pH was identified as between pH 6.0 and 7.0. Citric acid (highest pKa of 6.4) was selected as a pharmaceutically acceptable buffer capable of strongly buffering the formulation at the desired pH of 6.5. Citric acid was used to provide an acidic pH during addition of advantame, fostering its faster dissolution. A concentration of 20 mM citrate was selected to protect against pH drift during long-term storage.

The final F1 formulation also contains sodium bicarbonate and sodium hydroxide. These are needed to achieve the desired pH of 6.5. Compound A sesquifumarate contains 3 molecules of fumarate for every 2 drug substance molecules, leading to a fumarate concentration of 119 mM in a 50 mg/mL Compound A solution in water and a pH of approximately 3.5. Compound A citrate meets the USP definition of soluble across the entire physiological pH range from 1.2 through 6.8, but it has increased solubility at both the acidic and neutral ends of the range. During formulation development it was observed that a combination of citrate/citric acid could not overcome the low pH caused by fumarate counterion. During early formulation development, pH was adjusted using sodium hydroxide. Localized regions of high pH around sodium hydroxide droplets could promote base-induced degradation, so an alternate method of pH adjustment was explored.

Sodium bicarbonate was selected as a means of neutralizing fumaric acid without requiring addition of large quantities of sodium hydroxide. Sodium bicarbonate is added to the formulation at a concentration of 1.6% w/v prior to the addition of Compound A sesquifumarate. When Compound A sesquifumarate is added the bicarbonate reacts with hydrogen ions to form carbonic acid, which degrades to carbon dioxide and water. This neutralizes the majority of the acid, leading to a pH of approximately 6.0.

For the F1 formulation, sodium hydroxide is then added to achieve the final pH of 6.5. Small scale formulation studies showed it would be difficult to reproducibly achieve a pH of 6.5±0.2 with only the addition of bicarbonate. Thus, sodium hydroxide is added afterward to complete the pH adjustment. For the F2 formulation, sodium hydroxide is only added to achieve the final pH of 6.0 if needed. Compound A degradation is avoided by slow addition and vigorous stirring.

For the F3 formulation, the optimum pH was 3.0, based on solubility of drug substance and preservatives coupled with effective anti-microbial activity. Citrate was also a suitable buffering agent at this pH due to its pKa of 3.1.

### v. Selection of Compound A Concentration

The first parameter to be defined was Compound A drug substance concentration. Compound A sesquifumarate was quite soluble so it would be possible to achieve a high concentration. A concentration of 50 mg/mL (free base equivalents) was selected to accommodate a range of ages and a range of doses. At the time the oral solution formulation was being developed, pediatric compassionate use patients had been dosed up to 160 mg/m²/dose. At this dose a young child with body surface area (BSA) of 0.3 m² would receive 1 mL/dose and an older child with a BSA of 1.6 m² would receive 5 mL/dose.

Solubility of Compound A citrate was determined in 3 different types of matrices: unbuffered water, citrate-buffered solutions from pH 3 to 6, and formulations consisting of citrate, advantame, methylparaben sodium, potassium sorbate, and sodium chloride at pH ranging from 3 to 6. Solubility in unbuffered water at ambient temperature was 38.5 mg/mL. Solubility was affected by solution pH, with maxima at both pH 3 and 6. However, both the preservatives showed low recovery in these formulations, likely due to saturating solubilities of formulation components. The target concentration of Compound A citrate was therefore decreased to 40 mg/mL in order to achieve full dissolution of preservatives. A Compound A concentration of 40 mg/mL is sufficient to support acceptable dose volumes for a wide range of dose levels and ages.

### vi. Selection of pH

The initial F1 and F2 formulations utilized Compound A sesquifumarate. The pH stability of Compound A sesquifumarate solutions was studied in water at pH 5.0, 5.5, and 6.0 over 4 weeks at 25° C. and 40° C. The desired concentration of 50 mg/mL was soluble at all pH values, although Compound A dissolution occurred more rapidly at lower pH. There was a single major degradant, designated as Impurity A (relative retention time [RRT]~0.98). Impurity A increased in a temperature- and time-dependent manner, FIG. 32.

It was concluded from this study that formulation should be maintained close to neutral pH, and that Compound A sesquifumarate formulation should be stored refrigerated. Many of the subsequent formulation development studies were performed at pH 6.0 as a compromise between drug substance dissolution rate and stability. Later studies were performed at pH values from 6.0 to 7.0 and a combination of antimicrobial effectiveness testing (AET) and stability data led to the selection of pH 6.5 for the final F1 formulation and pH 6.0 for the F2 formulation.

Stability of formulations containing Compound A sesquifumarate was studied at 50 mg/mL in a formulation consisting of 0.07% advantame, 20 mM citrate, 1% grape flavor (FONA), 1% sodium chloride, and 0.02% propyl paraben at pH 6.5. Stability was measured over 4 weeks in glass vials at the accelerated condition of 25° C. Stability of the same formulation but at a pH of 6.5 was also studied at the supra-accelerated condition of 40° C. These studies were conducted prior to preservative effectiveness testing and therefore used propyl paraben instead of the methylparaben sodium:potassium sorbate combination used in the final formulation.

There were no meaningful changes in Compound A recovery at either condition, Table 25. There was an increase in Impurity A at the 40° C. condition, but not at the 25° C. condition. An impurity at RRT 0.94 was present at 0.08% area initially but did not change during storage. This short-term stability study at accelerated and supra-accelerated conditions suggested that Compound A oral solution formulation would remain within specifications at refrigerated conditions for extended periods of time.

TABLE 25
Stability of Compound A Solution at 25° C. and 40° C.

| | | | | Compound A | Compound A | |
|---|---|---|---|---|---|---|
| | | | | Sesquifumarate | Sesquifumarate | Impurity A |
| Temp. | Time | | | Recovery | Purity (Average | (Average % |
| (° C.) | Point | Appearance | pH | (Average %) | % area) | area) |
| 25° C. | 0 | Clear | 6.5 | 100.0 | 99.78 | 0.14 |
| | | solution | | | | |
| | 1 week | Clear | 6.6 | 100.7 | 99.77 | 0.14 |
| | | solution | | | | |
| | 2 weeks | Clear | 6.6 | 97.4 | 99.78 | 0.13 |
| | | solution | | | | |
| | 4 weeks | Clear | 6.6 | 99.0 | 99.78 | 0.14 |
| | | solution | | | | |
| 40° C. | 0 | Clear | 6.2 | 100.0 | 99.77 | 0.14 |
| | | solution | | | | |
| | 1 week | Clear | 6.2 | 100.1 | 99.75 | 0.17 |
| | | solution | | | | |
| | 2 weeks | Clear | 6.2 | 99.4 | 99.68 | 0.24 |
| | | solution | | | | |
| | 4 weeks | Clear | 6.3 | 100.2 | 99.58 | 0.34 |
| | | solution | | | | |

The target pH was reassessed when the F3 formulation was developed. Compound A citrate solubility studies were performed initially in simple buffered solutions to assess solubility trends as a function of pH. Solubility was highest at pH 3.0, decreased from pH 3.5 to 4.5 and then increased from pH 5.5 to 6.0. Formulation studies were conducted across pH ranges, and it was found that Compound A had the highest solubility (target concentration of 50 mg/mL) in the oral solution formulation at pH 3 and pH 6. However, both preservatives showed low recovery in these formulations, likely due to saturating solubilities of formulation components. The target concentration of Compound A was therefore changed to 40 mg/mL as a compromise between drug substance and preservatives dissolution rate and stability.

The final selection of pH 3.0 was made based on a combination of AET and ability to dissolve both the preservatives and Compound A citrate at target concentrations.

### vii. Selection of Preservatives

Compound A oral solution drug product is supplied as 100 mL per multi-use bottle. Anti-microbial effectiveness testing was performed as per USP <51> to select preservatives and levels. Three different preservative combinations were tested, each at 3 different levels, Table 26. The combination of propyl paraben/potassium sorbate did not meet USP <51> acceptance criteria for Pseudomonas aeruginosa at 14 days. The methyl paraben/propyl paraben combination also did not meet USP <51> acceptance criteria for P. aeruginosa at 28 days. The combination of methyl paraben and potassium sorbate met acceptance criteria at 14 and 28 days for all 3 concentrations tested.

TABLE 26
Results for Compound A Oral Solution (F1)
Antimicrobial Effectiveness Testing

| Preservatives | Concentrations (% w/v) | AET Results^{a} |
|---|---|---|
| Methyl paraben:Potassium | Level 1: 0.05%:0.05% | Level 1: Pass |
| sorbate | Level 2: 0.10%:0.10% | Level 2: Pass |
| | Level 3: 0.15%:0.15% | Level 3: Pass |
| Methyl paraben:Propyl | Level 1: 0.05%:0.01% | Level 1: Fail |
| paraben | Level 2: 0.10%:0.02% | Level 2: Pass |
| | Level 3: 0.15%:0.03% | Level 3: Pass |
| Propyl paraben:Potassium | Level 1: 0.01%:0.05% | Level 1: Pass |
| sorbate | Level 2: 0.02%:0.10% | Level 2: Fail |
| | Level 3: 0.03%:0.15% | Level 3: Fail |

^{a}The formulation used for the first set of results was made with Sigma grape flavor and not FONA.
AET = Anti-microbial effectiveness testing.

Propyl paraben is less desirable for pediatric use due to safety concerns; therefore, the methyl paraben/potassium sorbate concentration combination was selected. The methylparaben sodium salt was selected for the finalized F1 formulation instead of methylparaben free acid because of its greater aqueous solubility. The Compound A, 50 mg/mL F1 formulation contained 0.1% of each of methylparaben sodium and potassium sorbate.

Compound A, 50 mg/mL F2 oral solution contained 0.18% (w/w methylparaben sodium) and 0.20% (w/w) potassium sorbate. Anti-microbial effectiveness testing was conducted at methylparaben sodium concentrations of 0.12%, 0.15%, and 0.18%, with the potassium sorbate concentration held constant at 0.20%. At all 3 concentrations of methylparaben sodium, USP <51>, and Ph. Eur. 5.1.3 acceptance criteria were met for oral products made with aqueous bases or vehicles, as shown in Table 27.

TABLE 27
Results for Compound A Oral Solution (F2)
Antimicrobial Effectiveness Testing

| | Methylparaben | Potassium | |
|---|---|---|---|
| Formulation | Sodium (% w/w) | Sorbate (% w/w) | Results^{a} |
| 1 | 0.18 | 0.20 | Pass |
| 2 | 0.15 | 0.20 | Pass |
| 3 | 0.12 | 0.20 | Pass |

^{a}Passing result indicates AET data met both USP <51> and Ph. Eur. 5.1.3 acceptance criteria.
AET = Anti-microbial effectiveness testing; Ph. Eur. = European Pharmacopoeia; USP = United States Pharmacopeia.

Previous AET studies had identified methylparaben sodium and potassium sorbate as suitable antimicrobial preservatives for pediatric use. Anti-microbial effectiveness testing for F3 formulation was conducted at methylparaben sodium and potassium sorbate concentrations of 0.10%, 0.15%, and 0.20%, respectively. At all 3 concentrations USP <51> and Ph. Eur. 5.1.3 acceptance criteria were met for oral products made with aqueous bases or vehicles, as shown in Table 28.

TABLE 28
Results for Compound A Oral Solution (F3)
Antimicrobial Effectiveness Testing

| | Methylparaben | Potassium | |
|---|---|---|---|
| Formulation | Sodium (% w/w) | Sorbate (% w/w) | Results^{a} |
| 1 | 0.10 | 0.10 | Pass |
| 2 | 0.15 | 0.15 | Pass |
| 3 | 0.20 | 0.20 | Pass |

^{a}Passing result indicates AET data met both USP <51> and Ph. Eur. 5.1.3 acceptance criteria.
AET = Anti-microbial effectiveness testing; Ph. Eur. = European Pharmacopoeia; USP = United States Pharmacopeia.

Based on this study, 0.10% (w/w) methylparaben sodium and 0.10% (w/w) potassium sorbate were selected for the Compound A, 40 mg/mL F3 oral solution. The preservative limits for the Compound A 40 mg/mL formulation were set based on additional AET results.

### Conclusion of Formulation Development Studies

Dose Volume: A concentration of 40 mg/mL of Compound A citrate was achieved in the proposed formulation. This concentration equates to a 6.75 mL dose for an adult at a dose level of 270 mg and a 0.24 mL dose for a child with BSA 0.1 m² at a dose level of 95 mg/m². Both of those volumes can be measured using oral syringes.
Taste masking: Taste and organoleptic acceptance are important attributes especially for oral solution formulation intended for pediatric patient population. Compound A citrate has an inherent bitter taste and lingering aftertaste. The addition of advantame as sweetener, grape flavor, and sodium chloride for salty taste allowed for effective taste masking of Compound A drug substance in oral solution.
Manufacturability: Compound A oral solution is manufactured according to a specific process. The same steps have been used for all Compound A 40 mg/mL drug product batches, although the scale has increased through lab scale (0.1-2 kg), pilot scale (6-8 kg), registration scale (75 kg), and the proposed commercial scale of 200 kg. The manufacturing process has consistently produced an oral solution product that meets all product quality attributes.
Stability: Six Good Manufacturing Practice (GMP) batches and one non-GMP development batch of oral solution were placed on stability at the 5° C. long-term condition and 25° C./60% RH accelerated condition in a representative container closure configuration. Stability results are consistent with meeting the QTTP criteria.

### Comparative Assessment of Formulations

All 3 oral solution formulations contain fully soluble Compound A. None of the formulations contain excipients that may affect gastrointestinal transit, absorption, in vivo solubility, or in vivo stability of the active substance. F1, F2, and F3 formulations each contain the same amounts of advantame and sodium chloride. All 3 formulations contain the same preservatives, methylparaben sodium and potassium sorbate. The F1 and F3 formulations contain the same quantities of preservatives while the F2 formulation contains higher quantities. F1 and F3 formulations each contain the same amount of grape flavor. All components in the grape flavor used for F3 are generally recognized as safe (GRAS).

Oral solution dosing to date in trial study has used F1, F2, and F3 formulations. Since Compound A is fully soluble and the excipients used in all 3 oral solution formulations do not affect permeability, exposures were found to be comparable as expected. Comparable exposures were confirmed across solid and oral solution dose forms.

### Example 9. Manufacturing Process of the Liquid Formulation

### Development and Scale-Up of the Compound A Oral Solution Manufacturing Process

Compound A oral solution is a homogeneous, clear, colorless to light yellow solution. It is comprised of 7.83% of drug substance and excipients with purified water quantum sufficient (QS) to 100%. The excipients used in the oral solution are small molecules and are not viscosity building excipients. The specific gravity of the oral solution is 1.03 which is similar to the specific gravity of water (1.000). Thus, the oral solution is expected to behave similarly to water in the mixing and filling unit operations.

### i. Scale-Up

For an oral solution product, there are several areas of consideration during scale-up the process. These include solubility of solution components, pH sensitivity of solution components (solubility and stability), solution stability, preservative efficacy, and robustness of the manufacturing process. The process mixing parameters, including impeller speed and mixing time, are important for ensuring the ingredients are distributed so that the dissolution process can occur, resulting in a clear solution that is free of particulates.

During early non-GMP lab scale development the initial efforts included preparing stock solutions of the solid excipients in water with magnetic bar stirring. The order of excipient addition was selected based on solubility and pH considerations. The final order of addition was citric acid, advantame, methylparaben sodium, sodium chloride, potassium sorbate, sodium bicarbonate (for F1 and F2 compositions), grape flavor and drug substance. After addition of Compound A sesquifumarate the pH was approximately 6. Sodium hydroxide and hydrochloric acid were used as needed to adjust to the final pH of 6.5 (F1) and 6.0 (F2). The laboratory manufacturing process was scaled to 100 g (non-GMP) with pH of the solution closely monitored post-drug substance and post-sodium hydroxide addition. It was observed that achieving a stable pH of the dispersed/dissolved drug substance was necessary prior to sodium hydroxide addition.

Manufacturing process include at least one filtration step. The filtration step is conducted via manual filtration or using Cartridge filter, In-line Kleenpak Nova 10 inch Profile II 5 μm filter or Opticap XL Capsule filter, 5 μm. Compound A oral solution is filled into bottles to deliver 100 mL. The homogeneous nature of the Compound A monocitrate monohydrate oral solution was demonstrated and confirmed by sampling from the top and bottom of the mixing tank and testing for assay (Compound A citrate).

The Compound A citrate oral solution is filled into a 4 oz PET oval amber bottle with child resistant closure. In the Compound A citrate oral solution the drug is fully solubilized and there is no solid form present.

### Example 12. Compound A Monocitrate Monohydrate Polymorph Screen

In study details polymorph screening of Compound A monocitrate.

List of Abbreviations

| | |
|---|---|
| API | Active Pharmaceutical Ingredient |
| ACN, MeCN | Acetonitrile |
| CPME | Cyclopentyl methyl ether |
| DCM | Dichloromethane |
| DME | Dimethoxyethane |
| DMSO | Dimethyl sulfoxide |
| DSC | Differential scanning calorimetry |
| EtOAc | Ethyl acetate |
| EtOH | Ethanol, ethyl alcohol |
| FB | Free Base |
| h | Hour, hours |
| IPA | Isopropyl alcohol |
| IPAC | Isopropyl acetate |
| MEK | Methyl ethyl ketone |
| MeOH | Methanol |
| mg | Milligram, milligrams |
| MIBK | Methylisobutyl Ketone |
| mL | Milliliter, milliliters |
| ML | Mother liquor |
| MTBE | Methyl tert-butyl ether |
| NB | Notebook, notebook number |
| NMP | N-Methyl pyrrolidone |
| PLM | Polarized light microscopy |
| PSD | Particle size distribution |
| PVA | Polyvinyl alcohol |
| PVC | Polyvinyl chloride |
| PVP | Polyvinylpyrrolidone |
| rt | Room temperature |
| Compound A | API abbreviation |
| TGA | Thermogravimetric analysis |
| THF | Tetrahydrofuran |
| v/v % | volume/volume % |
| WC | wet cake |
| Wt. | weight |
| XRPD, PXRD | X-ray powder diffraction |

### X-Ray Powder Diffraction (XRPD)

Instrument: Panalytical Empyrean
Parameters: X-Ray tube Cu (Kα radiation); Power: 45 kV×40 mA
Scanning range: 2 to 40 2θ (degree)
Step size: 0.01 degree
Scanning speed: 6.33 degree (2θ) per minute

### Thermogravimetric Analysis (TGA)

Instrument: TA Instruments Discovery TGA
Parameters: Ramp 10° C. per minute, 25 to 300° C., 50 mL/min N₂ sweep

### Differential Scanning Calorimetry (DSC)

Instrument: TA Instruments Discovery DSC
Parameters: Ramp 10° C. per minute, up to 300° C.

### Polarized Light Microscopy (PLM)

Instrument: Nikon Eclipse Ci POL
Camera: Nikon DS-Fi3
Software: Nikon NIS Elements

### Nuclear Magnetic Resonance (NMR)

Instrument: Bruker 400 Ultrashield
Solvent: DMSO-d₆

### A. Solubility

The approximate solubility of Compound A monocitrate monohydrate was estimated in 24 solvents and results are given in Table 29. About 10 mg of Compound A monocitrate monohydrate was weighed into a 4.0 mL vial and 25 μL of solvent was added at RT. The solution was shaken and stirred. If no clear solution was obtained, another 25 μL was added and this was repeated until 4.0 mL solvent is added.

TABLE 29
Approximate solubility estimation results

| Exp | | Solubility | |
|---|---|---|---|
| ID | Solvents | (mg/mL) | Observation |
| 1 | 2-MeTHF | S < 2.9 | Hazy |
| 2 | Acetone/H₂O (9:1 v/v) | 15.0 < S < 17.5 | CL |
| 3 | Anisole | S < 3.0 | Hazy |
| 4 | Chloroform | S < 2.3 | Hazy |
| 5 | THF | S < 2.0 | Hazy |
| 6 | CPME | S < 2.1 | Hazy |
| 7 | Cyclohexane | S < 2.3 | Hazy |
| 8 | Diisopropyl ether | S < 2.3 | Hazy |
| 9 | DMA | 81.0 < S < 162.0 | CL |
| 10 | Dimethoxyethane (DME) | S < 2.4 | Hazy |
| 11 | EtOAc | S < 2.3 | Hazy |
| 12 | DMF | 80.0 < S < 160.0 | CL |
| 13 | Heptane | S < 2.8 | Hazy |
| 14 | IPA | S < 2.8 | Hazy |
| 15 | MEK | S < 2.4 | Hazy |
| 16 | Diethylamine | 170.0 < S < 340.0 | CL |
| 17 | MTBE | S < 2.8 | Hazy |
| 18 | NMP | 120.0 < S < 240.0 | CL |
| 19 | Benzene | S < 2.9 | Hazy |
| 20 | *Propylene glycol | 22.2 < S < 27.8 | CL |
| 21 | *THF/water (1/1) | 141.3 < S < 212.0 | CL |
| 22 | *1-PrOH/water (1/1) | 161.3 < S < 242.0 | CL |
| 23 | *MeOH/water (7/3) | 93.0 < S < 186.0 | CL |
| 24 | *1,4-dioxane/water (7/3) | 84.0 < S < 105.0 | CL |

*Heated for few seconds using heat gun

### B. Polymorph Screening

Experiments using 10 different techniques including using techniques such as anti-solvent addition, solid vapor diffusion, liquid vapor diffusion, slurry (at RT and 50° C.), slow evaporation, polymer induced crystallization, thermal cycling, sonication, and wet grinding. This analysis revealed three distinct forms of Compound A monocitrate: Form A (monohydrate), Form B (a metastable mono-methanol solvate), and Form C (a metastable MeCN solvate).
Anti-solvent addition—API was dissolved in different solvents to obtain saturated solutions (if API is highly soluble then highest concentration was limited to ~500 mg/mL) and anti-solvents were added up to 20 times in volume. Obtained solids were tested by XRPD (Table 30).
Solid vapor diffusion—About 30 mg API was transferred into 4 mL vials which are placed in 20 mL glass vials containing different solvents. The solids were tested by XRPD after 7-10 days (Table 31).
Liquid vapor diffusion About 30 mg API was transferred and dissolved in different solvents to obtain saturated solutions (if API is highly soluble then highest concentration was limited to ~500 mg/mL) in 4 mL vials which are placed in 20 mL glass vials containing anti-solvents. The obtained solids were characterized by XRPD after 7-10 days (Table 32).
Slurry at RT—About 40 mg API was suspended and stirred in different solvents in 4 mL vials at RT. The solids in the slurry were characterized by XRPD after 5-7 days (Table 33).
Slurry at 50° C.—About 40 mg API was suspended and stirred in different solvents in 4 mL vials at 50° C. The solids in the slurry were characterized by XRPD after 3 days (Table 34).
Slow evaporation—API was dissolved in different solvents to obtain saturated solutions (if API is highly soluble then highest concentration was limited to ~500 mg/mL) in 4 mL vials. The vials were covered with paraffin film with 3-5 holes and placed at specified temperature for evaporation. The obtained solids were characterized by XRPD (Table 35).
Polymer-induced crystallization—About 30 mg API is dissolved in different solvents to create saturated solutions (if API is highly soluble then highest concentration was limited to ~500 mg/mL). PVP, PVA, PVC, HPMC, and HPC were added to the saturated solutions to induce heteronuleation. The obtained solids were characterized by XRPD after 7-10 days (Table 36).
Temperature cycling—About 30 mg API was dissolved in different solvents to obtain a saturated solution (if API is highly soluble then highest concentration was limited to ~500 mg/mL) in 4 mL vials. The vials were placed in shakers, and mixed from 5° C. to 40° C. to 5° C., and circulated for 2 cycles. The obtained solids were characterized by XRPD (Table 37).
Sonication induced crystallization—About 40 mg API was suspended and sonicated in different solvents in 4 mL vials at RT. The solids in the slurry were characterized by XRPD after 1-day (Table 38).
Solvent drop grinding—About 30 mg API was ground with different solvents. The obtained solids were characterized by XRPD (Table 39).

TABLE 30
Results of Anti-Solvent Addition Experiments

| Exp. ID | Solvent | Antisolvent | Observation | Crystal Form |
|---|---|---|---|---|
| 1 | DMA | Toluene | CL→LT→ SE | Form A |
| | | | (RT)→Few solids | |
| 2 | | CPME | CL→LT→ SE | Form A |
| | | | (RT)→Few solids | |
| 3 | | EtOAc | CL→LT→ SE | Form A |
| | | | (RT)→Few solids | |
| 4 | DMSO | Diisopropyl | CL→LT→SE | Not applicable |
| | | ether | | |
| 5 | | Benzene | SL | Form A* |
| 6 | | MEK | CL→LT→SE | Not applicable |
| 7 | NMP | IPAc | Solids at bottom | Form A |
| 8 | | H2O | CL→LT→SE | Not applicable |
| 9 | | EtOH | CL→LT→SE | |
| 10 | Propylene | Acetone | CL→LT→SE | |
| 11 | glycol | THF | CL→LT→SE | |
| 12 | | MeCN | CL→LT→SE | |

*Loss of crystallinity;
CL: clear solution;
SL: slurry;
LT: low temp (5-8° C.);
SE: slow evaporation at 40-50° C.

TABLE 31
Results/Status Of Solid Vapor Diffusion Experiments

| Exp. ID | Solvent | Temp (° C.) | Observation | Crystal Form |
|---|---|---|---|---|
| 1 | 1,4-dioxane | RT | Solids | Form A |
| 2 | 2-Methyl THF | RT | Solids | Form A |
| 3 | Acetic acid | 40 | Solids → CL | Not applicable |
| 4 | CHCl3 | RT | Solids | Form A |
| 5 | CPME | 40 | Solids | Form A |
| 6 | DCM | RT | Solids | Form A |
| 7 | Dimethoxyethane | 40 | Solids | Form A |
| 8 | IPAc | RT | Solids | Form A |
| 9 | MEK | RT | Solids | Form A |
| 10 | MTBE | RT | Solids | Form A |
| 11 | THF | RT | Solids | Form A |
| 12 | Toluene | 40 | Solids | Form A |

CL: clear solution

TABLE 32
Results/Status Of Liquid Vapor Diffusion Experiments

| Exp. ID | Solvent | Antisolvent | Observation | Crystal Form |
|---|---|---|---|---|
| 1 | DCM | MeOH | CL | Not applicable |
| 2 | | MTBE | CL→Few | Form A |
| | | | Solids | |
| 3 | | Acetone | CL→Few | Form A |
| | | | Solids | |
| 4 | DMSO | EtOAc | CL | Not applicable |
| 5 | | Acetone | CL | |
| 6 | | H₂O | CL | |
| 7 | NMP | IPAc | CL | |
| 8 | | H₂O | CL | |
| 9 | | MTBE | CL→Few | Form A |
| | | | Solids | |
| 10 | Propylene | MeOH | CL | Not applicable |
| 11 | glycol | IPAc | CL | |
| 12 | | DCM | CL | |

CL: clear solution

TABLE 33
Results/Status Of Slurry At RT Based Experiments

| Exp. | | Solvent | Temp | | |
|---|---|---|---|---|---|
| ID | Solvent | (v:v) | (° C.) | Observation | Crystal Form |
| 1 | Acetic acid | N/A | RT | SL→CL→LT→SE | Not applicable |
| 2 | Acetone | N/A | RT | SL | Form A |
| 3 | Anisole:DMSO | 9:1 | RT | Hazy | Not applicable |
| 4 | Benzene | N/A | RT | Hazy | Form A |
| 5 | CHCl₃ | N/A | RT | SL | Form A |
| 6 | CPME | N/A | RT | SL | Form A |
| 7 | Diethylamine:EtOH | 3:7 | RT | SL | FB-2 |
| 8 | DMA:Benzene | 1:9 | RT | Hazy | Form A |
| 9 | DMA:CHCl₃ | 1:9 | RT | SL | Form A |
| 10 | DMA:EtOH | 1:9 | RT | SL | Form A |
| 11 | DMF:EtOAc | 1:9 | RT | SL | Form A |
| 12 | DMSO | N/A | RT | SL→Gel | Amorphous |
| 13 | THF:H₂O | (a_{w} = 0.2) | RT | SL | Form A |
| 14 | THF:H₂O | (a_{w} = 0.4) | RT | SL | Form A |
| 15 | THF:H₂O | (a_{w} = 0.6) | RT | SL | Form A |
| 16 | THF:H₂O | (a_{w} = 0.8) | RT | SL | Form A |
| 17 | DMSO:IPAc | 1:9 | RT | SL | Form A |
| 18 | IPA | N/A | RT | SL | Form A |
| 19 | MeOH:MeCN | 9:1 | RT | SL | Form A |
| 20 | MIBK | N/A | RT | SL | Form A |
| 21 | NMP:IPA | 1:9 | RT | SL | Form A |
| 22 | Propylene | 3:7 | RT | Hazy→CL→LT→SE | Poorly |
| | glycol:acetone | | | | crystalline; less |
| | | | | | solids afforded |
| 23 | Propylene | 3:7 | RT | SL→CL→LT→SE | Not applicable |
| | glycol:MeCN | | | | |
| 24 | Propylene | 9:1 | RT | SL→CL→LT→SE | |
| | glycol:MTBE | | | | |
| 25 | Toluene | N/A | RT | SL→CL→LT→ | Form A |
| | | | | Solids | |

CL: clear solution;
SL: slurry;
LT: low temp (5-8° C.);
SE: slow evaporation at 40-50° C.

TABLE 34
Results/Status Of Slurry At 50° C. Based Experiments

| Exp. | | Solvent | Temp | | Crystal |
|---|---|---|---|---|---|
| ID | Solvent | (v:v) | (° C.) | Observation | Form |
| 1 | 1-propanol | N/A | 50 | SL | Form A |
| 2 | 2-MeTHF | N/A | 50 | SL | Form A |
| 3 | ACN | 9:1 | 50 | SL | Form A |
| 4 | Anisole | N/A | 50 | waxy | Form A |
| 5 | Cyclohexane | N/A | 50 | SL | Form A |
| 6 | Dimethoxyethane | N/A | 50 | SL | Form A |
| 7 | DMA/H₂O | 1:9 | 50 | CL→LT→SE | Not |
| 8 | DMF/MTBE | 1:9 | 50 | Hazy→LT→SE | applicable |
| 9 | Ethyl | 3:1 | 50 | SL | Form A |
| | acetate/MeOH | | | | |
| 10 | Heptane | N/A | 50 | SL | Form A |
| 11 | IPAc | N/A | 50 | SL | Form A |
| 12 | MEK | N/A | 50 | SL | Form A |
| 13 | Methyl Acetate | N/A | 50 | SL | Form A |
| 14 | MTBE | N/A | 50 | SL | Form A |
| 15 | NMP:CPME | 3:7 | 50 | SL | Form A |
| | (3:7 v/v) | | | | |
| 16 | Propylene glycol | N/A | 50 | CL→LT→SE | Not |
| | | | | | applicable |
| 17 | Propylene | 1:1 | 50 | CL→LT→SE | Form A |
| | glycol:Acetone | | | | |
| 18 | Propylene glycol: | 1:1 | 50 | Hazy→LT→SE | Form A |
| | IPA | | | | |
| 19 | THF | 9:1 | 50 | SL | Form A |
| 20 | Toluene | N/A | 50 | SL | Form A |

CL: clear solution;
SL: slurry;
LT: low temp (5-8° C.));
SE: slow evaporation at 40-50° C.

TABLE 35
Results/Status of Slow Evaporation Experiments

| Exp. ID | Solvent | Temp (° C.) | Observation | Crystal Form |
|---|---|---|---|---|
| 1 | NMP | 50 | CL→ 50° C. | Not applicable |
| 2 | DMSO | 50 | CL→ 50° C. | |
| 3 | THF/200 μL | RT | CL→ 50° C. | |
| | Propylene glycol | | | |
| 4 | DMA | 50 | CL→ 50° C. | |
| 5 | Diethylamine | 50 | solids | FB-2 |

CL: clear solution

TABLE 36
Results/Status of Polymer Induced Crystallization Experiments

| Exp. | | | Solvent | Temp | | Crystal |
|---|---|---|---|---|---|---|
| ID | Polymer | Solvent | (v:v) | (° C.) | Observation | Form |
| 1 | PVP | DMA: | 1:9 | 40 | SL | Form A |
| | | H₂O | | | | |
| 2 | | NMP: | 1:9 | 40 | SL | Form A |
| | | H₂O | | | | |
| 3 | Brij | NMP: | 1:9 | 40 | CL→LT→ | Amorphous |
| | | H₂O | | | SE | |
| 4 | PVC | DMF: | 1:9 | RT | SL | Form A |
| | | DCM | | | | |
| 5 | HPMC | Acetone: | 1:1 | RT | CL→LT→ | Not |
| | | Propylene | | | SE | applicable |
| | | Glycol | | | | |
| 6 | | EtOAc: | 1:1 | RT | CL→LT→ | |
| | | Propylene | | | SE | |
| | | Glycol | | | | |
| 7 | HPC-L | MeCN: | 1:1 | RT | CL→LT→ | |
| | | H₂O | | | SE | |
| 8 | Poloxamer | Acetone: | 1:1 | RT | CL→LT→ | |
| | 407 | H₂O | | | SE | |

CL: clear solution;
SL: slurry;
LT: low temp (5-8° C.));
SE: slow evaporation at 40-50° C.

TABLE 37
Results/Status Of Temperature Cycling Experiments

| Exp. | | Solvent | Temp | | |
|---|---|---|---|---|---|
| ID | Solvent | (v:v) | (° C.) | Observation | Crystal Form |
| 1 | DMSO:Toluene | 1:9 | 50→40→30→20→5 | SL→Hazy | Form A* |
| 2 | 1,4- | 9:1 | total 2 cycles | SL→CL | Form A* |
| | dioxane:H₂O | | (Each step 4 h) | | |
| 3 | NMP:nPA | 1:9 | | SL | Form A* |
| 4 | 2- | 9:1 | | SL | Form A* |
| | MeTHF:DMF | | | | |
| 5 | DMA:EtOH | 1:9 | | SL | Mix Form A & |
| | | | | | Form B* |
| | | | | | (As wet-cake) |

CL: clear solution;
SL: slurry;
*Isolated at RT

TABLE 38
Results/Status Of Sonication-Based Experiments

| Exp. ID | Solvent | Solvent (v:v) | Observation | Crystal Form |
|---|---|---|---|---|
| 1 | THF | N/A | SL | Form A |
| 2 | Acetone:H₂O | 4:1 | CL→ SE | Not applicable |
| 3 | DMSO:EtOAc | 1:9 | SL | Form A |

CL: clear solution;
SL: slurry;
SE: slow evaporation at 40-50° C.

TABLE 39
Results/Status Of Solvent Drop Grinding Experiments

| Exp. ID | Solvent | Observation | Crystal Form |
|---|---|---|---|
| 1 | THF | solids | Form A |
| 2 | EtOH | solids | Form A |
| 3 | Acetone | solids | Form A |
| 4 | 1,4-Dioxane | solids | Form A |
| 5 | DCM | solids | Form A |

C. Form C

### C. Form C

Screening: The results of further screening studies for Form C are shown in Table 40 and Table 41.

TABLE 40
Form C Screening Experiments Set-Up And Observations

| | | Citric | | | | | |
|---|---|---|---|---|---|---|---|
| Exp. | | acid | | Solvent | Temp | | |
| ID | Material | (mg) | Solvent | (mL) | (° C.) | Notes | Observation |
| 1 | FB-2 | 51 | MeCN | 1.0 | RT | Reactive | Hazy→SL |
| 2 | (166 mg) | | EtOAc | | RT | crystallization | SL |
| 3 | | | MeCN | | 50 | | CL→SL→Thick |
| | | | | | | | cake due to SE |
| 4 | | | EtOAc | | 50 | | SL |
| 5 | FB-1 | | MeCN | | RT | Reactive | Hazy→SL |
| 6 | (166 mg) | | EtOAc | | RT | crystallization | SL |
| 7 | | | MeCN | | 50 | | CL→Waxy |
| | | | | | | | solids |
| 8 | | | EtOAc | | 50 | | SL |
| 9 | FB-2 | None | MeCN | 1.0 | RT | Slurry | SL |
| 10 | (166 mg) | | EtOAc | | | conditioning | SL |
| 11 | FB-1 | None | MeCN | 1.0 | 50 | Slurry | SL |
| 12 | (166 mg) | | EtOAc | | | conditioning | (Solvent |
| | | | | | | | evaporated after |
| | | | | | | | 3 days) |
| 13 | FB-2 | 15 | MeCN | 0.5 | RT | Reactive | Hazy→SL |
| | (166 mg) | | | | | crystallization. | |
| | | | | | | (Form C in | |
| | | | | | | MeCN; Hazy | |
| | | | | | | →FB + Acid) | |
| 14 | | | | | | Reactive | CL→SL |
| | | | | | | crystallization. | |
| | | | | | | (FB + Acid IN | |
| | | | | | | MeCN →CL→ | |
| | | | | | | Form C) | |
| 15 | | 30 | MeCN | 0.5 + 1.5 | RT | 1:2 of FB:Acid | CL→ HSL |
| 16 | Form C | 40 | Water | 0.1 | RT | CL to begin; | CL→ HSL |
| | | | | | | solubility >400 | |
| | | | | | | mg/mL | |
| 17 | Form A | 45 | MeCN | 0.5 | RT | Added seed of | SL |
| | | | | | | Form C→ then | |
| | | | | | | added Form A | |

CL: clear solution;
SL: slurry;

TABLE 41
Form C screening experiments result

| Exp. ID | Crystal Form | HNMR (FB:Acid) |
|---|---|---|
| 1 | Form A, w/pattern shift | NA |
| 2 | FB-2 w/extra peaks | NA |
| 3 | Form A → Form C | 1:1 |
| 4 | Form A | NA |
| 5 | Form A, w/pattern shift | NA |
| 6 | FB-1 w/extra peaks | NA |
| 7 | Form A → Form C | NA |
| 8 | Essentially Form C | NA |
| 9 | FB-2 | NA |
| 10 | FB-2 | NA |
| 11 | FB-1 | NA |
| 12 | FB-1 | NA |
| 13 | Form C | 1:1 |
| 14 | Form C | 1:1 |
| 15 | Form C | 1:2* |
| 16 | Form A | NA |
| 17 | Form C | NA |

CL: clear solution;
SL: slurry;
TSL—Thin slurry;
HSL—Heavy slurry;
*solids were not filtered, vial was spinned and mL was removed;
SE—solvent evaportaion;
NA—no tested;
MeCN has ~0.3 wt % water

Scale Up: The scale-up for Form C was initiated through the slurry conversion of Form A, utilizing MeCN as the solvent and introducing seeds derived from Form C. Analysis indicated the presence of Form C; however, the XRPD profile exhibited additional peaks, potentially attributable to Form A. To rectify this, the isolated solids were subjected to re-slurring in MeCN, which yielded Form C (Table 42).

TABLE 42
Form C Scale Up Experiment Details And Results

| Exp. | | Form C | | Temp | | Crystal |
|---|---|---|---|---|---|---|
| ID | Material | seed | Solvent | (° C.) | Observation | Form |
| 1 | Form A | (50 mg) | MeCN | RT | Slurry | Form C |
| | (500 mg) | | (5.0 | | | w/extra |
| | | | mL) | | | peaks |
| 2 | Form A | (20 mg) | MeCN | RT | Slurry | Form C |
| | (420 mg) | | (5.0 | | | |
| | | | mL) | | | |

Water activity study in MeCN/water: A water activity investigation was conducted utilizing a MeCN/water system. Water activities were assessed ranging from 0.01 to 0.8 at room temperature, with Form C serving as the starting material. After an overnight stirring, the wet cake underwent XRPD analysis, confirming that Form C remained stable. Subsequently, Form A seeds were introduced into vials with water activities between 0.01 and 0.6. Post-an overnight stirring period, XRPD analysis was performed on the material, revealing a match with the Form C. The solids were isolated and subjected to vacuum drying at room temperature overnight. XRPD analysis showed that the powder pattern of solids from water activity levels of 0.01-0.6 was consistent with Form C. However, the solids from a water activity of 0.8 indicated a combination of Form C and Form A. Experimental detail and observations are summarized in Table 43.

TABLE 43
Form C Water Activity Experiment Details And Results

| | | Form A | MeCN/ | | | |
|---|---|---|---|---|---|---|
| Exp. | Form C | Seed | water | Temp | | |
| ID | (mg) | (mg) | (a_{w}) | (° C.) | Observation | XRPD |
| 1 | 40 | — | 0.01 | RT | Hazy →SL | Form C |
| 1a | | 20 | | | SL →SL | Form C |
| 2 | 40 | — | 0.22 | RT | Hazy →SL | Form C |
| 2a | | 20 | | | SL →SL | Form C |
| 3 | 40 | — | 0.41 | RT | Hazy →SL | Form C |
| 3a | | 20 | | | SL →SL | Form C |
| 4 | 40 | — | 0.59 | RT | Hazy →SL | Form C |
| 4a | | 20 | | | SL →SL | Form C |
| 5 | 40 | — | 0.80 | RT | Hazy →SL | Mix of |
| | | | | | (excess solids | Form C + |
| | | | | | added) | Form A |

SL: slurry

Slurry conditioning in EtOH/water (95/5): Form C was slurry conditioned in EtOH/water (95/5), current crystallization solvent system to assess the stability of the Form C in this system. After overnight stirring material was analyzed by XRPD confirming Form C converted to Form A (Table 44).

TABLE 44
Form C slurry conditioning in EtOH/water
experiment details and results

| | | Form A | EtOH/ | | | |
|---|---|---|---|---|---|---|
| Exp. | Form C | Seed | water | Temp | | |
| ID | (mg) | (mg) | (95/5) | (° C.) | Observation | XRPD |
| 1 | 40 | — | 0.2 | RT | SL | Form A |
| 2 | | 20 | | | SL | Form A |

SL: slurry

### CONCLUSION

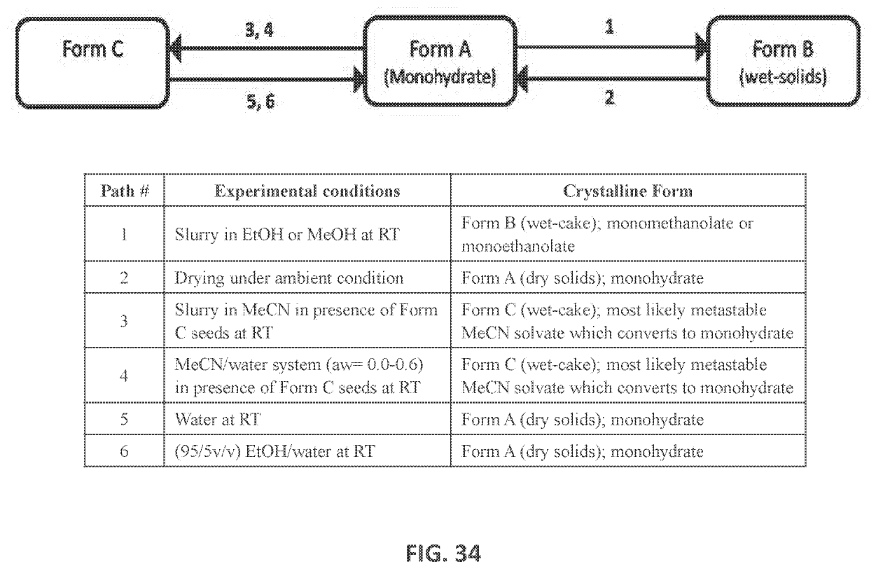

Three distinct forms of Compound A monocitrate were identified: Form A (monohydrate), Form B (a metastable mono-methanol solvate), and Form C (a metastable MeCN solvate). Form B, being a metastable solvate, transitions to Form A in ambient conditions. Form C, though not initially found in screenings, was later produced via reactive salt crystallization from a freebase and remained stable after drying. In specific MeCN conditions, with a water activity ranging from 0.0 to 0.6, the presence of Form C seeds can cause Form A to revert to Form C. However, Form C transitions to Form A in both water and EtOH/water (95/5 v/v) (FIG. 34). Based on the available data, Form A stands out as the thermodynamically most stable, with the other forms showing variable behavior based on conditions.

Form C was reproduced by reactive salt crystallization using freebase as starting material. Preparation of Form C was further achieved by conversion of Form A slurry in MeCN with Form C seeds. Subsequent XRPD analysis confirmed that the pattern of Form C remained unchanged post-vacuum drying at room temperature. TGA data revealed a weight reduction of 2.0 wt % from RT to 125° C. Moreover, HNMR studies validated a 1:1 stoichiometry between the freebase and acid. On heating Form C to 110° C., the XRPD profile of the resultant material aligned with that of Form C. When subjected to a competitive slurry experiment in MeCN, it was observed that Form A transitioned to Form C. Analyzing this resultant material by GC and oven KF showed a content of 0.15 wt % MeCN and 2.5 wt % water, respectively. Furthermore, Form C showed an approximate solubility of >400.0 mg/mL in water; however, Form A was recrystallized after overnight stirring in water at RT. Relative stability of Form C and Form A was tested in 95/5 (v/v) EtOH/water solution in which Form C converted to Form A. Form C, which behaves as a channel MeCN solvate, loses its solvent when exposed to ambient conditions. However, upon desolvation, it absorbs atmospheric water, maintaining its original structure indicating its isostructural nature. In a MeCN/water system, Form C remains stable at water activity ranging from 0.01 to 0.6. However, at a water activity of 0.8, the XRPD pattern presented certain peaks characteristic of Form A.

### Example 13. Compound A Monocitrate Monohydrate Excipient Compatibility Study

### i. Purpose

The Compound A monocitrate tablets showed excellent stability. The list of excipients for this study was restricted to those already being used in the tablet formulation provided in Example 7. OpaDry Orange was further added.

### ii. Study Design

Binary mixtures of Compound A monocitrate monohydrate and each excipient, were placed in closed 20 mL borosilicate glass vials (Tables 45-47). The vials were stored at 25° C./60% RH, 30° C./65% RH, and 40° C./75% RH over 8 weeks and later analyzed using methods for Appearance, Compound A Assay, Related Substances, XRPD, and Differential Scanning Calorimetry (DSC).

TABLE 45
Excipient List and Ratios

| | | | | Mixture Ratios |
|---|---|---|---|---|
| Function | Material | Trade Name | Grade | (API:Excipient) |
| Filler | Microcrystalline | Avicel ® PH 101 | NF, JP | 1:10 |
| | Cellulose | | Ph. Eur. | |
| | Microcrystalline | Avicel ® DG | N/A¹ | 1:10 |
| | Cellulose and | | | |
| | Anhydrous Dibasic | | | |
| | Calcium Phosphate | | | |
| | Microcrystalline | Avicel ® PH 102 | NF, EP, JP | 1:10 |
| | Cellulose | | | |
| | Anhydrous Dibasic | Anhydrous | NF, EP, JP | 1:10 |
| | Calcium Phosphate | Emcompress ® | | |
| Binder | Hypromellose | Methocel ™ E50 | NF, EP | 1:1 |
| | (50 mPa · s) | Premium LV | | |
| Disintegrant | Crospovidone | Polyplasdone ™ | EP, NF | 1:1 |
| | | XL-10 | | |
| | Sodium | N/A | NF, EP | 1:1 |
| | Bicarbonate | | | |
| Glidant | Hydrophobic | Aerosil ® R972 | NF, EP | 10:1 |
| | Colloidal Silica | | | |
| Lubricant | Magnesium | Hyqual ® | EP, NF | 10:1 |
| | Stearate | | | |
| Coating | Polyvinyl Alcohol | Opadry ® II | N/A¹ | 1:1 |
| | Film | 85F90035 Purple | | |
| | Polyvinyl Alcohol | Opadry ® II | N/A¹ | 1:1 |
| | Film | 85F97253 Beige | | |
| | Polyvinyl Alcohol | Opadry ® II | N/A¹ | 1:1 |
| | Film | 85F94190 Pink | | |
| | Polyvinyl Alcohol | Opadry ® II | N/A¹ | 1:1 |
| | Film | 85F18422 White | | |
| | Polyvinyl Alcohol | Opadry ® II | N/A¹ | 1:1 |
| | Film | 85F130168 | | |
| | | Orange | | |

¹Individual components used to manufacture film coat meet USP, NF, Ph. Eur, EP, and/or JP compendia.

TABLE 46
Excipient Compatibility Sample Mixtures, Samples A-J

| | Excipient Compatibility Sample Mixtures | | | | | | | | | |
|---|---|---|---|---|---|---|---|---|---|---|
| Ingredient | Weight to be Dispensed (mg) | | | | | | | | | |
| (API) | 50 | 50 | 50 | 50 | 50 | 50 | 50 | 50 | 50 | 50 |
| Microcrystalline Cellulose | | 500 | | | | | | | | |
| PH 101 | | | | | | | | | | |
| Co-Processed | | | 500 | | | | | | | |
| Microcrystalline Cellulose | | | | | | | | | | |
| and | | | | | | | | | | |
| Anhydrous Dibasic Calcium | | | | | | | | | | |
| Phosphate | | | | | | | | | | |
| Microcrystalline Cellulose | | | | 500 | | | | | | |
| PH 102 | | | | | | | | | | |
| Anhydrous Dibasic Calcium | | | | | 500 | | | | | |
| Phosphate | | | | | | | | | | |
| Hypromellose 50 mPa · s | | | | | | 50 | | | | |
| Crospovidone XL-10 | | | | | | | 50 | | | |
| Sodium Bicarbonate | | | | | | | | 50 | | |
| Colloidal Silicon Dioxide | | | | | | | | | 5 | |
| Magnesium Stearate | | | | | | | | | | 5 |

| Sample ID | A | B | C | D | E | F | G | H | I | J |
|---|---|---|---|---|---|---|---|---|---|---|
| Total Weight, mg | 50 | 550 | 550 | 550 | 550 | 100 | 100 | 100 | 55 | 55 |
| No. of Vials | 20 | 20 | 20 | 20 | 20 | 20 | 20 | 20 | 20 | 20 |

| | |
|---|---|
| Storage | 25° C./60% RH closed, 30° C./65% RH |
| Conditions | closed 40° C./75% RH closed |
| Time Points | Initial (t = 0) and 8 week |

TABLE 47
Excipient Compatibility Sample Mixtures, Samples K-O

| | Excipient Compatibility Sample Mixtures | | | | |
|---|---|---|---|---|---|
| Ingredient | Weight to be Dispensed (mg) | | | | |
| API | 50 | 50 | 50 | 50 | 50 |
| Opadry ® II Purple | 50 | | | | |
| Opadry ® II Beige | | 50 | | | |
| Opadr ®y II Pink | | | 50 | | |
| Opadry ® II White | | | | 50 | |
| Opadry ® II Orange | | | | | 50 |
| Sample ID | K | L | M | N | O |
| Total Weight, mg | 100 | 100 | 100 | 100 | 100 |
| No. of Vials | 20 | 20 | 20 | 20 | 20 |

| | |
|---|---|
| Storage Conditions | 25° C./60% RH closed, 30° C./65% |
| | RH closed, 40° C./75% RH closed |
| Time Points | Initial (t = 0) and 8 week |

### iii. Results

Appearance: Appearance remained unchanged for all samples A through O at 8 weeks.

Assay: Assay had some variability at 8 weeks compared to t=0, however this could be attributed to using smaller mg amounts of mixture to perform Assay testing than the method normally requires. Sample weight was decreased for this study to minimize API requirements (Table 48).

TABLE 48
Assay (%) Results

| | Time point | | | |
|---|---|---|---|---|
| | | 8 week | | |
| Sample | Initial | 25° C./60% RH | 40° C./75% RH | 30° C./65% RH |
| A | 98.8 | 100.3 | 100.0 | 101.4 |
| B | 98.4 | 99.4 | 99.0 | 100.5 |
| C | 101.3 | 103.1 | 101.5 | 100.9 |
| D | 99.4 | 98.7 | 99.8 | 100.0 |
| E | 101.0 | 101.5 | 100.3 | 102.7 |
| F | 99.8 | 100.1 | 99.0 | 90.9 |
| G | 99.3 | 100.6 | 100.1 | 101.4 |
| H | 99.8 | 103.8 | 101.4 | 97.7 |
| I | 97.6 | 100.7 | 99.2 | 100.3 |
| J | 99.5 | 100.7 | 102.5 | 102.1 |
| K | 99.5 | 104.9 | 101.4 | 99.7 |
| L | 100.2 | 99.6 | 98.8 | 99.2 |
| M | 98.8 | 100.8 | 101.7 | 98.7 |
| N | 100.9 | 100.9 | 103.5 | 101.0 |
| O | 96.9 | 101.6 | 101.6 | 104.3 |

Related Substances: There were no related substance peaks observed above the detection limit for any sample A through O at any storage condition at initial or 8 weeks.

DSC: The thermal event profiles did not change for any sample when comparing initial to 8 weeks at each of the 3 storage conditions.

XRPD: The reflections did not change for any sample when comparing initial to 8 weeks at each of the 3 storage conditions.

### iv. Conclusions

This study with Compound A monocitrate monohydrate API confirmed conclusions from the previous study using Compound A sesquifumarate, that each excipient tested was suitable for use in Compound A tablet drug product development.

### Example 14. Pharmacokinetics of Compound A

The pharmacokinetics of Compound A were characterized in patients with relapsed or refractory acute leukemia following single and multiple oral administration of Compound A with or without strong CYP3A4 inhibitors. Steady-state pharmacokinetic parameters are presented as geometric mean [geometric coefficient of variation (CV)] unless otherwise specified.

TABLE 49
Compound A Pharmacokinetics in Patients with
Relapsed or Refractory Acute Leukemia

| | Dosage | |
|---|---|---|
| | 163 mg twice daily | 276 mg twice daily |
| | (with strong CYP3A4 | (without strong CYP3A4 |
| Parameter | inhibitors)^{a} | inhibitors)^{a} |

General Information
Exposure^{b}

| Cₘₐₓ (ng/mL) | 3220 | (34%) | 2052 | (79%) |
|---|---|---|---|---|
| AUC_{0-12 h} (ng · | 22610 | (50%) | 10,150 | (69%) |
| h/mL) | | | | |
| Dose | Dose proportional increases in Cₘₐₓ and AUC_{0-12 h} | | | |
| Proportionality^{c} | | | | |
| Time to Steady- | 2-3 days | | | |
| State | | | | |

| | |
|---|---|
| Accumulation^{b} | 2-fold |

Absorption

| | | | | |
|---|---|---|---|---|
| Tₘₐₓ Median (range) | 2 | (0-6) | 1 | (0.5-4) |
| hours | | | | |

Effect of Food

| | |
|---|---|
| Low fat meal^{d} | No clinically significant differences in |
| | Compound A pharmacokinetics observed (Cₘₐₓ and |
| | AUC decreased by 27% and 12% respectively) |

Distribution

| Apparent Volume of | 78 (50%) |
|---|---|
| Distribution^{b} (L) | |
| Protein Binding^{e} | 90% |
| Blood to plasma | 0.8 |
| ratio | |

Elimination

| | | | | |
|---|---|---|---|---|
| Half-Life^{b} (hours) | 7.5 | (57%) | 3.6 | (36%) |
| Apparent Clearance^{b} | 7 | (51%) | 27 | (69%) |
| (L/h) | | | | |

Metabolism

| | |
|---|---|
| Primary Pathway | CYP3A4 |
| Active Metabolite | M1^{f} |

Excretion^{g}

| | |
|---|---|
| Feces | Approximately 49% (7% unchanged) |
| Urine | Approximately 27% (7% unchanged) |

Abbreviations:
Cₘₐₓ = maximum plasma concentration;
AUC = area under the time concentration curve;
Tₘₐₓ = time to peak concentration
^{a}1.02 times the highest adult recommended dosages
^{b}Steady-state
^{c}Dosage range of 113 mg to 339 mg (1.26 times the highest adult recommended dosage)
^{d}Approximately 400-500 calories, 25% of calories from fat
^{e}Independent of concentration
^{f}M1 contributes to Compound A's clinically significant effects on QTc but does not contribute to its efficacy at the approved recommended dosage
^{g}A single dose of radiolabeled Compound A 276 mg (1.02 times the highest adult approved recommended dosage) to adult patients with relapsed/refractory acute leukemia

### Specific Populations

Body weight and body surface area (BSA) has a significant effect on the pharmacokinetics of Compound A, with higher Compound A exposures in patients with lower body weight and BSA. This supports the approved recommended BSA dosage in patients weighing less than 40 kg.

### Pediatric Patients

Compound A geometric mean (geometric CV %) steady-state Cₘₐₓ is 3137 (39%) ng/mL and AUC₀₋ₜₐᵤ was 14632 (55%) ng·hr/mL following 95 mg/m² (approximately 1.02 times the highest recommended adult dose) with strong CYP3A4 inhibitors.

Additionally, 2 pediatric patients received Compound A 160 mg/m² (approximately 1.02 times the highest recommended adult dose) without strong CYP3A4 inhibitors. Compound A steady-state Cₘₐₓ of these 2 patients are 4950 and 4790 ng/mL and AUC₀₋ₜₐᵤ were 25600 and 16300 ng·hr/mL.

### Drug Interaction Studies (Clinical Studies)

Strong CYP3A4 Inhibitors: Compound A AUC and Cₘₐₓ is increased by 2-fold following concomitant use of multiple doses of Compound A with certain azole antifungals that are strong CYP3A4 inhibitors (i.e., posaconazole, itraconazole, and voriconazole). Similarly, Compound A AUC and Cₘₐₓ is increased by 2.5-fold following concomitant use of multiple doses of Compound A with cobicistat (strong CYP3A4 inhibitor).

Strong and Moderate CYP3A4 Inducers: Compound A exposure is expected to decrease and M1 exposure is expected to increase with strong and moderate CYP3A4 inducers.

Other Drugs: No clinically significant differences in Compound A pharmacokinetics were observed when used concomitantly with fluconazole (moderate CYP3A4 inhibitor), isavuconazole (moderate CYP3A4 inhibitor).

### Example 15. Safey and Efficacy of Compound A Monocitrate Monohydrate Salt

The safety and efficacy of Compound A monocitrate monohydrate salt have been established in pediatric patients 7 months and older with relapsed or refractory acute leukemia with a KMT2A translocation. Use of Compound A monocitrate monohydrate salt for this indication is supported by evidence from adequate and well-controlled trials in adults and pediatric patients and additional pharmacokinetic and safety data in pediatric patients. The patients included 7 infants (age <2 years), 20 children (age 2 to <12 years) and 4 adolescents (age 12 to <17 years). The recommended dosage in patients weighing less than 40 kg is BSA-based.

Additionally, in single-patient protocols, the patients included 1 infant (age <2 years), 11 children (age 2 to <12 years) and 5 adolescents (age 12 to <17 years).

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. A method of treating a leukemia in a human in need thereof comprising orally administering a citrate salt of Compound A: 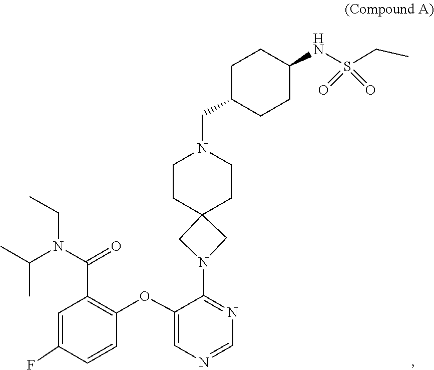 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]
wherein Compound A is administered at a dosage of about 270 mg twice daily, further wherein Compound A is administered without a CYP3A4 inhibitor.

2. The method of claim 1, wherein the leukemia is mixed lineage leukemia (MLL), MLL-related leukemia, MLL-associated leukemia, MLL-positive leukemia, MLL-induced leukemia, lysine methyltransferase 2A gene rearrangement (KMT2Ar or KMT2A), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, Hodgkin's lymphoma, non Hodgkin's lymphoma (malignant lymphoma), or Waldenstrom's macroglobulinemia.

3. The method of claim 1, wherein the leukemia is an abstract nucleophosmin 1 (NPM1)-mutated acute myeloid leukemia or a lysine methyltransferase 2A gene (KMT2A) rearrangement acute leukemia.

4. The method of claim 1, wherein the leukemia is a lysine methyltransferase 2A gene (KMT2A) translocation acute leukemia.

5. The method of claim 1, wherein Compound A is a citrate hydrate salt.

6. The method of claim 1, wherein Compound A is a monocitrate monohydrate salt.

7. The method of claim 1, wherein Compound A is a crystalline monocitrate monohydrate salt of Form A, characterized by PXRD reflections at 5.2°2θ, 11.8°2θ, and 16.9°2θ (±0.2°2θ; ±0.1°2θ; or ±0.0°2θ; Cu Kα1 radiation).

8. The method of claim 1, wherein Compound A is a crystalline monocitrate monohydrate salt of Form A, characterized by three or more PXRD signals selected from the group consisting of 5.2°2θ, 11.8°2θ, 16.2°2θ, 16.9°2θ, and 19.2°2θ (±0.2°2θ; ±0.1°2θ; or ±0.0°2θ; Cu Kα1 radiation).

9. The method of claim 1, wherein Compound A is a crystalline monocitrate monohydrate salt of Form A, characterized by three or more PXRD signals selected from the group consisting of 5.2°2θ, 11.8°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).

10. The method of claim 1, wherein Compound A is a crystalline monocitrate monohydrate salt of Form A, characterized by three or more PXRD signals selected from the group consisting of 5.2°2θ, 11.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).

11. The method of claim 1, wherein Compound A is a crystalline monocitrate monohydrate salt of Form A, characterized by one, two, three, four, five, six, seven, eight, nine, ten, eleven, twelve, thirteen, fourteen, fifteen, sixteen, seventeen, eighteen, or nineteen PXRD signals selected from those set forth in Table 1A.

12. The method of claim 1, wherein the leukemia is relapsed or refractory.

13. The method of claim 3, wherein the leukemia is relapsed or refractory.

14. The method of claim 4, wherein the leukemia is relapsed or refractory.

15. A method of treating a leukemia in a human in need thereof, comprising orally administering a citrate salt of Compound A: 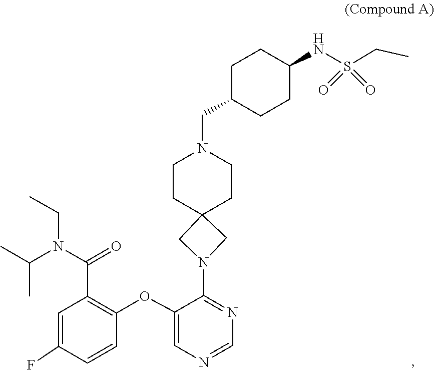 [SMILES: CCN(C(=O)c1cc(F)ccc1Oc1cncnc1N1CC2(CCN(C[C@H]3CC[C@H](NS(=O)(=O)CC)CC3)CC2)C1)C(C)C]
wherein the human weighs less than 40 kg and Compound A is administered at a dosage of about 160 mg/m² twice daily, further wherein Compound A is administered without a CYP3A4 inhibitor.

16. The method of claim 15, wherein the leukemia is mixed lineage leukemia (MLL), MLL-related leukemia, MLL-associated leukemia, MLL-positive leukemia, MLL-induced leukemia, lysine methyltransferase 2A gene rearrangement (KMT2Ar or KMT2A), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, Hodgkin's lymphoma, non Hodgkin's lymphoma (malignant lymphoma), or Waldenstrom's macroglobulinemia.

17. The method of claim 15, wherein the leukemia is an abstract nucleophosmin 1 (NPM1)-mutated acute myeloid leukemia or a lysine methyltransferase 2A gene (KMT2A) rearrangement acute leukemia.

18. The method of claim 15, wherein the leukemia is a lysine methyltransferase 2A gene (KMT2A) translocation acute leukemia.

19. The method of claim 15, wherein Compound A is a citrate hydrate salt.

20. The method of claim 15, wherein Compound A is a monocitrate monohydrate salt.

21. The method of claim 15, wherein Compound A is a crystalline monocitrate monohydrate salt of Form A, characterized by PXRD reflections at 5.2°2θ, 11.8°2θ, and 16.9°2θ (±0.2°2θ; ±0.1°2θ; or ±0.0°2θ; Cu Kα1 radiation).

22. The method of claim 15, wherein Compound A is a crystalline monocitrate monohydrate salt of Form A, characterized by three or more PXRD signals selected from the group consisting of 5.2°2θ, 11.8°2θ, 16.2°2θ, 16.9°2θ, and 19.2°2θ (±0.2°2θ; ±0.1°2θ; or ±0.0°2θ; Cu Kα1 radiation).

23. The method of claim 15, wherein Compound A is a crystalline monocitrate monohydrate salt of Form A, characterized by three or more PXRD signals selected from the group consisting of 5.2°2θ, 11.8°2θ, 16.2°2θ, 16.9°2θ, 17.2°2θ, 19.2°2θ, and 20.9°2θ (±0.2°2θ; ±0.1°2θ; or ±0.0°2θ; Cu Kα1 radiation).

24. The method of claim 15, wherein Compound A is a crystalline monocitrate monohydrate salt of Form A, characterized by three or more PXRD signals selected from the group consisting of 5.2°2θ, 11.2°2θ, 11.8°2θ, 12.6°2θ, 16.2°2θ, 16.9°2θ, 17.2°2θ, 19.2°2θ, 20.4°2θ, and 20.9°2θ (±0.2°2θ; ±0.1°2θ; or ±0.0°2θ; Cu Kα1 radiation).

25. The method of claim 15, wherein Compound A is a crystalline monocitrate monohydrate salt of Form A, characterized by one, two, three, four, five, six, seven, eight, nine, ten, eleven, twelve, thirteen, fourteen, fifteen, sixteen, seventeen, eighteen, or nineteen PXRD signals selected from those set forth in Table 1A.

26. The method of claim 15, wherein the leukemia is relapsed or refractory.

27. The method of claim 17, wherein the leukemia is relapsed or refractory.

28. The method of claim 18, wherein the leukemia is relapsed or refractory.
